(19) United States
(12) Patent Application Publication
(10) Pub. No.: US 2008/0171737 A1
(43) Pub. Date: Jul. 17, 2008

(54) CYCLIC SULFONAMIDE DERIVATIVES AND METHODS OF THEIR USE

(75) Inventors: Andrew Fensome, Wayne, PA (US); Joel Adam Goldberg, Philadelphia, PA (US); Casey Cameron McComas, Phoenixville, PA (US); Charles William Mann, Plymouth Meeting, PA (US); Edward George Melenski, Collegeville, PA (US); Joseph Peter Sabatucci, Collegeville, PA (US); Richard Page Woodworth, North Wales, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/954,466

(22) Filed: Dec. 12, 2007

Related U.S. Application Data
(60) Provisional application No. 60/869,642, filed on Dec. 12, 2006.

(51) Int. Cl. A61K 31/551; C07D 279/02; A61K 31/5415; C07D 291/08; A61P 1/00; A61P 25/00; A61P 13/00; A61P 15/00; C07D 285/15; C07D 243/08

(52) U.S. Cl. 514/218; 544/49; 514/226.5; 544/2; 514/222.8; 544/5; 540/575

(57) ABSTRACT

The present invention is directed to cyclic sulfonamide derivatives of formula I:

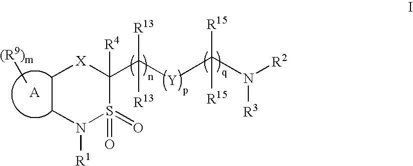

or a pharmaceutically acceptable salt thereof, which are monoamine reuptake inhibitors, compositions containing these derivatives, and methods of their use for the prevention and treatment of conditions, including, inter alia, vasomotor symptoms, sexual dysfunction, gastrointestinal disorders and genitourinary disorder, depression disorders, endogenous behaviorial disorder, cognitive disorder, diabetic neuropathy, pain, and other diseases or disorders.

### CROSS-REFERENCE TO RELATED APPLICATIONS

This application claims the benefit of U.S. Provisional Application No. 60/869,642, filed Dec. 12, 2006, the contents of which are incorporated herein by reference in their entirety.

### FIELD

The present invention is directed to cyclic sulfonamide derivatives, which are monoamine reuptake inhibitors, compositions containing these derivatives, and methods of their use for the prevention and treatment of diseases or disorders, including vasomotor symptoms, depression disorders, endogenous behavioral disorders, cognitive disorders, sexual dysfunction or pain conditions, in particular vasomotor symptoms.

### BACKGROUND

Vasomotor symptoms (VMS), referred to as hot flushes and night sweats, are the most common symptoms associated with menopause, occurring in 60% to 80% of all women following natural or surgically-induced menopause. VMS are likely to be an adaptive response of the central nervous system (CNS) to declining sex steroids. To date, the most effective therapies for VMS are hormone-based treatments, including estrogens and/or some progestins. Hormonal treatments are very effective at alleviating VMS, but they are not appropriate for all women.

VMS are caused by fluctuations of sex steroid levels and can be disruptive and disabling in both males and females. A hot flush can last up to thirty minutes and vary in their frequency from several times a week to multiple occurrences per day. The patient experiences a hot flush as a sudden feeling of heat that spreads quickly from the face to the chest and back and then over the rest of the body. It is usually accompanied by outbreaks of profuse sweating and may sometimes occur several times an hour, and it often occurs at night. Hot flushes and outbreaks of sweats occurring during the night can cause sleep deprivation. Psychological and emotional symptoms are also observed, such as nervousness, fatigue, irritability, insomnia, depression, memory loss, headache, anxiety, nervousness or inability to concentrate and are caused by the sleep deprivation following hot flush and night sweats (Kramer et al., In: Murphy et al., 3^{rd} Int'l Symposium on Recent Advances in Urological Cancer Diagnosis and Treatment-Proceedings, Paris, France: SCI: 3-7 (1992)).

Hot flushes may be even more severe in women treated for breast cancer for several reasons. Many survivors of breast cancer are given tamoxifen, the most prevalent side effect of which is hot flush, and many women treated for breast cancer undergo premature menopause from chemotherapy. Women with a history of breast cancer have also generally been denied estrogen therapy because of concerns about potential recurrence of breast cancer (Loprinzi, et al., Lancet, 2000, 356(9247): 2059-2063).

Men also experience hot flushes following steroid hormone (androgen) withdrawal. This is true in cases of age-associated androgen decline (Katovich, et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54. As many as one-third of these patients will experience persistent and frequent symptoms severe enough to cause significant discomfort and inconvenience.

The precise mechanism of these vasomotor symptoms is unknown but generally is thought to represent disturbances to normal homeostatic mechanisms controlling thermoregulation and vasomotor activity (Kronenberg et al., “Thermoregulatory Physiology of Menopausal Hot Flashes: A Review,” Can. J. Physiol. Pharmacol., 1987, 65:1312-1324).

The fact that estrogen treatment (e.g. estrogen replacement therapy) relieves the symptoms establishes the link between these symptoms and an estrogen deficiency. For example, the menopausal stage of life is associated with a wide range of other acute symptoms as described above and these symptoms are generally estrogen responsive.

It has been suggested that estrogens may stimulate the activity of both the norepinephrine (NE) and/or serotonin (5-HT) systems (J. Pharmacology & Experimental Therapeutics, 1986, 236(3) 646-652). It is hypothesized that estrogens modulate NE and 5-HT levels providing homeostasis in the thermoregulatory center of the hypothalamus. The descending pathways from the hypothalamus via brainstem/spinal cord and the adrenals to the skin are involved in maintaining normal skin temperature. The action of NE and 5-HT reuptake inhibitors is known to impinge on both the CNS and peripheral nervous system (PNS). The pathophysiology of VMS is mediated by both central and peripheral mechanisms and, therefore, the interplay between the CNS and PNS may account for the efficacy of dual acting SRI/NRIs in the treatment of thermoregulatory dysfunction. In fact, the physiological aspects and the CNS/PNS involvement in VMS may account for the lower doses proposed to treat VMS (Loprinzi, et al., Lancet, 2000, 356:2059-2063; Stearns et al., JAMA, 2003, 289:2827-2834) compared to doses used to treat the behavioral aspects of depression. The interplay of the CNS/PNS in the pathophysiology of VMS was used to support the claims that the norepinephrine system could be targeted to treat VMS.

Although VMS are most commonly treated by hormone therapy (orally, transdermally, or via an implant), some patients cannot tolerate estrogen treatment (Berendsen, Maturitas, 2000, 36(3): 155-164, Fink et al., Nature, 1996, 383(6598): 306). In addition, hormone replacement therapy is usually not recommended for women or men with or at risk for hormonally sensitive cancers (e.g. breast or prostate cancer). Thus, non-hormonal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flashes, including steroids, alpha-adrenergic agonists, and beta-blockers, with varying degree of success (Waldinger et al., Maturitas, 2000, 36(3): 165-168).

α₂-Adrenergic receptors play a role in thermoregulatory dysfunctions (Freedman et al., Fertility & Sterility, 2000, 74(1): 20-3). These receptors are located both pre- and post-synaptically and mediate an inhibitory role in the central and peripheral nervous system. There are four distinct subtypes of the adrenergic_{α2} receptors, i.e., are α_{2A}, α_{2B}, α_{2C} and α_{2D} (Mackinnon et al., TIPS, 1994, 15: 119; French, Pharmacol. Ther., 1995, 68: 175). A non-select α₂-adrenoceptor antagonist, yohimbine, induces a flush and an α₂-adrenergic receptor agonist, clonidine, alleviates the yohimbine effect (Katovich, et al., Proceedings of the Society for Experimental Biology & Medicine, 1990, 193(2): 129-35, Freedman et al., Fertility & Sterility, 2000, 74(1): 20-3). Clonidine has been used to treat hot flush. However, using such treatment is associated with a number of undesired side effects caused by high doses necessary to abate hot flash described herein and known in the related arts.

Chronic pain comes in many forms including visceral, inflammatory or neuropathic and crosses all therapeutic areas. It is a debilitating condition that exerts a high social cost in terms of productivity, economic impact and quality of life and current therapies have limited efficacy. Currently, first-line pharmacological treatments for neuropathic pain (i.e., diabetic neuropathy and post-herpetic neuralgia) and fibromyalgia include off-label use of the tricyclic (TCA) antidepressants (e.g., amytriptyline) and anticonvulsants (e.g., gabapentin) (Collins et al., J. Pain Symptom Manage. 2000, 20(6):449-58; and Marcus Expert Opin Pharmacother. 2003, 4(10): 1687-95.). However, these therapies are only effective in 30-50% of patients and produce only a partial reduction in pain (˜50%). In addition, the clinical benefits of these therapies are often outweighed by the side effects, including dry mouth and sedation. Therefore, newer classes of compounds, including non-TCA antidepressants, are being evaluated preclinically and clinically for chronic pain indications, and recently duloxetine was approved for the treatment of diabetic neuropathy. Although more tolerable than the older tricyclic antidepressants, these newer compounds are not devoid of side effects that include sexual dysfunction, weight gain and nausea.

While the precise pathophysiological mechanisms involved in the development and maintenance of chronic pain states are not fully understood, the pathways involved in pain perception and modulation have been well described and characterized (Gebhart, In: Yaksh TL, editor. Spinal afferent processing, New York: Plenum, 1986. pp 391-416; Fields, et al., Annual Review of Neuroscience 1991, 14: 219-245; Fields, et al. In: Wall P D, Melzack R, editors. Textbook of pain, London: Churchill Livingstone, 1999, pp 309-329; Millan, et al. Progress in Neurobiology; 2002, 66:355-474). A major component of this descending pain inhibitory system involves the noradrenergic pathway (Zhuo, et al., Brain Research 1991; 550:35-48; Holden, et al. Neuroscience 1999; 91: 979-990). It is assumed that norepinephrine (NE) and to a lesser extent serotonin (5-HT) reuptake inhibitors (NRIs and SRIs) attenuate pain by preventing presynaptic reuptake of NE/5-HT leading to increased postsynaptic NE/5-HT levels and sustained activation of this descending pain inhibitory pathway. A meta-analysis of antidepressants and neuropathic pain comparing the efficacy of known NRIs, mixed NRI/SRIs and SRIs determined that compounds with NRI activity were more effective in reducing pain, and that select SRIs did not significantly differ from placebo (Collins et al., J. Pain Symptom Manage. 2000, 20(6): 449-58). This analysis suggests that compounds with greater NRI versus SRI activity will be more effective for the treatment of pain.

Given the complex multifaceted nature of pain and of thermoregulation and the interplay between the CNS and PNS in maintaining thermoregulatory the homeostasis, multiple therapies and approaches can be developed to target the treatment of pain and vasomotor symptoms. The present invention provides novel compounds and compositions containing these compounds directed to these and other important uses.

### SUMMARY

The present invention is directed to cyclic sulfonamide derivatives, which are monoamine reuptake inhibitors, compositions containing these derivatives, and methods of their use for the prevention and treatment of conditions, including, inter alia, vasomotor symptoms (such as hot flush), sexual dysfunction (such as desire-related or arousal-related dysfunction), gastrointestinal disorders and genitourinary disorder (such as stress incontinence or urge incontinence), chronic fatigue syndrome, fibromyalgia syndrome, depression disorders (such as major depressive disorder, generalized anxiety disorder, panic disorder, attention deficit disorder with or without hyperactivity, sleep disturbance, and social phobia), diabetic neuropathy, pain, and combinations thereof.

In one embodiment, the present invention is directed to compounds of formula I:

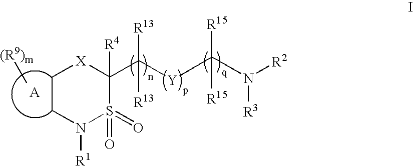

or a pharmaceutically acceptable salt thereof;

wherein:
m is an integer from 0 to 4;
n is an integer from 0 to 2;
p is an integer from 0 to 1;
q is an integer from 1 to 2;
v is an integer from 0 to 2;
X is C(R¹¹)₂, N(R¹²), O, or S(O)ᵥ;
   Y is C(R¹¹)₂, N(R¹²), O, or

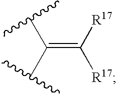

R¹ is aryl substituted with 0-3 R⁵ or heteroaryl substituted with 0-3 R⁵;
R² is H, straight or branched C₁-C₆ alkyl, C₃-C₆ cycloalkyl, or aryl-C₁-C₆ alkyl where said aryl portion is substituted with 0-3 R⁶;
R³ is H, straight or branched C₁-C₆ alkyl, C₁-C₆ alkanol, C₃-C₆ cycloalkyl, or aryl-C₁-C₆ alkyl where said aryl portion is substituted with 0-3 R⁷; or
R² and R³, together with the nitrogen through which they are attached, form a mono- or bicyclic heterocyclic ring of 3 to 12 ring atoms, where one carbon may be optionally replaced with N, O, S, or SO₂, and where any carbon ring atom may be optionally substituted with C₁-C₄ alkyl, F, or CF₃ or where any additional N atom may be optionally substituted with C₁-C₄ alkyl, with the proviso that if Y is O or N(R¹²), q is not 2;
R⁴ is H, straight or branched C₁-C₆ alkyl, aryl substituted with 0-3 R⁸, aryl-C₁-C₆ alkyl where said aryl portion is substituted with 0-3 R⁸, heteroaryl substituted with 0-3 R⁸, or heteroaryl-C₁-C₆ alkyl where said heteroaryl portion is substituted with 0-3 R⁸;
R⁵ is, independently at each occurrence, C₁-C₆ alkyl, alkoxy, halo, CF₃, OCF₃, hydroxy, alkanoyloxy, nitro, nitrile, alkenyl, alkynyl, aryl substituted with 0-3 R⁵ or heteroaryl substituted with 0-3 R⁵;
R⁶ is, independently at each occurrence, C₁-C₄ alkyl, halo or H;
R⁷ is, independently at each occurrence, C₁-C₄ alkyl, halo or H;
R⁸ is, independently at each occurrence, C₁-C₄ alkyl, halo or H;
R⁹ is, independently at each occurrence, C₁-C₆ alkyl, alkoxy, halo, H, CF₃, OCF₃, hydroxy, alkanoyloxy, nitro, nitrile, alkenyl, alkynyl, aryl substituted with 0-3 R¹⁰, heteroaryl substituted with 0-3 R¹⁰, alkylsulfoxide, alkylsulfone, alkylsulfonamide, phenylsulfonamide substituted with 0-3 R¹⁰, alkylamido, or arylamido substituted with 0-3 R¹⁰; or
two adjacent R⁹, together with the ring atoms to which they are attached, form a fused ring of 5 or 6 ring atoms;
R¹⁰ is, independently at each occurrence, H, C₁-C₆ alkyl, or halo;
R¹¹ is, independently at each occurrence, H, C₁-C₆ alkyl, halo, hydroxy, O—(C₁-C₆ alkyl), or aryl substituted with 0-3 R⁸;
R¹² is, independently at each occurrence, H, C₁-C₆ alkyl, or aryl substituted with 0-3 R⁸;
R¹³ is, independently at each occurrence, H, C₁-C₆ alkyl, halide, hydroxy, or aryl substituted with 0-3 R¹⁶;
R¹⁵ is, independently at each occurrence, H, C₁-C₄ alkyl or halide;
R¹⁶ is, independently at each occurrence, H, C₁-C₄ alkyl, or halo;
R¹⁷ is, independently at each occurrence, H or C₁-C₄ alkyl;
wherein ring A is phenyl, naphthyl, pyridyl, pyrimidinyl, thienyl, thiazolyl, or pyrrolyl.

In yet other embodiments, the present invention is directed to compositions, comprising:

a. at least one compound of formula I; and
b. at least one pharmaceutically acceptable carrier.

In another embodiment, the invention is directed to methods for treating or preventing a condition selected from the group consisting of a vasomotor symptom, sexual dysfunction, gastrointestinal disorder, genitourinary disorder, chronic fatigue syndrome, fibromyalgia syndrome, depression disorder, diabetic neuropathy, pain, and combinations thereof in a subject in need thereof, comprising the step of:
administering to said subject an effective amount of a compound of formula I or a pharmaceutically acceptable salt thereof.

The present invention further provides a process for the preparation, of a compound according to formula Ia

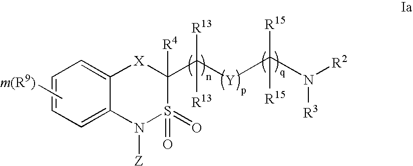

or a pharmaceutically acceptable salt thereof;

wherein:

m is an integer from 0 to 4;

n is an integer from 0 to 2;

p is an integer from 0 to 1;

q is an integer from 1 to 2;

v is an integer from 0 to 2;

X is C(R¹¹)₂, N(R¹²), O, or S(O)ᵥ;

Y is C(R¹¹)₂, N(R¹²), O, or

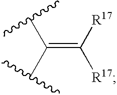

Z is H or an aryl substituted with 0-3 R⁵ or heteroaryl substituted with 0-3 R⁵;

R² is H, straight or branched C₁-C₆ alkyl, C₃-C₆ cycloalkyl, or aryl-C₁-C₆ alkyl where said aryl portion is substituted with 0-3 R⁶;

R³ is H, straight or branched C₁-C₆ alkyl, C₁-C₆ alkanol, C₃-C₆ cycloalkyl, or aryl-C₁-C₆ alkyl where said aryl portion is substituted with 0-3 R⁷; or

R² and R³, together with the nitrogen through which they are attached, form a mono- or bicyclic heterocyclic ring of 3 to 12 ring atoms, where one carbon may be optionally replaced with N, O, S, or SO₂, and where any carbon ring atom may be optionally substituted with C₁-C₄ alkyl, F, or CF₃ or where any additional N atom may be optionally substituted with C₁-C₄ alkyl, with the proviso that if Y is O or N(R¹²), q is not 2;

R⁴ is H, straight or branched C₁-C₆ alkyl, aryl substituted with 0-3 R⁸, aryl-C₁-C₆ alkyl where said aryl portion is substituted with 0-3 R⁸, heteroaryl substituted with 0-3 R⁸, or heteroaryl-C₁-C₆ alkyl where said heteroaryl portion is substituted with 0-3 R⁸;

R⁵ is, independently at each occurrence, C₁-C₆ alkyl, alkoxy, halo, CF₃, OCF₃, hydroxy, alkanoyloxy, nitro, nitrile, alkenyl, alkynyl, aryl substituted with 0-3 R⁵ or heteroaryl substituted with 0-3 R⁵;

R⁶ is, independently at each occurrence, C₁-C₄ alkyl, halo or H;

R⁷ is, independently at each occurrence, C₁-C₄ alkyl, halo or H;

R⁸ is, independently at each occurrence, C₁-C₄ alkyl, halo or H;

R⁹ is, independently at each occurrence, C₁-C₆ alkyl, alkoxy, halo, H, CF₃, OCF₃, hydroxy, alkanoyloxy, nitro, nitrile, alkenyl, alkynyl, aryl substituted with 0-3 R¹⁰, heteroaryl substituted with 0-3 R¹⁰, alkylsulfoxide, alkylsulfone, alkylsulfonamide, phenylsulfonamide substituted with 0-3 R¹⁰, alkylamido, or arylamido substituted with 0-3 R¹⁰; or

two adjacent R⁹, together with the ring atoms to which they are attached, form a fused ring of 5 or 6 ring atoms;

R¹⁰ is, independently at each occurrence, H, C₁-C₆ alkyl, or halo;

R¹¹ is, independently at each occurrence, H, C₁-C₆ alkyl, halo, hydroxy, O—(C₁-C₆ alkyl), or aryl substituted with 0-3 R⁸;

R¹² is, independently at each occurrence, H, C₁-C₆ alkyl, or aryl substituted with 0-3 R⁸;

R¹³ is, independently at each occurrence, H, C₁-C₆ alkyl, halide, hydroxy, or aryl substituted with 0-3 R¹⁶;

R¹⁵ is, independently at each occurrence, H, C₁-C₄ alkyl or halide;

R¹⁶ is, independently at each occurrence, H, C₁-C₄ alkyl, or halo; and

R¹⁷ is, independently at each occurrence, H or C₁-C₄ alkyl;

which process comprises:

(a) performing either of i) or ii) below:
i) coupling a compound according to formula II below with an electrophile according to formula a below:

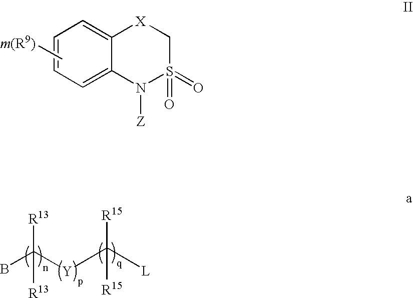

thereby forming a compound according to formula III below:

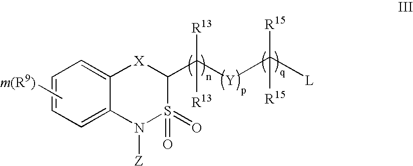

ii) coupling a compound according to formula II hereinabove with an allyl halide according to formula b below:

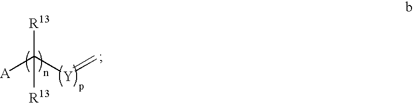

subjecting the compound formed by the coupling with the allyl halide to hydroboration-oxidation to form an alcohol; and

activating the alcohol with an activating agent, thereby forming a compound according to formula III hereinabove;

wherein R⁹, R¹³, R¹⁵, m, n, p, q, Z, X and Y are as defined hereinabove for formula Ia, B is a functional group, L is a leaving group and A is a halogen; and

b) reacting the compound of formula III formed in a) with an amine:

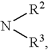

thereby forming a compound according to formula Ia hereinabove, wherein R² and R³ of the amine are as defined hereinabove for formula Ia.

### DETAILED DESCRIPTION OF THE INVENTION

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and Δ “ED₅₀ value” means dose which results in 50% alleviation of the observed condition or effect (50% mean maximum endpoint).

“Norepinephrine transporter” is abbreviated NET.

“Human norepinephrine transporter” is abbreviated hNET.

“Serotonin transporter” is abbreviated SERT.

“Human serotonin transporter” is abbreviated hSERT.

“Norepinephrine reuptake inhibitor” is abbreviated NRI.

“Selective norepinephrine reuptake inhibitor” is abbreviated SNRI.

“Serotonin reuptake inhibitor” is abbreviated SRI.

“Selective serotonin reuptake inhibitor” is abbreviated SSRI.

“Norepinephrine” is abbreviated NE.

“Serotonin is abbreviated 5-HT.

“Subcutaneous” is abbreviated sc.

“Intraperitoneal” is abbreviated ip.

“Oral” is abbreviated po.

In the context of this disclosure, a number of terms shall be utilized. The terms “treat”, “treatment” and “treating” as used herein includes preventative (e.g., prophylactic), curative or palliative treatment.

The term “effective amount,” as used herein, refers to an amount effective, at dosages, and for periods of time necessary, to achieve the desired result with respect to treatment of a given disease or disorder. An effective amount is also one in which any toxic or detrimental effects of the components are outweighed by the therapeutically beneficial effects. In particular, with respect to vasomotor symptoms, “effective amount” refers to the amount of compound or composition of compounds that would increase norepinephrine levels to compensate in part or total for the lack of steroid availability in subjects subject afflicted with a vasomotor symptom. Varying hormone levels will influence the amount of compound required in the present invention. For example, the pre-menopausal state may require a lower level of compound due to higher hormone levels than the peri-menopausal state.

The effective amount of components of the present invention will vary from patient to patient not only with the particular compound, component or composition selected, the route of administration, and the ability of the components (alone or in combination with one or more additional active agents) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response.

Preferably, the compounds of the present invention are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment. With respect to sexual dysfunction, gastrointestinal disorder, genitourinary disorder, chronic fatigue syndrome, fibromyalgia syndrome, depression disorder, endogenous behavioral disorder, cognitive disorder, diabetic neuropathy, or pain, the compounds of the present invention are administered at a dosage and for a time sufficient to treat the symptom or condition.

For example, for a patient, compounds of formula I, or a pharmaceutically acceptable salt thereof, may be administered, preferably, at a dosage of from about 0.1 mg/day to about 1500 mg/day, dosed one or two times daily, more preferably from about 1 mg/day to about 200 mg/day and most preferably from about 1 mg/day to 100 mg/day for a time sufficient to reduce and/or substantially eliminate the number and/or severity of hot flushes or symptom or condition of the sexual dysfunction, gastrointestinal disorder, genitourinary disorder, chronic fatigue syndrome, fibromyalgia syndrome, depression disorder, endogenous behavioral disorder, cognitive disorder, diabetic neuropathy, or pain.

The terms “component,” “composition,” “composition of compounds,” “compound,” “drug,” or “pharmacologically active agent” or “active agent” or “medicament” are used interchangeably herein to refer to a compound or compounds or composition of matter which, when administered to a subject (human or animal) induces a desired pharmacological and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, the term “inhibitor” refers to any agent that inhibits, suppresses, represses, or decreases a specific activity, such as norepinephrine reuptake activity. The term “inhibitor” is intended to comprise any compound, e.g., antibody, small molecule, peptide, oligopeptide, polypeptide, or protein (preferably small molecule or peptide) that exhibits a partial, complete, competitive and/or inhibitory effect on mammalian, preferably human, norepinephrine reuptake or both serotonin reuptake and the norepinephrine reuptake, thus diminishing or blocking (preferably diminishing) some or all of the biological effects of endogenous norepinephrine reuptake or of both serotonin reuptake and the norepinephrine reuptake.

Within the present invention, the compounds of formula I may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

“Administering,” as used herein, means either directly administering a compound or composition of the present invention, or administering a prodrug, derivative or analog which will form an equivalent amount of the active compound or substance within the body.

The term “subject” or “patient” refers to an animal, including the human species, that is treatable with the compounds, compositions, and/or methods of the present invention. The term “subject” or “subjects” is intended to refer to both the male and female gender unless one gender is specifically indicated. Accordingly, the term “patient” comprises any mammal which may benefit from treatment a disease or disorder, such as a human, especially if the mammal is female, either in the pre-menopausal, peri-menopausal, or post-menopausal period. Furthermore, the term patient includes female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis. However, the term “patient” is not intended to be limited to a woman.

“Side effect” refers to a consequence other than the one(s) for which an agent or measure is used, such as one or more adverse effects produced by a drug, especially on a tissue or organ system other than the one sought to be benefited by its administration. In the case, for example, of high doses of NRIs or NRI/SRI compounds alone, the term “side effect” may refer to such conditions as, for example, vomiting, nausea, sweating, and hot flushes (Janowsky, et al., Journal of Clinical Psychiatry, 1984, 45(10 Pt 2): 3-9).

“Vasomotor symptoms,” “vasomotor instability symptoms” and “vasomotor disturbances” include, but are not limited to, hot flushes (flashes), insomnia, sleep disturbances, mood disorders, irritability, excessive perspiration, night sweats, fatigue, and the like, caused by, inter alia, thermoregulatory dysfunction.

The term “hot flush” (also called “hot flash”) is an art-recognized term that refers to an episodic disturbance in body temperature typically consisting of a sudden skin flushing, usually accompanied by perspiration in a subject.

The terms “premature menopause” or “artificial menopause” refer to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

The term “pre-menopausal” means before the menopause, the term “peri-menopausal” means during the menopause and the term “post-menopausal” means after the menopause. “Ovariectomy” means removal of an ovary or ovaries and can be effected according to Merchenthaler et al., Maturitas, 1998, 30(3): 307-316.

The term “sexual dysfunction” includes, but is not limited to, a condition relating to defects in sexual desire and/or arousal.

As used herein, “gastrointestinal and genitourinary disorders” includes irritable bowel syndrome, symptomatic GERD, hypersensitive esophagus, nonulcer dyspepsia, noncardiac chest pain, biliary dyskinesia, sphincter of Oddi dysfunction, incontinence (i.e., urge incontinence, stress incontinence, genuine stress incontinence, and mixed incontinence, including the involuntary voiding of feces or urine, and dribbling or leakage or feces or urine which may be due to one or more causes including but not limited to pathology altering sphincter control, loss of cognitive function, overdistention of the bladder, hyperreflexia and/or involuntary urethral relaxation, weakness of the muscles associated with the bladder or neurologic abnormalities), interstitial cystitis (irritable bladder), and chronic pelvic pain (including, but not limited to vulvodynia, prostatodynia, and proctalgia).

As used herein, “chronic fatigue syndrome” (CFS) is a condition characterized by physiological symptoms selected from weakness, muscle aches and pains, excessive sleep, malaise, fever, sore throat, tender lymph nodes, impaired memory and/or mental concentration, insomnia, disordered sleep, localized tenderness, diffuse pain and fatigue, and combinations thereof, whether or not correlated with Epstein-Barr virus infection.

As used herein, “fibromyalgia syndrome” (FMS) includes FMS and other somatoform disorders, including FMS associated with depression, somatization disorder, conversion disorder, pain disorder, hypochondriasis, body dysmorphic disorder, undifferentiated somatoform disorder, and somatoform NOS. FMS and other somatoform disorders are accompanied by physiological symptoms selected from a generalized heightened perception of sensory stimuli, abnormalities in pain perception in the form of allodynia (pain with innocuous stimulation), abnormalities in pain perception in the form of hyperalgesia (increased sensitivity to painful stimuli), and combinations thereof.

As used herein, the term “depression disorder” includes major depressive disorder, generalized anxiety disorder, panic disorder, attention deficit disorder with or without hyperactivity, sleep disturbance, social phobia, and combinations thereof.

The compounds of the present invention can also be used to treat a cognitive disorder or an endogenous behavioral disorder. As used herein, a “cognitive disorder” includes changes or defects in alertness; mild cognitive impairment (MCI), characterized by problems with memory, language, or other mental functions which is severe enough to be noticeable or be detected by tests, but not serious enough to significantly interfere with daily life; cognitive disorder NOS (not otherwise specified), characterized by a syndrome of cognitive impairment that does not meet the criteria for delerium, dementia or amnesic disorders; age-related cognitive decline (ARCD); and cognitive arousal (such as increased arousal states). A cognition disorder can be ideopathic, or can be caused by a variety of other factors such as a congenital defect, alcohol or drug addiction, transient or permanent pharmacologic effects of drugs, organic or infectious disease (e.g., Alzheimer's disease, Parkinson's disease, AIDS), trauma (e.g., brain injury, stroke) or advanced age. As used herein, an “endogenous behavioral disorder” includes attention deficit disorder/attention deficit hyperactivity disorder (ADD/ADHD, including adult and pediatric forms of predominantly inattentive, predominantly hyperactive, or combined types), obsessive-compulsive disorder (OCD), oppositional or oppositional explosive defiant disorder (ODD/OEDD), anxiety and panic disorders (APD) and temper, rage and outburst behavior disorder (TROBD).

As used herein, “pain” includes both acute and chronic nociceptic or neuropathic pain, which may be centralized pain, peripheral pain, or combination thereof. The term includes many different types of pain including, but not limited to, visceral pain, musculoskeletal pain, bony pain, cancer pain, inflammatory pain, and combinations thereof, such as lower back pain, atypical chest pain, headache such as cluster headache, migraine, herpes neuralgia, phantom limb pain, pelvic pain, myofascial face pain, abdominal pain, neck pain, central pain, dental pain, opioid resistant pain, visceral pain, surgical pain, bone injury pain, pain during labor and delivery, pain resulting from burns, post partum pain, angina pain, peripheral neuropathy and diabetic neuropathy, post-operative pain, and pain which is co-morbid with nervous system disorders described herein.

As used herein, the term “acute pain” refers to centralized or peripheral pain that is intense, localized, sharp, or stinging, and/or dull, aching, diffuse, or burning in nature and that occurs for short periods of time.

As used herein, the term “chronic pain” refers to centralized or peripheral pain that is intense, localized, sharp, or stinging, and/or dull, aching, diffuse, or burning in nature and that occurs for extended periods of time (i.e., persistent and/or regularly reoccurring), including, for the purpose of the present invention, neuropathic pain and cancer pain. Chronic pain includes neuropathic pain, hyperalgesia, and/or allodynia.

As used herein, the term “neuropathic pain” refers to chronic pain caused by damage to or pathological changes in the peripheral or central nervous systems. Examples of pathological changes related to neuropathic pain include prolonged peripheral or central neuronal sensitization, central sensitization related damage to nervous system inhibitory and/or exhibitory functions and abnormal interactions between the parasympathetic and sympathetic nervous systems. A wide range of clinical conditions may be associated with or form the basis for neuropathic pain including, for example, diabetes, post traumatic pain of amputation (nerve damage cause by injury resulting in peripheral and/or central sensitization such as phantom limb pain), lower back pain, cancer, chemical injury, toxins, other major surgeries, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, or viral or bacterial infections such as shingles or human immunodeficiency virus (HIV), and combinations thereof. Also included in the definition of neuropathic pain is a condition secondary to metastatic infiltration, adiposis dolorosa, burns, central pain conditions related to thalamic conditions, and combinations thereof.

As used herein, the term “hyperalgesia” refers to pain where there is an increase in sensitivity to a typically noxious stimulus.

As used herein, the term “allodynia” refers to an increase in sensitivity to a typically non-noxious stimulus.

As used herein, the term “visceral pain” refers to pain associated with or resulting from maladies of the internal organs, such as, for example, ulcerative colitis, irritable bowel syndrome, irritable bladder, Crohn's disease, rheumatologic (arthralgias), tumors, gastritis, pancreatitis, infections of the organs, biliary tract disorders, and combinations thereof.

As used herein, the term “female-specific pain” refers to pain that may be acute and/or chronic pain associated with female conditions. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes, and combinations thereof.

The term “alkyl” refers to an optionally substituted, saturated, straight-chain or branched hydrocarbon group, having from about 1 to about 20 carbon atoms. Examples of alkyl groups include methyl (Me), ethyl (Et), propyl (e.g., n-propyl and isopropyl), butyl (e.g., n-butyl, isobutyl, s-butyl, t-butyl), pentyl groups (e.g., n-pentyl, isopentyl, neopentyl), and the like. A lower alkyl group typically has up to 6 carbon atoms. In various embodiments, an alkyl group has 1-6 carbon atoms, and is referred to as a “C₁₋₆ alkyl group.” Examples of C₁₋₆ alkyl groups include, but are not limited to, methyl, ethyl, propyl (e.g., n-propyl and isopropyl), butyl (e.g., n-butyl, isobutyl, s-butyl, t-butyl), pentyl (e.g., n-pentyl, neopentyl, isopentyl, t-pentyl), and hexyl groups (e.g., n-hexyl, isohexyl). A branched alkyl group has at least 3 carbon atoms (e.g., an isopropyl group), and in various embodiments, has up to 6 carbon atoms, i.e., a branched lower alkyl group. Examples of branched lower alkyl groups include, but are not limited to:

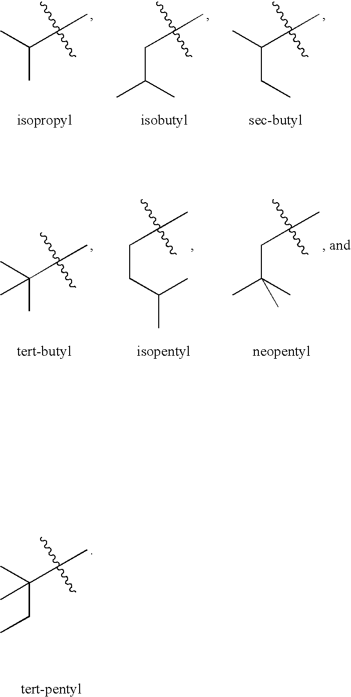

The term “cycloalkyl” as used herein, refers to a cyclic hydrocarbon group containing 3 to 12 carbon atoms, preferably 3 to 6 carbon atoms. Cycloalkyl groups may be monocyclic or bicyclic, and may be saturated or partially saturated. The term “cycloalkyl” includes bicyclic cycloalkyl groups, and bridged cycloalkyl groups which contain at least one carbon-carbon bond between two non-adjacent carbon atoms of the cycloalkyl ring.

“Alkenyl,” as used herein, refers to an alkyl group of at least two carbon atoms having one or more double bonds, wherein alkyl is as defined herein. Alkenyl groups can be optionally substituted.

“Alkynyl,” as used herein, refers to an alkyl group of at least two carbon atoms having one or more triple bonds, wherein alkyl is as defined herein. Alkynyl groups can be optionally substituted.

“Halo,” as used herein, refers to chloro, bromo, fluoro, and iodo.

“Aryl” as used herein, refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

“Heteroaryl,” as used herein, refers to an optionally substituted, mono-, di-, tri-, or other multicyclic aromatic ring system that includes at least one, and preferably from 1 to about 4 heteroatom ring members selected from sulfur, oxygen and nitrogen. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

“Heterocyclic ring,” as used herein, refers to a stable 4- to 12-membered monocyclic or bicyclic or 7- to 10-membered bicyclic heterocyclic ring that is saturated, partially unsaturated or unsaturated (aromatic), and which contains carbon atoms and from 1 to 4 heteroatoms independently selected from the group consisting of N, O and S and including any bicyclic group in which any of the above defined heterocyclic rings is fused to a benzene ring. The nitrogen and sulfur heteroatoms may optionally be oxidized. The heterocyclic ring may be attached to its pendant group at any heteroatom or carbon atom that results in a stable structure. The heterocyclic rings described herein may be substituted on carbon or on a nitrogen atom if the resulting compound is stable. If specifically noted, a nitrogen atom in the heterocycle may optionally be quaternized. It is preferred that when the total number of S and O atoms in the heterocycle exceeds one, then these heteroatoms are not adjacent to one another. It is preferred that the total number of S and O atoms in the heterocycle is not more than two. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4H-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4H-carbazolyl, α-, β-, or γ-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylpyrimidinyl, phenanthridinyl, phenanthrolinyl, phenoxazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinyl. Also included are fused ring and spiro compounds containing, for example, the above heterocycles.

“Alkoxy,” as used herein, refers to the group R—O— where R is an alkyl group as defined herein.

“Alkanoyloxy,” as used herein, refers to the group R—C(═O)—O— where R is an alkyl group of 1 to 5 carbon atoms.

“Alkylsulfoxide,” as used herein, refers to as used herein, refers to —S(═O)—R, where R is alkyl, as defined above.

“Alkylsulfone,” as used herein, refers to —S(═O)₂—R, where R is alkyl, as defined above.

“Alkylsulfonamide,” as used herein, refers to —NR—S(═O)₂—R, where each R is independently, alkyl, as defined above or the NR part may also be NH.

“Phenylsulfonamide,” as used herein, refers to —NR—S(═O)₂-phenyl, where R is H or alkyl, as defined above.

“Alkylamido,” as used herein, refers to —NR—C(═O)—R, where each R is independently, alkyl, as defined above, or the NR part may also be NH.

“Phenylamido,” as used herein, refers to —NR—C(═O)-phenyl, where R is H or alkyl, as defined above.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl. By way of another example, the term “5-9 membered heteroaryl group” is specifically intended to individually disclose a heteroaryl group having 5, 6, 7, 8, 9, 5-9, 5-8, 5-7, 5-6, 6-9, 6-8, 6-7, 7-9, 7-8, and 8-9 ring atoms.

In one embodiment, the present invention is directed to compounds of formula I:

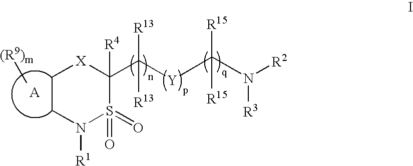

or a pharmaceutically acceptable salt thereof;

wherein:

m is an integer from 0 to 4;

n is an integer from 0 to 2;

p is an integer from 0 to 1;

q is an integer from 1 to 2;

v is an integer from 0 to 2;

X is C(R¹¹)₂, N(R¹²), O, or S(O)ᵥ;
Y is C(R¹¹)₂, N(R¹²), O, or

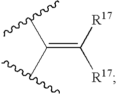

R¹ is aryl substituted with 0-3 R⁵ or heteroaryl substituted with 0-3 R⁵;

R² is H, straight or branched C₁-C₆ alkyl, C₃-C₆ cycloalkyl, or aryl-C₁-C₆ alkyl where said aryl portion is substituted with 0-3 R⁶;

R³ is H, straight or branched C₁-C₆ alkyl, C₁-C₆ alkanol, C₃-C₆ cycloalkyl, or aryl-C₁-C₆ alkyl where said aryl portion is substituted with 0-3 R⁷; or

R² and R³, together with the nitrogen through which they are attached, form a mono- or bicyclic heterocyclic ring of 3 to 12 ring atoms, where one carbon may be optionally replaced with N, O, S, or SO₂, and where any carbon ring atom may be optionally substituted with C₁-C₄ alkyl, F, or CF₃ or where any additional N atom may be optionally substituted with C₁-C₄ alkyl, with the proviso that if Y is O or N(R¹²), q is not 2;

R⁴ is H, straight or branched C₁-C₆ alkyl, aryl substituted with 0-3 R⁸, aryl-C₁-C₆ alkyl where said aryl portion is substituted with 0-3 R⁸, heteroaryl substituted with 0-3 R⁸, or heteroaryl-C₁-C₆ alkyl where said heteroaryl portion is substituted with 0-3 R⁸;

R⁵ is, independently at each occurrence, C₁-C₆ alkyl, alkoxy, halo, CF₃, OCF₃, hydroxy, alkanoyloxy, nitro, nitrile, alkenyl, alkynyl, aryl substituted with 0-3 R⁵ or heteroaryl substituted with 0-3 R⁵;

R⁶ is, independently at each occurrence, C₁-C₄ alkyl, halo or H;

R⁷ is, independently at each occurrence, C₁-C₄ alkyl, halo or H;

R⁸ is, independently at each occurrence, C₁-C₄ alkyl, halo or H;

R⁹ is, independently at each occurrence, C₁-C₆ alkyl, alkoxy, halo, H, CF₃, OCF₃, hydroxy, alkanoyloxy, nitro, nitrile, alkenyl, alkynyl, aryl substituted with 0-3 R¹⁰, heteroaryl substituted with 0-3 R¹⁰, alkylsulfoxide, alkylsulfone, alkylsulfonamide, phenylsulfonamide substituted with 0-3 R¹⁰, alkylamido, or arylamido substituted with 0-3 R¹⁰; or

two adjacent R⁹, together with the ring atoms to which they are attached, form a fused ring of 5 or 6 ring atoms;

R¹⁰ is, independently at each occurrence, H, C₁-C₆ alkyl, or halo;

R¹¹ is, independently at each occurrence, H, C₁-C₆ alkyl, halo, hydroxy, O—(C₁-C₆ alkyl), or aryl substituted with 0-3 R⁸;

R¹² is, independently at each occurrence, H, C₁-C₆ alkyl, or aryl substituted with 0-3 R⁸;

R¹³ is, independently at each occurrence, H, C₁-C₆ alkyl, halide, hydroxy, or aryl substituted with 0-3 R¹⁶;

R¹⁵ is, independently at each occurrence, H, C₁-C₄ alkyl, or halide;

R¹⁶ is, independently at each occurrence, H, C₁-C₄ alkyl, or halo;

R¹⁷ is, independently at each occurrence, H or C₁-C₄ alkyl;

wherein ring A is phenyl, naphthyl, pyridyl, pyrimidinyl, thienyl, thiazolyl, or pyrrolyl.

In certain embodiments of the compounds of formula I,
m is an integer from 0 to 4;
n is an integer from 0 to 2;
p is 0 or 1;
q is 1 or 2;
X is C(R¹¹)₂, N(R¹²), O, or S(O)ᵥ;
v is an integer from 0 to 2;

Y is C(R¹¹)₂, N(R¹²), O, or

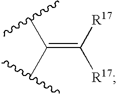

R¹ is C₆-C₁₄ aryl substituted with 0-3 R⁵ or 5-10 membered heteroaryl substituted with 0-3 R⁵;
R² is H, C₁-C₆ alkyl, C₃-C₆ cycloalkyl, or C₆-C₁₄ aryl-C₁-C₆ alkyl, where said aryl portion is substituted with 0-3 R⁶;
R³ is H, C₁-C₆ alkyl, C₁-C₆ hydroxyalkyl, C₃-C₆ cycloalkyl, or C₆-C₁₄ aryl-C₁-C₆ alkyl where said aryl portion is substituted with 0-3 R⁷; or
R² and R³, together with the nitrogen through which they are attached, form a 3-12 membered heterocyclic ring optionally containing 1-4 additional heteroatoms N, O, S, or SO₂, and where any carbon ring atom may be optionally substituted with C₁-C₄ alkyl, F, or CF₃ or where any additional N atom may be optionally substituted with C₁-C₄ alkyl with the proviso that if Y is O or N(R¹²), q is not 2;
R⁴ is H, C₁-C₆ alkyl, C₆-C₁₄ aryl substituted with 0-3 R⁸, C₆-C₁₄ aryl-C₁-C₆ alkyl where said aryl portion is substituted with 0-3 R⁸, 5-10 membered heteroaryl substituted with 0-3 R⁸, or 5-10 membered heteroaryl-C₁-C₆ alkyl where said heteroaryl portion is substituted with 0-3 R⁸;
R⁵ is, independently at each occurrence, C₁-C₆ alkyl, alkoxy, halo, CF₃, OCF₃, hydroxy, C₁-C₆ alkanoyloxy, nitro, nitrile, C₂-C₆ alkenyl, C₂-C₆ alkynyl, C₆-C₁₄ aryl substituted with 0-3 R⁵ or 5-10 membered heteroaryl substituted with 0-3 R⁵;
R⁶ is, independently at each occurrence, C₁-C₄ alkyl, halo or H;
R⁷ is, independently at each occurrence, C₁-C₄ alkyl, halo or H;
R⁸ is, independently at each occurrence, C₁-C₄ alky, halo or H;
R⁹ is, independently at each occurrence, C₁-C₆ alkyl, C₁-C₆ alkoxy, halo, H, CF₃, OCF₃, hydroxy, C₁-C₆ alkanoyloxy, nitro, nitrile, C₂-C₆ alkenyl, C₂-C₆ alkynyl, C₆-C₁₄ aryl substituted with 0-3 R¹⁰, 5-10 membered heteroaryl substituted with 0-3 R¹⁰, C₁-C₆ alkylsulfoxy, C₁-C₆ alkylsulfonyl, C₁-C₆ alkylsulfonamido, C₆-C₁₄ arylsulfonamido substituted with 0-3 R¹⁰, C₁-C₆ alkylamido, or C₆-C₁₄ arylamido substituted with 0-3 R¹⁰; or
two adjacent R⁹, together with the ring atoms to which they are attached, form a 5 or 6-membered fused ring;
R¹⁰ is, independently at each occurrence, H, C₁-C₆ alkyl, or halo;
R¹¹ is, independently at each occurrence, H, C₁-C₆ alkyl, halo, hydroxy, C₁-C₆ alkoxy, or C₆-C₁₄ aryl substituted with 0-3 R⁸;
R¹² is, independently at each occurrence, H, C₁-C₆ alkyl, or C₆-C₁₄ aryl substituted with 0-3 R⁸;
R¹³ is, independently at each occurrence, H, C₁-C₆ alkyl, halide, hydroxy, or C₆-C₁₄ aryl substituted with 0-3 R¹⁶;
R¹⁵ is, independently at each occurrence, H, C₁-C₄ alkyl or halide;
R¹⁶ is, independently at each occurrence, H, C₁-C₄ alkyl, or halo;
R¹⁷ is, independently at each occurrence, H or C₁-C₄ alkyl;
wherein ring A is phenyl, naphthyl, pyridyl, pyrimidinyl, thienyl, thiazolyl, or pyrrolyl.

In preferred embodiments of the compounds of formula I, A is phenyl.

In preferred embodiments of the compounds of formula I, n is 1. In other preferred embodiments, n is 0.

In preferred embodiments of the compounds of formula I, m is an integer from 0 to 2. In other preferred embodiments, m is 0. In yet other embodiments, m is 1. In yet further preferred embodiments, m is 2.

In preferred embodiments of the compounds of formula I, p is 1.

In preferred embodiments of the compounds of formula I, q is 1.

In preferred embodiments of the compounds of formula I, X is C(R¹¹)₂, O, or S. In certain other preferred embodiments, X is N(R¹²). In certain other preferred embodiments, X is O. In yet other preferred embodiments, X is S.

In preferred embodiments of the compounds of formula I, R¹ is aryl (e.g., C₆-C₁₀ aryl) substituted with 0-3 R⁵, especially phenyl, methyl-phenyl, dimethyl-phenyl, methoxy-phenyl, fluoro-phenyl, chloro-phenyl, fluoro-chloro-phenyl, trifluoromethyl-phenyl, naphthyl, or fluoro-fluoro-phenyl.

In preferred embodiments of the compounds of formula I, R¹ is heteroaryl (e.g., 5-10 membered heteroaryl) substituted with 0-3 R⁵, especially pyridinyl or quinolinyl.

In preferred embodiments of the compounds of formula I, R² is H or straight or branched C₁-C₆ alkyl. In other preferred embodiments of the compounds of formula I, R² is C₃-C₆ cycloalkyl or C₆-C₁₀aryl-C₁-C₆alkyl. In certain preferred embodiments of the compounds of formula I, R² is hydrogen, methyl, ethyl, n-propyl, isopropyl, cyclopropyl, n-butyl, isobutyl, cyclobutyl, cyclopentyl, cyclohexyl, or benzyl. In certain preferred embodiments, R² is methyl.

In preferred embodiments of the compounds of formula I, R³ is H, methyl, ethyl, n-propyl, isopropyl, cyclopropyl, n-butyl, isobutyl, cyclobutyl, cyclopentyl, cyclohexyl, benzyl, or propanol. In preferred embodiments of the compounds of formula I, R³ is H.

In preferred embodiments of the compounds of formula I, R² and R³, together with the nitrogen through which they are attached, form pyrrolidinyl, piperidinyl, or piperazinyl.

In preferred embodiments of the compounds of formula I, R⁴ is H or straight or branched C₁-C₆ alkyl, especially R⁴ is H or methyl.

In preferred embodiments of the compounds of formula I, R⁵ is, independently at each occurrence, C₁-C₆ alkyl, halo, C₁-C₆ alkoxy or CF₃. In certain preferred embodiments, R⁵ is, independently at each occurrence, methyl, methoxy, fluoro, chloro, or trifluoromethyl.

In preferred embodiments of the compounds of formula I, R⁶ is, independently at each occurrence, methyl, fluoro or chloro.

In preferred embodiments of the compounds of formula I, R⁷ is, independently at each occurrence, methyl, fluoro, chloro or hydrogen.

In preferred embodiments of the compounds of formula I, R⁸ is, independently at each occurrence, methyl, fluoro, or chloro.

In certain preferred embodiments of the compounds of formula I, R⁹ is C₁-C₆ alkyl, halo or hydrogen. In other preferred embodiments of the compounds of formula I, R⁹ is C₁-C₆ alkoxy or CF₃. In certain other preferred embodiments, R⁹ is methyl, methoxy, fluoro, chloro, trifluoromethyl, or hydrogen.

In preferred embodiments of the compounds of formula I, R¹¹ is, independently at each occurrence, H, C₁-C₆ alkyl, or halo.

In preferred embodiments of the compounds of formula I, R¹² is, independently at each occurrence, H or C₁-C₆ alkyl.

In preferred embodiments of the compounds of formula I, R¹³ is, independently at each occurrence, H, C₁-C₆ alkyl, or aryl (e.g., C₆-C₁₀ aryl).

In preferred embodiments of the compounds of formula I, R¹⁵ is, independently at each occurrence, H, methyl, ethyl, n-propyl, isopropyl, n-butyl, or iso-butyl.

In certain preferred embodiments of the compounds of formula I,
n is 1;
p is 1;
q is 1;
X is C(R¹¹)₂ or O;
Y is C(R¹¹)₂; and
R³ is H.

In certain preferred embodiments of the compounds of formula I,
n is 1;
p is 1;
q is 1;
X is C(R¹¹)₂ or O;
Y is C(R¹¹)₂;
R¹ is phenyl; and
R³ is H.

In certain preferred embodiments of the compounds of formula I,
n is 1;
p is 1;
q is 1;
X is C(R¹¹)₂ or O;
Y is C(R¹¹)₂;
R¹ is phenyl;
R² is methyl;
R³ is H; and
R⁵ is H or F.

In certain preferred embodiments of the compounds of formula I,
n is 1;
p is 1;
q is 1;
X is C(R¹¹)₂ or O;
Y is C(R¹¹)₂;
R¹ is phenyl;
R² is methyl;
R³ is H;
R⁴ is H;
R⁵ is H or F; and
R⁹ is H or F.

Preferred compounds of formula I include:
3-(2,2-dioxido-1-phenyl-3,4-dihydro-1H-2,1-benzothiazin-3-yl)-N-methylpropan-1-amine;
3-(2,2-dioxido-1-phenyl-3,4-dihydro-1H-2,1-benzothiazin-3-yl)propan-1-amine;
3-(2,2-dioxido-1-phenyl-3,4-dihydro-1H-2,1-benzothiazin-3-yl)-N,N-dimethylpropan-1-amine;
3-(2,2-dioxido-1-phenyl-3,4-dihydro-1H-2,1-benzothiazin-3-yl)-N-ethylpropan-1-amine;
3-(2,2-dioxido-1-phenyl-3,4-dihydro-1H-2,1-benzothiazin-3-yl)-N-isopropyl propan-1-amine;
1-phenyl-3-(3-pyrrolidin-1-ylpropyl)-3,4-dihydro-1H-2,1-benzothiazine 2,2-dioxide;
N-benzyl-3-(2,2-dioxido-1-phenyl-3,4-dihydro-1H-2,1-benzothiazin-3-yl)propan-1-amine;
N-[3-(2,2-dioxido-1-phenyl-3,4-dihydro-1H-2,1-benzothiazin-3-yl)propyl]cyclohexanamine;
N-methyl-3-[3-methyl-2,2-dioxido-1-phenyl-3,4-dihydro-1H-2,1-benzothiazin-3-yl]propan-1-amine;
3-[2,2-dioxido-1-phenyl-3,4-dihydro-1H-2,1-benzothiazin-3-yl]-N-methylpropan-1-amine;
3-(2,2-dioxido-1-phenyl-1H-4,2,1-benzoxathiazin-3-yl)-N-methylpropan-1-amine;
3-[2,2-dioxido-1-phenyl-1H-4,2,1-benzoxathiazin-3-yl]-N-methylpropan-1-amine;
3-[2,2-dioxido-1-phenyl-1H-4,2,1-benzoxathiazin-3-yl]-N-methylpropan-1-amine;
3-[1-(4-fluorophenyl)-2,2-dioxido-1H-4,2,1-benzoxathiazin-3-yl]-N-methylpropan-1-amine;
N-methyl-3-[1-(3-methyl phenyl)-2,2-dioxido-1H-4,2,1-benzoxathiazin-3-yl]propan-1-amine;
N-methyl-3-[1-(4-methyl phenyl)-2,2-dioxido-1H-4,2,1-benzoxathiazin-3-yl]propan-1-amine;
3-[1-(3-methoxyphenyl)-2,2-dioxido-1H-4,2,1-benzoxathiazin-3-yl]-N-methylpropan-1-amine;
3-[1-(4-methoxyphenyl)-2,2-dioxido-1H-4,2,1-benzoxathiazin-3-yl]-N-methylpropan-1-amine;
3-[1-(3-fluorophenyl)-2,2-dioxido-1H-4,2,1-benzoxathiazin-3-yl]-N-methylpropan-1-amine;
N-methyl-3-[1-(2-naphthyl)-2,2-dioxido-1H-4,2,1-benzoxathiazin-3-yl]propan-1-amine;
3-[1-(3,5-dimethylphenyl)-2,2-dioxido-1H-4,2,1-benzoxathiazin-3-yl]-N-methylpropan-1-amine;
2-(2,2-dioxido-1-phenyl-1H-4,2,1-benzoxathiazin-3-yl)-N-ethylethanamine;
2-(2,2-dioxido-1-phenyl-1H-4,2,1-benzoxathiazin-3-yl)ethanamine;
2-(2,2-dioxido-1-phenyl-1H-4,2,1-benzoxathiazin-3-yl)-N-methylethanamine;
2-(2,2-dioxido-1-phenyl-1H-4,2,1-benzoxathiazin-3-yl)-N,N-dimethylethanamine;
N-[2-(2,2-dioxido-1-phenyl-1H-4,2,1-benzoxathiazin-3-yl)ethyl]propan-1-amine;
N-[2-(2,2-dioxido-1-phenyl-1H-4,2,1-benzoxathiazin-3-yl)ethyl]-2-methylpropan-1-amine;
1-phenyl-3-(2-pyrrolidin-1-ylethyl)-1H-4,2,1-benzoxathiazine 2,2-dioxide;
3-[2-(4-methylpiperazin-1-yl)ethyl]-1-phenyl-1H-4,2,1-benzoxathiazine 2,2-dioxide;
N-[2-(2,2-dioxido-1-phenyl-1H-4,2,1-benzoxathiazin-3-yl)ethyl]butan-1-amine;
N-[2-(2,2-dioxido-1-phenyl-1H-4,2,1-benzoxathiazin-3-yl)ethyl]cyclobutanamine;
3-(2,2-dioxido-1-phenyl-1H-4,2,1-benzoxathiazin-3-yl)propan-1-amine;
3-(2,2-dioxido-1-phenyl-1H-4,2,1-benzoxathiazin-3-yl)-N-ethylpropan-1-amine;
3-(2,2-dioxido-1-phenyl-1H-4,2,1-benzoxathiazin-3-yl)-N-propylpropan-1-amine;
N-[3-(2,2-dioxido-1-phenyl-1H-4,2,1-benzoxathiazin-3-yl)propyl]butan-1-amine;
3-(2,2-dioxido-1-phenyl-1H-4,2,1-benzoxathiazin-3-yl)-N-isopropylpropan-1-amine;
N-[3-(2,2-dioxido-1-phenyl-1H-4,2,1-benzoxathiazin-3-yl)propyl]-2-methylpropan-1-amine;
3-{[3-(2,2-dioxido-1-phenyl-1H-4,2,1-benzoxathiazin-3-yl)propyl]amino}propan-1-ol;
N-[3-(2,2-dioxido-1-phenyl-1H-4,2,1-benzoxathiazin-3-yl)propyl]cyclo propanamine;
N-[3-(2,2-dioxido-1-phenyl-1H-4,2,1-benzoxathiazin-3-yl)propyl]cyclobutanamine;
N-[3-(2,2-dioxido-1-phenyl-1H-4,2,1-benzoxathiazin-3-yl)propyl]cyclopentanamine;
N-[3-(2,2-dioxido-1-phenyl-1H-4,2,1-benzoxathiazin-3-yl)propyl]cyclohexanamine;
3-(7-fluoro-2,2-dioxido-1-phenyl-1H-4,2,1-benzoxathiazin-3-yl)-N-methylpropan-1-amine;
3-(6-chloro-2,2-dioxido-1-phenyl-1H-4,2,1-benzoxathiazin-3-yl)-N-methylpropan-1-amine;
3-(6-fluoro-2,2-dioxido-1-phenyl-1H-4,2,1-benzoxathiazin-3-yl)-N-methylpropan-1-amine;
3-(5-fluoro-2,2-dioxido-1-phenyl-1H-4,2,1-benzoxathiazin-3-yl)-N-methylpropan-1-amine;
3-(8-fluoro-2,2-dioxido-1-phenyl-1H-4,2,1-benzoxathiazin-3-yl)-N-methylpropan-1-amine;
N-methyl-3-(8-methyl-2,2-dioxido-1-phenyl-1H-4,2,1-benzoxathiazin-3-yl)propan-1-amine;
N-methyl-3-(7-methyl-2,2-dioxido-1-phenyl-1H-4,2,1-benzoxathiazin-3-yl)propan-1-amine;
N-methyl-3-(6-methyl-2,2-dioxido-1-phenyl-1H-4,2,1-benzoxathiazin-3-yl)propan-1-amine;
3-(6-methoxy-2,2-dioxido-1-phenyl-1H-4,2,1-benzoxathiazin-3-yl)-N-methylpropan-1-amine;
3-(7-chloro-2,2-dioxido-1-phenyl-1H-4,2,1-benzoxathiazin-3-yl)-N-methylpropan-1-amine;
3-(2,2-dioxido-1,7-diphenyl-1H-4,2,1-benzoxathiazin-3-yl)-N-methylpropan-1-amine;
3-(2,2-dioxido-1-phenyl-1H-naphtho[2,3-e][1,3,4]oxathiazin-3-yl)-N-methylpropan-1-amine;
3-(2,2-dioxido-1-pyridin-3-yl-1H-4,2,1-benzoxathiazin-3-yl)-N-methylpropan-1-amine;
3-(2,2-dioxido-1-quinolin-3-yl-1H-4,2,1-benzoxathiazin-3-yl)-N-methylpropan-1-amine;
N-benzyl-3-(2,2-dioxido-1-phenyl-1H-4,2,1-benzoxathiazin-3-yl)-N-methylpropan-1-amine;
3-(2,2-dioxido-1-phenyl-1H-2,4,1-benzodithiazin-3-yl)-N-methylpropan-1-amine;
N-methyl-3-[1-(3-methyl phenyl)-2,2-dioxido-1H-2,4,1-benzodithiazin-3-yl]propan-1-amine;
3-[1-(3-fluorophenyl)-2,2-dioxido-1H-2,4,1-benzodithiazin-3-yl]-N-methylpropan-1-amine;
3-[1-(3-methoxyphenyl)-2,2-dioxido-1H-2,4,1-benzodithiazin-3-yl]-N-methylpropan-1-amine;
N-methyl-3-[1-(4-methyl phenyl)-2,2-dioxido-1H-2,4,1-benzodithiazin-3-yl]propan-1-amine;
3-[1-(4-fluorophenyl)-2,2-dioxido-1H-2,4,1-benzodithiazin-3-yl]-N-methylpropan-1-amine;
3-[1-(4-methoxyphenyl)-2,2-dioxido-1H-2,4,1-benzodithiazin-3-yl]-N-methylpropan-1-amine;
3-[1-(3-chlorophenyl)-2,2-dioxido-1H-2,4,1-benzodithiazin-3-yl]-N-methylpropan-1-amine;
3-[1-(4-chlorophenyl)-2,2-dioxido-1H-2,4,1-benzodithiazin-3-yl]-N-methylpropan-1-amine;
3-[1-(3-chloro-4-fluorophenyl)-2,2-dioxido-1H-2,4,1-benzodithiazin-3-yl]-N-methylpropan-1-amine;
N-methyl-3-[1-(4-methylphenyl)-2,2-dioxido-1H-naphtho[2,3-e][1,3,4]oxathiazin-3-yl]propan-1-amine;
3-[1-(3-fluorophenyl)-2,2-dioxido-1H-naphtho[2,3-e][1,3,4]oxathiazin-3-yl]-N-methylpropan-1-amine;
3-[2,2-dioxido-1-phenyl-1H-naphtho[2,3-e][1,3,4]oxathiazin-3-yl]-N-methylpropan-1-amine;
3-[2,2-dioxido-1-phenyl-1H-naphtho[2,3-e][1,3,4]oxathiazin-3-yl]-N-methylpropan-1-amine;
3-[1-(2-fluorophenyl)-2,2-dioxido-3,4-dihydro-1H-2,1-benzothiazin-3-yl]-N-methylpropan-1-amine;
3-[1-(2-fluorophenyl)-2,2-dioxido-3,4-dihydro-1H-2,1-benzothiazin-3-yl]-N-methylpropan-1-amine;
3-[1-(2-fluorophenyl)-2,2-dioxido-3,4-dihydro-1H-2,1-benzothiazin-3-yl]-N-methylpropan-1-amine;
1-phenyl-3-(3-piperazin-1-ylpropyl)-1H-4,2,1-benzoxathiazine-2,2-dioxide;
3-[2-(1,4-diazepan-1-yl)ethyl]-1-phenyl-1H-4,2,1-benzoxathiazine-2,2-dioxide;
1-phenyl-3-(2-piperidin-1-ylethyl)-1H-4,2,1-benzoxathiazine 2,2-dioxide;
3-[2-(3,5-dimethylpiperazin-1-yl)ethyl]-1-phenyl-1H-4,2,1-benzoxathiazine 2,2-dioxide;
3-(2,2-dioxido-1-phenyl-1H-4,2,1-benzoxathiazin-3-yl)-N,N-dimethylpropan-1-amine;
3-(2,2-dioxido-1-phenyl-1H-4,2,1-benzoxathiazin-3-yl)-N-ethyl-N-methylpropan-1-amine;
3-(2,2-dioxido-1-phenyl-1H-4,2,1-benzoxathiazin-3-yl)-N,N-diethylpropan-1-amine;
2-{[3-(2,2-dioxido-1-phenyl-1H-4,2,1-benzoxathiazin-3-yl)propyl](ethyl)amino}ethanol;
3-(2,2-dioxido-1-phenyl-1H-4,2,1-benzoxathiazin-3-yl)-N-isopropyl-N-methylpropan-1-amine;
N-[3-(2,2-dioxido-1-phenyl-1H-4,2,1-benzoxathiazin-3-yl)propyl]-N-methylcyclohexanamine;
1-phenyl-3-(2-pyrrolidin-1-yl-ethyl)-1H-benzo[1,3,4]oxathiazine 2,2-dioxide;
3-[2-(4-methyl-piperazin-1-yl)-ethyl]-1-phenyl-1H-benzo[1,3,4]oxathiazine 2,2-dioxide;
N-butyl-[2-(2,2-dioxo-1-phenyl-2,3-dihydro-1H-2λ⁶-benzo[1,3,4]oxathiazin-3-yl)-ethyl]-amine;
3-[1-(2-fluorophenyl)-2,2-dioxido-3,4-dihydro-1H-2,1-benzothiazin-3-yl]-N-methylpropan-1-amine;
3-[6-fluoro-1-(4-fluoro-2-methylphenyl)-2,2-dioxido-3,4-dihydro-1H-2,1-benzothiazin-3-yl]-N-methylpropan-1-amine;
1-(2,2-dioxido-1-phenyl-1H-4,2,1-benzoxathiazin-3-yl)-3-(methylamino)propan-2-ol;
3-[1-(2,4-difluorophenyl)-2,2-dioxido-3,4-dihydro-1H-2,1-benzothiazin-3-yl]-N-methylpropan-1-amine;
2-(2,2-dioxido-1-phenyl-1H-4,2,1-benzoxathiazin-3-yl)ethanamine;
3-(6-fluoro-2,2-dioxido-1-phenyl-1H-4,2,1-benzoxathiazin-3-yl)-N-methylpropan-1-amine;
3-[6-fluoro-1-(2-fluorophenyl)-2,2-dioxido-3,4-dihydro-1H-2,1-benzothiazin-3-yl]-N-methylpropan-1-amine;
3-[1-(2,4-difluorophenyl)-6-fluoro-2,2-dioxido-3,4-dihydro-1H-2,1-benzothiazin-3-yl]-N-methylpropan-1-amine;
1-(2,2-dioxido-1-phenyl-3,4-dihydro-1H-2,1-benzothiazin-3-yl)-3-(methylamino)propan-1-ol;
2-(6-fluoro-2,2-dioxido-1-phenyl-1H-4,2,1-benzoxathiazin-3-yl)-N-methylethanamine;
3-[1-(2,6-difluorophenyl)-2,2-dioxido-3,4-dihydro-1H-2,1-benzothiazin-3-yl]-N-methylpropan-1-amine;
3-(2,2-dioxido-1-pyridin-3-yl-3,4-dihydro-1H-2,1-benzothiazin-3-yl)-N-methylpropan-1-amine;
3-(2,2-dioxido-1-pyridin-2-yl-3,4-dihydro-1H-2,1-benzothiazin-3-yl)-N-methylpropan-1-amine;
3-[6-fluoro-1-(2-fluorophenyl)-2,2-dioxido-1H-4,2,1-benzoxathiazin-3-yl]-N-methylpropan-1-amine;
2-[(2,2-dioxido-1-phenyl-3,4-dihydro-1H-2,1-benzothiazin-3-yl)methyl]-N-methylprop-2-en-1-amine;
3-(2,2-dioxido-1-pyridin-4-yl-3,4-dihydro-1H-2,1-benzothiazin-3-yl)-N-methylpropan-1-amine;
1-(2,2-dioxido-1-phenyl-1H-4,2,1-benzoxathiazin-3-yl)-N,N-dimethylmethanamine;
1-phenyl-3-(piperazin-1-ylmethyl)-1H-4,2,1-benzoxathiazine 2,2-dioxide;
N-[(2,2-dioxido-1-phenyl-1H-4,2,1-benzoxathiazin-3-yl)methyl]ethanamine;
N-[(2,2-dioxido-1-phenyl-1H-4,2,1-benzoxathiazin-3-yl)methyl]ethane-1,2-diamine;
N′-[(2,2-dioxido-1-phenyl-1H-4,2,1-benzoxathiazin-3-yl)methyl]-N,N-dimethylethane-1,2-diamine;
N-[(2,2-dioxido-1-phenyl-1H-4,2,1-benzoxathiazin-3-yl)methyl]-N,N′-dimethylethane-1,2-diamine;
N-[(2,2-dioxido-1-phenyl-1H-4,2,1-benzoxathiazin-3-yl)methyl]-N,N′,N′-trimethylethane-1,2-diamine;
3-[(4-methyl piperazin-1-yl)methyl]-1-phenyl-1H-4,2,1-benzoxathiazine 2,2-dioxide;
3-[(3,5-dimethylpiperazin-1-yl)methyl]-1-phenyl-1H-4,2,1-benzoxathiazine 2,2-dioxide;
3-(2,5-diazabicyclo[2.2.1]hept-2-ylmethyl)-1-phenyl-1H-4,2,1-benzoxathiazine 2,2-dioxide;
1-(2,2-dioxido-1-phenyl-1H-4,2,1-benzoxathiazin-3-yl)-N-methylmethanamine;
3-[1-(2,3-difluorophenyl)-6-fluoro-2,2-dioxido-3,4-dihydro-1H-2,1-benzothiazin-3-yl]-N-methylpropan-1-amine;
3-[6-fluoro-1-(2-fluoro-4-methylphenyl)-2,2-dioxido-3,4-dihydro-1H-2,1-benzothiazin-3-yl]-N-methylpropan-1-amine;
3-[1-(2,5-difluorophenyl)-6-fluoro-2,2-dioxido-3,4-dihydro-1H-2,1-benzothiazin-3-yl]-N-methylpropan-1-amine;
3-[1-(2,6-difluorophenyl)-6-fluoro-2,2-dioxido-3,4-dihydro-1H-2,1-benzothiazin-3-yl]-N-methylpropan-1-amine;
3-[1-(2-chlorophenyl)-6-fluoro-2,2-dioxido-3,4-dihydro-1H-2,1-benzothiazin-3-yl]-N-methylpropan-1-amine;
3-[6-fluoro-1-(2-methylphenyl)-2,2-dioxido-3,4-dihydro-1H-2,1-benzothiazin-3-yl]-N-methylpropan-1-amine;
2-{6-fluoro-3-[3-(methylamino)propyl]-2,2-dioxido-3,4-dihydro-1H-2,1-benzothiazin-1-yl}benzonitrile;
3-[6-fluoro-1-(2-methoxyphenyl)-2,2-dioxido-3,4-dihydro-1H-2,1-benzothiazin-3-yl]-N-methylpropan-1-amine;
3-[6-fluoro-1-(4-fluoro-2-methylphenyl)-2,2-dioxido-3,4-dihydro-1H-2,1-benzothiazin-3-yl]-N-methylpropan-1-amine;
3-(6-fluoro-2,2-dioxido-1-phenyl-3,4-dihydro-1H-2,1-benzothiazin-3-yl)-N-methylpropan-1-amine; and
3-[6-chloro-1-(2-fluorophenyl)-2,2-dioxido-3,4-dihydro-1H-2,1-benzothiazin-3-yl]-N-methylpropan-1-amine
   and
   pharmaceutically acceptable salts thereof, particularly the hydrochloride and dihydrochloride salts thereof.

Particularly preferred compounds of formula I include:
N-methyl-3-[(3R)-3-methyl-2,2-dioxido-1-phenyl-3,4-dihydro-1H-2,1-benzothiazin-3-yl]propan-1-amine;
3-[(3S)-2,2-dioxido-1-phenyl-3,4-dihydro-1H-2,1-benzothiazin-3-yl]-N-methylpropan-1-amine;
3-[(3R)-2,2-dioxido-1-phenyl-3,4-dihydro-1H-2,1-benzothiazin-3-yl]-N-methylpropan-1-amine;
3-[(3S)-2,2-dioxido-1-phenyl-1H-4,2,1-benzoxathiazin-3-yl]-N-methylpropan-1-amine;
3-[(3R)-2,2-dioxido-1-phenyl-1H-4,2,1-benzoxathiazin-3-yl]-N-methylpropan-1-amine;
3-[(3S)-2,2-dioxido-1-phenyl-1H-naphtho[2,3-e][1,3,4]oxathiazin-3-yl]-N-methylpropan-1-amine;
3-[(3R)-2,2-dioxido-1-phenyl-1H-naphtho[2,3-e][1,3,4]oxathiazin-3-yl]-N-methylpropan-1-amine;
3-[(3S)-1-(2-fluorophenyl)-2,2-dioxido-3,4-dihydro-1H-2,1-benzothiazin-3-yl]-N-methylpropan-1-amine;
3-[(3R)-1-(2-fluorophenyl)-2,2-dioxido-3,4-dihydro-1H-2,1-benzothiazin-3-yl]-N-methylpropan-1-amine;
3-[(3S)-1-(2-fluorophenyl)-2,2-dioxido-3,4-dihydro-1H-2,1-benzothiazin-3-yl]-N-methylpropan-1-amine;
3-[(3R)-1-(2-fluorophenyl)-2,2-dioxido-3,4-dihydro-1H-2,1-benzothiazin-3-yl]-N-methylpropan-1-amine;
3-[(3S)-6-fluoro-1-(4-fluoro-2-methylphenyl)-2,2-dioxido-3,4-dihydro-1H-2,1-benzothiazin-3-yl]-N-methylpropan-1-amine;
3-[(3R)-6-fluoro-1-(4-fluoro-2-methyl phenyl)-2,2-dioxido-3,4-dihydro-1H-2,1-benzothiazin-3-yl]-N-methylpropan-1-amine;
(2S)-1-[(3R)-2,2-dioxido-1-phenyl-1H-4,2,1-benzoxathiazin-3-yl]-3-(methylamino)propan-2-ol;
(2R)-1-[(3R)-2,2-dioxido-1-phenyl-1H-4,2,1-benzoxathiazin-3-yl]-3-(methylamino)propan-2-ol;
(2R)-1-[(3S)-2,2-dioxido-1-phenyl-1H-4,2,1-benzoxathiazin-3-yl]-3-(methylamino)propan-2-ol;
(2S)-1-[(3S)-2,2-dioxido-1-phenyl-1H-4,2,1-benzoxathiazin-3-yl]-3-(methylamino)propan-2-ol;
3-[(3S)-1-(2,4-difluorophenyl)-2,2-dioxido-3,4-dihydro-1H-2,1-benzothiazin-3-yl]-N-methylpropan-1-amine;
3-[(3R)-1-(2,4-difluorophenyl)-2,2-dioxido-3,4-dihydro-1H-2,1-benzothiazin-3-yl]-N-methylpropan-1-amine;
2-[(3S)-2,2-dioxido-1-phenyl-1H-4,2,1-benzoxathiazin-3-yl]ethanamine;
2-[(3R)-2,2-dioxido-1-phenyl-1H-4,2,1-benzoxathiazin-3-yl]ethanamine;
2-[(3S)-2,2-dioxido-1-phenyl-1H-4,2,1-benzoxathiazin-3-yl]-N-methylethanamine;
2-[(3R)-2,2-dioxido-1-phenyl-1H-4,2,1-benzoxathiazin-3-yl]-N-methylethanamine;
3-[(3S)-6-fluoro-2,2-dioxido-1-phenyl-1H-4,2,1-benzoxathiazin-3-yl]-N-methylpropan-1-amine;
3-[(3R)-6-fluoro-2,2-dioxido-1-phenyl-1H-4,2,1-benzoxathiazin-3-yl]-N-methylpropan-1-amine;
3-[(3S)-6-fluoro-1-(2-fluorophenyl)-2,2-dioxido-3,4-dihydro-1H-2,1-benzothiazin-3-yl]-N-methylpropan-1-amine;
3-[(3R)-6-fluoro-1-(2-fluorophenyl)-2,2-dioxido-3,4-dihydro-1H-2,1-benzothiazin-3-yl]-N-methylpropan-1-amine;
3-[(3S)-1-(2,4-difluorophenyl)-6-fluoro-2,2-dioxido-3,4-dihydro-1H-2,1-benzothiazin-3-yl]-N-methylpropan-1-amine;
3-[(3R)-1-(2,4-difluorophenyl)-6-fluoro-2,2-dioxido-3,4-dihydro-1H-2,1-benzothiazin-3-yl]-N-methylpropan-1-amine;
(1S)-1-[(3S)-2,2-dioxido-1-phenyl-3,4-dihydro-1H-2,1-benzothiazin-3-yl]-3-(methylamino)propan-1-ol;
(1R)-1-[(3S)-2,2-dioxido-1-phenyl-3,4-dihydro-1H-2,1-benzothiazin-3-yl]-3-(methylamino)propan-1-ol;
(1R)-1-[(3R)-2,2-dioxido-1-phenyl-3,4-dihydro-1H-2,1-benzothiazin-3-yl]-3-(methylamino)propan-1-ol;
(1S)-1-[(3R)-2,2-dioxido-1-phenyl-3,4-dihydro-1H-2,1-benzothiazin-3-yl]-3-(methylamino)propan-1-ol;
2-[(3S)-6-fluoro-2,2-dioxido-1-phenyl-1H-4,2,1-benzoxathiazin-3-yl]-N-methylethanamine;
2-[(3R)-6-fluoro-2,2-dioxido-1-phenyl-1H-4,2,1-benzoxathiazin-3-yl]-N-methylethanamine;
3-[(3S)-1-(2,6-difluorophenyl)-2,2-dioxido-3,4-dihydro-1H-2,1-benzothiazin-3-yl]-N-methylpropan-1-amine;
3-[(3R)-1-(2,6-difluorophenyl)-2,2-dioxido-3,4-dihydro-1H-2,1-benzothiazin-3-yl]-N-methylpropan-1-amine;
3-[(3S)-2,2-dioxido-1-pyridin-3-yl-3,4-dihydro-1H-2,1-benzothiazin-3-yl]-N-methylpropan-1-amine;
3-[(3R)-2,2-dioxido-1-pyridin-3-yl-3,4-dihydro-1H-2,1-benzothiazin-3-yl]-N-methylpropan-1-amine;
3-[(3S)-2,2-dioxido-1-pyridin-2-yl-3,4-dihydro-1H-2,1-benzothiazin-3-yl]-N-methylpropan-1-amine;
3-[(3R)-2,2-dioxido-1-pyridin-2-yl-3,4-dihydro-1H-2,1-benzothiazin-3-yl]-N-methylpropan-1-amine;
3-[(3S)-1-(2,3-difluorophenyl)-6-fluoro-2,2-dioxido-3,4-dihydro-1H-2,1-benzothiazin-3-yl]-N-methylpropan-1-amine;
3-[(3R)-1-(2,3-difluorophenyl)-6-fluoro-2,2-dioxido-3,4-dihydro-1H-2,1-benzothiazin-3-yl]-N-methylpropan-1-amine;
3-[(3S)-6-fluoro-1-(2-fluoro-4-methylphenyl)-2,2-dioxido-3,4-dihydro-1H-2,1-benzothiazin-3-yl]-N-methylpropan-1-amine;
3-[(3R)-6-fluoro-1-(2-fluoro-4-methyl phenyl)-2,2-dioxido-3,4-dihydro-1H-2,1-benzothiazin-3-yl]-N-methylpropan-1-amine;
3-[(3S)-1-(2,5-difluorophenyl)-6-fluoro-2,2-dioxido-3,4-dihydro-1H-2,1-benzothiazin-3-yl]-N-methylpropan-1-amine;
3-[(3R)-1-(2,5-difluorophenyl)-6-fluoro-2,2-dioxido-3,4-dihydro-1H-2,1-benzothiazin-3-yl]-N-methylpropan-1-amine;
3-[(3S)-1-(2,6-difluorophenyl)-6-fluoro-2,2-dioxido-3,4-dihydro-1H-2,1-benzothiazin-3-yl]-N-methylpropan-1-amine;
3-[(3R)-1-(2,6-difluorophenyl)-6-fluoro-2,2-dioxido-3,4-dihydro-1H-2,1-benzothiazin-3-yl]-N-methylpropan-1-amine;
3-[(3S)-1-(2-chlorophenyl)-6-fluoro-2,2-dioxido-3,4-dihydro-1H-2,1-benzothiazin-3-yl]-N-methylpropan-1-amine;
3-[(3R)-1-(2-chlorophenyl)-6-fluoro-2,2-dioxido-3,4-dihydro-1H-2,1-benzothiazin-3-yl]-N-methylpropan-1-amine;
3-[(3S)-6-fluoro-1-(2-methylphenyl)-2,2-dioxido-3,4-dihydro-1H-2,1-benzothiazin-3-yl]-N-methylpropan-1-amine;
3-[(3R)-6-fluoro-1-(2-methylphenyl)-2,2-dioxido-3,4-dihydro-1H-2,1-benzothiazin-3-yl]-N-methylpropan-1-amine;
2-{(3S)-6-fluoro-3-[3-(methylamino)propyl]-2,2-dioxido-3,4-dihydro-1H-2,1-benzothiazin-1-yl}benzonitrile;
2-{(3R)-6-fluoro-3-[3-(methylamino)propyl]-2,2-dioxido-3,4-dihydro-1H-2,1-benzothiazin-1-yl}benzonitrile;
3-[(3S)-6-fluoro-1-(2-methoxyphenyl)-2,2-dioxido-3,4-dihydro-1H-2,1-benzothiazin-3-yl]-N-methylpropan-1-amine;
3-[(3R)-6-fluoro-1-(2-methoxyphenyl)-2,2-dioxido-3,4-dihydro-1H-2,1-benzothiazin-3-yl]-N-methylpropan-1-amine;
3-[(3S)-6-fluoro-1-(4-fluoro-2-methylphenyl)-2,2-dioxido-3,4-dihydro-1H-2,1-benzothiazin-3-yl]-N-methylpropan-1-amine;
3-[(3R)-6-fluoro-1-(4-fluoro-2-methyl phenyl)-2,2-dioxido-3,4-dihydro-1H-2,1-benzothiazin-3-yl]-N-methylpropan-1-amine;
3-[(3S)-6-fluoro-2,2-dioxido-1-phenyl-3,4-dihydro-1H-2,1-benzothiazin-3-yl]-N-methylpropan-1-amine;
3-[(3R)-6-fluoro-2,2-dioxido-1-phenyl-3,4-dihydro-1H-2,1-benzothiazin-3-yl]-N-methylpropan-1-amine;
3-[(3S)-6-chloro-1-(2-fluorophenyl)-2,2-dioxido-3,4-dihydro-1H-2,1-benzothiazin-3-yl]-N-methylpropan-1-amine; and
3-[(3R)-6-chloro-1-(2-fluorophenyl)-2,2-dioxido-3,4-dihydro-1H-2,1-benzothiazin-3-yl]-N-methylpropan-1-amine
   and
   pharmaceutically acceptable salts thereof, particularly the hydrochloride and dihydrochloride salts thereof.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972), the entire disclosures of which are herein incorporated by reference.

The present invention includes prodrugs of the compounds of formula I. “Prodrug,” as used herein, means a compound which is convertible in vivo by metabolic means (e.g. by hydrolysis) to a compound of formula I. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975), the entire disclosures of which are herein incorporated by reference.

Further, the compounds of formula I may exist in unsolvated as well as in solvated forms with pharmaceutically acceptable solvents such as water, ethanol, and the like. In general, the solvated forms are considered equivalent to the unsolvated forms for the purpose of the present invention.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

The present invention provides a process for the preparation of a compound according to formula Ia

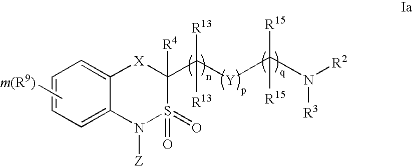

or a pharmaceutically acceptable salt thereof;
wherein:
m is an integer from 0 to 4;
n is an integer from 0 to 2;
p is an integer from 0 to 1;
q is an integer from 1 to 2;
v is an integer from 0 to 2;
X is C(R¹¹)₂, N(R¹²), O, or S(O)ᵥ;
Y is C(R¹¹)₂, N(R¹²), O, or

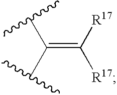

Z is H or an aryl substituted with 0-3 R⁵ or heteroaryl substituted with 0-3 R⁵;
R² is H, straight or branched C₁-C₆ alkyl, C₃-C₆ cycloalkyl, or aryl-C₁-C₆ alkyl where said aryl portion is substituted with 0-3 R⁶;
R³ is H, straight or branched C₁-C₆ alkyl, C₁-C₆ alkanol, C₃-C₆ cycloalkyl, or aryl-C₁-C₆ alkyl where said aryl portion is substituted with 0-3 R⁷; or
R² and R³, together with the nitrogen through which they are attached, form a mono- or bicyclic heterocyclic ring of 3 to 12 ring atoms, where one carbon may be optionally replaced with N, O, S, or SO₂, and where any carbon ring atom may be optionally substituted with C₁-C₄ alkyl, F, or CF₃ or where any additional N atom may be optionally substituted with C₁-C₄ alkyl, with the proviso that if Y is O or N(R¹²), q is not 2;
R⁴ is H, straight or branched C₁-C₆ alkyl, aryl substituted with 0-3 R⁸, aryl-C₁-C₆ alkyl where said aryl portion is substituted with 0-3 R⁸, heteroaryl substituted with 0-3 R⁸, or heteroaryl-C₁-C₆ alkyl where said heteroaryl portion is substituted with 0-3 R⁸;
R⁵ is, independently at each occurrence, C₁-C₆ alkyl, alkoxy, halo, CF₃, OCF₃, hydroxy, alkanoyloxy, nitro, nitrile, alkenyl, alkynyl, aryl substituted with 0-3 R⁵ or heteroaryl substituted with 0-3 R⁵;
R⁶ is, independently at each occurrence, C₁-C₄ alkyl, halo or H;
R⁷ is, independently at each occurrence, C₁-C₄ alkyl, halo or H;
R⁸ is, independently at each occurrence, C₁-C₄ alkyl, halo or H;
R⁹ is, independently at each occurrence, C₁-C₆ alkyl, alkoxy, halo, H, CF₃, OCF₃, hydroxy, alkanoyloxy, nitro, nitrile, alkenyl, alkynyl, aryl substituted with 0-3 R¹⁰, heteroaryl substituted with 0-3 R¹⁰, alkylsulfoxide, alkylsulfone, alkylsulfonamide, phenylsulfonamide substituted with 0-3 R¹⁰, alkylamido, or arylamido substituted with 0-3 R¹⁰; or
two adjacent R⁹, together with the ring atoms to which they are attached, form a fused ring of 5 or 6 ring atoms;
R¹⁰ is, independently at each occurrence, H, C₁-C₆ alkyl, or halo;
R¹¹ is, independently at each occurrence, H, C₁-C₆ alkyl, halo, hydroxy, O—(C₁-C₆ alkyl), or aryl substituted with 0-3 R⁸;
R¹² is, independently at each occurrence, H, C₁-C₆ alkyl, or aryl substituted with 0-3 R⁸;
R¹³ is, independently at each occurrence, H, C₁-C₆ alkyl, halide, hydroxy, or aryl substituted with 0-3 R¹⁶;
R¹⁵ is, independently at each occurrence, H, C₁-C₄ alkyl or halide;
R¹⁶ is, independently at each occurrence, H, C₁-C₄ alkyl, or halo; and
R¹⁷ is, independently at each occurrence, H or C₁-C₄ alkyl;

which process comprises:

(a) performing either of i) or ii) below:

i) coupling a compound according to formula II below with an electrophile according to formula a below:

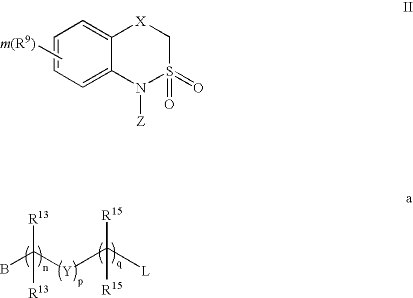

thereby forming a compound according to formula III below:

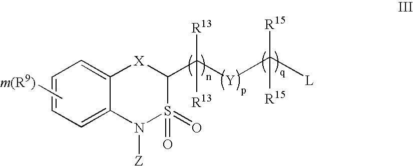

ii) coupling a compound according to formula II hereinabove with an allyl halide according to formula b below:

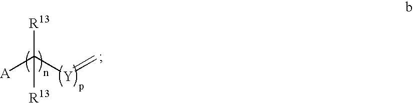

subjecting the compound formed by the coupling with the allyl halide to hydroboration-oxidation to form an alcohol; and

activating the alcohol with an activating agent, thereby forming a compound according to formula III hereinabove;

wherein R⁹, R¹³, R¹⁵, m, n, p, q, Z, X and Y are as defined hereinabove for formula Ia, B is a functional group, L is a leaving group and A is a halogen; and

b) reacting the compound of formula III formed in a) with an amine:

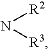

thereby forming a compound according to formula Ia hereinabove, wherein R² and R³ of the amine are as defined hereinabove for formula Ia.

In some embodiments, Z of the compound of formula II is H, and the process further comprises subjecting said compound to an N-arylation reaction prior to or subsequent to said coupling i) or said coupling ii).

In some other embodiments, L of the compound of formula a is a chloride, a bromide, an iodide, a mesylate or a tosylate derived from the activating agent.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991, the entire disclosure of which is herein incorporated by reference.

Compounds of the present invention are suitably prepared in accordance with the following general description and specific examples. Variables used are as defined for formula I, unless otherwise noted. The reagents used in the preparation of the compounds of this invention can be either commercially obtained or can be prepared by standard procedures described in the literature. In accordance with this invention, compounds of formula I may be produced by the following reaction schemes (Schemes 1 to 13).

Compounds containing the carbocyclic core (X═C(R¹¹)₂) may be prepared according to Scheme 1.

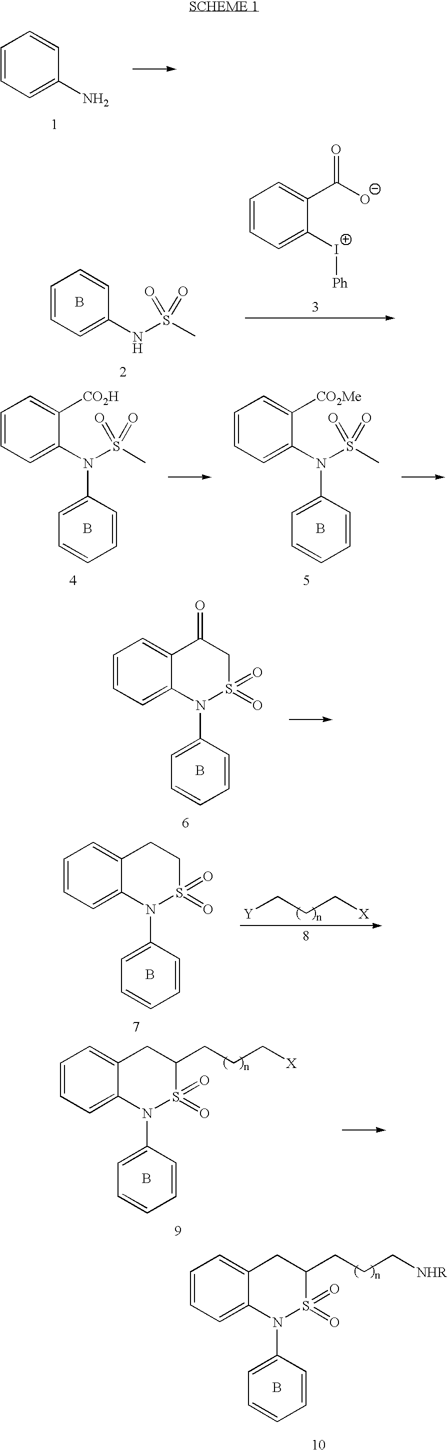

Thus aniline (1) (optionally substituted or heteroaromatic for example) may be reacted with methane sulfonyl chloride or anhydride under basic conditions to afford sulfonamide (2). Suitable solvents may include chloroform, dichloromethane, pyridine, THF, and toluene for example. Bases may include triethylamine, pyridine, sodium carbonate, diisopropylethylamine for example. The sulfonamide (2) may then be reacted with a strong base (sodium hydride) in an ethereal solvent and then treated with diphenyliodonium carboxylate to afford compound (4). Compound (4) can then be esterified under standard conditions (e.g. methyl iodide, potassium carbonate, DMF) to give the ester (5). Ester (5) may then be reacted with a base such as sodium bis(trimethylsilyl)amide in a solvent such as N,N-dimethylacetamide to afford compound (6). Compound (6) can then be reduced to afford compound (7) (e.g. trialkylsilane/trifluoroacteic acid). Compound (7) may then be treated with a base (e.g. lithium or sodium bis(trimethylsilyl)amide) in a suitable solvent (e.g. THF or DMF) and then reacted with a bifunctional electrophile (8) to afford compound (9), which may then be reacted with a primary or secondary amine, in a solvent such as an alcohol or an ether, to afford compound (10).

Alternatively compound (7) may be treated with a base (e.g. lithium or sodium bis(trimethylsilyl)amide) in a suitable solvent (e.g. THF or DMF) and then reacted with allyl bromide to afford compound (11), as shown in Scheme 2. Hydroboration of compound (11) (e.g. catechol borane or 9-borabicylo[3.3.1]nonane in a solvent such as THF) followed by an oxidative workup procedure would provide alcohol (12). This compound could then be activated (e.g. chlorinated [NCS/Ph₃P], mesylated [MeSO₂Cl/pyridine] or tosylated [TsCl/pyridine}) and subsequently treated with a primary or secondary amine, in a solvent such as an alcohol or an ether, to afford compound (10).

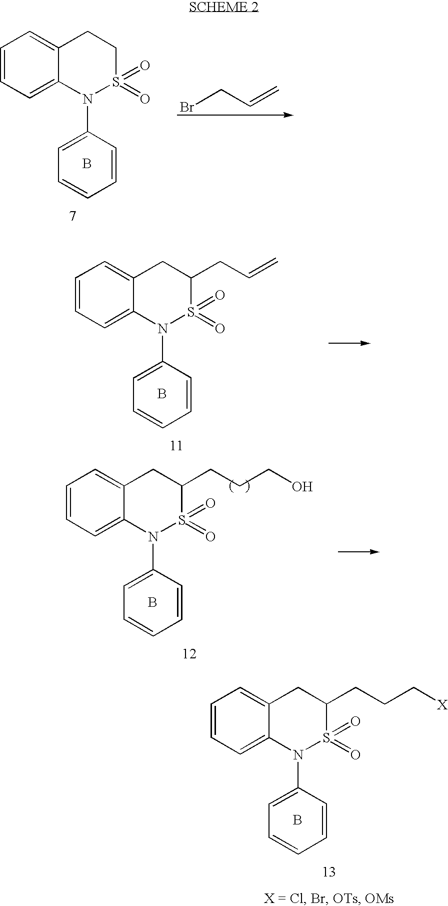

An alternate entry into ring system (7) may also be envisioned, as shown in Scheme 3.

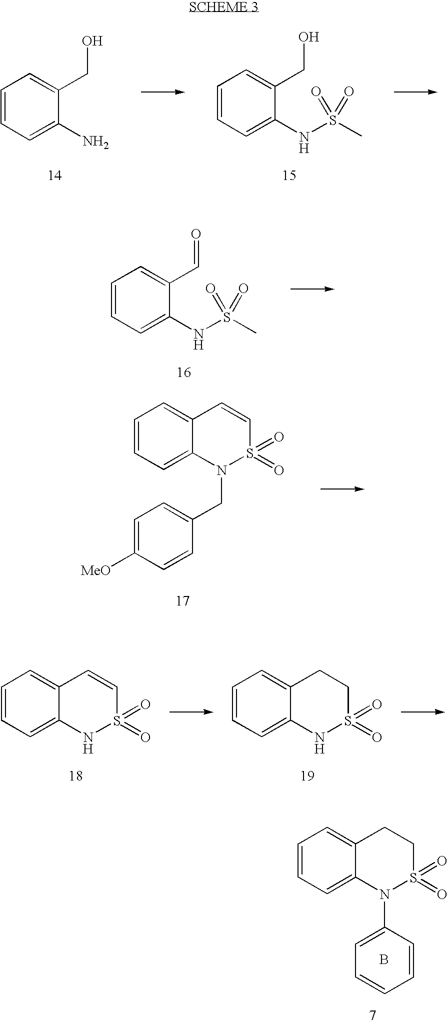

Thus the aniline (14) may be reacted with methane sulfonyl chloride or anhydride to afford sulfonamide (15). Treatment of (15) under oxidizing conditions (e.g. MnO₂ in dichloromethane, or Swern oxidation) would afford the aldehyde (16). Treatment of aldehyde (16) with base (e.g. sodium hydride followed by p-methoxybenzyl chloride) would afford an intermediate protected derivative which could then be treated with a strong base (e.g. lithium or sodium bistrimethylsilylamide or lithium diisopropylamide in a suitable solvent e.g. THF or DMF) would then afford compound (17). Removal of the protecting group under acidic conditions (TFA or HCl) would then afford compound (18) which could be reduced (e.g. hydrogenation over a palladium catalyst in a solvent such as methanol, dichloromethane or THF) to afford (19). Compound (19) could then be coupled with an aromatic boronic acid in the presence of a copper catalyst to give system (7). This methodology may then provide access to compounds (7) wherein pendant ring system “B” may be further substituted or indeed may be a heteroaromatic system.

Compounds containing the X═O may be prepared according to Scheme 4.

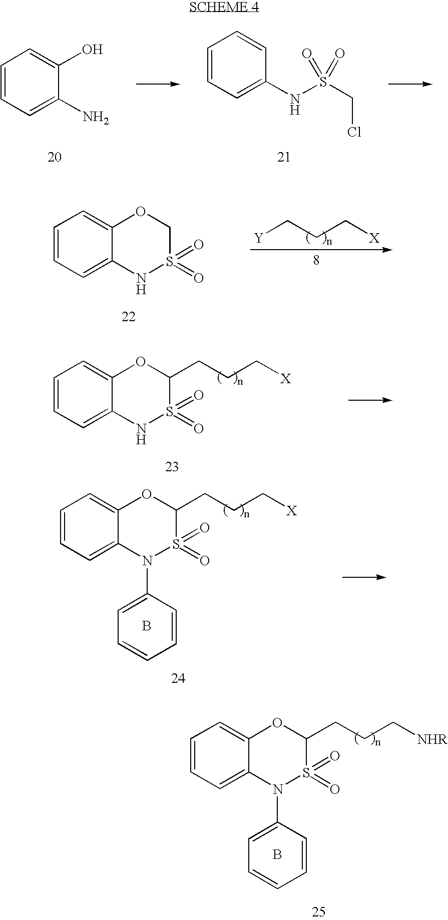

The amino phenol (20) may be reacted with methane sulfonyl chloride or anyhydride in the presence of a suitable base (e.g. pyridine or a tertiary amine base) in a suitable solvent such as THF or dichloromethane to give the sulfonamide (21). Reaction of compound (21) under basic conditions (potassium carbonate in methanol for example) would give ring system (22). Reaction of compound (22) with electrophile (8) (e.g. lithium or sodium bis(trimethylsilyl)amide) in a suitable solvent (e.g. THF or DMF) would afford compound (23). Treatment of compound (23) with a phenyl boronic acid in the presence of copper(II) acetate and pyridine N-oxide and triethylamine, would then afford compound (24), provided that group X were compatible to the conditions employed. (Other conditions for transformations of this type are also available (Deng, Wei, et al., Copper-catalyzed cross-coupling of sulfonamides with aryl iodides and bromides facilitated by amino acid ligands, Tetrahedron Letters (2005), 46(43), 7295-7298; Burton, G. et al., Palladium-Catalyzed Intermolecular Coupling of Aryl Chlorides and Sulfonamides under Microwave Irradiation. Organic Letters (2003), 5(23), 4373-4376; He, H. and Wu, Y-J., Copper-catalyzed N-arylation of sulfonamides with aryl bromides and iodides using microwave heating. Tetrahedron Letters (2003), 44(16), 3385-3386; Yin, J. and Buchwald, S. L. Pd-Catalyzed Intermolecular Amidation of Aryl Halides: The Discovery that Xantphos Can Be Trans-Chelating in a Palladium Complex. Journal of the American Chemical Society (2002), 124 (21), 6043-6048. Combs, A. P. and Rafalski, M. N-Arylation of Sulfonamides on Solid Supports. Journal of Combinatorial Chemistry (2000), 2(1), 29-32; Rafalski, M., et al., Cupric acetate-mediated N-arylation by arylboronic acids: Solid-supported C—N cross-coupling reaction. Book of Abstracts, 218th ACS National Meeting, New Orleans, Aug. 22-26 (1999), the entire disclosures of which are incorporated herein by reference). This would also apply to the thio ether series and also to the all carbon series. Thus group X could be a chloride, suitable for further homologation to the product of type (25) via treatment with a primary or secondary amine in a suitable solvent such as an alcohol or ether: the deprotected alcohol would then be activated via the methane or tosylsulfonate as described previously, prior to displacement with a suitable amine in an alcoholic solvent. Group X may also be an alcohol or protected alcohol (T. W. Greene and P. G. M. Wutts eds., Protective groups in organic synthesis, 3^{rd} ed., John Wiley and Sons, 1999), the entire disclosure of which is incorporated herein by reference. Group X may also be a protected amine for example a methyl-carbamic acid tert-butyl ester residue, which upon standard deprotection conditions (TFA or HCl in dioxane or water) would provide compound (25), where R=methyl.

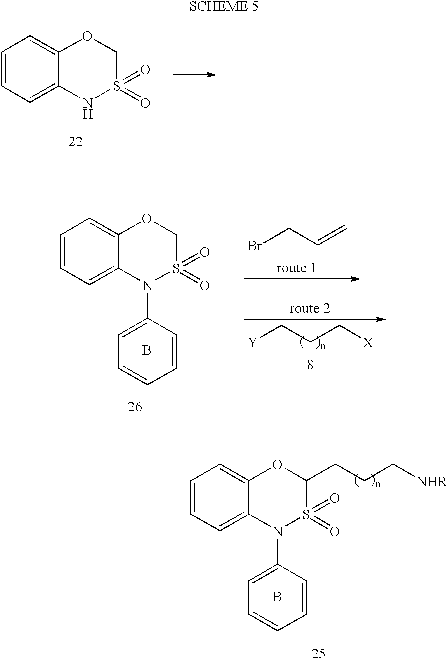

In an alternative, as shown in Scheme 5, compound (22) could first be reacted with a phenyl boronic acid in the presence of copper (II) acetate and pyridine N-oxide and triethylamine to afford compound (26). Compound (26) could then be reacted with allyl bromide and converted to the product (25) as per the conditions described above (Scheme 2) for the conversion of compound (7) through to target molecule (10). In analogy to the sequence described for the conversion of compound (7) through to compound (10) in Scheme 1, compound (26) through a similar sequence of manipulations could give compound (25) via the electrophile (8).

Compounds containing the X═S may be prepared according to Scheme 6 Compounds in the thioether class can be prepared using methodologies similar to those described above for the carbon and oxygen analogs.

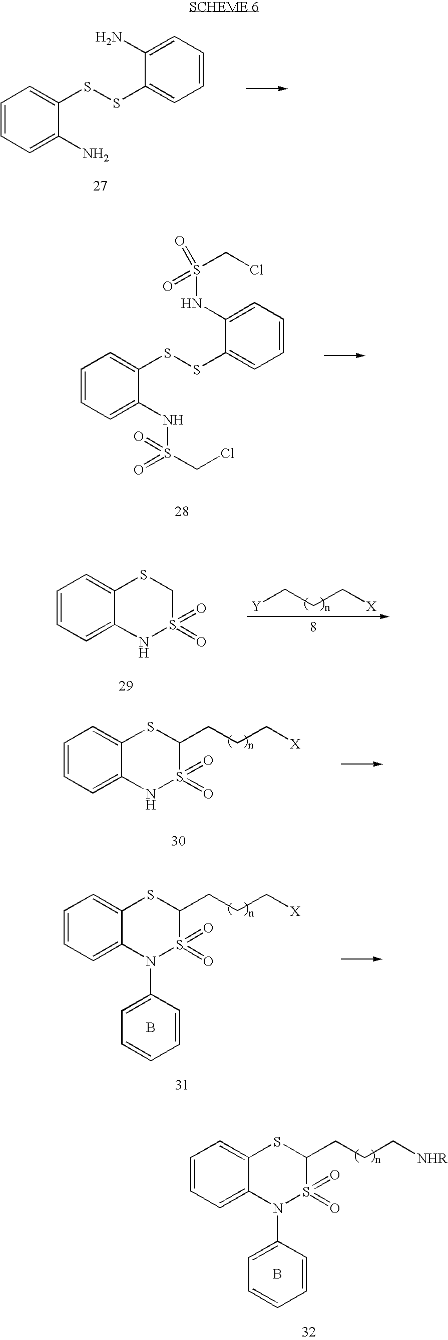

Compounds (29) may be prepared as described in Scheme 6 (See also WO 92/05164, incorporated herein by reference). Thus the bis-aniline (27) is treated with chloromethanesulfonyl chloride in a suitable solvent (e.g. THF or dichloromethane) in the presence of a base such as diisoproylethylamine, triethylamine, or pyridine, to give the sulfonamide (28). Reaction of (28) with sodium borohydride then gives compound (29). Compound (29) may then be elaborated via one of the previously described sequences. Hence reaction with electrophile (8) in the presence of a base (e.g. lithium or sodium bis(trimethylsilyl)amide) in a suitable solvent (e.g. THF or DMF) would afford compound (30). Treatment of compound (30) with a phenyl boronic acid in the presence of copper(II) acetate and pyridine N-oxide and triethylamine, would then afford compound (31). Reaction of compound (31) (X=chloride) with a suitable primary or secondary amine would then give the product (32).

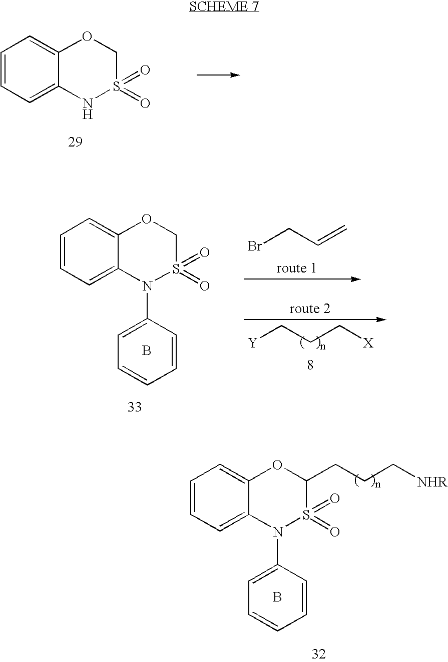

Alternatively, according to Scheme 7, compound (29) could first be converted into compound (33) followed by elaboration to the target (32) via route 1 (allylation, hydroboration, activation as tosylate or mesylate, then displacement with primary or secondary amine) or via route 2 (reaction with electrophile (8), followed by displacement of group X with a primary or secondary amine.

An alternative synthesis of the ethylamine side chain compounds (34) may also be employed, as shown in Scheme 8.

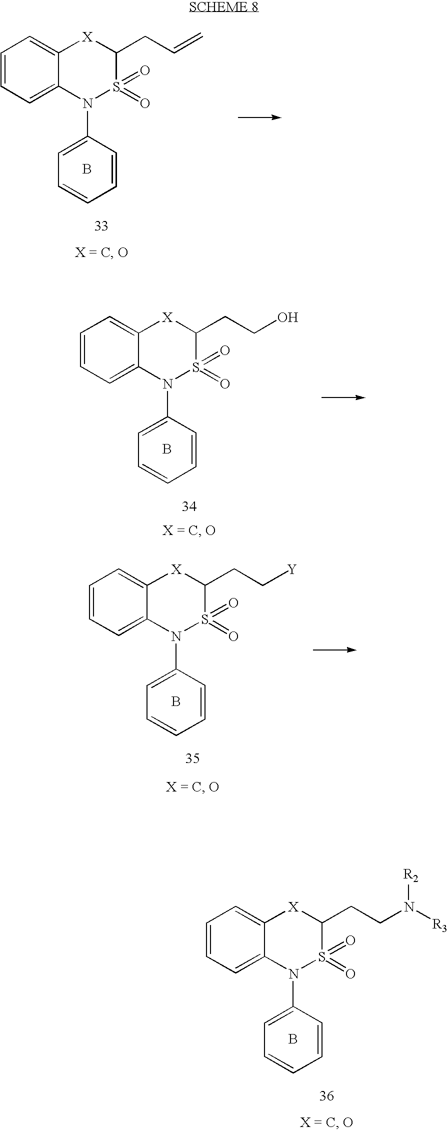

According to Scheme 8, allylic compounds (33) are first converted to alcohols (34). This transformation may be accomplished by reaction of (33) with ozone at low temperature in a suitable solvent such as methanol, followed by treatment with sodium borohydride. Alternatively combinations of sodium periodate and osmium tetroxide may also be employed. Subsequently compounds (34) may be activated (e.g. chlorinated [NCS/Ph₃P], mesylated [MeSO₂Cl/pyridine] or tosylated [TsCl/pyridine}) and subsequently treated with a primary or secondary amine, in a solvent such as an alcohol or an ether, to afford compounds (36).

Methods to incorporate the R⁴ group are described below in Scheme 9.

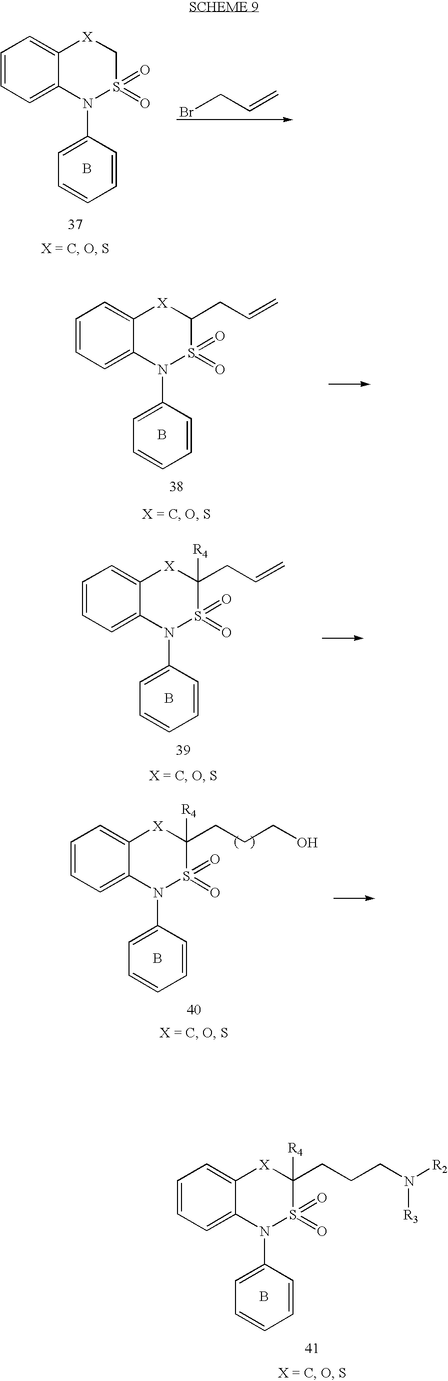

Compound (37) are first treated with base (e.g. lithium or sodium bis(trimethylsilyl)amide) in a suitable solvent (e.g. THF or DMF) and then reacted with allyl bromide to afford compounds (38). Treatment of compounds (32) with base (e.g. lithium or sodium bis(trimethylsilyl)amide) in a suitable solvent (e.g. THF or DMF) followed by reaction with an electrophile (e.g. an alkyl halide (iodide, or bromide) or a triflate, mesylate or tosylate, would provide access to products (39). Conversion of the allyl group in compounds (39) through hydroboration would provide the alcohols (40), which could then be activated (e.g. chlorinated [NCS/Ph₃P], mesylated [MeSO₂Cl/pyridine] or tosylated [TsCl/pyridine}) and subsequently treated with a primary or secondary amine, in a solvent such as an alcohol or an ether, to afford compounds (41).

An alternate route to ring system (7) may also be envisioned, Scheme 10.

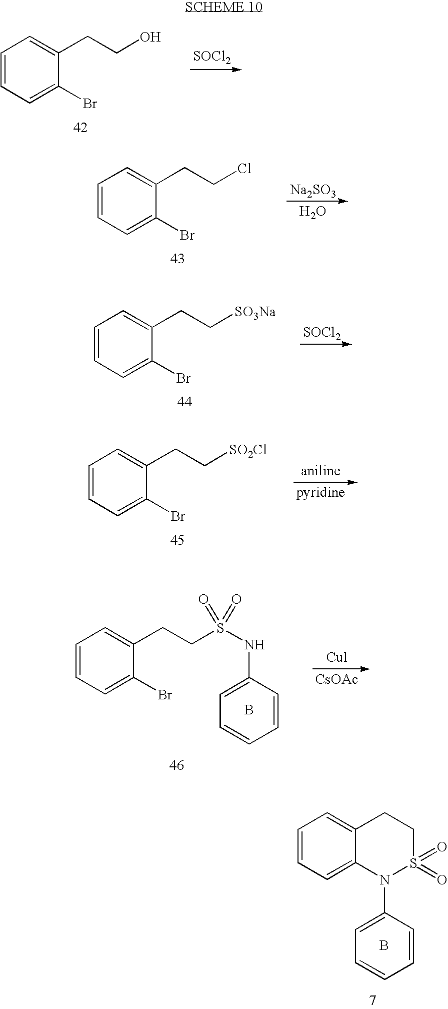

Thus the alcohol (42) is activated by standard means, for example conversion the chloride (43) via reaction with thionyl chloride. Alternative leaving groups would also include bromides, iodides, mesylates and tosylates, for example. Reaction of (43) with sodium sulfite then provides the salt (44) which can be converted to the acid chloride (45) by reaction with thionyl chloride. Treatment of (45) with an appropriately substituted aniline would then afford the amide (46). Ring closure of the amide (46) would then provide template (7). Suitable conditions for the ring closure include reaction with copper (I) iodide in the presence of cesium acetate.

A synthesis of the nitrogen containing ring system (X═N) could be executed using a similar ring closure, Scheme 11.

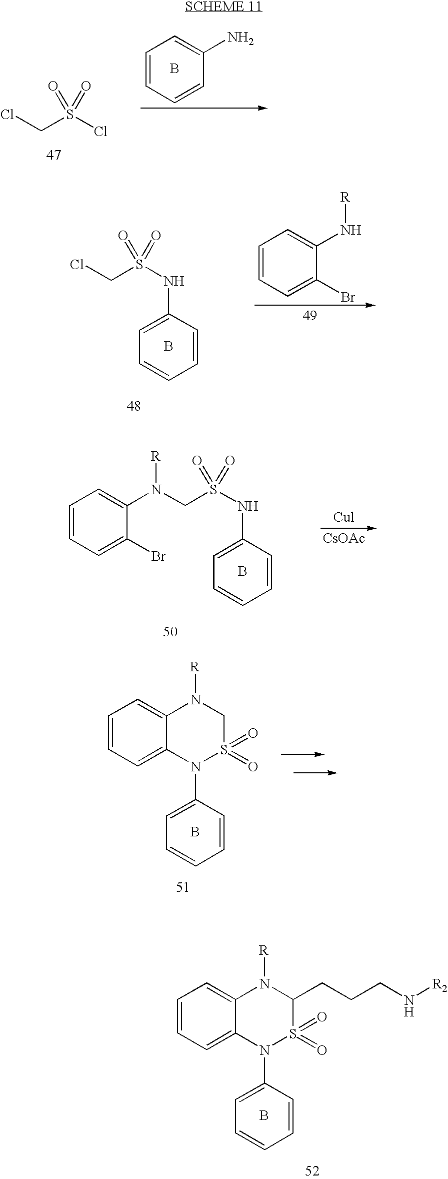

Thus reaction of chloromethylsulfonyl chloride with an appropriately substituted aniline would afford compound (48). 2-bromoaniline (49) would then be reacted with compound (48) to afford derivative (50). The R group present within (49) could be a hydrogen atom, or a suitable alkyl or protecting group (Greene, T. W. and Wuts, P. G. M., Protective Groups in Organic Synthesis 2d. Ed., Wiley & Sons, 1991), the entire disclosure of which is incorporated herein by reference. Depending upon the relative reactivities of (48) and (49), it may also be necessary to install a protecting group on the nitrogen atom of compound (48). A suitable protecting group could be a trimethylsilylethoxymethyl group, although other suitable groups could also be applied (Greene, T. W. and Wuts, P. G. M., Protective Groups in Organic Synthesis 2d. Ed., Wiley & Sons, 1991). Ring closure of compound (50) to (51) could then be accomplished by treatment with copper (I) iodide in the presence of cesium acetate. Compound (51) could then be elaborated into the target compounds (52) utilizing approaches described above.

A similar approach may be envisioned for the synthesis of the ether (X═O) core system (26), Scheme 12.

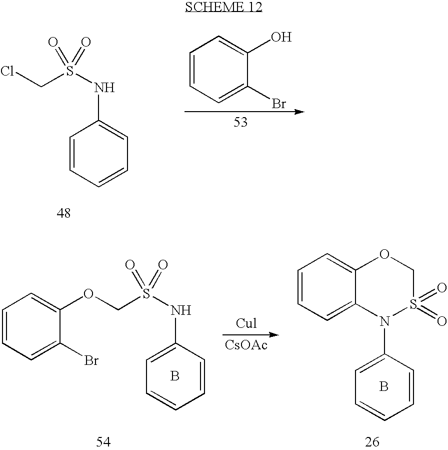

Thus reaction of an appropriately substituted reagent (48) with the phenol (53) would afford ether (54). Depending upon the conditions used and the relative reactivities of (48) and (53), the nitrogen atom of (48) may need to be protected. A suitable protecting group could be a trimethylsilylethoxymethyl group, although other suitable groups could also be applied (Greene, T. W. and Wuts, P. G. M., Protective Groups in Organic Synthesis 2d. Ed., Wiley & Sons, 1991). Treatment of compounds (54) with copper (I) iodide in the presence of cesium acetate would then afford ring system (26). Appropriate elaboration as described above would then provide the target compounds (25).

The higher oxidation states of I where X═S and v=1 or 2, may be accessed from intermediate (31), Scheme 13. Thus reaction of compound (31) with for example aqueous sodium periodate or 1 equivalent of a peracid such as m-chloroperoxybenzoic acid would provide intermediates (55) with v=1 (sulfoxide oxidation state). Further elaboration of (55) as above would then provide target compounds (56). Alternatively to access the higher oxidation state, compounds (31) could be treated with excess peracid, for example m-chloroperoxybenzoic acid to afford intermediates (57), or indeed further oxidation of (55) with m-chloroperoxybenzoic acid (for example, although other reagents are readily available from the literature) would also provide access to intermediates (57). Compounds (57) may then be converted into target compounds (58) as described above.

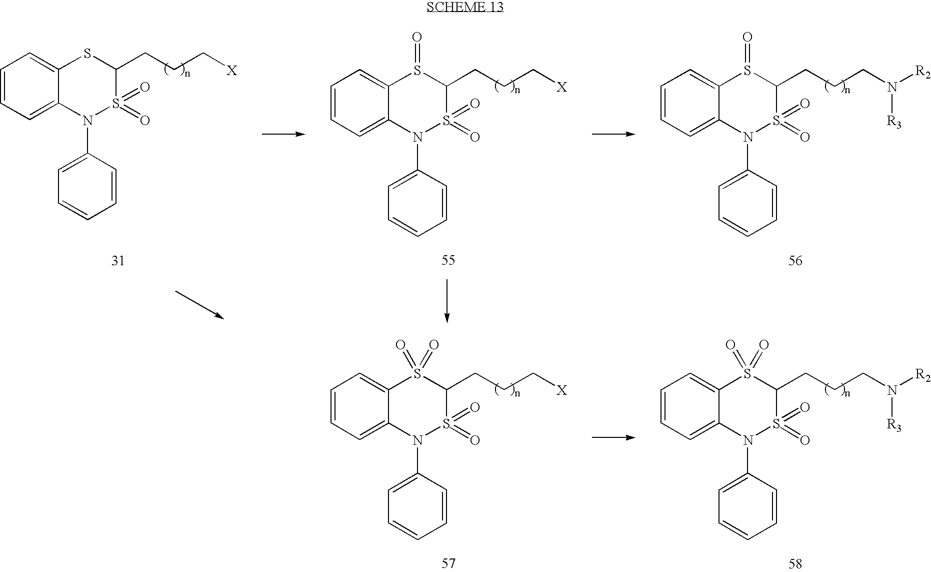

In other embodiments, the invention is directed to pharmaceutical compositions, comprising:

a. at least compound of formula I, or pharmaceutically acceptable salt thereof; and
b. at least one pharmaceutically acceptable carrier.

Generally, the compound of formula I, or a pharmaceutically acceptable salt thereof, will be present at a level of from about 0.1%, by weight, to about 90% by weight, based on the total weight of the pharmaceutical composition. Preferably, the compound of formula I, or a pharmaceutically acceptable salt thereof, will be present at a level of at least about 1%, by weight, based on the total weight of the pharmaceutical composition. More preferably, the compound of formula I, or a pharmaceutically acceptable salt thereof, will be present at a level of at least about 5%, by weight, based on the total weight of the pharmaceutical composition. Even more preferably, the compound of formula I or a pharmaceutically acceptable salt thereof will be present at a level of at least about 10%, by weight, based on the total weight of the pharmaceutical composition. Yet even more preferably, the compound of formula I, or a pharmaceutically acceptable salt thereof, will be present at a level of at least about 25%, by weight, based on the total weight of the pharmaceutical composition.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985), the entire disclosure of which is herein incorporated by reference. Pharmaceutically acceptable carriers are those that are compatible with the other ingredients in the formulation and biologically acceptable.

The compounds of this invention may be administered enterally (e.g., orally) or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to about 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration.

Liquid pharmaceutical compositions for parenteral administration, which are sterile solutions or suspensions, can be administered by, for example, intramuscular, intraperitoneal or subcutaneous injection. Sterile solutions can also be administered intravenously. Compositions for oral administration may be either liquid or solid composition forms.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compounds useful in the present invention may be administered to a mammal with one or more other pharmaceutically active agents such as those agents being used to treat any other medical condition present in the mammal. Examples of such pharmaceutically active agents include pain relieving agents, anti-angiogenic agents, anti-neoplastic agents, anti-diabetic agents, anti-infective agents, or gastrointestinal agents, or combinations thereof.

The one or more other pharmaceutically active agents may be administered in a therapeutically effective amount simultaneously (such as individually at the same time, or together in a pharmaceutical composition), and/or successively with one or more compounds of the present invention.

The term “combination therapy” refers to the administration of two or more therapeutic agents or compounds to treat a therapeutic condition or disorder described in the present disclosure, for example hot flush, sweating, thermoregulatory-related condition or disorder, or other condition or disorder. Such administration includes use of each type of therapeutic agent in a concurrent manner. In either case, the treatment regimen will provide beneficial effects of the drug combination in treating the conditions or disorders described herein.

The route of administration may be any enteral or parenteral route, which effectively transports the active compound of formula I, or a pharmaceutically acceptable salt thereof, to the appropriate or desired site of action, such as oral, nasal, pulmonary, transdermal, such as passive or iontophoretic delivery, or parenteral, e.g. rectal, depot, subcutaneous, intravenous, intraurethral, intra-articular, intramuscular, intranasal, ophthalmic solution or an ointment. Furthermore, the administration of compound of formula I, or pharmaceutically acceptable salt thereof, with other active ingredients may be consecutive or simultaneous.

In one embodiment, the present invention is directed to methods for treating or preventing a condition selected from the group consisting of a vasomotor symptom, sexual dysfunction, gastrointestinal disorder, genitourinary disorder, chronic fatigue syndrome, fibromyalgia syndrome, depression disorder, endogeneous behavioral disorder, cognitive disorder, diabetic neuropathy, pain, and combinations thereof in a subject in need thereof, comprising the step of:

administering to said subject an effective amount of a compound of formula I or a pharmaceutically acceptable salt thereof.

In certain embodiments, the vasomotor symptom is hot flush.

In certain embodiments, the sexual dysfunction is desire-related or arousal-related.

In certain embodiments, the gastrointestinal disorder or the genitourinary disorder is stress incontinence or urge incontinence.

In certain embodiments, the condition is chronic fatigue syndrome.

In certain embodiments, the condition is fibromyalgia syndrome.

In certain embodiments, the condition is a depression disorder selected from the group consisting of major depressive disorder, generalized anxiety disorder, panic disorder, attention deficit disorder with or without hyperactivity, sleep disturbance, social phobia, and combinations thereof.

In certain embodiments, the condition is diabetic neuropathy.

In certain embodiments, the condition is pain.

In certain embodiments, the pain is acute centralized pain, acute peripheral pain, or a combination thereof.

In certain embodiments, the pain is chronic centralized pain, chronic peripheral pain, or a combination thereof.

In certain embodiments, the pain is neuropathic pain, visceral pain, musculoskeletal pain, bony pain, cancer pain, inflammatory pain, or a combination thereof.

In certain embodiments, the neuropathic pain is associated with diabetes, post traumatic pain of amputation, lower back pain, cancer, chemical injury, toxins, major surgery, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions, or a combination thereof.

In certain embodiments, the neuropathic pain is post-herpetic neuralgia.

In certain embodiments, the visceral pain is associated with ulcerative colitis, irritable bowel syndrome, irritable bladder, Crohn's disease, rheumatologic (arthralgias), tumors, gastritis, pancreatitis, infections of the organs, biliary tract disorders, or a combination thereof.

In certain embodiments, the pain is female-specific pain.

The present invention provides for the treatment of vasomotor symptoms by methods of recovering the reduced activity of norepinephrine. Without wishing to be bound by any theory, norepinephrine activity in the hypothalamus or in the brainstem can be elevated by (i) blocking the activity of the NE transporter, (ii) blocking the activity of the presynaptic adrenergic α₂ receptor with an antagonist, or (iii) blocking the activity of 5-HT on NE neurons with a 5-HT₂ₐ antagonist.

The compounds of the invention are also useful to prevent and treat pain. The pain may be, for example, acute pain or chronic pain. The pain may also be centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain or dental pain, or headaches such as migraines or tension headaches, or combinations of these pains. One skilled in the art will recognize that these pains may overlap one another. For example, a pain caused by inflammation may also be visceral or musculoskeletal in nature.

In a preferred embodiment of the present invention the compounds useful in the present invention are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromyalgia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In a more preferred embodiment, the compounds useful in this invention are used to treat chronic pain that is neuropathic pain, visceral pain, musculoskeletal pain, bony pain, cancer pain or inflammatory pain or combinations thereof, in accordance with the methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. The methods of use for compounds of this invention further include treatments in which the neuropathic pain is a condition secondary to metastatic infiltration, adiposis dolorosa, burns, or central pain conditions related to thalamic conditions.

As mentioned previously, the methods of the present invention may be used to treat pain that is somatic and/or visceral in nature. For example, somatic pain that can be treated in accordance with the methods of the present invention include pains associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, the chronic pain may be with or without peripheral or central sensitization.

The compounds useful in this invention may also be used to treat acute and/or chronic pain associated with female conditions, which may also be referred to as female-specific pain. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

### 3-(2,2-dioxido-1-phenyl-3,4-dihydro-1H-2,1-benzothiazin-3-yl)-N-methylpropan-1-amine

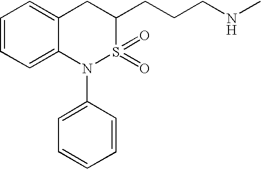

### Step 1: N-phenyl-methanesulfonamide

To a stirred solution of aniline (10 mL, 110 mmol) and pyridine (11.5 mL, 143 mmol) in dichloromethane (200 mL), under nitrogen at 0° C., was added methane sulfonyl chloride (10.2 mL, 132 mmol) dropwise. The reaction solution was stirred for one hour at 0° C., then allowed to warm to room temperature and stirred for 18 hours. The reaction was cooled in an ice bath and 6N NaOH (200 mL) was added to quench the reaction, then transferred to a separatory funnel with H₂O (200 mL) and washed with dichloromethane (200 mL). The organic phase was separated and the aqueous cooled in an ice bath and acidified to pH=2 with concentrated HCl extracted with diethyl ether (150 mL×3). The organic extracts were combined, dried (MgSO₄), filtered and the solvent removed, in vacuo, to give a white solid (15.93 g), which was recrystallized from boiling toluene (50 mL) to give white crystals (13.25 g, 70% yield).

### Step 2: 2-(Methanesulfonyl-phenyl-amino)-benzoic Acid

To a stirred mixture of sodium hydride (60% oil dispersion, 561 mg, 14.02 mmol) in dimethoxyethane (200 mL), under nitrogen, was added in portions N-phenyl-methanesulfonamide (2.40 g, 14.02 mmol) After hydrogen evolution had ceased, diphenyliodonium carboxylate (5.00 g, 15.4 mmol) and copper (II) acetate (117 mg, 0.64 mmol) were added and the mixture heated to 80° C. for 48 hours. H₂O (50 mL) and 2N NaOH solution (100 mL) was added and the mixture filtered through Celite. The filtrate was washed with diethyl ether (100 mL) and the aqueous phase was acidified with concentrated hydrochloric acid (pH=2) and extracted three times with diethyl ether (150 mL). The organic extracts were combined, dried (MgSO₄), filtered and the solvent removed, to give a peach colored solid (3.81 g, 85% yield). This crude material was used directly in the next reaction.

### Step 3: 2-(Methanesulfonyl-phenyl-amino)-benzoic Acid Methyl Ester

To a stirred mixture of 2-(methanesulfonyl-phenyl-amino)-benzoic acid (3.50 g, 12.01 mmol) and potassium carbonate (3.32 g, 24.0 mmol) in anhydrous dimethylformamide (15 mL), under nitrogen, was added iodomethane (3.75 mL, 60.1 mmol) and the mixture heated to 60° C. for 18 hours. Additional iodomethane (4.00 mL, 64.1 mmol) was added and the mixture heated, under nitrogen, for a further 5 hours. The cooled reaction mixture was extracted with diethyl ether (150 mL) and washed with saturated aqueous sodium bicarbonate solution (100 mL). The aqueous washing was separated and extracted twice with diethyl ether (75 mL). The organic extracts were combined and washed with brine (150 mL), dried (MgSO₄), filtered and the solvent removed, in vacuo, to give a lightly amber colored liquid (3.61 g). This material was adsorbed onto silica and purified by SiO₂ column chromatography, eluting with a gradient of 0-50% ethyl acetate in hexane to afford a light yellow colored solid (2.90 g, 79% yield).

### Step 4: 1-phenyl-1H-2,1-benzothiazin-4(3H)-one 2,2-dioxide

To a stirred solution of 2-(methanesulfonyl-phenyl-amino)-benzoic acid methyl ester (6.3 g, 20.6 mmol) in anhydrous N,N-dimethylacetamide (20 mL), under nitrogen, was added sodium bis(trimethylsilyl)amide (1M in THF, 26 mL, 26 mmol) and the solution was stirred at room temperature for 18 hours. The solution was extracted with diethyl ether (150 mL) and washed with saturated aqueous sodium bicarbonate (150 mL). The organic layer was separated and the aqueous washing extracted twice with diethyl ether (100 mL). The organic extracts were combined and washed twice with H₂O (150 mL), brine (150 mL), dried (MgSO₄), filtered, then treated with activated charcoal and filtered through Celite, then a silica plug and the filtrate concentrated, in vacuo, to give a lightly yellow colored solid (4.47 g, 79% yield).

### Step 5: 1-phenyl-3,4-dihydro-1H-2,1-benzothiazine 2,2-dioxide

To a stirred solution of 1-phenyl-1H-2,1-benzothiazin-4(3H)-one 2,2-dioxide (2.00 g, 7.32 mmol) in trifluoroacetic acid (20 mL), under nitrogen, was added triethyl silane (8 mL, 50.1 mmol). After 18 hours at room temperature, the reaction was diluted with dichloromethane (150 mL) and washed three times with H₂O (100 mL). The organic layer was separated and the combined aqueous washings were extracted twice with dichloromethane (75 mL). The organic extracts were combined and washed twice with 1N NaOH solution (100 mL). The sodium hydroxide washings were combined and extracted twice with dichloromethane (75 mL). All organic extracts were combined, dried (MgSO₄), filtered, and the solvent removed, in vacuo, to give a yellow solid (3.79 g). This material was adsorbed onto silica and purified by SiO₂ column chromatography, eluting with a solution gradient of 0-50% ethyl acetate in hexane to afford the product as a yellow solid (1.71 g, 90% yield).

### Step 6: 3-Allyl-1-phenyl-3,4-dihydro-1H-benzo[c][1,2]thiazine 2,2-dioxide

To a stirred solution of 1-phenyl-3,4-dihydro-1H-benzo[c][1,2]thiazine 2,2-dioxide (760 mg, 2.93 mmol) in anhydrous tetrahydrofuran (5 mL), under nitrogen at −78° C., was added lithium bis(trimethylsilyl)amide (1M in THF, 3.22 mL, 3.22 mmol) dropwise. After 30 minutes, allyl bromide (279 μL, 3.22 mmol) was added dropwise and the solution warmed to room temperature. After 18 hours, the reaction mixture was extracted with diethyl ether (150 mL) and washed with aqueous sodium bicarbonate (100 mL). The organic extract was separated and the aqueous washing extracted with diethyl ether (100 mL). The organic extracts were combined and washed with brine (100 mL), dried (MgSO₄), filtered and the solvent removed, in vacuo, to give an amber colored oil. This material was adsorbed onto silica and purified by SiO2 column chromatography, eluting with a solution gradient of 0-30% ethyl acetate in hexane to afford a lightly yellow colored solid (530 mg, 60% yield).

### Step 7: 3-(2,2-dioxido-1-phenyl-3,4-dihydro-1H-2,1-benzothiazin-3-yl)propan-1-ol

To a stirred solution of 3-allyl-1-phenyl-3,4-dihydro-1H-benzo[c][1,2]thiazine 2,2-dioxide (440 mg, 1.47 mmol) in anhydrous tetrahydrofuran (15 mL), at 0° C. under nitrogen, was added 9-borabicyclo[3.3.1]nonane (0.5 M in THF, 7.35 mL, 3.67 mmol) dropwise and the solution warmed to room temperature and stirred for 18 hours. The reaction was cooled to 0° C. and quenched with ethanol (2 mL), then aqueous sodium hydroxide (1M, 3.31 mL, 3.31 mmol) and 30% aqueous hydrogen peroxide (1 mL). The reaction was heated to reflux for 2.5 hours, cooled and extracted with diethyl ether (150 mL) and washed twice with H₂O (75 mL). The aqueous washings were combined and extracted with diethyl ether (150 mL). The organic extracts were combined, dried (MgSO₄), filtered and the solvent removed, in vacuo, to give a clear oil. This material was adsorbed onto silica and purified by SiO₂ column chromatography, eluting with a gradient of 0-90% ethyl acetate in hexane to afford a white solid (350 mg, 75% yield).

### Step 8: 3-(2,2-dioxido-1-phenyl-3,4-dihydro-1H-2,1-benzothiazin-3-yl)-N-methylpropan-1-amine

To a stirred solution of 3-(2,2-dioxido-1-phenyl-3,4-dihydro-1H-2,1-benzothiazin-3-yl)propan-1-ol (350 mg, 1.10 mmol) and p-toluenesulfonyl chloride (273 mg, 1.43 mmol) in dichloromethane (10 mL) under nitrogen at room temperature, was added triethyl amine (307 μL, 2.21 mmol) and the solution stirred for 72 hours. Methylamine (33% in ethanol, 6 mL) was added and the solution stirred for 18 hours. The reaction mixture was extracted with dichloromethane (100 mL) and washed twice with H₂O (75 mL). The aqueous washings were combined and extracted twice with dichloromethane (75 mL). The organic extracts were combined, dried (MgSO₄), and evaporated to give a clear oil. This material was adsorbed onto silica and purified by SiO₂ column chromatography, eluting with a gradient of 0-30% methanol in dichloromethane to afford a white solid. This material was dissolved in diethyl ether (25 mL) and 1 equivalent of HCl (1N in Et₂O) was added. The solid was filtered, dissolved in H₂O and lyophilized to afford 3-(2,2-dioxido-1-phenyl-3,4-dihydro-1H-2,1-benzothiazin-3-yl)-N-methyl propan-1-amine hydrochloride as a white solid (64 mg, 18% yield). HPLC purity 100% at 210-370 nm, 6.7 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes. HRMS: calculated for C₁₈H₂₂N₂O₂S+H+, 331.14747; found (ESI, [M+H]⁺), 331.1488.

Alternate synthesis of 1-phenyl-3,4-dihydro-1H-2,1-benzothiazine 2,2-dioxide

### Step 1: N-[2-(hydroxymethyl)phenyl]methanesulfonamide

To a solution of (2-aminophenyl)methanol 12.3 g, 100 mmol) and pyridine (100 mL) in chloroform under nitrogen was added a solution of methane sulfonyl chloride (8.5 mL, 110 mmol) in chloroform (100 mL) over 1 hour. After 12 hours at room temperature, the mixture was washed with hydrochloric acid (2 N, 200 mL), dried (MgSO₄) and evaporated. The residue was purified by column chromatography (SiO₂, 3:97 to 100:0, EtOAc:hexanes, gradient elution) to afford the product (13.7 g) as a yellow oil:

MS (ES) m/z 200.0;

HPLC purity 100.0% at 210-370 nm, 4.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Step 2: N-(2-formylphenyl)methanesulfonamide

To solution of N-[2-(hydroxymethyl)phenyl]methanesulfonamide (1.08 g, 5.1 mmol) in dry dichloromethane (20 mL), was added manganese dioxide (85% Aldrich Chemical Co., 5.0 g) and the mixture stirred at room temperature under nitrogen. After 16 hours, the mixture was filtered through Celite, the pad washed with dichloromethane/methanol (1:1) and the combined organic solutions evaporated to afford the product (1.05 g) as a yellow solid:

MS (ES) m/z 197.9;

HPLC purity 100.0% at 210-370 nm, 5.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Step 3: 1-(4-methoxybenzyl)-1H-2,1-benzothiazine 2,2-dioxide

To a solution of N-(2-formylphenyl)methanesulfonamide (2.0 g, 10 mmol) in dry acetonitrile (40 mL) was added cesium carbonate (6.5 g, 20 mmol) and 4-methoxybenzyl chloride (2.7 mL, 20 mmol), and the mixture heated to 50° C. under nitrogen. After 16 hours, the cooled reaction mixture was diluted with ethyl acetate (100 mL), filtered, and the precipitate washed with ethyl acetate (200 mL). The combined organic solutions were evaporated and the residue purified by column chromatography (SiO₂, 10:90 to 100:0 dichloromethane:hexanes) to afford the product (2.6 g) as a colorless oil.

HPLC purity 100.0% at 210-370 nm, 8.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Step 4: 1H-2,1-benzothiazine 2,2-dioxide

To a solution of 1-(4-methoxybenzyl)-1H-2,1-benzothiazine 2,2-dioxide (200 mg) in dry dichloromethane (2 mL) was added trifluoroacetic acid (3 mL) at room temperature. After 3 hours the mixture was evaporated and the residue purified by column chromatography (SiO₂, 3:97 to 55:45 ethylacetate:hexanes, gradient elution) to afford the product (123 mg) as a white solid:

HPLC purity 100.0% at 210-370 nm, 5.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Step 5: 3,4-dihydro-1H-2,1-benzothiazine 2,2-dioxide

A mixture of 1H-2,1-benzothiazine 2,2-dioxide (0.36 g, 2 mmol) and palladium on carbon (10%, 36 mg) in methanol (5 mL) was stirred under a ballon of hydrogen at room temperature. After 3 hours, the mixture was filtered and evaporated to afford the product (0.349 g) which was used without further purification:

HPLC purity 100.0% at 210-370 nm, 5.0 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Step 6: 1-phenyl-3,4-dihydro-1H-2,1-benzothiazine 2,2-dioxide

To a stirred solution of 1H-2,1-benzothiazine 2,2-dioxide (0.292 g, 1.6 mmol) in dichloromethane (6 mL), was added phenyl boronic acid (0.586 g, 4.8 mmol), pyridine (0.386 g, 4.8 mmol), copper II acetate (0.582 g, 3.2 mmol) and powdered molecular sieves (4 angstrom, 292 mg). After 72 hours methanol (2 mL) was added, the mixture was filtered, the pad washed with dichloromethane, the combined solutions were washed with ammonium hydroxide solution, dried (MgSO₄) and evaporated. The residue was purified by column chromatography (SiO₂, 0:100 to 40:60, ethylacetate:hexanes, gradient elution, to afford the product (276 mg) as a white powder:

HPLC purity 100% at 210-370 nm, 8.3 minutes; Xterra RP18, 3.5μ, 150×4.6 mm column, 1.2 mL/minutes. 85/15-5/95 (ammonium formate buffer pH=3.5/ACN+MeOH) for 10 minutes. hold 4 minutes.

### Example 2

### 3-(2,2-dioxido-1-phenyl-3,4-dihydro-1H-2,1-benzothiazin-3-yl)propan-1-amine

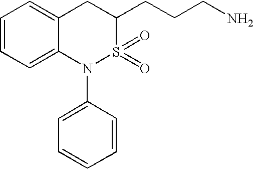

To a stirred solution of 3-(2,2-dioxido-1-phenyl-3,4-dihydro-1H-2,1-benzothiazin-3-yl)propan-1-ol (60 mg, 0.19 mmol) and p-toluenesulfonyl chloride (47 mg, 0.25 mmol) in dichloromethane (10 mL), under nitrogen at room temperature, was added triethylamine (53 μL, 0.38 mmol) and the solution stirred for 18 hours. Ammonia (7N in methanol, 10 mL) was added and the solution stirred for 18 hours. The solvent was removed and the material purified by reverse phase-HPLC (10-100% CH₃CN:H₂O+1% CF₃CO₂H buffer), the fractions collected and lyophilized to afford 3-(2,2-dioxido-1-phenyl-3,4-dihydro-1H-2,1-benzothiazin-3-yl)propan-1-amine trifluoroacetic acid salt as a clear oil (2 mg, 3% yield).

HPLC purity 100% at 210-370 nm, 6.6 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₂₀N₂O₂S+H+, 317.13182; found (ESI, [M+H]⁺), 317.1316

### Example 3

### 3-(2,2-dioxido-1-phenyl-3,4-dihydro-1H-2,1-benzothiazin-3-yl)-N,N dimethylpropan-1-amine

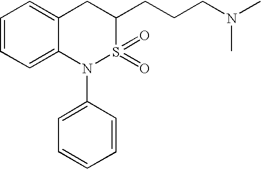

In an analogous manner to Example 2 3-(2,2-dioxido-1-phenyl-3,4-dihydro-1H-2,1-benzothiazin-3-yl)-N,N-dimethylpropan-1-amine trifluoroacetic acid salt (11 mg) prepared from 3-(2,2-dioxido-1-phenyl-3,4-dihydro-1H-2,1-benzothiazin-3-yl)propan-1-ol and dimethylamine.

HPLC purity 96.7% at 210-370 nm, 6.7 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₂S+H+, 345.16312; found (ESI, [M+H]⁺), 345.1634

### Example 4

### 3-(2,2-dioxido-1-phenyl-3,4-dihydro-1H-2,1-benzothiazin-3-yl)-N-ethylpropan-1-amine

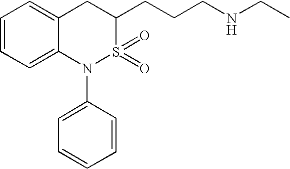

In an analogous manner to Example 2 3-(2,2-dioxido-1-phenyl-3,4-dihydro-1H-2,1-benzothiazin-3-yl)-N-ethylpropan-1-amine trifluoroacetic acid salt (12 mg) was prepared from 3-(2,2-dioxido-1-phenyl-3,4-dihydro-1H-2,1-benzothiazin-3-yl)propan-1-ol and ethylamine.

HPLC purity 92.5% at 210-370 nm, 6.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₂S+H+, 345.16312; found (ESI, [M+H]⁺), 345.1648

### Example 5

### 3-(2,2-dioxido-1-phenyl-3,4-dihydro-1H-2,1-benzothiazin-3-yl)-N-isopropyl propan-1-amine

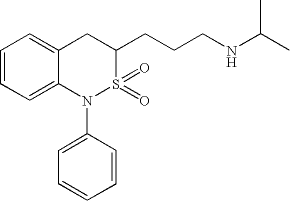

In an analogous manner to Example 2 3-(2,2-dioxido-1-phenyl-3,4-dihydro-1H-2,1-benzothiazin-3-yl)-N-isopropylpropan-1-amine trifluoroacetic acid salt (37 mg) was prepared from 3-(2,2-dioxido-1-phenyl-3,4-dihydro-1H-2,1-benzothiazin-3-yl)propan-1-ol and isopropylamine.

HPLC purity 100% at 210-370 nm, 7.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₀H₂₆N₂O₂S+H+, 359.17877; found (ESI, [M+H]⁺), 359.178

### Example 6

### 1-phenyl-3-(3-pyrrolidin-1-ylpropyl)-3,4-dihydro-1H-2,1-benzothiazine 2,2-dioxide

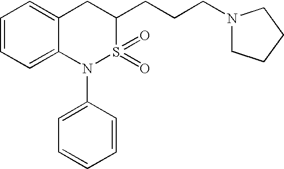

In an analogous manner to Example 2 1-phenyl-3-(3-pyrrolidin-1-ylpropyl)-3,4-dihydro-1H-2,1-benzothiazine 2,2-dioxide trifluoroacetic acid salt (3 mg) was prepared from 3-(2,2-dioxido-1-phenyl-3,4-dihydro-1H-2,1-benzothiazin-3-yl)propan-1-ol and pyrrolidine.

HPLC purity 95.8% at 210-370 nm, 7.0 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₁H₂₆N₂O₂S+H+, 371.17877; found (ESI, [M+H]⁺), 371.177

### Example 7

### N-benzyl-3-(2,2-dioxido-1-phenyl-3,4-dihydro-1H-2,1-benzothiazin-3-yl)propan-1-amine

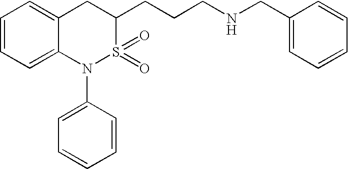

In an analogous manner to Example 2N-benzyl-3-(2,2-dioxido-1-phenyl-3,4-dihydro-1H-2,1-benzothiazin-3-yl)propan-1-amine trifluoroacetic acid salt (12 mg) was prepared from 3-(2,2-dioxido-1-phenyl-3,4-dihydro-1H-2,1-benzothiazin-3-yl)propan-1-ol and benzylamine.

HPLC purity 100% at 210-370 nm, 7.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₄H₂₆N₂O₂S+H+, 407.17877; found (ESI, [M+H]⁺), 407.1798

### Example 8

### N-[3-(2,2-dioxido-1-phenyl-3,4-dihydro-1H-2,1-benzothiazin-3-yl)propyl]cyclohexanamine

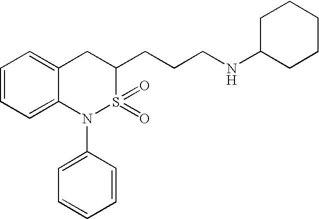

In an analogous manner to Example 2 N-[3-(2,2-dioxido-1-phenyl-3,4-dihydro-1H-2,1-benzothiazin-3-yl)propyl]cyclohexanamine trifluoroacetic acid salt (24 mg) was prepared from 3-(2,2-dioxido-1-phenyl-3,4-dihydro-1H-2,1-benzothiazin-3-yl)propan-1-ol and cyclohexylamine.

HPLC purity 99.0% at 210-370 nm, 8.0 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₃H₃₀N₂O₂S+H+, 399.21007; found (ESI, [M+H]⁺), 399.21

### Example 9

### N-methyl-3-[(3R)-3-methyl-2,2-dioxido-1-phenyl-3,4-dihydro-1H-2,1-benzothiazin-3-yl]propan-1-amine

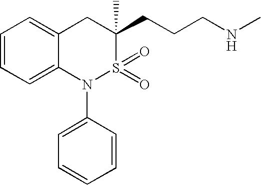

### Step 1:

### 3-allyl-3-methyl-1-phenyl-3,4-dihydro-1H-2,1-benzothiazine 2,2-dioxide

To a stirred solution of 3-allyl-1-phenyl-3,4-dihydro-1H-2,1-benzothiazine 2,2-dioxide (400 mg, 1.34 mmol) in anhydrous tetrahydrofuran (10 mL) at −78° C., under nitrogen, was added lithium bis(trimethylsilyl)amide (1M in THF, 1.6 mL) dropwise and the solution stirred for 30 minutes. Iodomethane (83 μL, 1.6 mmol) was added and the solution warmed to room temperature and stirred for 72 hours. The reaction was quenched with water and extracted with ethyl acetate (150 mL) and washed twice with H₂O (100 mL). The aqueous washings were combined and extracted twice with ethyl acetate (75 mL). The organic extracts were combined, dried (MgSO₄), filtered and evaporated, in vacuo, to give an amber colored solid. This material was adsorbed onto silica and purified by SiO₂ column chromatography eluting with a gradient of 0-30% ethyl acetate in hexane to afford the racemic product as a white solid (380 mg, 91% yield). The racemic material, was dissolved in 11 mL of methanol, and the resulting solution was injected onto the Supercritical Fluid Chromatography (SFC) instrument with a volume of 0.5 mL per injection. The baseline resolved enantiomers were collected using a Berger MultiGram Prep SFC (Berger Instruments, Inc. Newark, Del.) under the following conditions: Chiralcel OJ-H SFC column (5μ, 250 mm L×20 mm ID, Chiral Technologies, Inc, Exton, Pa.), 35° C. column temperature, 20% MeOH as CO₂ modifier, 50 mL/min flow rate, 100 bar outlet pressure, 220 nm UV detection. The chiral purity of each enantiomer was determined under the same SFC conditions using a Chiralcel OJ-H column (5μ, 250 mm L×4.6 mm ID) at 2.0 mL/min flow rate on a Berger Analytical SFC instrument.

Peak 1 Rt 5.0 minutes

Peak 2 (190 mg) Rt 6.7 minutes arbitrarily assigned as (3R)-3-allyl-3-methyl-1-phenyl-3,4-dihydro-1H-2,1-benzothiazine 2,2-dioxide

### Step 2: 3-[(3R)-3-methyl-2,2-dioxido-1-phenyl-3,4-dihydro-1H-2,1-benzothiazin-3-yl]propan-1-ol

To a stirred solution of (3R)-3-allyl-3-methyl-1-phenyl-3,4-dihydro-1H-2,1-benzothiazine 2,2-dioxide (190 mg, 0.61 mmol) in anhydrous tetrahydrofuran (9 mL), at 0° C. under nitrogen, was added 9-borabicyclo[3.3.1]nonane (0.5M in THF, 3.03 mL, 1.52 mmol) dropwise, and the solution warmed to room temperature and stirred for 18 hours. The reaction was cooled to 0° C. and quenched with ethanol (1 mL), then aqueous sodium hydroxide (1 M, 3.31 mL, 1.36 mmol) and 30% aqueous hydrogen peroxide (1 mL) were added. The reaction was heated to reflux for 5 hours, cooled to room temperature, then extracted with diethyl ether (150 mL) and washed twice with H₂O (75 mL). The aqueous washings were combined and extracted with diethyl ether (100 mL). The organic extracts were combined, dried (MgSO₄), filtered and the solvent removed in vacuo, to give a yellow oil. This material was adsorbed onto silica and purified by SiO₂ column chromatography, eluting with a solution gradient of 0-10% ethyl acetate in hexane. Further purification by SiO₂ column chromatography, eluting with a gradient of 0-5% methanol in dichloromethane gave the product as a clear oil (120 mg, 60% yield).

### Step 3: N-methyl-3-[(3R)-3-methyl-2,2-dioxido-1-phenyl-3,4-dihydro-1H-2,1-benzothiazin-3-yl]propan-1-amine

To a stirred solution of 3-[(3R)-3-methyl-2,2-dioxido-1-phenyl-3,4-dihydro-1H-2,1-benzothiazin-3-yl]propan-1-ol (100 mg, 0.30 mmol) and p-toluenesulfonyl chloride (75 mg, 0.39 mmol) in dichloromethane (5 mL) under nitrogen at room temperature, was added triethylamine (84 μL, 0.60 mmol). After 18 hours additional p-toluenesulfonyl chloride (75 mg) and triethylamine (100 μL) was added and the solution stirred for 5 hours. Methylamine (33% in ethanol, 30 mL) was added and the solution stirred under nitrogen at room temperature. After 18 hours, the reaction was diluted with ethyl acetate (150 mL) and washed with 1N NaOH (100 mL). The aqueous washing was extracted with ethyl acetate (100 mL). The organic extracts were combined and washed twice with 2N HCl (100 mL). The aqueous HCl washings were combined and extracted with ethyl acetate (100 mL), then treated with 2N NaOH until pH=14. This basic aqueous phase was extracted three times with ethyl acetate (150 mL), dried (MgSO₄), filtered, and the solvent removed in vacuo, to give a lightly yellow colored liquid. This material was purified by preparative RP-HPLC (10-100% CH₃CN:H₂O) to afford a white solid. This solid was dissolved in diethyl ether (25 mL) and 1 equivalent of HCl (1N in Et₂O) was added. The solid was filtered, dissolved in H₂O and lyophilized to afford N-methyl-3-[(3R)-3-methyl-2,2-dioxido-1-phenyl-3,4-dihydro-1H-2,1-benzothiazin-3-yl]propan-1-amine hydrochloride as a white solid (7 mg, 6% yield). The stereochemistry of this product was arbitrarily assigned.

HPLC purity 100.0% at 210-370 nm, 7.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₂S+H+, 345.1632; found (ESI, [M+H]⁺), 345.1625.

### Example 10

### 3-[(3S)-2,2-dioxido-1-phenyl-3,4-dihydro-1H-2,1-benzothiazin-3-yl]-N-methylpropan-1-amine

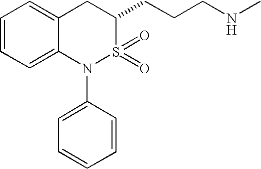

### Step 1: 3-Allyl-1-phenyl-3,4-dihydro-1H-benzo[c][1,2]thiazine 2,2-dioxide

3-Allyl-1-phenyl-3,4-dihydro-1H-benzo[c][1,2]thiazine 2,2-dioxide (2.19 g) was dissolved in 50 mL of 1:1 methanol/acetonitrile, and the resulting solution was injected onto the Supercritical Fluid Chromatography (SFC) instrument with a volume of 1.0 mL per injection. The baseline resolved enantiomers were collected using a Berger MultiGram Prep SFC (Berger Instruments, Inc. Newark, Del.) under the following conditions: Chiralcel OJ-H SFC column (5μ, 250 mm L×20 mm ID, Chiral Technologies, Inc, Exton, Pa.), 35° C. column temperature, 20% MeOH as CO₂ modifier, 50 mL/min flow rate, 100 bar outlet pressure, 220 nm UV detection. The chiral purity of each enantiomer was determined under the same SFC conditions using a Chiralcel OJ-H column (5μ, 250 mm L×4.6 mm ID) at 2.0 mL/min flow rate on a Berger Analytical SFC instrument. Both compounds were determined to be >99.8% chirally pure (Rₜ 5.3 and 6.6 min).

Enantiomer 1, Rt 5.3 minutes. arbitrarily assigned as (3S)-3-allyl-1-phenyl-3,4-dihydro-1H-2,1-benzothiazine 2,2-dioxide (940 mg)

Enantiomer 2, Rt 6.6.minutes. arbitrarily assigned as (3R)-3-allyl-1-phenyl-3,4-dihydro-1H-2,1-benzothiazine 2,2-dioxide (820 mg)

### Step 2: 3-[(3S)-2,2-dioxido-1-phenyl-3,4-dihydro-1H-2,1-benzothiazin-3-yl]propan-1-ol

To a stirred solution of (3S)-3-allyl-1-phenyl-3,4-dihydro-1H-2,1-benzothiazine 2,2-dioxide (940 mg, 3.14 mmol) in anhydrous tetrahydrofuran (30 mL), at 0° C. under nitrogen was added, 9-borabicyclo[3.3.1]nonane (0.5 M in THF, 15.7 mL, 7.85 mmol) dropwise and the solution warmed to room temperature and stirred for 18 hours. Additional 9-borabicyclo[3.3.1]nonane (0.5 M in THF, 10 mL, 5 mmol) was added and the solution stirred for 2 hours. The reaction was cooled to 0° C. and quenched with ethanol (5 mL), then aqueous lithium hydroxide (1M, 7.50 mL, 7.50 mmol) and 50% aqueous hydrogen peroxide (3 mL) were added. The reaction was heated to reflux for 5 hours, cooled to room temperature, then diluted with diethyl ether (150 mL) and washed three times with H₂O (100 mL). The aqueous washings were combined and extracted twice with diethyl ether (100 mL). The organic extracts were combined, dried (MgSO₄), filtered and the solvent removed in vacuo, to give a yellow oil (1.20 g). This material was adsorbed onto silica and purified by SiO₂ column chromatography, eluting with a solution gradient of 0-100% ethyl acetate in hexane to afford the product as a clear oil (650 mg, 65% yield).

### Step 3: 3-[(3S)-2,2-dioxido-1-phenyl-3,4-dihydro-1H-2,1-benzothiazin-3-yl]-N-methylpropan-1-amine

To a stirred solution of 3-[(3S)-2,2-dioxido-1-phenyl-3,4-dihydro-1H-2,1-benzothiazin-3-yl]propan-1-ol (650 mg, 2.05 mmol) and p-toluenesulfonyl chloride (508 mg, 2.66 mmol) in dichloromethane (20 mL) under nitrogen at room temperature, was added triethyl amine (570 μL, 4.10 mmol). After 18 hours, methylamine (33% in ethanol, 25 mL) was added and the solution stirred at room temperature for 18 hours. The reaction was diluted with dichloromethane (150 mL) and washed three times with H₂O (100 mL). The aqueous washings were combined and extracted twice with dichloromethane (100 mL). The organic extracts were combined and washed with brine (150 mL), dried (Na₂SO₄), filtered, and the solvent removed in vacuo, to give a clear oil. This material was adsorbed onto silica and purified by SiO₂ column chromatography, eluting with 0-10% ammonia-methanol solution (7N) in dichloromethane to afford a clear oil (510 mg). This material was dissolved in methanol (8 mL) and 1.2 equivalents of HCl (4N in dioxane) was added. The solution was concentrated and the oil dissolved in H₂O (5 mL) and lyophilized to afford 3-[(3S)-2,2-dioxido-1-phenyl-3,4-dihydro-1H-2,1-benzothiazin-3-yl]-N-methylpropan-1-amine hydrochloride as a white solid (533 mg, 71% yield). HPLC purity 100.0% at 210-370 nm, 6.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₂S+H+, 331.14747; found (ESI, [M+H]⁺), 331.1425.

### Example 11

### 3-[(3R)-2,2-dioxido-1-phenyl-3,4-dihydro-1H-2,1-benzothiazin-3-yl]-N-methylpropan-1-amine

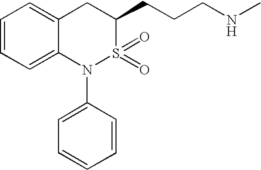

### Step 1: 3-[(3R)-2,2-dioxido-1-phenyl-3,4-dihydro-1H-2,1-benzothiazin-3-yl]propan-1-ol

3-[(3R)-2,2-dioxido-1-phenyl-3,4-dihydro-1H-2,1-benzothiazin-3-yl]propan-1-ol (680 mg) was prepared from (3R)-3-allyl-1-phenyl-3,4-dihydro-1H-2,1-benzothiazine 2,2-dioxide in an analogous procedure to that used in Example 10, Step 2.

Step 2: 3-[(3R)-2,2-dioxido-1-phenyl-3,4-dihydro-1H-2,1-benzothiazin-3-yl]-N-methylpropan-1-amine 3-[(3R)-2,2-dioxido-1-phenyl-3,4-dihydro-1H-2,1-benzothiazin-3-yl]-N-methylpropan-1-amine hydrochloride (548 mg, 75% yield) was prepared from 3-[(3R)-2,2-dioxido-1-phenyl-3,4-dihydro-1H-2,1-benzothiazin-3-yl]propan-1-ol in an analogous procedure to that used in Example 10 step 3.

HPLC purity 100.0% at 210-370 nm, 6.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₂S+H+, 331.14747; found (ESI, [M+H]⁺), 331.1375.

### Example 12

### 3-(2,2-dioxido-1-phenyl-1H-4,2,1-benzoxathiazin-3-yl)-N-methylpropan-1-amine

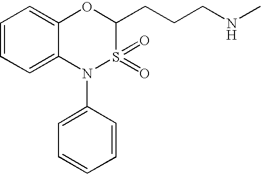

### Step 1: 1-chloro-N-(2-hydroxyphenyl)methanesulfonamide

O-aminophenol (17.6 g, 163.9 mmol) was dissolved in tetrahydrofuran (160 mL). Chloromethanesulfonyl chloride (16 mL, 176 mmol) and pyridine (18 mL) were added to the solution at room temperature, and the mixture was stirred overnight. The reaction mixture was acidified with 1N hydrochloric acid and extracted with ethyl acetate. The combined organic layers were dried over magnesium sulfate and concentrated. The residue was taken directly to the next step.

Step 2: 1H-4,2,1-benzoxathiazine 2,2-dioxide. The above sulfamido residue and potassium carbonate (90.5 g, 656 mmol) in methanol (400 mL) were heated under reflux for 48 hours. The solvent was removed in vacuo and the residue acidified with 1N hydrochloric acid and extracted with ethyl acetate. The combined organic layers were dried over magnesium sulfate and concentrated. The residue was triturated with hexane/ethyl ether to afford the title compound (16 g, 53%).

HPLC purity=100% at 210-370 nm; RT=5.3 min; 85/15-5/95 (ammonium formate buffer pH=3.5/ACN+MeOH) for 10 minutes, hold 4 minutes Xterra RP18, 3.5μ, 150×4.6 mm, 1.2 mL/minutes.

### Step 3: 1-phenyl-1H-4,2,1-benzoxathiazine 2,2-dioxide

A mixture of 1H-4,2,1-benzoxathiazine 2,2-dioxide (4.5 g, 24.3 mmol), phenylboronic acid (6.56 g, 54 mmol), copper(II) acetate (0.720 g, 3.97 mmol), pyridine-N-oxide (2.86 g, 30 mmol), and 4 A molecular sieves in dichloromethane (200 mL) was stirred for 10 minutes, Triethylamine (8.37 mL) was added and the mixture stirred for 3 days. The mixture was filtered through Celite and concentrated in vacuo onto silica gel and purified by SiO₂ column chromatography (ethyl acetate/hexane; gradient, 0 to 40%) to afford the title compound (2.6 g, 41%).

HPLC purity component=99.7% at 210-370 nm; RT=8.5 min; 85/15-5/95 (ammonium formate buffer pH=3.5/ACN+MeOH) for 10 minutes, hold 4 minutes Xterra RP18, 3.5μ, 150×4.6 mm, 1.2 mL/minutes.

### Step 4: 3-allyl-1-phenyl-1H-4,2,1-benzoxathiazine 2,2-dioxide

To a solution of 1-phenyl-1H-4,2,1-benzoxathiazine 2,2-dioxide (2.3 g, 8.8 mmol) in tetrahydrofuran (30 mL) at 0° C. was added lithium bis(trimethylsilyl)amide (1 M in tetrahydrofuran, 9 mL, 9 mmol). After 15 minutes, allyl bromide (0.78 mL, 9 mmol) was added and the mixture was slowly warmed to room temperature and stirred overnight. The reaction was quenched with saturated ammonium chloride solution and extracted with ethyl acetate, and the organic layers were dried over magnesium sulfate, and concentrated in vacuo. The residue was purified by SiO₂ column chromatography (Hexane/ethyl acetate; 9:1) to afford the title compound (1.3 g, 42%). HPLC purity=100% at 210-370 nm; RT=10.2 min; 85/15-5/95 (ammonium formate buffer pH=3.5/ACN+MeOH) for 10 minutes, hold 4 minutes Xterra RP18, 3.5μ, 150×4.6 mm, 1.2 mL/minutes.

### Step 5: 3-(2,2-dioxido-1-phenyl-1H-4,2,1-benzoxathiazin-3-yl)propan-1-ol

To a solution of 3-allyl-1-phenyl-1H-4,2,1-benzoxathiazine 2,2-dioxide (2.7 g, 8.9 mmol) in tetrahydrofuran (75 mL) at 0° C. was added 9-borabicyclo[3.3.1]nonane (0.5M in tetrahydrofuran, 54 mL, 27 mmol). After 10 minutes, the solution was slowly warmed to room temperature and stirred overnight. The reaction was cooled to 0° C. and quenched with ethanol (12 mL), followed by sodium hydroxide (2N solution, 10.5 mL). Hydrogen peroxide (7 mL) was added and the reaction was slowly warmed to room temperature. The reaction was then poured into water and extracted with ethyl acetate. The organic layers were dried over magnesium sulfate, and concentrated in vacuo. The residue was purified by SiO₂ column chromatography (hexane/ethyl acetate; 7:3) to afford the title compound (1.8 g, 64%). HPLC purity component=100% at 210-370 nm; RT=9.9 min; 85/15-5/95 (ammonium formate buffer pH=3.5/ACN+MeOH) for 10 minutes, hold 4 minutes Xterra RP18, 3.5μ, 150×4.6 mm, 1.2 mL/minutes.

Step 6: 3-(2,2-dioxido-1-phenyl-1H-4,2,1-benzoxathiazin-3-yl)-N-methylpropan-1-amine To a solution of 3-(2,2-dioxido-1-phenyl-1H-4,2,1-benzoxathiazin-3-yl)propan-1-ol (0.300 g, 0.94 mmol) in dichloromethane (5 mL) at room temperature was added triethylamine (0.5 mL). p-Toluenesulfonyl chloride (0.192 g, 1.01 mmol) was added and the reaction stirred overnight. Methylamine (33% solution in ethanol, 10 mL) was added and the reaction stirred overnight. The reaction was then poured into 2N hydrochloric acid and extracted with diethyl ether. The aqueous layer was basified with potassium carbonate, extracted with ethyl acetate, the combined organic layers were dried over magnesium sulfate, and concentrated in vacuo. The residue was purified by SiO₂ column chromatography (96.5:3.5, dichloromethane: 7N ammonia/methanol). The residue was dissolved in methanol and treated with hydrogen chloride (4N in dioxane) to afford 3-(2,2-dioxido-1-phenyl-1H-4,2,1-benzoxathiazin-3-yl)-N-methylpropan-1-amine hydrochloride (0.120 g, 38%) as a solid.

HPLC purity=100% at 210-370 nm; RT=6.7 min; 85/15-5/95 (ammonium formate buffer pH=3.5/ACN+MeOH) for 10 minutes, hold 4 minutes Xterra RP18, 3.5μ, 150×4.6 mm, 1.2 mL/minutes.

HRMS: calculated for C₁₇H₂₀N₂O₃S+H+, 333.12674; found (ESI, [M+H]⁺), 333.1267.

### Example 13

### 3-[(3S)-2,2-dioxido-1-phenyl-1H-4,2,1-benzoxathiazin-3-yl]-N-methylpropan-1-amine

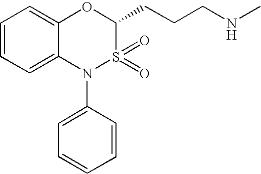

Step 1. Approximately 1.8 g of racemic 3-(2,2-dioxido-1-phenyl-1H-4,2,1-benzoxathiazin-3-yl)propan-1-ol, was dissolved in 40 mL of 1:1 methanol/acetonitrile, and the resulting solution was injected onto the Supercritical Fluid Chromatography (SFC) instrument with a volume of 1.5 mL per injection. The baseline resolved enantiomers were collected using a Berger MultiGram Prep SFC (Berger Instruments, Inc. Newark, Del.) under the following conditions: Chiralpak AD-H SFC column (5μ, 250 mm L×20 mm ID, Chiral Technologies, Inc, Exton, Pa.), 35° C. column temperature, 20% MeOH as CO₂ modifier, 50 mL/min flow rate, 100 bar outlet pressure, 220 nm UV detection.

The chiral purity of each enantiomer was determined under the same SFC conditions using a Chiralpak AD-H column (5μ, 250 mm L×4.6 mm ID) at 2.0 mL/min flow rate on a Berger Analytical SFC instrument. Both compounds were determined to be >99.9% chirally pure (Rₜ 5.5 and 11.6 min). S isomer CD analysis: MeOH at 25.2° C. +at 248 nm, −at 221 nm, +at 205 nm; Jasco J-715. R isomer CD analysis: MeOH at 25.1° C. −at 248 nm, +at 221 nm, −at 204 nm

Enantiomer 1, arbitrarily assigned as 3-[(3S)-2,2-dioxido-1-phenyl-1H-4,2,1-benzoxathiazin-3-yl]propan-1-ol: Rt 5.5 minutes. CD: MeOH at 25.2° C. +at 248 nm, −at 221 nm, +at 205 nm; Jasco J-715

Enantiomer 2, arbitrarily assigned as 3-[(3R)-2,2-dioxido-1-phenyl-1H-4,2,1-benzoxathiazin-3-yl]propan-1-ol: Rt 11.6 min: MeOH at 25.1° C. −at 248 nm, +at 221 nm, −at 204 nm

Step 2: to a solution of 3-[(3S)-2,2-dioxido-1-phenyl-1H-4,2,1-benzoxathiazin-3-yl]propan-1-ol (enantiomer 1, Rt 5.5 minutes. CD: MeOH at 25.2° C. +at 248 nm, −at 221 nm, +at 205 nm; Jasco J-715) (0.900 g, 2.82 mmol) in dichloromethane (15 mL) at room temperature was added triethylamine (1.5 mL). p-Toluenesulfonyl chloride (0.600 g, 3.18 mmol) added the reaction stirred overnight. Methylamine (33% solution in ethanol, 50 mL) was added and the reaction stirred overnight. The reaction was then poured into 2N hydrochloric acid and extracted with diethyl ether. The aqueous layer was basified with potassium carbonate, extracted with ethyl acetate, the combined organic layers were dried over magnesium sulfate, and concentrated in vacuo. The residue was purified by SiO₂ column chromatography (96.5:3.5 dichloromethane:7N ammonia/methanol). The residue was dissolved in methanol and treated with hydrogen chloride (4N in dioxane) to afford 3-[(3S)-2,2-dioxido-1-phenyl-1H-4,2,1-benzoxathiazin-3-yl]-N-methylpropan-1-amine hydrochloride (0.225 g, 21%).

Chiral HPLC purity component=99.5%/0.5% at 220 nm; RT=3.8 minutes and 7.6 min; 20% MeOH w/0.2% DEA Chiralpak OJ-H (4.6×250 mm), 2 mL/minutes.

HRMS: calculated for C₁₇H₂₀N₂O₃S+H+, 333.1204; found (ESI, [M+H]⁺), 333.1267.

CD analysis: MeOH at 25.3° C. +at 247 nm, −at 220 nm, +at 203 nm; Jasco J-715

### Example 14

### 3-[(3R)-2,2-dioxido-1-phenyl-1H-4,2,1-benzoxathiazin-3-yl]-N-methylpropan-1-amine

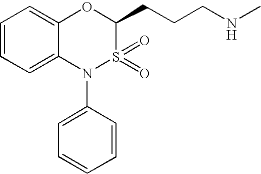

To a solution of 3-[(3R)-2,2-dioxido-1-phenyl-1H-4,2,1-benzoxathiazin-3-yl]propan-1-ol (0.900 g, 2.82 mmol) (enantiomer 2, Rt 11.6 min: MeOH at 25.1° C. −at 248 nm, +at 221 nm, −at 204 nm) in dichloromethane (15 mL) at room temperature was added triethylamine (1.5 mL). p-Toluenesulfonyl chloride (0.600 g, 3.18 mmol) was added and the reaction stirred overnight. Methylamine (33% solution in ethanol, 50 mL) was added and the reaction stirred overnight. The reaction was then poured into 2N hydrochloric acid and extracted with diethyl ether. The aqueous layer was basified with potassium carbonate, extracted with ethyl acetate; the combined organic layers were dried over magnesium sulfate, and concentrated in vacuo. The residue was purified by SiO₂ column chromatography (96.5:3.5, dichloromethane:7N ammonia/methanol). The residue was dissolved in methanol and treated with hydrogen chloride (4N in dioxane) to afford 3-[(3R)-2,2-dioxido-1-phenyl-1H-4,2,1-benzoxathiazin-3-yl]-N-methylpropan-1-amine hydrochloride (0.180 g, 17%).

Chiral HPLC purity=96.3%/3.7% at 220 nm; RT=7.6 min and 3.8 min; 20% MeOH w/0.2% DEA Chiralpak OJ-H (4.6×250 mm), 2 mL/minutes.

HRMS: calculated for C₁₇H₂₀N₂O₃S+H+, 333.1267;

CD analysis: MeOH at 25.3° C. −at 246 nm, +at 220 nm, −at 202 nm; Jasco J-715

### Example 15

### 3-[1-(4-fluorophenyl)-2,2-dioxido-1H-4,2,1-benzoxathiazin-3-yl]-N-methylpropan-1-amine

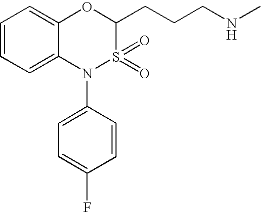

### Step 1: 1-(4-Methoxy-benzyl)-1H-benzo[1,3,4]oxathiazine 2,2-dioxide

To a stirred solution of 1H-benzo[1,3,4]oxathiazine 2,2-dioxide (8.25 g, 44.5 mmol) in anhydrous N,N-dimethylformamide (140 mL), under nitrogen was added sodium hydride (60% oil dispersion, 1.81 g, 45.4 mmol) and the reaction stirred for 30 minutes. 4-methoxybenzyl chloride (7.6 mL, 56.2 mmol) was added and the solution heated to 50° C. for 18 hours. The reaction was cooled to room temperature and quenched with saturated solution of ammonium chloride (500 mL) and extracted three times with ethyl acetate (250 mL). The organic extracts were combined and washed five times with H₂O (200 mL), dried (MgSO₄), filtered and the solvent removed, in vacuo, to give an amber colored oil (14.56 g). The residue was purified by SiO2 column chromatography, eluting with a gradient of 0-30% ethyl acetate in hexane to afford the product as a lightly yellow colored oil (13.39 g, 99% yield).

### Step 2: 3-allyl-1-(4-methoxybenzyl)-3,4-dihydro-1H-2,1-benzothiazine 2,2-dioxide

To a stirred solution of 1-(4-methoxy-benzyl)-1H-benzo[1,3,4]oxathiazine 2,2-dioxide (13.3 g, 43.4 mmol) in anhydrous tetrahydrofuran (150 mL), under nitrogen, at 0° C., was added lithium bis(trimethylsilyl)amide (1M in THF, 47.7 mL, 47.7 mmol) dropwise. After 20 minutes allyl bromide (4.13 mL, 47.7 mmol) in THF (15 mL) was added drop wise, and the solution warmed to room temperature and stirred under nitrogen for 18 hours. The reaction was quenched with saturated ammonium chloride (400 mL) and extracted with ethyl acetate (150 mL). The aqueous phase was separated and the organic layer washed with saturated ammonium chloride (250 mL). The aqueous washings were combined and extracted twice with ethyl acetate (150 mL). The organic extracts were combined and washed with brine (100 mL), dried (MgSO₄), filtered and the solvent removed, in vacuo, to give an orange-red oil (14.2 g). This material was adsorbed onto silica and purified by SiO₂ column chromatography, eluting with a gradient of 0-20% ethyl acetate in hexane. The residue was re-crystallized from ethyl acetate/hexane to afford the product as an off white solid (6.79 g, 45% yield).

### Step 4: 3-[1-(4-methoxybenzyl)-2,2-dioxido-1H-4,2,1-benzoxathiazin-3-yl]propan-1-ol

In an analogous procedure to that in Example 12, step 5, 3-[1-(4-methoxybenzyl)-2,2-dioxido-1H-4,2,1-benzoxathiazin-3-yl]propan-1-ol 7.68 g (96% yield) was prepared from 3-allyl-1-(4-methoxybenzyl)-3,4-dihydro-1H-2,1-benzothiazine 2,2-dioxide.

### Step 5: 3-(3-Chloro-propyl)-1-(4-methoxy-benzyl)-1H-benzo[1,3,4]oxathiazine 2,2-dioxide

To a stirred solution of 3-[1-(4-methoxybenzyl)-2,2-dioxido-1H-4,2,1-benzoxathiazin-3-yl]propan-1-ol (200 mg, 0.55 mmol) in anhydrous tetrahydrofuran (3 mL), under nitrogen at room temperature, was added triphenyl phosphine (217 mg, 0.83 mmol). N-Chlorosuccinimide (110 mg, 0.83 mmol) was added, portion wise, and the solution stirred for 1.5 hours. The reaction was concentrated to dryness and the material adsorbed onto silica and purified by SiO₂ column chromatography, eluting with a gradient of 0-35% ethyl acetate in hexane to afford the product as a white solid (170 mg, 81% yield).

### Step 6: 3-(3-chloropropyl)-1H-4,2,1-benzoxathiazine 2,2-dioxide

To a stirred solution of 3-(3-Chloro-propyl)-1-(4-methoxy-benzyl)-1H-benzo[1,3,4]oxathiazine 2,2-dioxide (200 mg, 0.52 mmol) and anisole (250 μL, 2.30 mmol) in dichloromethane (4 mL) was added trifluoroacetic acid (2 mL) and the solution stirred, under nitrogen at room temperature. After 18 hours the reaction was diluted with dichloromethane (100 mL) and washed four times with H₂O (75 mL). The aqueous washings were combined and extracted three times with dichloromethane (75 mL). The organic extracts were combined, dried (MgSO₄), filtered and evaporated, in vacuo, to give a brown solid (220 mg). This solid was adsorbed onto silica and purified by SiO₂ column chromatography, eluting with a gradient of 0-30% ethyl acetate in hexane to afford the product as a white solid (70 mg, 51% yield).

### Step 7: 3-(3-Chloro-propyl)-1-(4-fluoro-phenyl)-1H-benzo[1,3,4]oxathiazine 2,2-dioxide

To a stirred mixture of 3-(3-chloropropyl)-1H-4,2,1-benzoxathiazine 2,2-dioxide (100 mg, 0.38 mmol), copper (II) acetate (104 mg, 0.57 mmol), 4-fluorophenyl boronic acid (107 mg, 7.64 mmol) and 4 A molecular sieves in dichloromethane (5 mL), under nitrogen, at 25° C., was added pyridine (76 μL, 7.64 mmol) and the mixture stirred for 18 hours. The solvent was removed, in vacuo, and the material adsorbed onto silica and purified by SiO2 column chromatography, eluting with a gradient of 0-30% ethyl acetate in hexane to afford the product as an off white wax (104 mg, 76% yield).

### Step 8: 3-[1-(4-fluorophenyl)-2,2-dioxido-1H-4,2,1-benzoxathiazin-3-yl]-N-methylpropan-1-amine

To a vial containing 3-(3-chloro-propyl)-1-(4-fluoro-phenyl)-1H-benzo[1,3,4]oxathiazine 2,2-dioxide (125 mg, 0.35 mmol) and a catalytic amount of sodium iodide was added methylamine solution (33% in ethanol, 25 mL), the vial was capped and shaken at 60° C. for 18 hours. The solvent was removed, in vacuo, and the material adsorbed onto silica and purified by SiO2 column chromatography, eluting with a gradient of 0-10% NH₃-MeOH in dichloromethane to give an amber colored oil. This oil was dissolved in methanol and treated with activated charcoal, filtered through Celite and the solvent removed to give a clear oil. This oil was dissolved in diethyl ether (20 mL) and HCl (1N in diethyl ether 1 mL) was added. The diethyl ether was removed to give a brown solid (68 mg, 50% yield). HPLC purity 97.7% at 210-370 nm, 7.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C17H19FN2O3S+H+, 351.1173; found (ESI, [M+H]+), 351.116

### Example 16

### N-methyl-3-[1-(3-methylphenyl)-2,2-dioxido-1H-4,2,1-benzoxathiazin-3-yl]propan-1-amine

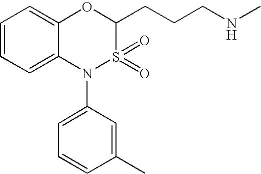

In an analogous manner to Example 15, step 7 and step 8, N-methyl-3-[1-(3-methyl phenyl)-2,2-dioxido-1H-4,2,1-benzoxathiazin-3-yl]propan-1-amine hydrochloride (22 mg, 30% yield) was prepared from 3-(3-chloro-propyl)-1H-benzo[1,3,4]oxathiazine 2,2-dioxide and 3-methylphenyl boronic acid.

HPLC purity 100.0% at 210-370 nm, 7.6 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₃S+H+, 347.1424; found (ESI, [M+H]⁺), 347.1419

### Example 17

### N-methyl-3-[1-(4-methylphenyl)-2,2-dioxido-1H-4,2,1-benzoxathiazin-3-yl]propan-1-amine

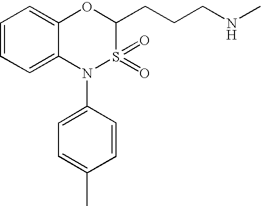

In an analogous manner to Example 15, step 7 and step 8, N-methyl-3-[1-(4-methyl phenyl)-2,2-dioxido-1H-4,2,1-benzoxathiazin-3-yl]propan-1-amine hydrochloride (28 mg, 38% yield) was prepared from 3-(3-chloro-propyl)-1H-benzo[1,3,4]oxathiazine 2,2-dioxide and 4-methylphenyl boronic acid.

HPLC purity 100.0% at 210-370 nm, 7.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₃S+H+, 347.1424; found (ESI, [M+H]⁺), 347.1424

### Example 18

### 3-[1-(3-methoxyphenyl)-2,2-dioxido-1H-4,2,1-benzoxathiazin-3-yl]-N-methylpropan-1-amine

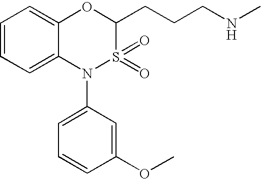

In an analogous manner to Example 15, step 7 and step 8, 3-[1-(3-methoxyphenyl)-2,2-dioxido-1H-4,2,1-benzoxathiazin-3-yl]-N-methylpropan-1-amine hydrochloride (79 mg, 66% yield) was prepared from 3-(3-chloro-propyl)-1H-benzo[1,3,4]oxathiazine 2,2-dioxide and 3-methoxyphenyl boronic acid.

HPLC purity 100.0% at 210-370 nm, 7.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₄S+H+, 363.1373; found (ESI, [M+H]⁺), 363.1373

### Example 19

### 3-[1-(4-methoxyphenyl)-2,2-dioxido-1H-4,2,1-benzoxathiazin-3-yl]-N-methylpropan-1-amine

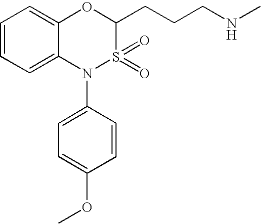

In an analogous manner to Example 15, step 7 and step 8, 3-[1-(4-methoxyphenyl)-2,2-dioxido-1H-4,2,1-benzoxathiazin-3-yl]-N-methylpropan-1-amine hydrochloride (108 mg, 91% yield) was prepared from 3-(3-chloro-propyl)-1H-benzo[1,3,4]oxathiazine 2,2-dioxide and 4-methoxyphenyl boronic acid.

HPLC purity 100.0% at 210-370 nm, 7.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₄S+H+, 363.1373; found (ESI, [M+H]⁺), 363.1362.

### Example 20

### 3-[1-(3-fluorophenyl)-2,2-dioxido-1H-4,2,1-benzoxathiazin-3-yl]-N-methylpropan-1-amine

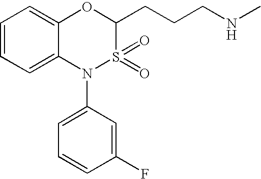

In an analogous manner to Example 15, step 7 and step 8, 3-[1-(3-fluorophenyl)-2,2-dioxido-1H-4,2,1-benzoxathiazin-3-yl]-N-methylpropan-1-amine hydrochloride (63 mg, 58% yield) was prepared from 3-(3-chloro-propyl)-1H-benzo[1,3,4]oxathiazine 2,2-dioxide and 3-fluorophenyl boronic acid.

HPLC purity 98.4% at 210-370 nm, 7.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉FN₂O₃S+H+, 351.1173; found (ESI, [M+H]⁺), 351.1175.

### Example 21

### N-methyl-3-[1-(2-naphthyl)-2,2-dioxido-1H-4,2,1-benzoxathiazin-3-yl]propan-1-amine

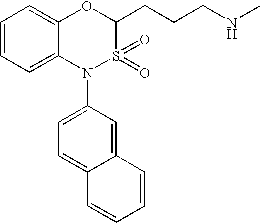

In an analogous manner to Example 15, step 7 and step 8, N-methyl-3-[1-(2-naphthyl)-2,2-dioxido-1H-4,2,1-benzoxathiazin-3-yl]propan-1-amine hydrochloride (14 mg, 22% yield) was prepared from 3-(3-chloro-propyl)-1H-benzo[1,3,4]oxathiazine 2,2-dioxide and 2-naphthyl boronic acid.

HPLC purity 100.0% at 210-370 nm, 8.6 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C21H₂₂N₂O₃S+H+, 383.1424; found (ESI, [M+H]⁺), 383.1439.

### Example 22

### 3-[1-(3,5-dimethylphenyl)-2,2-dioxido-1H-4,2,1-benzoxathiazin-3-yl]-N-methylpropan-1-amine

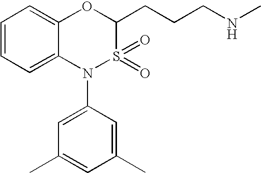

In an analogous manner to Example 15, step 7 and step 8, 3-[1-(3,5-dimethylphenyl)-2,2-dioxido-1H-4,2,1-benzoxathiazin-3-yl]-N-methylpropan-1-amine hydrochloride (107 mg, 82% yield) was prepared from 3-(3-chloro-propyl)-1H-benzo[1,3,4]oxathiazine 2,2-dioxide and 3,5-dimethylphenyl boronic acid.

HPLC purity 100.0% at 210-370 nm, 8.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₃S+H+, 361.1581; found (ESI, [M+H]⁺), 361.1586.

### Example 23

### 2-(2,2-dioxido-1-phenyl-1H-4,2,1-benzoxathiazin-3-yl)-N-ethylethanamine

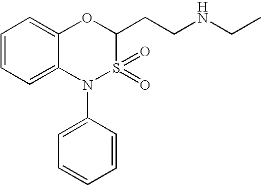

### Step 1: 2-(2,2-dioxido-1-phenyl-3,4-dihydro-1H-2,1-benzothiazin-3-yl)ethanol

Nitrogen was bubbled into a stirring solution of 3-allyl-1-phenyl-3,4-dihydro-1H-2,1-benzothiazine 2,2-dioxide (200 mg, 0.66 mmol) in methanol at −78° C. for 20 minutes, then the nitrogen line was replace and ozone was bubbled in for 45 minutes and the solution turned a light blue color. The reaction mixture was flushed with nitrogen and then sodium borohydride was added and the mixture warmed to room temperature. After 2 hours the solvent was removed and the residue taken into ethyl acetate (100 mL) and washed three times with a saturated solution of ammonium chloride (100 mL). The aqueous washings were combined and extracted twice with ethyl acetate (100 mL). The organic extracts were combined, dried (MgSO₄), filtered and the solvent removed, in vacuo, to give a white foam (200 mg). This foam was adsorbed onto silica and purified by SiO₂ column chromatography, eluting with a gradient of 0-50% ethyl acetate in hexane to afford the product as a white oil (113 mg, 56% yield).

### Step 2: 3-(2-bromoethyl)-1-phenyl-3,4-dihydro-1H-2,1-benzothiazine 2,2-dioxide

To a stirred solution of 2-(2,2-dioxido-1-phenyl-3,4-dihydro-1H-2,1-benzothiazin-3-yl)ethanol (1.10 g, 3.60 mmol) in anhydrous tetrahydrofuran (40 mL) was added triphenyl phosphine (1.42 g, 6.14 mmol) and N-bromosuccinamide (962 mg, 1.42 mmol) under nitrogen at room temperature. After 18 hours the solvent was removed and the resultant oil adsorbed onto silica and purified by SiO₂ column chromatography, eluting with a gradient of 0-20% ethyl acetate in hexane to afford the product as a white solid (1.23 g, 93% yield).

HPLC purity 100.0% at 210-370 nm, 11.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₅H₁₄BrNO₃S+H+, 367.9950; found (ESI, [M+H—SO₂]⁺), 304.0301.

### Step 3: 2-(2,2-dioxido-1-phenyl-1H-4,2,1-benzoxathiazin-3-yl)-N-ethylethanamine

A vial containing 3-(2-bromo-ethyl)-1-phenyl-1H-benzo[1,3,4]oxathiazine 2,2-dioxide (79 mg, 0.22 mmol) and ethylamine solution (2M in methanol, 25 mL) was capped and stirred at 60° C. for 18 hours. The mixture was diluted with diethyl ether (150 mL) and washed five times with H₂O (50 mL). The aqueous washings were combined and extracted twice with diethyl ether (100 mL). The organic extracts were combined, dried (MgSO₄), filtered and the solvent removed, in vacuo, to give an amber colored oil (79 mg). This oil was adsorbed onto silica and purified by SiO₂ column chromatography, eluting with a gradient of 0-10% 33% NH₃-MeOH in dichloromethane to give a clear oil (44 mg). This oil was dissolved in diethyl ether (90 mL) and 2 equivalents of HCl (2M in Et₂O) were added and allowed to stir for 1 hour. 2-(2,2-dioxido-1-phenyl-1H-4,2,1-benzoxathiazin-3-yl)-N-ethylethanamine hydrochloride was collected and dried in vacuo for 18 hours as a white solid (29 mg, 37% yield).

HPLC purity 100.0% at 210-370 nm, 6.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₂₀N₂O₃S+H+, 333.12674; found (ESI, [M+H]⁺), 333.1259.

### Example 24

### 2-(2,2-dioxido-1-phenyl-1H-4,2,1-benzoxathiazin-3-yl)ethanamine

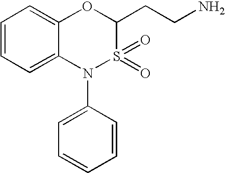

In an analogous procedure to that used in Example 23, step 3, 2-(2,2-dioxido-1-phenyl-1H-4,2,1-benzoxathiazin-3-yl)ethanamine hydrochloride (61 mg, 66% yield) was prepared from 3-(2-bromoethyl)-1-phenyl-3,4-dihydro-1H-2,1-benzothiazine 2,2-dioxide and ammonia.

HPLC purity 97.6% at 210-370 nm, 6.7 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₁₅H₁₆N₂O₃S+H+, 305.09544; found (ESI, [M+H]⁺), 305.095.

### Example 25

### 2-(2,2-dioxido-1-phenyl-1H-4,2,1-benzoxathiazin-3-yl)-N-methylethanamine

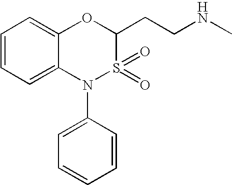

In an analogous procedure to that used in Example 23, step 3, 2-(2,2-dioxido-1-phenyl-1H-4,2,1-benzoxathiazin-3-yl)-N-methylethanamine hydrochloride (69 mg, 71% yield) was prepared from 3-(2-bromoethyl)-1-phenyl-3,4-dihydro-1H-2,1-benzothiazine 2,2-dioxide and methylamine.

HPLC purity 100.0% at 210-370 nm, 6.8 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₁₆H₁₈N₂O₃S+H+, 319.11109; found (ESI, [M+H]⁺), 319.1116.

### Example 26

### 2-(2,2-dioxido-1-phenyl-1H-4,2,1-benzoxathiazin-3-yl)-N,N-dimethylethanamine

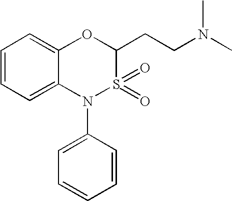

In an analogous procedure to that used in Example 23, step 3, 2-(2,2-dioxido-1-phenyl-1H-4,2,1-benzoxathiazin-3-yl)-N,N-dimethylethanamine hydrochloride (37 mg, 37% yield) was prepared from 3-(2-bromoethyl)-1-phenyl-3,4-dihydro-1H-2,1-benzothiazine 2,2-dioxide and dimethylamine.

HPLC purity 100.0% at 210-370 nm, 6.8 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₁₇H₂₀N₂O₃S+H+, 333.12674; found (ESI, [M+H]⁺), 333.1274.

### Example 27

### N-[2-(2,2-dioxido-1-phenyl-1H-4,2,1-benzoxathiazin-3-yl)ethyl]propan-1-amine

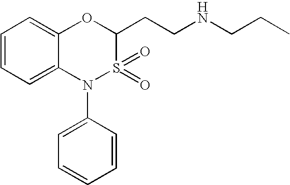

In an analogous procedure to that used in Example 23, step 3, N-[2-(2,2-dioxido-1-phenyl-1H-4,2,1-benzoxathiazin-3-yl)ethyl]propan-1-amine hydrochloride (65 mg, 63% yield) was prepared from 3-(2-bromoethyl)-1-phenyl-3,4-dihydro-1H-2,1-benzothiazine 2,2-dioxide and propylamine.

HPLC purity 100.0% at 210-370 nm, 7.4 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₁₈H₂₂N₂O₃S+H+, 347.14239; found (ESI, [M+H]⁺), 347.142.

### Example 28

### N-[2-(2,2-dioxido-1-phenyl-1H-4,2,1-benzoxathiazin-3-yl)ethyl]-2-methylpropan-1-amine

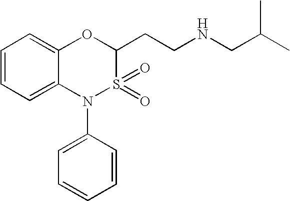

In an analogous procedure to that used in Example 23, step 3, N-[2-(2,2-dioxido-1-phenyl-1H-4,2,1-benzoxathiazin-3-yl)ethyl]-2-methylpropan-1-amine hydrochloride (83 mg, 77% yield) was prepared from 3-(2-bromoethyl)-1-phenyl-3,4-dihydro-1H-2,1-benzothiazine 2,2-dioxide and sec-butylamine.

HPLC purity 98.2% at 210-370 nm, 7.8 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₁₉H₂₄N₂O₃S+H+, 361.15804; found (ESI, [M+H]⁺), 361.1574.

### Example 29

### 1-phenyl-3-(2-pyrrolidin-1-ylethyl)-1H-4,2,1-benzoxathiazine 2,2-dioxide

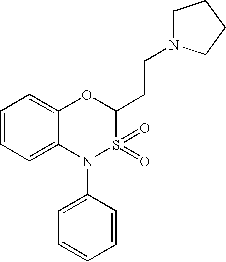

In an analogous procedure to that used in Example 23, step 3, 1-phenyl-3-(2-pyrrolidin-1-ylethyl)-1H-4,2,1-benzoxathiazine 2,2-dioxide hydrochloride (101 mg, 94% yield) was prepared from 3-(2-bromoethyl)-1-phenyl-3,4-dihydro-1H-2,1-benzothiazine 2,2-dioxide and pyrrolidine.

HPLC purity 95.1% at 210-370 nm, 7.1 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₁₉H₂₂N₂O₃S+H+, 359.14239; found (ESI, [M+H]⁺), 359.1428.

### Example 30

### 3-[2-(4-methylpiperazin-1-yl)ethyl]-1-phenyl-1H-4,2,1-benzoxathiazine 2,2-dioxide

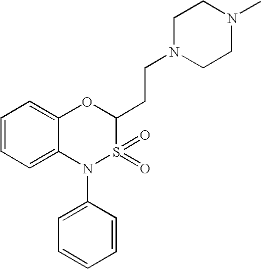

In an analogous procedure to that used in Example 23, step 3, 3-[2-(4-methylpiperazin-1-yl)ethyl]-1-phenyl-1H-4,2,1-benzoxathiazine 2,2-dioxide hydrochloride (100 mg, 87% yield) was prepared from 3-(2-bromoethyl)-1-phenyl-3,4-dihydro-1H-2,1-benzothiazine 2,2-dioxide and 1-methylpiperazine.

HPLC purity 98.6% at 210-370 nm, 7.2 minutes; Xterra RP18, 3.5μ, 150×4.6 mm column, 1.2 mL/minutes. 85/15-5/95 (ammonium bicarbonate buffer pH=9.5/ACN+MeOH) for 10 minutes, hold 4 minutes.

HRMS: calculated for C₂₀H₂₅N₃O₃S+H+, 388.16894; found (ESI, [M+H]⁺), 388.1709.

### Example 31

### N-[2-(2,2-dioxido-1-phenyl-1H-4,2,1-benzoxathiazin-3-yl)ethyl]butan-1-amine

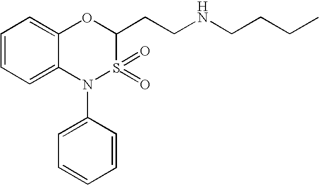

In an analogous procedure to that used in Example 23, step 3, N-[2-(2,2-dioxido-1-phenyl-1H-4,2,1-benzoxathiazin-3-yl)ethyl]butan-1-amine hydrochloride (41 mg, 38% yield) was prepared from 3-(2-bromoethyl)-1-phenyl-3,4-dihydro-1H-2,1-benzothiazine 2,2-dioxide and n-butylamine.

HPLC purity 100.0% at 210-370 nm, 7.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₃S+H+, 361.15804; found (ESI, [M+H]⁺), 361.1588.

### Example 32

### N-[2-(2,2-dioxido-1-phenyl-1H-4,2,1-benzoxathiazin-3-yl)ethyl]cyclobutanamine

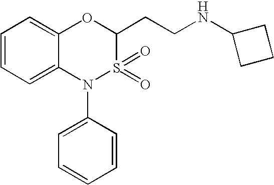

In an analogous procedure to that used in Example 23, step 3, N-[2-(2,2-dioxido-1-phenyl-1H-4,2,1-benzoxathiazin-3-yl)ethyl]butan-1-amine hydrochloride (79 mg, 74% yield) was prepared from 3-(2-bromoethyl)-1-phenyl-3,4-dihydro-1H-2,1-benzothiazine 2,2-dioxide and cyclobutylamine.

HPLC purity 95.0% at 210-370 nm, 7.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₂N₂O₃S+H+, 359.14239; found (ESI, [M+H]⁺), 359.1545.

### Example 33

### 3-(2,2-dioxido-1-phenyl-1H-4,2,1-benzoxathiazin-3-yl)propan-1-amine

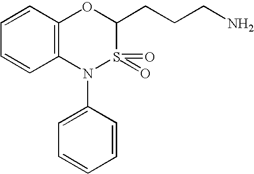

Step 1: A solution of 3-(3-chloropropyl)-1-phenyl-1H-4,2,1-benzoxathiazine 2,2-dioxide (0.34 g, 1.0 mmol) in dimethylformamide (3.0 mL) was treated with potassium phthalimide (0.28 g, 1.5 mmol) and heated at 65° C. for 16 hours. The reaction mixture was diluted with diethyl ether (25 mL), washed with 2 M aqueous sodium hydroxide (25 mL), dried (Na₂SO₄), and evaporated. Column chromatography (SiO₂, 3-100% ethyl acetate/hexanes) provided 2-[3-(2,2-dioxido-1-phenyl-1H-4,2,1-benzoxathiazin-3-yl)propyl]-1H-isoindole-1,3(2H)-dione (0.23 g, 51%) as a white powder:

HPLC purity 100.0% at 210-370 nm, 11.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₄H₂₀N₂O₅S+H+, 449.11657; found (ESI, [M+H—SO2]⁺), 385.1483.

Step 2: A solution of 2-[3-(2,2-dioxido-1-phenyl-1H-4,2,1-benzoxathiazin-3-yl)propyl]-1H-isoindole-1,3(2H)-dione (0.18 g, 0.42 mmol) in ethanol (2.0 mL) was treated with hydrazine (0.26 mL, 8.4 mmol) and heated to 78° C. for 6 hours. The reaction mixture was filtered, and the filtrate evaporated and purified by column chromatography (SiO₂, 0-5% 7 M NH₃-methanol/dichloromethane). The purified free-base was dissolved in ethyl ether (10 mL) and treated with hydrogen chloride (1.0 mL of a 2 M solution in ethyl ether), resulting in a white precipitate that was isolated by decantation and dried under vacuum to afford 3-(2,2-dioxido-1-phenyl-1H-4,2,1-benzoxathiazin-3-yl)propan-1-amine hydrochloride (0.10 g, 67%) as a white powder:

HPLC purity 100.0% at 210-370 nm, 6.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₆H₁₈N2O3S+H+, 319.11109; found (ESI, [M+H]⁺), 319.1114.

### Example 34

### 3-(2,2-dioxido-1-phenyl-1H-4,2,1-benzoxathiazin-3-yl)-N-ethylpropan-1-amine

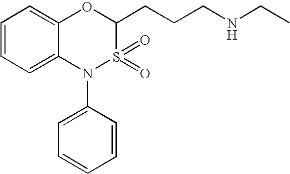

Step 1: A solution of 1-phenyl-1H-4,2,1-benzoxathiazine 2,2-dioxide (4.56 g, 17.4 mmol) in tetrahydrofuran (40 mL) was cooled to −78° C., treated with Lithium bis(trimethylsilyl)-amide (17.5 mL of a 1.0 M tetrahydrofuran solution, 1.75 mmol), stirred at −78° C. for two hours and then was allowed to warm to 23° C. The reaction mixture was quenched by the addition of 2 M hydrochloric acid (200 mL) and extracted with dichloromethane (3×300 mL), dried (MgSO₄), and evaporated. Column chromatography (SiO₂, 0-100% hexane/dichloromethane) provided 3-(3-bromopropyl)-1-phenyl-1H-4,2,1-benzoxathiazine 2,2-dioxide (5.213 g, 78%) as a tan solid:

HPLC purity 91.7% at 210-370 nm, 10.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

MS (ES) m/z 317.8.

Step 2: A solution of 3-(3-bromopropyl)-1-phenyl-1H-4,2,1-benzoxathiazine 2,2-dioxide (0.2 g, 0.525 mmol) and ethylamine (2.0 M in tetrahydrofuran, 1 mL, 2.0 mmol) were combined and stirred in a capped vial for 16 hours. The content of vial was absorbed on silica gel and purified by column chromatography (SiO₂, 0-15% NH₃-methanol/dichloromethane) to provide a tan residue. The residue was dissolved in diethyl ether (5 mL) and treated with hydrogen chloride (2 M solution in ethyl ether, 1.0 mL) to afford 3-(2,2-dioxido-1-phenyl-1H-4,2,1-benzoxathiazin-3-yl)-N-ethylpropan-1-amine hydrochloride (0.1156 g, 58%) as a white solid which was dried in vacuo:

MS (ES) m/z 346.9;

HPLC purity 100.0% at 210-370 nm, 7.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₃S+H+, 347.14239; found (ESI, [M+H]⁺), 347.1421;

### Example 35

### 3-(2,2-dioxido-1-phenyl-1H-4,2,1-benzoxathiazin-3-yl)-N-propylpropan-1-amine

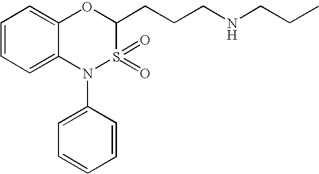

In an analogous manner to Example 34 step 2, 3-(3-bromopropyl)-1-phenyl-1H-4,2,1-benzoxathiazine 2,2-dioxide (0.2 g, 0.525 mmol) was treated with propylamine (430 μL, 5.25 mmol) to provide 3-(2,2-dioxido-1-phenyl-1H-4,2,1-benzoxathiazin-3-yl)-N-propylpropan-1-amine hydrochloride (0.1231 g, 59%) as a white solid:

MS (ES) m/z 360.9;

HPLC purity 97.6% at 210-370 nm, 8.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₃S+H+, 361.15804; found (ESI, [M+H]⁺), 361.1579.

### Example 36

### N-[3-(2,2-dioxido-1-phenyl-1H-4,2,1-benzoxathiazin-3-yl)propyl]butan-1-amine

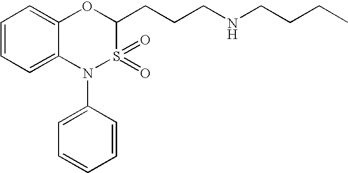

In an analogous manner to Example 34 step 2, 3-(3-bromopropyl)-1-phenyl-1H-4,2,1-benzoxathiazine 2,2-dioxide (0.2 g, 0.525 mmol) was treated with butylamine (520 μL, 5.26 mmol) to provide N-[3-(2,2-dioxido-1-phenyl-1H-4,2,1-benzoxathiazin-3-yl)propyl]butan-1-amine hydrochloride (0.103 g, 48%) as a white solid.

MS (ES) m/z 375.0;

HPLC purity 99.1% at 210-370 nm, 8.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₀H₂₆N₂O₃S+H+, 375.17369; found (ESI, [M+H]⁺), 375.1747.

### Example 37

### 3-(2,2-dioxido-1-phenyl-1H-4,2,1-benzoxathiazin-3-yl)-N-isopropylpropan-1-amine

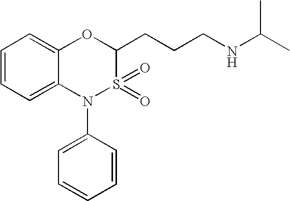

In an analogous manner to Example 34 step 2, 3-(3-bromopropyl)-1-phenyl-1H-4,2,1-benzoxathiazine 2,2-dioxide (0.2 g, 0.525 mmol) was treated with isopropylamine (450 μL, 5.28 mmol) to provide 3-(2,2-dioxido-1-phenyl-1H-4,2,1-benzoxathiazin-3-yl)-N-isopropylpropan-1-amine hydrochloride (0.0987 g, 47%)

MS (ES) m/z 361.0;

HPLC purity 99.2% at 210-370 nm, 8.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₃S+H+, 361.15804; found (ESI, [M+H]⁺), 361.1585.

### Example 38

### N-[3-(2,2-dioxido-1-phenyl-1H-4,2,1-benzoxathiazin-3-yl)propyl]-2-methylpropan-1-amine

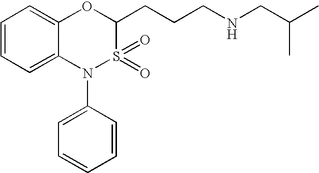

In an analogous manner to Example 34 step 2, 3-(3-bromopropyl)-1-phenyl-1H-4,2,1-benzoxathiazine 2,2-dioxide (0.2 g, 0.525 mmol) was treated with isobutylamine (525 μL, 5.23 mmol) to provide N-[3-(2,2-dioxido-1-phenyl-1H-4,2,1-benzoxathiazin-3-yl)propyl]-2-methylpropan-1-amine hydrochloride (0.1317 g, 61%) as a white solid:

MS (ES) m/z 375.0;

HPLC purity 100.0% at 210-370 nm, 8.6 minutes; Xterra RP18, 3.5μ, 150×4.6 mm column, 1.2 mL/minutes. 85/15-5/95 (ammonium formate buffer pH=3.5/ACN+MeOH) for

10 minutes, hold 4 minutes.

HRMS: calculated for C₂₀H₂₆N₂O₃S+H+, 375.17369; found (ESI, [M+H]⁺), 375.1733.

### Example 39

### 3-{[3-(2,2-dioxido-1-phenyl-1H-4,2,1-benzoxathiazin-3-yl)propyl]amino}propan-1-ol

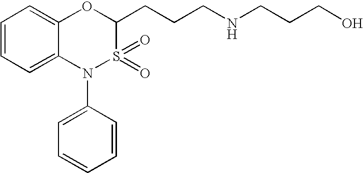

In an analogous manner to Example 34 step 2, 3-(3-bromopropyl)-1-phenyl-1H-4,2,1-benzoxathiazine 2,2-dioxide (0.2 g, 0.525 mmol) was treated with 3-amino-1-propanol (400 μL, 5.26 mmol) to provide 3-{[3-(2,2-dioxido-1-phenyl-1H-4,2,1-benzoxathiazin-3-yl)propyl]amino}propan-1-ol hydrochloride (0.0962 g, 44%) as a white solid:

MS (ES) m/z 377.2;

HPLC purity 100.0% at 210-370 nm, 7.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₄N₂O₄S+H+, 377.15295; found (ESI, [M+H]⁺), 377.1530.

### Example 40

### N-[3-(2,2-dioxido-1-phenyl-1H-4,2,1-benzoxathiazin-3-yl)propyl]cyclopropanamine

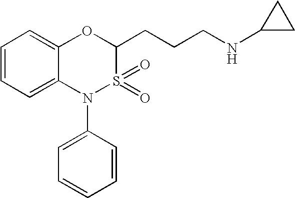

In an analogous manner to Example 34 step 2, 3-(3-bromopropyl)-1-phenyl-1H-4,2,1-benzoxathiazine 2,2-dioxide (0.2 g, 0.525 mmol) was treated with cyclopropylamine (360 μL, 5.2 mmol) to provide N-[3-(2,2-dioxido-1-phenyl-1H-4,2,1-benzoxathiazin-3-yl)propyl]cyclopropanamine hydrochloride (0.1416 g, 68%) as a white solid:

MS (ES) m/z 359.2;

HPLC purity 100.0% at 210-370 nm, 8.0 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₉H₂₂N₂O₃S+H+, 359.14239; found (ESI, [M+H]⁺), 359.1416.

### Example 41

### N-[3-(2,2-dioxido-1-phenyl-1H-4,2,1-benzoxathiazin-3-yl)propyl]cyclobutanamine

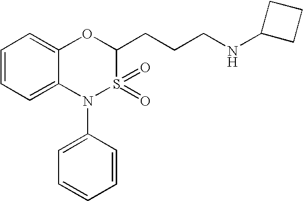

In an analogous manner to Example 34 step 2, 3-(3-bromopropyl)-1-phenyl-1H-4,2,1-benzoxathiazine 2,2-dioxide (0.2 g, 0.525 mmol) was treated with cyclobutylamine (450 μL, 5.27 mmol) to provide N-[3-(2,2-dioxido-1-phenyl-1H-4,2,1-benzoxathiazin-3-yl)propyl]cyclobutanamine hydrochloride (0.1113 g, 52%) as a white solid:

MS (ES) m/z 373.2;

HPLC purity 100.0% at 210-370 nm, 8.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₀H₂₄N₂O₃S+H+, 373.15804; found (ESI, [M+H]⁺), 373.1564.

### Example 42

### N-[3-(2,2-dioxido-1-phenyl-1H-4,2,1-benzoxathiazin-3-yl)propyl]cyclopentanamine

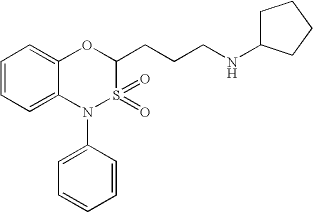

In an analogous manner to Example 34 step 2, 3-(3-bromopropyl)-1-phenyl-1H-4,2,1-benzoxathiazine 2,2-dioxide (0.2 g, 0.525 mmol) was treated with cyclopentylamine (520 μL, 5.27 mmol) to provide N-[3-(2,2-dioxido-1-phenyl-1H-4,2,1-benzoxathiazin-3-yl)propyl]cyclopentanamine hydrochloride (0.027 g, 12%) as a white solid:

MS (ES) m/z 387.2;

HPLC purity 97.0% at 210-370 nm, 8.7 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₁H₂₆N₂O₃S+H+, 387.17369; found (ESI, [M+H]⁺), 387.1750.

### Example 43

### N-[3-(2,2-dioxido-1-phenyl-1H-4,2,1-benzoxathiazin-3-yl)propyl]cyclohexanamine

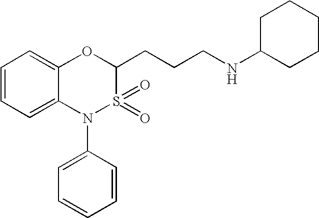

In an analogous manner to Example 34 step 2, 3-(3-bromopropyl)-1-phenyl-1H-4,2,1-benzoxathiazine 2,2-dioxide (0.2 g, 0.525 mmol) was treated with cyclohexylamine (600 μL, 5.25 mmol) to provide N-[3-(2,2-dioxido-1-phenyl-1H-4,2,1-benzoxathiazin-3-yl)propyl]cyclohexanamine hydrochloride (0.132 g, 57%) as a white solid:

MS (ES) m/z 401.1;

HPLC purity 100.0% at 210-370 nm, 9.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₂₂H₂₈N₂O₃S+H+, 401.18934; found (ESI, [M+H]⁺), 401.1892.

### Example 44

### 3-(7-fluoro-2,2-dioxido-1-phenyl-1H-4,2,1-benzoxathiazin-3-yl)-N-methylpropan-1-amine

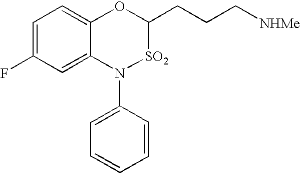

### Step 1: 7-fluoro-1H-4,2,1-benzoxathiazine 2,2-dioxide

To a solution of 2-amino-4-fluorophenol (1.0 g, 7.9 mmol) in 20 mL THF was added dropwise chloromethanesulfonyl chloride (1.3 g, 8.7 mmol). The mixture was stirred for 30 minutes under nitrogen at room temperature, whereupon pyridine (0.7 g, 8.7 mmol) was added in one portion. The mixture was stirred for 18 hours at room temperature, then poured into 100 mL of 2N HCl solution. The solution was extracted 3 times with ethyl acetate and the combined extracts were washed once with water. The organic layer was dried over anhydrous magnesium sulfate, filtered through a plug of silica gel and concentrated in vacuo. The crude product was triturated with hexane, forming a solid. The solid was dissolved in 20 mL of methanol and potassium carbonate (2.2 g, 15.8 mmol) added. The mixture was heated several hours at 60° C. until complete by LC/MS monitoring. The solution was concentrated and carefully quenched with 2N HCl, then extracted 3 times with ethyl acetate. The combined extracts were dried over magnesium sulfate then filtered through a plug of silica gel. The filtrate was concentrated in vacuo and the solid triturated and washed with hexane. The solid was collected by filtration to yield 7-fluoro-1H-4,2,1-benzoxathiazine 2,2-dioxide (0.65 g) as a tan solid.

MS (ES) m/z 201.9.

### Step 2: 3-(3-chloropropyl)-7-fluoro-1H-4,2,1-benzoxathiazine 2,2-dioxide

7-fluoro-1H-4,2,1-benzoxathiazine 2,2-dioxide (0.65 g, 3.2 mmol), was dissolved in 10 mL of dry THF (10 mL) and cooled to −78° C. A solution of n-butyl lithium (1.6 N in hexanes, 5.0 mL, 8.0 mmol) was added via syringe and the solution stirred for 15 minutes. 1-Bromo-3-chloro propane (0.35 mL, 3.6 mmol) was added in one portion, and the mixture allowed to warm to room temperature. After 1 hour, the reaction was quenched with saturated ammonium chloride solution and extracted 3 times with ethyl acetate. The combined ethyl acetate layers were dried over magnesium sulfate then filtered through a plug of silica gel and concentrated. The solid was washed with hexane and collected by filtration to yield of 3-(3-chloropropyl)-7-fluoro-1H-4,2,1-benzoxathiazine 2,2-dioxide (0.72 g) as a tan solid.

MS (ES) m/z 277.8

### Step 3: 3-(3-chloropropyl)-7-fluoro-1-phenyl-1H-4,2,1-benzoxathiazine 2,2-dioxide

3-(3-chloropropyl)-7-fluoro-1H-4,2,1-benzoxathiazine 2,2-dioxide (0.28 g, 1.0 mmol), copper (II) acetate (0.27 g, 1.51 mmol), phenylboronic acid (0.24 g, 2.01 mmol) and 4 A molecular sieves were placed in a 25 mL flask. 10 mL of dichloromethane (10 mL) and of pyridine (0.16 mL, 2.01 mmol) were added and the solution stirred at room temperature for 36 hr. The solution was then filtered through a plug of silica gel by elution with 10% ethyl acetate:hexane. The solution was concentrated and the residue was purified by SiO₂ column chromatography (10-35% gradient ethyl acetate/hexane) to yield 0.25 g of 3-(3-chloropropyl)-7-fluoro-1-phenyl-1H-4,2,1-benzoxathiazine 2,2-dioxide (0.25 g).

Step 4: To a suspension of sodium hydride (0.056 g, 1.41 mmol) in DMF (3 mL) was added a solution of methylcarbamic acid t-butyl ester (0.184 g, 1.41 mmol) in DMF (2 mL). After stirring 1 hr, a solution of 3-(3-chloropropyl)-7-fluoro-1-phenyl-1H-4,2,1-benzoxathiazine 2,2-dioxide (0.25 g, 0.72 mmol) in DMF (4 mL) was added. The mixture was stirred for 2 hr then poured into 2N HCl and extracted twice with ethyl acetate. The organic layers are dried over magnesium sulfate then concentrated and the residue purified by SiO₂ column chromatography (10-35% gradient ethyl acetate/hexane). The purified residue was then dissolved in 5 mL of 2N HCl in ether and 0.1 mL of MeOH and the solution allowed to stand 18 hr whereupon crystals formed. The crystals were collected by filtration to yield 3-(7-fluoro-2,2-dioxido-1-phenyl-1H-4,2,1-benzoxathiazin-3-yl)-N-methylpropan-1-amine hydrochloride (0.15 g):

MS (ES) m/z 350.9;

HPLC purity 100.0% at 210-370 nm, 7.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉FN₂O₃S+H+, 351.11732; found (ESI, [M+H]⁺), 351.1161.

### Example 45

### 3-(6-chloro-2,2-dioxido-1-phenyl-1H-4,2,1-benzoxathiazin-3-yl)-N-methylpropan-1-amine

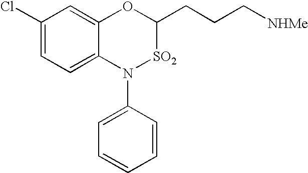

In an analogous manner to Example 44, step 1, 6-chloro-1H-4,2,1-benzoxathiazine 2,2-dioxide (0.35 g) was prepared from of 2-amino-5-chlorophenol (1.0 g).

MS (ES) m/z 217.9

In an analogous manner to Example 44, step 2, 6-chloro-3-(3-chloropropyl)-1H-4,2,1-benzoxathiazine 2,2-dioxide (0.25 g) was prepared from 6-chloro-1H-4,2,1-benzoxathiazine 2,2-dioxide (0.27 g).

MS (ES) m/z 293.7

In an analogous manner to Example 44, step 3, 3-(3-chloropropyl)-6-chloro-1-phenyl-1H-4,2,1-benzoxathiazine 2,2-dioxide (0.15 g) was prepared from 6-chloro-1H-4,2,1-benzoxathiazine 2,2-dioxide (0.20 g) and phenylboronic acid.

In an analogous manner to Example 44, step 4, 3-(6-chloro-2,2-dioxido-1-phenyl-1H-4,2,1-benzoxathiazin-3-yl)-N-methylpropan-1-amine hydrochloride (0.035 g) was prepared from 3-(3-chloropropyl)-6-chloro-1-phenyl-1H-4,2,1-benzoxathiazine 2,2-dioxide (0.080 g).

MS (ES) m/z 366.7.

HPLC purity 100.0% at 210-370 nm, 9.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 46

### 3-(6-fluoro-2,2-dioxido-1-phenyl-1H-4,2,1-benzoxathiazin-3-yl)-N-methylpropan-1-amine

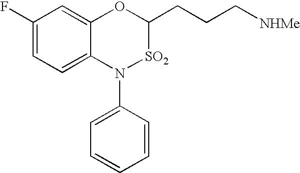

In an analogous manner to Example 44, step 1, 6-fluoro-1H-4,2,1-benzoxathiazine 2,2-dioxide (0.44 g) was prepared from 2-amino-5-fluorophenol (1.0 g).

MS (ES) m/z 201.9

In an analogous manner to Example 44, step 2, 3-(3-chloropropyl)-6-fluoro-1H-4,2,1-benzoxathiazine 2,2-dioxide (0.32 g) was prepared from 6-fluoro-1H-4,2,1-benzoxathiazine 2,2-dioxide (0.35 g).

MS (ES) m/z 277.8

In an analogous manner to Example 44, step 3, 3-(3-chloropropyl)-6-fluoro-1-phenyl-1H-4,2,1-benzoxathiazine 2,2-dioxide (0.23 g) was prepared from 3-(3-chloropropyl)-6-fluoro-1H-4,2,1-benzoxathiazine 2,2-dioxide (0.26 g) and phenylboronic acid.

In an analogous manner to 44, step 4, 3-(6-fluoro-2,2-dioxido-1-phenyl-1H-4,2,1-benzoxathiazin-3-yl)-N-methylpropan-1-amine hydrochloride (0.045 g) was prepared from 3-(3-chloropropyl)-6-fluoro-1-phenyl-1H-4,2,1-benzoxathiazine 2,2-dioxide (0.120 g).

MS (ES) m/z 350.9.

HPLC purity 100.0% at 210-370 nm, 8.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 47

### 3-(5-fluoro-2,2-dioxido-1-phenyl-1H-4,2,1-benzoxathiazin-3-yl)-N-methylpropan-1-amine

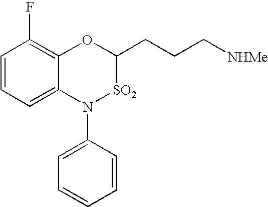

In an analogous manner to Example 44, step 1, 5-fluoro-1H-4,2,1-benzoxathiazine 2,2-dioxide (0.34 g) was prepared from 2-amino-6-fluorophenol (1.0 g).

MS (ES) m/z 201.9

In an analogous manner to Example 44, step 2, of 3-(3-chloropropyl)-5-fluoro-1H-4,2,1-benzoxathiazine 2,2-dioxide (0.21 g) was prepared from 5-fluoro-1H-4,2,1-benzoxathiazine 2,2-dioxide (0.25 g).

MS (ES) m/z 277.8

In an analogous manner to Example 44, step 3, 3-(3-chloropropyl)-5-fluoro-1-phenyl-1H-4,2,1-benzoxathiazine 2,2-dioxide (0.16 g) was prepared from 3-(3-chloropropyl)-5-fluoro-1H-4,2,1-benzoxathiazine 2,2-dioxide (0.21 g).

In an analogous manner to Example 44, step 4, 3-(5-fluoro-2,2-dioxido-1-phenyl-1H-4,2,1-benzoxathiazin-3-yl)-N-methylpropan-1-amine hydrochloride (0.025 g) was prepared from 3-(3-chloropropyl)-5-fluoro-1-phenyl-1H-4,2,1-benzoxathiazine 2,2-dioxide (0.12 g).

MS (ES) m/z 350.9.

HPLC purity 100.0% at 210-370 nm, 8.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 48

### 3-(8-fluoro-2,2-dioxido-1-phenyl-1H-4,2,1-benzoxathiazin-3-yl)-N-methylpropan-1-amine

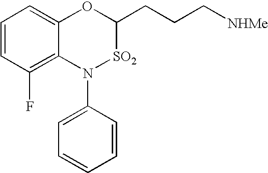

In an analogous manner to Example 44, step 1, 8-fluoro-1H-4,2,1-benzoxathiazine 2,2-dioxide (0.36 g) was prepared from 2-amino-3-fluorophenol (1.0 g).

MS (ES) m/z 201.9.

In an analogous manner to Example 44, step 2, 3-(3-chloropropyl)-8-fluoro-1H-4,2,1-benzoxathiazine 2,2-dioxide (0.28 g) was prepared from 8-fluoro-1H-4,2,1-benzoxathiazine 2,2-dioxide (0.30 g).

MS (ES) m/z 277.8

In an analogous manner to Example 44, step 3, 3-(3-chloropropyl)-8-fluoro-1-phenyl-1H-4,2,1-benzoxathiazine 2,2-dioxide (0.12 g) was prepared from 3-(3-chloropropyl)-8-fluoro-1H-4,2,1-benzoxathiazine 2,2-dioxide (0.16 g).

In an analogous manner to Example 44, step 4, 3-(8-fluoro-2,2-dioxido-1-phenyl-1H-4,2,1-benzoxathiazin-3-yl)-N-methylpropan-1-amine hydrochloride (0.020 g) was prepared from 3-(3-chloropropyl)-8-fluoro-1-phenyl-1H-4,2,1-benzoxathiazine 2,2-dioxide (0.13 g).

MS (ES) m/z 350.9.

HPLC purity 100.0% at 210-370 nm, 7.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 49

### N-methyl-3-(8-methyl-2,2-dioxido-1-phenyl-1H-4,2,1-benzoxathiazin-3-yl)propan-1-amine

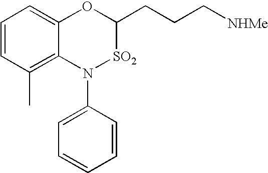

In an analogous manner to Example 44, step 1, 8-methyl-1H-4,2,1-benzoxathiazine 2,2-dioxide (0.36 g) was prepared from 2-amino-3-methylphenol (1.0 g).

MS (ES) m/z 197.9

In an analogous manner to Example 44, step 2, 3-(3-chloropropyl)-8-methyl-1H-4,2,1-benzoxathiazine 2,2-dioxide (0.25 g) was prepared from 8-methyl-1H-4,2,1-benzoxathiazine 2,2-dioxide (0.27 g).

MS (ES) m/z 273.8

In an analogous manner to Example 44, step 3, 3-(3-chloropropyl)-8-methyl-1-phenyl-1H-4,2,1-benzoxathiazine 2,2-dioxide (0.18 g) was prepared from 3-(3-chloropropyl)-8-methyl-1H-4,2,1-benzoxathiazine 2,2-dioxide (0.21 g) and phenylboronic acid.

In an analogous manner to Example 44, step 4, 3-(8-methyl-2,2-dioxido-1-phenyl-1H-4,2,1-benzoxathiazin-3-yl)-N-methylpropan-1-amine hydrochloride (0.021 g) was prepared from 3-(3-chloropropyl)-8-methyl-1-phenyl-1H-4,2,1-benzoxathiazine 2,2-dioxide (0.14 g).

MS (ES) m/z 346.9.

HPLC purity 100.0% at 210-370 nm, 7.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 50

### N-methyl-3-(7-methyl-2,2-dioxido-1-phenyl-1H-4,2,1-benzoxathiazin-3-yl)propan-1-amine

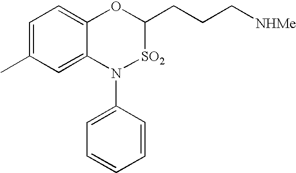

In an analogous manner to Example 44, step 1, 7-methyl-1H-4,2,1-benzoxathiazine 2,2-dioxide (0.44 g) was prepared from 2-amino-4-methylphenol (1.0 g).

MS (ES) m/z 197.9

In an analogous manner to Example 44, step 2, 3-(3-chloropropyl)-7-methyl-1H-4,2,1-benzoxathiazine 2,2-dioxide (0.31 g) was prepared from 7-methyl-1H-4,2,1-benzoxathiazine 2,2-dioxide (0.34 g).

MS (ES) m/z 273.8

In an analogous manner to Example 44, step 3, 3-(3-chloropropyl)-7-methyl-1-phenyl-1H-4,2,1-benzoxathiazine 2,2-dioxide (0.34 g) was prepared from 3-(3-chloropropyl)-7-methyl-1H-4,2,1-benzoxathiazine 2,2-dioxide (0.28 g) and phenylboronic acid.

In an analogous manner to Example 44, step 4, 3-(7-methyl-2,2-dioxido-1-phenyl-1H-4,2,1-benzoxathiazin-3-yl)-N-methylpropan-1-amine hydrochloride (0.10 g) was prepared from 3-(3-chloropropyl)-7-methyl-1-phenyl-1H-4,2,1-benzoxathiazine 2,2-dioxide (0.23 g).

MS (ES) m/z 347.0.

HPLC purity 100.0% at 210-370 nm, 7.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 51

### N-methyl-3-(6-methyl-2,2-dioxido-1-phenyl-1H-4,2,1-benzoxathiazin-3-yl)propan-1-amine

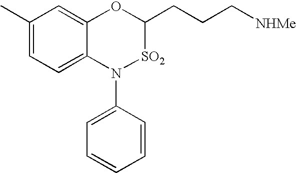

In an analogous manner to Example 44, step 1, 6-methyl-1H-4,2,1-benzoxathiazine 2,2-dioxide (0.44 g) was prepared from 2-amino-5-methylphenol (1.0 g).

MS (ES) m/z 197.9

In an analogous manner to Example 44, step 2, 3-(3-chloropropyl)-6-methyl-1H-4,2,1-benzoxathiazine 2,2-dioxide (0.31 g) was prepared from of 6-methyl-1H-4,2,1-benzoxathiazine 2,2-dioxide (0.32 g). MS (ES) m/z 273.8

In an analogous manner to Example 44, step 3, 3-(3-chloropropyl)-6-methyl-1-phenyl-1H-4,2,1-benzoxathiazine 2,2-dioxide (0.32 g) was prepared from 3-(3-chloropropyl)-6-methyl-1H-4,2,1-benzoxathiazine 2,2-dioxide (0.32 g) and phenylboronic acid.

In an analogous manner to Example 44, step 4, 3-(6-methyl-2,2-dioxido-1-phenyl-1H-4,2,1-benzoxathiazin-3-yl)-N-methylpropan-1-amine hydrochloride (0.13 g) was prepared from 3-(3-chloropropyl)-6-methyl-1-phenyl-1H-4,2,1-benzoxathiazine 2,2-dioxide (0.31 g).

MS (ES) m/z 346.9.

HPLC purity 100.0% at 210-370 nm, 8.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 52

### 3-(6-methoxy-2,2-dioxido-1-phenyl-1H-4,2,1-benzoxathiazin-3-yl)-N-methylpropan-1-amine

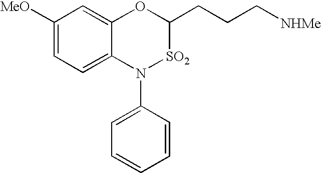

In an analogous manner to Example 44, step 1, 6-methoxy-1H-4,2,1-benzoxathiazine 2,2-dioxide (0.30 g) was prepared from 2-amino-5-methoxyphenol (1.0 g).

MS (ES) m/z 213.9

In an analogous manner to Example 44, step 2, 3-(3-chloropropyl)-6-methoxy-1H-4,2,1-benzoxathiazine 2,2-dioxide (0.26 g) was prepared from 6-methoxy-1H-4,2,1-benzoxathiazine 2,2-dioxide (0.28 g).

In an analogous manner to Example 44, step 3, 3-(3-chloropropyl)-6-methoxy-1-phenyl-1H-4,2,1-benzoxathiazine 2,2-dioxide (0.19 g) was prepared from 3-(3-chloropropyl)-6-methoxy-1H-4,2,1-benzoxathiazine 2,2-dioxide (0.31 g) and phenylboronic acid.

In an analogous manner to Example 44, step 4, 3-(6-methoxy-2,2-dioxido-1-phenyl-1H-4,2,1-benzoxathiazin-3-yl)-N-methylpropan-1-amine hydrochloride (0.021 g) was prepared from 3-(3-chloropropyl)-6-methoxy-1-phenyl-1H-4,2,1-benzoxathiazine 2,2-dioxide (0.19 g).

MS (ES) m/z 363.0.

HPLC purity 97.5% at 210-370 nm, 7.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 53

### 3-(7-chloro-2,2-dioxido-1-phenyl-1H-4,2,1-benzoxathiazin-3-yl)-N-methylpropan-1-amine

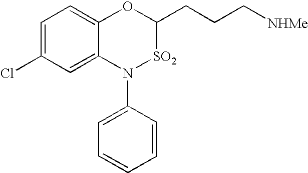

In an analogous manner to Example 44, step 1, 7-chloro-1H-4,2,1-benzoxathiazine 2,2-dioxide (0.45 g) was prepared from 2-amino-4-chlorophenol (1.0 g).

MS (ES) m/z 217.9

In an analogous manner to Example 44, step 2, 3-(3-chloropropyl)-7-chloro-1H-4,2,1-benzoxathiazine 2,2-dioxide (0.31 g) was prepared from 7-chloro-1H-4,2,1-benzoxathiazine 2,2-dioxide (0.30 g).

MS (ES) m/z 293.8.

In an analogous manner to Example 44, step 3, 3-(3-chloropropyl)-7-chloro-1-phenyl-1H-4,2,1-benzoxathiazine 2,2-dioxide (0.23 g) was prepared from 3-(3-chloropropyl)-7-chloro-1H-4,2,1-benzoxathiazine 2,2-dioxide (0.26 g) and phenylboronic acid.

In an analogous manner to Example 44, step 4, 3-(7-chloro-2,2-dioxido-1-phenyl-1H-4,2,1-benzoxathiazin-3-yl)-N-methylpropan-1-amine hydrochloride (0.04 g) was prepared from 3-(3-chloropropyl)-7-chloro-1-phenyl-1H-4,2,1-benzoxathiazine 2,2-dioxide (0.13 g).

MS (ES) m/z 366.9;

HPLC purity 98.2% at 210-370 nm, 7.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉ClN₂O₃S+H+, 367.08777; found (ESI, [M+H]⁺), 367.0856.

### Example 54

### 3-(2,2-dioxido-1,7-diphenyl-1H-4,2,1-benzoxathiazin-3-yl)-N-methylpropan-1-amine

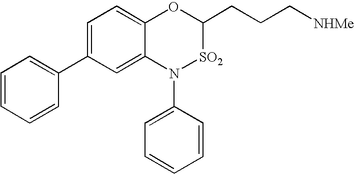

In an analogous manner to Example 44, step 1, 7-phenyl-1H-4,2,1-benzoxathiazine 2,2-dioxide (0.31 g) was prepared from 2-amino-4-phenylphenol (1.0 g).

MS (ES) m/z 259.8

In an analogous manner to Example 44, step 2, 3-(3-chloropropyl)-7-phenyl-1H-4,2,1-benzoxathiazine 2,2-dioxide (0.27 g) was prepared from 7-phenyl-1H-4,2,1-benzoxathiazine 2,2-dioxide (0.28 g).

In an analogous manner to Example 44, step 3, 3-(3-chloropropyl)-1,7-diphenyl-1H-4,2,1-benzoxathiazine 2,2-dioxide (0.21 g) was prepared from 3-(3-chloropropyl)-7-phenyl-1H-4,2,1-benzoxathiazine 2,2-dioxide (0.24 g) and phenylboronic acid.

In an analogous manner to Example 44, step 4, 3-(2,2-dioxido-1,7-diphenyl-1H-4,2,1-benzoxathiazin-3-yl)-N-methylpropan-1-amine hydrochloride (0.05 g) was prepared from 3-(3-chloropropyl)-1,7-diphenyl-1H-4,2,1-benzoxathiazine 2,2-dioxide (0.15 g).

MS (ES) m/z 408.8;

HPLC purity 95.4% at 210-370 nm, 8.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 min

HRMS: calculated for C₂₃H₂₄N₂O₃S+H+, 409.15804; found (ESI, [M+H]⁺), 409.1536.

### Example 55

### 3-(2,2-dioxido-1-phenyl-1H-naphtho[2,3-e][1,3,4]oxathiazin-3-yl)-N-methylpropan-1-amine hydrochloride

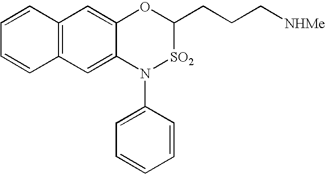

In an analogous manner to Example 44, step 1, 1H-naphtho[2,3-e][1,3,4]oxathiazine 2,2-dioxide (2.6 g) was prepared from 3-amino-2-naphthol (3.2 g).

MS (ES) m/z 233.9

In an analogous manner to Example 44, step 2, 3-(3-chloropropyl)-1H-naphtho[2,3-e][1,3,4]oxathiazine 2,2-dioxide (0.46 g) was prepared from 1H-naphtho[2,3-e][1,3,4]oxathiazine 2,2-dioxide (0.48 g).

MS (ES) m/z 309.8

In an analogous manner to Example 44, step 3, 3-(2,2-dioxido-1-phenyl-1H-naphtho[2,3-e][1,3,4]oxathiazin-3-yl)-1-chloropropane (0.13 g) was prepared from 3-(3-chloropropyl)-1H-naphtho[2,3-e][1,3,4]oxathiazine 2,2-dioxide (0.25 g).

In an analogous manner to Example 44, step 4, 3-(2,2-dioxido-1-phenyl-1H-naphtho[2,3-e][1,3,4]oxathiazin-3-yl)-N-methylpropan-1-amine hydrochloride (0.015 g) was prepared from 3-(2,2-dioxido-1-phenyl-1H-naphtho[2,3-e][1,3,4]oxathiazin-3-yl)-1-chloropropane (0.90 g).

MS (ESI) m/z 383.

HPLC purity 100.0% at 210-370 nm, 9.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 56

### 3-(2,2-dioxido-1-pyridin-3-yl-1H-4,2,1-benzoxathiazin-3-yl)-N-methylpropan-1-amine

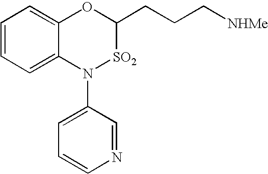

In an analogous manner to Example 44, step 2, 3-(3-chloropropyl)-1H-4,2,1-benzoxathiazine 2,2-dioxide (0.75 g) was prepared from 1H-4,2,1-benzoxathiazine 2,2-dioxide (0.74 g).

MS (ES) m/z 259.9

In an analogous manner to Example 44, step 3, 3-(3-chloropropyl)-1-pyridine-3-yl-1H-4,2,1-benzoxathiazine 2,2-dioxide (0.12 g) was prepared from 3-(3-chloropropyl)-1H-4,2,1-benzoxathiazine 2,2-dioxide (0.21 g) and pyridine-3-boronic acid.

In an analogous manner to Example 44, step 4, 3-(2,2-dioxido-1-pyridin-3-yl-1H-4,2,1-benzoxathiazin-3-yl)-N-methylpropan-1-amine dihydrochloride (0.03 g) was prepared from 3-(3-chloropropyl)-1-pyridine-3-yl-1H-4,2,1-benzoxathiazine 2,2-dioxide (0.10 g). MS (ES) m/z 333.8.

HPLC purity 100.0% at 210-370 nm, 5.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 57

### 3-(2,2-dioxido-1-quinolin-3-yl-1H-4,2,1-benzoxathiazin-3-yl)-N-methylpropan-1-amine

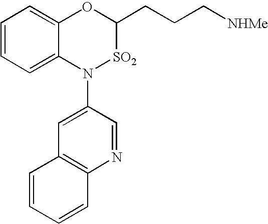

In an analogous manner to Example 44, step 3, 3-(3-chloropropyl)-1-quinolin-3-yl-1H-4,2,1-benzoxathiazine 2,2-dioxide (0.11 g) was prepared from 3-(3-chloropropyl)-1H-4,2,1-benzoxathiazine 2,2-dioxide (0.13 g) and quinoline-3-boronic acid.

In an analogous manner to Example 44, step 4, 3-(2,2-dioxido-1-quinolin-3-yl-1H-4,2,1-benzoxathiazin-3-yl)-N-methylpropan-1-amine dihydrochloride (0.027 g) was prepared from 3-(3-chloropropyl)-1-quinolin-3-yl-1H-4,2,1-benzoxathiazine 2,2-dioxide (0.10 g). MS (ES) m/z 383.7.

HPLC purity 80.4% at 210-370 nm, 7.0 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

### Example 58

### N-benzyl-3-(2,2-dioxido-1-phenyl-1H-4,2,1-benzoxathiazin-3-yl)-N-methylpropan-1-amine hydrochloride

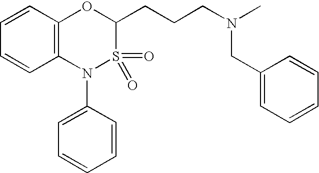

A solution of 3-(3-chloropropyl)-1-phenyl-1H-4,2,1-benzoxathiazine 2,2-dioxide (0.25 g, 0.75 mmol) in N-methylbenzylamine (0.87 mL, 6.8 mmol) was heated to 105° C. for 4 hours. The reaction mixture was diluted with H₂O (50 mL), extracted with dichloromethane (2×50 mL), dried (Na₂SO₄), and evaporated. The residue was dissolved in ethyl ether (10 mL) and treated with hydrogen chloride (2 M solution in ethyl ether, 1.0 mL), resulting in a white precipitate that was isolated by decantation and dried under vacuum to afford N-benzyl-3-(2,2-dioxido-1-phenyl-1H-4,2,1-benzoxathiazin-3-yl)-N-methylpropan-1-amine hydrochloride (0.16 g, 50%) as a white powder:

HPLC purity 100.0% at 210-370 nm, 9.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes. HRMS: calculated for C₂₄H₂₆N₂O₃S+H+, 423.17369; found (ESI, [M+H]⁺), 423.1736.

### Example 59

### 3-(2,2-dioxido-1-phenyl-1H-2,4,1-benzodithiazin-3-yl)-N-methylpropan-1-amine Hydrochloride

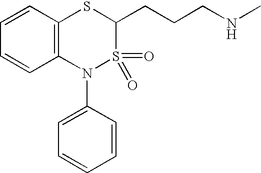

### Step 1 3-(3-chloropropyl)-1H-2,4,1-benzodithiazine 2,2-dioxide

A solution of 1H-2,4,1-benzodithiazine 2,2-dioxide (WO 92/05164) (5.0 g, 25 mmol) in tetrahydrofuran (150 mL) was cooled to −78° C., treated with n-butyl lithium (2.5 M in hexanes, 20 mL, 50 mmol), and stirred under N₂ for 1 hour. 1-Bromo-3-chloropropane (2.5 mL, 25 mmol) was added and the reaction mixture was allowed to warm to room temperature over 1 hour. The reaction mixture was quenched after 3 hours by the addition of 2 M hydrochloric acid (200 mL) and extracted with dichloromethane (3×300 mL), dried (Na₂SO₄), and evaporated. Column chromatography (SiO₂, 3-50% ethyl acetate/hexanes) provided 3-(3-chloropropyl)-1H-2,4,1-benzodithiazine 2,2-dioxide (4.2 g, 61%) as a tan solid:

HPLC purity 98.8% at 210-370 nm, 8.6 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes. HRMS: calculated for C₁₀H₁₂ClNO₂S₂+H+, 278.00707; found (ESI, [M+H—SO2]⁺), 214.033.

### Step 2: 3-(3-chloropropyl)-1-phenyl-1H-2,4,1-benzodithiazine 2,2-dioxide

A solution of 3-(3-chloropropyl)-1H-2,4,1-benzodithiazine 2,2-dioxide (0.28 g, 1.0 mmol) in dichloromethane (5.0 mL) was treated with pyridine (0.16 mL, 2.0 mmol), phenylboronic acid (0.24 g, 2.0 mmol) and copper(II) acetate (0.27 g, 1.5 mmol) and stirred at 23° C. for 16 hours. The reaction mixture was quenched by the addition of ammonium hydroxide (20 mL), diluted with methanol (5.0 mL), extracted with dichloromethane (3×20 mL), dried (Na₂SO₄), and evaporated. Column chromatography (SiO₂, 5-50% ethyl acetate/hexanes) provided 3-(3-chloropropyl)-1-phenyl-1H-2,4,1-benzodithiazine 2,2-dioxide (0.15 g, 42%) as a yellow film:

HPLC purity 98.0% at 210-370 nm, 10.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₆H₁₆ClNO₂S₂+H⁺, 354.03837; found (ESI, [M+H—SO2]⁺), 290.0658.

Step 3: 3-(2,2-dioxido-1-phenyl-1H-2,4,1-benzodithiazin-3-yl)-N-methylpropan-1-amine 3-(3-Chloropropyl)-1-phenyl-1H-2,4,1-benzodithiazine 2,2-dioxide (0.096 g, 0.27 mmol) was dissolved in an 8 M solution of methylamine in tetrahydrofuran (10 mL), treated with potassium iodide (0.20 g, 1.2 mmol), and stirred in a capped vial at 55° C. for 16 hours. The reaction mixture was evaporated and the residue purified by column chromatography (SiO₂, 0-5% 7 M NH₃-methanol/dichloromethane). The purified free-base was dissolved in ethyl ether (10 mL) and treated with hydrogen chloride (1.0 mL of a 2 M solution in ethyl ether), resulting in a white precipitate that was isolated by decantation and dried under vacuum to afford 3-(2,2-dioxido-1-phenyl-1H-2,4,1-benzodithiazin-3-yl)-N-methylpropan-1-amine hydrochloride (0.078 g, 75%) as a yellow powder:

HPLC purity 100.0% at 210-370 nm, 8.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes. HRMS: calculated for C₁₇H₂₀N₂O₂S₂+H⁺, 349.10389; found (ESI, [M+H]⁺), 349.1042.

### Example 60

### N-methyl-3-[1-(3-methylphenyl)-2,2-dioxido-1H-2,4,1-benzodithiazin-3-yl]propan-1-amine Hydrochloride

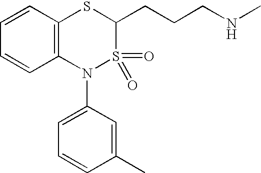

### Step 1, 3-(3-chloropropyl)-1-(3-methylphenyl)-1H-2,4,1-benzodithiazine 2,2-dioxide

In an analogous manner to Example 60 step 2, 3-(3-chloropropyl)-1H-2,4,1-benzodithiazine 2,2-dioxide (0.20 g) was coupled to m-tolylboronic acid to provide 3-(3-chloropropyl)-1-(3-methylphenyl)-1H-2,4,1-benzodithiazine 2,2-dioxide (84 mg):

HPLC purity 100.0% at 210-370 nm, 12.0 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₈ClNO₂S₂+H⁺, 368.05402; found (ESI, [M+H—SO2]⁺), 304.0807.

### Step 2, N-methyl-3-[1-(3-methylphenyl)-2,2-dioxido-1H-2,4,1-benzodithiazin-3-yl]propan-1-amine

In an analogous manner to Example 60, step 3, 3-(3-chloropropyl)-1-(3-methylphenyl)-1H-2,4,1-benzodithiazine 2,2-dioxide (25 mg) was reacted with methylamine and then treated with HCl to provide N-methyl-3-[1-(3-methylphenyl)-2,2-dioxido-1H-2,4,1-benzodithiazin-3-yl]propan-1-amine hydrochloride (19 mg):

HPLC purity 100.0% at 210-370 nm, 8.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₂S₂+H⁺, 363.11954; found (ESI, [M+H]⁺), 363.1202.

### Example 61

### 3-[1-(3-fluorophenyl)-2,2-dioxido-1H-2,4,1-benzodithiazin-3-yl]-N-methylpropan-1-amine hydrochloride

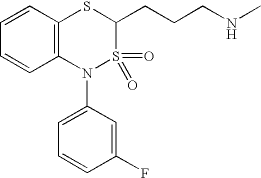

### Step 1, 3-(3-chloropropyl)-1-(3-fluorophenyl)-1H-2,4,1-benzodithiazine 2,2-dioxide

In an analogous manner to Example 59 step 2, 3-(3-chloropropyl)-1H-2,4,1-benzodithiazine 2,2-dioxide (0.20 g) was coupled to 3-fluorophenylboronic acid to provide 3-(3-chloropropyl)-1-(3-fluorophenyl)-1H-2,4,1-benzodithiazine 2,2-dioxide (0.13 g):

HPLC purity 100.0% at 210-370 nm, 10.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes. HRMS: calculated for C₁₆H₁₅ClFNO₂S₂+H⁺, 372.02895; found (ESI, [M+H—SO2]⁺), 308.0635.

### Step 2: 3-[1-(3-fluorophenyl)-2,2-dioxido-1H-2,4,1-benzodithiazin-3-yl]-N-methylpropan-1-amine

In an analogous manner to Example 59, step 3, 3-(3-chloropropyl)-1-(3-fluorophenyl)-1H-2,4,1-benzodithiazine 2,2-dioxide (53 mg) was reacted with methylamine and then treated with HCl to provide 3-[1-(3-fluorophenyl)-2,2-dioxido-1H-2,4,1-benzodithiazin-3-yl]-N-methylpropan-1-amine hydrochloride (34 mg):

HPLC purity 100.0% at 210-370 nm, 8.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉FN₂O₂S₂+H⁺, 367.09447; found (ESI, [M+H]⁺), 367.096.

### Example 62

### 3-[1-(3-methoxyphenyl)-2,2-dioxido-1H-2,4,1-benzodithiazin-3-yl]-N-methylpropan-1-amine Hydrochloride

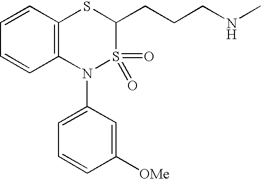

### Step, 3-(3-chloropropyl)-1-(3-methoxyphenyl)-1H-2,4,1-benzodithiazine 2,2-dioxide

In an analogous manner to Example 59 step 2, 3-(3-chloropropyl)-1H-2,4,1-benzodithiazine 2,2-dioxide (0.20 g) was coupled to 3-methoxyphenylboronic acid to provide 3-(3-chloropropyl)-1-(3-methoxyphenyl)-1H-2,4,1-benzodithiazine 2,2-dioxide (0.16 g):

HPLC purity 87.9% at 210-370 nm, 10.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C17H18ClNO3S2+H⁺, 384.04894; found (ESI, [M+H—SO2]⁺), 320.0873;

Step 2: In an analogous manner to Example 59, step 3, 3-(3-chloropropyl)-1-(3-methoxyphenyl)-1H-2,4,1-benzodithiazine 2,2-dioxide (86 mg) was reacted with methylamine and then treated with HCl to provide N-methyl-3-[1-(3-methoxylphenyl)-2,2-dioxido-1H-2,4,1-benzodithiazin-3-yl]propan-1-amine hydrochloride (34 mg):

HPLC purity 100.0% at 210-370 nm, 8.5 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₃S₂+H⁺, 379.11446; found (ESI, [M+H]⁺), 379.1149.

### Example 63

### N-methyl-3-[1-(4-methylphenyl)-2,2-dioxido-1H-2,4,1-benzodithiazin-3-yl]propan-1-amine Hydrochloride

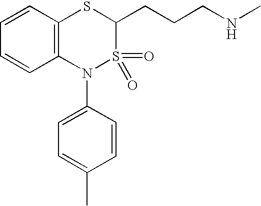

### Step 1, 3-(3-chloropropyl)-1-(4-methylphenyl)-1H-2,4,1-benzodithiazine 2,2-dioxide

In an analogous manner to Example 59, step 2, 3-(3-chloropropyl)-1H-2,4,1-benzodithiazine 2,2-dioxide (0.20 g) was coupled to 4-tolyllboronic acid to provide 3-(3-chloropropyl)-1-(4-methylphenyl)-1H-2,4,1-benzodithiazine 2,2-dioxide (0.14 g):

HPLC purity 100.0% at 210-370 nm, 10.7 minutes; Xterra RP18, 3.5μ, 150×4.6 mm column, 1.2 mL/minutes. 85/15-5/95 (ammonium formate buffer pH=3.5/ACN+MeOH) for 10 minutes. hold 4 minutes.

HRMS: calculated for C₁₇H₁₈ClNO₂S₂+H⁺, 368.05402; found (ESI, [M+H—SO₂]⁺), 304.1102.

Step 2: In an analogous manner to Example 59, step 3, 3-(3-chloropropyl)-1-(4-methylphenyl)-1H-2,4,1-benzodithiazine 2,2-dioxide (0.13 g) was reacted with methylamine and then treated with HCl to provide N-methyl-3-[1-(4-methylphenyl)-2,2-dioxido-1H-2,4,1-benzodithiazin-3-yl]propan-1-amine hydrochloride (92 mg):

HPLC purity 100.0% at 210-370 nm, 7.7 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₂S₂+H⁺, 363.11954; found (ESI, [M+H]⁺), 363.1192.

### Example 64

### 3-[1-(4-fluorophenyl)-2,2-dioxido-1H-2,4,1-benzodithiazin-3-yl]-N-methylpropan-1-amine Hydrochloride

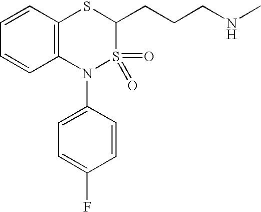

### Step 1, 3-(3-chloropropyl)-1-(4-fluorophenyl)-1H-2,4,1-benzodithiazine 2,2-dioxide

In an analogous manner to Example 59, step 2, 3-(3-chloropropyl)-1H-2,4,1-benzodithiazine 2,2-dioxide (0.20 g) was coupled to 4-fluorophenylboronic acid to provide 3-(3-chloropropyl)-1-(4-fluorophenyl)-1H-2,4,1-benzodithiazine 2,2-dioxide (0.14 g):

HPLC purity 100.0% at 210-370 nm, 10.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₆H₁₅ClFNO₂S₂+H⁺, 372.02895; found (ESI, [M+H—SO₂]⁺), 308.0512.

Step 2: In an analogous manner to Example 59, step 3, 3-(3-chloropropyl)-1-(4-fluorophenyl)-1H-2,4,1-benzodithiazine 2,2-dioxide (0.13 g) was reacted with methylamine and then treated with HCl to provide N-methyl-3-[1-(4-fluorophenyl)-2,2-dioxido-1H-2,4,1-benzodithiazin-3-yl]propan-1-amine hydrochloride (88 mg):

HPLC purity 100.0% at 210-370 nm, 7.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉FN₂O₂S₂+H+, 367.09447; found (ESI, [M+H]⁺), 367.0961

### Example 65

### 3-[1-(4-methoxyphenyl)-2,2-dioxido-1H-2,4,1-benzodithiazin-3-yl]-N-methylpropan-1-amine hydrochloride

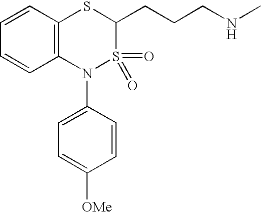

Step 1, 3-(3-chloropropyl)-1-(4-methoxyphenyl)-1H-2,4,1-benzodithiazine 2,2-dioxide: In an analogous manner to Example 59 step 2, 3-(3-chloropropyl)-1H-2,4,1-benzodithiazine 2,2-dioxide (0.20 g) was coupled to 4-methoxyphenylboronic acid to provide 3-(3-chloropropyl)-1-(4-methoxyphenyl)-1H-2,4,1-benzodithiazine 2,2-dioxide (0.18 g):

HPLC purity 96.4% at 210-370 nm, 10.3 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₈ClNO₃S₂+H⁺, 384.04894; found (ESI, [M+H—SO₂]⁺), 320.0766;

Step 2: In an analogous manner to Example 59, step 3, 3-(3-chloropropyl)-1-(4-methoxyphenyl)-1H-2,4,1-benzodithiazine 2,2-dioxide (0.17 g) was reacted with methylamine and then treated with HCl to provide N-methyl-3-[1-(4-methylphenyl)-2,2-dioxido-1H-2,4,1-benzodithiazin-3-yl]propan-1-amine hydrochloride (122 mg):

HPLC purity 100.0% at 210-370 nm, 7.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₈H₂₂N₂O₃S₂+H⁺, 379.11446; found (ESI, [M+H]⁺), 379.1185.

### Example 66

### 3-[1-(3-chlorophenyl)-2,2-dioxido-1H-2,4,1-benzodithiazin-3-yl]-N-methylpropan-1-amine Hydrochloride

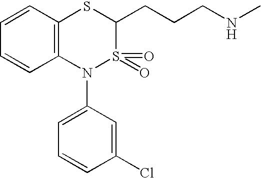

Step 1, 3-(3-chloropropyl)-1-(3-chlorophenyl)-1H-2,4,1-benzodithiazine 2,2-dioxide: In an analogous manner to Example 59 step 2, 3-(3-chloropropyl)-1H-2,4,1-benzodithiazine 2,2-dioxide (0.28 g) was coupled to 3-chlorophenylboronic acid to provide 3-(3-chloropropyl)-1-(3-chlorophenyl)-1H-2,4,1-benzodithiazine 2,2-dioxide (0.15 g):

HPLC purity 100.0% at 210-370 nm, 10.8 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

Step 2: In an analogous manner to Example 60, step 3, 3-(3-chloropropyl)-1-(3-chlorophenyl)-1H-2,4,1-benzodithiazine 2,2-dioxide (0.13 g) was reacted with methylamine and then treated with HCl to provide N-methyl-3-[1-(3-chlorophenyl)-2,2-dioxido-1H-2,4,1-benzodithiazin-3-yl]propan-1-amine hydrochloride (90 mg):

HPLC purity 100.0% at 210-370 nm, 9.1 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉ClN₂O₂S₂+H⁺, 383.06492; found (ESI, [M+H]⁺), 383.0637.

### Example 67

### 3-[1-(4-chlorophenyl)-2,2-dioxido-1H-2,4,1-benzodithiazin-3-yl]-N-methylpropan-1-amine Hydrochloride

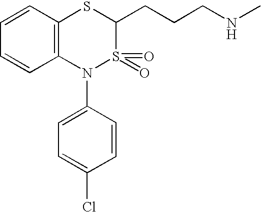

### Step 1 3-(3-chloropropyl)-1-(4-chlorophenyl)-1H-2,4,1-benzodithiazine 2,2-dioxide

In an analogous manner to Example 59 step 2, 3-(4-chloropropyl)-1H-2,4,1-benzodithiazine 2,2-dioxide (0.28 g) was coupled to 4-chlorophenylboronic acid to provide 3-(3-chloropropyl)-1-(4-chlorophenyl)-1H-2,4,1-benzodithiazine 2,2-dioxide (0.19 g):

HPLC purity 100.0% at 210-370 nm, 10.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₆H₁₅Cl₂NO₂S₂+H⁺, 387.99940; found (ESI, [M+H—SO₂]+), 324.0271.

Step 2: In an analogous manner to Example 59, step 3, 3-(3-chloropropyl)-1-(4-chlorophenyl)-1H-2,4,1-benzodithiazine 2,2-dioxide (0.18 g) was reacted with methylamine and then treated with HCl to provide N-methyl-3-[1-(4-chlorophenyl)-2,2-dioxido-1H-2,4,1-benzodithiazin-3-yl]propan-1-amine hydrochloride (0.14 g):

HPLC purity 100.0% at 210-370 nm, 9.2 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₉ClN₂O₂S₂+H⁺, 383.06492; found (ESI, [M+H]⁺), 383.0636.

### Example 68

### 3-[1-(3-chloro-4-fluorophenyl)-2,2-dioxido-1H-2,4,1-benzodithiazin-3-yl]-N-methylpropan-1-amine Hydrochloride

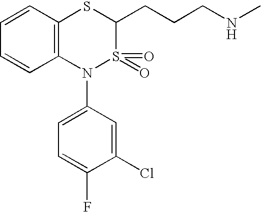

### Step 1, 3-(3-chloropropyl)-1-(3-chloro-4-fluorophenyl)-1H-2,4,1-benzodithiazine 2,2-dioxide

In an analogous manner to Example 59 step 2, 3-(3-chloropropyl)-1H-2,4,1-benzodithiazine 2,2-dioxide (0.28 g) was coupled to 3-chloro-4-fluorophenylboronic acid to provide 3-(3-chloropropyl)-1-(3-chloro-4-fluorophenyl)-1H-2,4,1-benzodithiazine 2,2-dioxide (0.25 g):

HPLC purity 100.0% at 210-370 nm, 10.9 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₆H₁₄Cl₂FNO₂S₂+H⁺, 405.98998; found (ESI, [M+H—SO₂]⁺), 342.0095.

Step 2: In an analogous manner to Example 59, step 3, 3-(3-chloropropyl)-1-(3-chloro-4-fluorophenyl)-1H-2,4,1-benzodithiazine 2,2-dioxide (0.23 g) was reacted with methylamine and then treated with HCl to provide N-methyl-3-[1-(3-chloro-4-fluorophenyl)-2,2-dioxido-1H-2,4,1-benzodithiazin-3-yl]propan-1-amine hydrochloride (0.16 g):

HPLC purity 90.1% at 210-370 nm, 9.4 minutes; Xterra RP18, 3.5μ, 150×4.6 mm column, 1.2 mL/minutes. 85/15-5/95 (ammonium formate buffer pH=3.5/ACN+MeOH) for 10 minutes, hold 4 minutes.

HRMS: calculated for C₁₇H₁₈ClFN₂O₂S₂+H⁺, 401.05550; found (ESI, [M+H]⁺), 401.0547.

### Example 69

### N-methyl-3-[1-(4-methyl phenyl)-2,2-dioxido-1H-naphtho[2,3-e][1,3,4]oxathiazin-3-yl]propan-1-amine Hydrochloride

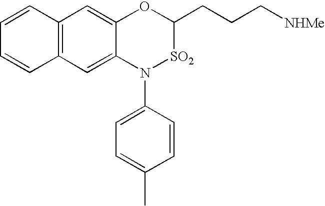

In an analogous manner to Example 44, step 3, 0.19 g of 3-(2,2-dioxido-1-(4-methylphenyl)-1H-naphtho[2,3-e][1,3,4]oxathiazin-3-yl)-1-chloropropane was prepared from 0.28 g of 3-(3-chloropropyl)-1H-naphtho[2,3-e][1,3,4]oxathiazine 2,2-dioxide (See Example 55, step 2).

In an analogous manner to Example 44, step 4, 0.055 g of N-methyl-3-[1-(4-methylphenyl)-2,2-dioxido-1H-naphtho[2,3-e][1,3,4]oxathiazin-3-yl]propan-1-amine hydrochloride was prepared from 0.15 g of 3-(2,2-dioxido-1-(4-methylphenyl)-1H-naphtho[2,3-e][1,3,4]oxathiazin-3-yl)-1-chloropropane.

MS (ES) m/z 397.1;

HPLC purity 100.0% at 210-370 nm, 10.4 minutes; Xterra RP18, 3.5 u, 150×4.6 mm column, 1.2 mL/minutes, 85/15-5/95 (ammonium bicarbonate buffer

pH=9.5/ACN+MeOH) for 10 minutes, hold 4 minutes.

HRMS: calculated for C₂₂H₂₄N₂O₃S+H⁺, 397.15804; found (ESI, [M+H]⁺), 397.1597

### Example 70

### 3-[1-(3-fluorophenyl)-2,2-dioxido-1H-naphtho[2,3-e][1,3,4]oxathiazin-3-yl]-N-methylpropan-1-amine Hydrochloride

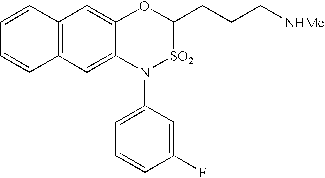

In an analogous manner to Example 44, step 3, 0.15 g of 3-(2,2-dioxido-1-(3-fluorophenyl)-1H-naphtho[2,3-e][1,3,4]oxathiazin-3-yl)-1-chloropropane was prepared from 0.21 g of 3-(3-chloropropyl)-1H-naphtho[2,3-e][1,3,4]oxathiazine 2,2-dioxide (See Example 55, step 2).

In an analogous manner to Example 44, step 4, 0.070 g of 3-[1-(3-fluorophenyl)-2,2-dioxido-1H-naphtho[2,3-e][1,3,4]oxathiazin-3-yl]-N-methylpropan-1-amine hydrochloride was prepared from 0.13 g of 3-(2,2-dioxido-1-(3-fluorophenyl)-1H-naphtho[2,3-e][1,3,4]oxathiazin-3-yl)-1-chloropropane.

MS (ES) m/z 401.1;

HPLC purity 100.0% at 210-370 nm, 10.1 minutes; Xterra RP18, 3.5 u, 150×4.6 mm column, 1.2 mL/minutes, 85/15-5/95 (ammonium bicarbonate buffer pH=9.5/ACN+MeOH) for 10 minutes, hold 4 minutes.

HRMS: calculated for C₂₁H₂₁FN₂O₃S+H⁺, 401.13297; found (ESI, [M+H]⁺), 401.1321.

### Example 71

### 3-[(3S)-2,2-dioxido-1-phenyl-1H-naphtho[2,3-e][1,3,4]oxathiazin-3-yl]-N-methylpropan-1-amine Hydrochloride

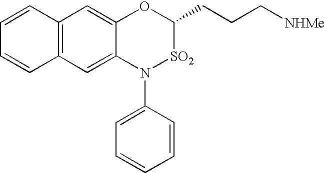

Racemic tert-butyl[3-(2,2-dioxido-1-phenyl-1H-naphtho[2,3-e][1,3,4]oxathiazin-3-yl)propyl]methylcarbamate (Example 55 step 4) (0.80 g) was dissolved in 15 mL of 1:1 methanol/acetonitrile, and the resulting solution was injected onto the Supercritical Fluid Chromatography (SFC) instrument with a volume of 1.2 mL per injection. The baseline resolved enantiomers were collected using a Berger MultiGram Prep SFC (Berger Instruments, Inc. Newark, Del.) under the following conditions: Chiralpak AD-H (5 micron, 250 mm L×20 mm ID, Chiral Technologies, Inc, Exton, Pa.), 35° C. column temperature, 25% MeOH as CO₂ modifier, 50 mL/min flow rate, 100 bar outlet pressure, 220 nm UV detection. The chiral purity of each enantiomer was determined under the same SFC conditions using a Chiralpak AD-H column (5 micron, 250 mm L×4.6 mm ID) at 2.0 mL/min flow rate on a Berger Analytical SFC instrument.

Peak 1 (360 mg) Rt 4.7 min

Peak 2 (370 mg) Rt 9.3 minutes.

The residue isolated from peak 1 was subjected to 2N HCl as described in Example 44, step 4 to yield 0.16 g of white solid arbitrarily assigned as 3-[(3S)-2,2-dioxido-1-phenyl-1H-naphtho[2,3-e][1,3,4]oxathiazin-3-yl]-N-methylpropan-1-amine hydrochloride

[alpha]_{D}²⁵=−114.4° (c=10 mg/mL, MeOH);

MS (ES) m/z 383.1;

HPLC purity 100.0% at 210-370 nm, 8.3 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

### Example 72

### 3-[(3R)-2,2-dioxido-1-phenyl-1H-naphtho[2,3-e][1,3,4]oxathiazin-3-yl]-N-methylpropan-1-amine Hydrochloride

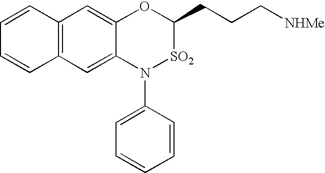

The residue isolated from Example 71 peak 2 was subjected to 2N HCl as described in Example 44, step 4 to yield 0.22 g of white solid arbitrarily assigned as 3-[(3R)-2,2-dioxido-1-phenyl-1H-naphtho[2,3-e][1,3,4]oxathiazin-3-yl]-N-methylpropan-1-amine hydrochloride

[alpha]_{D}²⁵=+108.5° (c=10 mg/mL, MeOH);

MS (ES) m/z 383.1;

HPLC purity 100.0% at 210-370 nm, 8.3 minutes; Xterra RP18, 3.5 u, 150×4.6 mm column, 1.2 mL/minutes, 85/15-5/95 (ammonium formate buffer

pH=3.5/ACN+MeOH) for 10 minutes, hold 4 minutes.

### Example 73

### 3-[1-(2-fluorophenyl)-2,2-dioxido-3,4-dihydro-1H-2,1-benzothiazin-3-yl]-N-methylpropan-1-amine Hydrochloride

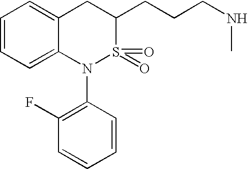

Step 1: A solution of 2-bromophenethylalcohol (10 g, 50 mmol) in thionyl chloride (45 mL) was treated with dimethylformamide (0.1 mL) and heated to reflux for 6 hours. The reaction mixture was cooled to 0° C., quenched by the addition of H₂O (100 mL), extracted with ethyl ether (250 mL), dried (Na₂SO₄), and evaporated to provide 2-bromophenethylchloride (9.0 g, 82%) as a yellow oil:

HPLC purity 99.2% at 210-370 nm, 10.4 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

Step 2: A suspension of 2-bromophenethylchloride (2.2 g, 10 mmol) in H₂O (12 mL) was treated with sodium sulfite (1.3 g, 10 mmol), and sodium iodide (100 mg) and heated in a sealed glass tube at 130° C. for 16 hours. The reaction mixture was cooled to 0° C., forming a white precipitate, which was isolated by filtration, and washed with ice-water and hexanes to provide sodium 2-bromophenethylsulfonate (1.7 g, 60%) as a white solid:

HPLC purity 97.7% at 210-370 nm, 5.0 minutes; Xterra RP18, 3.5 u, 150×4.6 mm column, 1.2 mL/minutes, 85/15-5/95 (ammonium. formate. buffer. pH=3.5/ACN+MeOH) for 10 minutes, hold 4 min

Step 3: A suspension of sodium 2-bromophenethylsulfonate (2.87 g, 10 mmol) in toluene (70 mL) was treated with thionyl chloride (50 mL) and dimethylformamide (0.3 mL) and heated to 80° C. for 16 hours. The reaction mixture was cooled to room temperature, and evaporated. The residue was suspended in dichloromethane (100 mL) and filtered to remove inorganic material. Concentration provided 2-bromophenethylsulfonyl chloride (2.5 g, 88%) as a yellow oil:

HPLC purity 96.1% at 210-370 nm, 5.0 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 min

Step 4: A solution of 2-bromophenethylsulfonyl chloride (2.6 g, 9.2 mmol) in dichloromethane (25 mL) was added dropwise to a solution of 2-fluoroaniline (1.65 mL, 17.1 mmol) and pyridine (1.2 mL, 14.8 mmol) in dichloromethane (25 mL) over 30 minutes, and stirred at 22° C. for 14 hours. The reaction mixture was diluted with 2 N hydrochloric acid (150 mL), extracted with dichloromethane (2×150 mL), dried (Na₂SO₄), and evaporated. Flash chromatography (SiO₂, 10-100% dichloromethane/hexanes) provided 2-(2-bromophenyl)-N-(2-fluorophenyl)ethanesulfonamide (2.1 g, 64%) as a white solid:

HPLC purity 99.6% at 210-370 nm, 9.8 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 min

Step 5: A solution of 2-(2-bromophenyl)-N-(2-fluorophenyl) ethanesulfonamide (1.79 g, 5 mmol) in dimethylsulfoxide (40 mL) and benzene (4 mL) was treated with cesium acetate (4.8 g, 25 mmol) and copper(I) iodide (1.9 g, 10 mmol) and stirred at 22° C. for 22 hours. The reaction mixture was diluted with ethyl ether (200 mL), washed with sat. ammonium hydroxide (200 mL), dried (Na₂SO₄), and evaporated. Flash chromatography (SiO₂, 5-50% ethyl acetate/hexanes) provided 1-(2-fluorophenyl)-3,4-dihydro-1H-benzo[c][1,2]thiazine 2,2-dioxide (1.1 g, 80%) as a white solid:

HPLC purity 96.1% at 210-370 nm, 8.5 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 min

HRMS: calculated for C₁₄H₁₃BrFNO₂S+H⁺, 277.05728; found (ESI, [M+H—SO₂]⁺), 277.0569.

Step 6: A solution of 1-(2-fluorophenyl)-3,4-dihydro-1H-benzo[c][1,2]thiazine 2,2-dioxide (0.52 g, 1.9 mmol) in tetrahydrofuran (8 mL) was treated with lithium hexamethyldisilazide (2.2 mL of a 1.0 M solution in tetrahydrofuran, 2.2 mmol) at −78° C. After 3 h, 3-bromo-1-chloropropane (0.4 mL, 4 mmol) was added and the reaction mixture was allowed to warm to room temperature. After 3 h, the reaction mixture was quenched by the addition of hydrogen chloride (2 mL of a 2 M solution in ethyl ether, 4 mmol) at −78° C. Flash chromatography (SiO₂, 5-30% ethyl acetate/hexanes) provided 3-(3-chloropropyl)-1-(2-fluorophenyl)-3,4-dihydro-1H-benzo[c][1,2]thiazine 2,2-dioxide (0.55 g, 83%) as a yellow oil.:

HPLC purity 96.1% at 210-370 nm, 8.5 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 min

HRMS: calculated for C₁₇H₁₇ClFNNaO₂S+Na⁺, 376.0550; found (ESI, [M+Na]⁺), 376.054.

Step 7: A solution of 3-(3-chloropropyl)-1-(2-fluorophenyl)-3,4-dihydro-1H-benzo[c][1,2]thiazine 2,2-dioxide (1.1 g, 3.1 mmol) in an 8 M methyl amine solution in ethanol (80 mL) and heated to 70° C. in a sealed tube for 24 hours. The reaction mixture was cooled to room temperature and evaporated to provide crude 3-[1-(2-fluorophenyl)-2,2-dioxido-3,4-dihydro-1H-2,1-benzothiazin-3-yl]-N-methylpropan-1-amine hydrochloride (1.2 g, 100%) as a tan solid:

HPLC purity 99.2% at 210-370 nm, 6.7 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₁₈H₂₁FN₂O₂S+H⁺, 349.13805; found (ESI, [M+H]⁺), 349.1382.

### Example 74

### 3-[(3S)-1-(2-fluorophenyl)-2,2-dioxido-3,4-dihydro-1H-2,1-benzothiazin-3-yl]-N-methylpropan-1-amine Hydrochloride

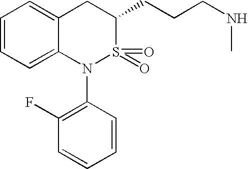

Approximately 1.1 g of a racemic mixture of 3-[1-(2-fluorophenyl)-2,2-dioxido-3,4-dihydro-1H-2,1-benzothiazin-3-yl]-N-methylpropan-1-amine hydrochloride was dissolved in 17 mL of methanol. 0.4 mL of the resulting solution was repetitively injected onto the Supercritical Fluid Chromatography instrument, and the baseline resolved enantiomers were collected using a Berger MultiGram Prep SFC (Berger Instruments, Inc. Newark, Del.) under the following conditions: Chiralpak AD-H column ( [symbol] micron, 250 mm L×20 mm ID, Chiral Technologies, Inc, Exton, Pa.), 35° C. column temperature, 25% methanol w/0.2% dimethylethylamine/75% CO₂, 50 mL/min flow rate, 100 bar outlet pressure, 220 nm UV detection. The chiral purity of each enantiomer was determined under the same SFC conditions using a Chiralcel AD-H column ( [symbol] μ, 250 mm L×4.6 mm ID) at 2.0 mL/min flow rate on a Berger Analytical SFC instrument. Both compounds were determined to be >99.9% chiraly pure (Rₜ 4.5 and 6.6 min).

Enantiomer 1: Rt 4.5 minutes, was dissolved in methanol, treated with excess 4N HCl in dioxan and evaporated to provide the product arbitrarily assigned as 3-[(3S)-1-(2-fluorophenyl)-2,2-dioxido-3,4-dihydro-1H-2,1-benzothiazin-3-yl]-N-methylpropan-1-amine hydrochloride (360 mg):

HPLC purity 100.0% at 210-370 nm, 6.8 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₁₈H₂₁FN₂O₂S+H⁺, 349.13805; found (ESI, [M+H]⁺), 349.1388.

### Example 75

### 3-[(3R)-1-(2-fluorophenyl)-2,2-dioxido-3,4-dihydro-1H-2,1-benzothiazin-3-yl]-N-methylpropan-1-amine

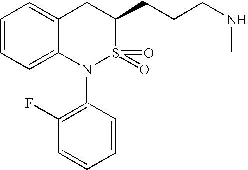

The second eluting enantiomer isolated from Example 74, enantiomer 2 isolated with Rt 6.6.minutes, was dissolved in methanol, treated with excess 4N HCl in dioxan and evaporated to provide the product arbitrarily assigned was arbitrarily assigned as (3R)-3-allyl-1-phenyl-3,4-dihydro-1H-2,1-benzothiazine 2,2-dioxide (420 mg):

HPLC purity 95.8% at 210-370 nm, 6.8 minutes; Xterra RP18, 3.5 u, 150×4.6 mm column, 1.2 mL/minutes, 85/15-5/95 (ammonium formate buffer pH=3.5/ACN+MeOH)

for 10 minutes, hold 4 minutes. HRMS: calculated for C₁₈H₂₁FN₂O₂S+H⁺, 349.13805; found (ESI, [M+H]⁺), 349.1383.

### Example 76

### 1-phenyl-3-(3-piperazin-1-ylpropyl)-1H-4,2,1-benzoxathiazine2,2-dioxide Dihydrochloride

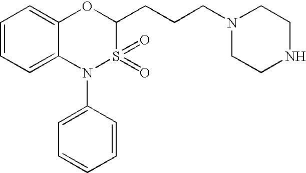

Step 1: A solution of 3-(2-bromoethyl)-1-phenyl-3,4-dihydro-1H-2,1-benzothiazine 2,2-dioxide (example 23, step 2) (100 mg) in dichloromethane (1 mL) and ethanol (1 mL) was treated with N-Boc-piperazine (76 mg)) and i-Pr₂NEt (70 mL) and heated in a capped vial at 80° C. for 16 hours. The reaction mixture was cooled to room temperature, diluted with dichloromethane, washed with H₂O, dried (Na₂SO₄), and evaporated. Flash chromatography (SiO₂, 0-100% ethyl acetate/hexanes) provided tert-butyl 4-[2-(2,2-dioxido-1-phenyl-1H-4,2,1-benzoxathiazin-3-yl)ethyl]piperazine-1-carboxylate (144 mg) as a yellow oil.

Step 2: A solution of tert-butyl 4-[2-(2,2-dioxido-1-phenyl-1H-4,2,1-benzoxathiazin-3-yl)ethyl]piperazine-1-carboxylate (100 mg) in dichloromethane (2 mL) was treated with 4 M HCl-dioxane (4 mL) and stirred at 22° C. for 16 hours. The reaction mixture was evaporated to provide 1-phenyl-3-(3-piperazin-1-ylpropyl)-1H-4,2,1-benzoxathiazine 2,2-dioxide hydrochloride (77 mg) as a white powder:

HPLC purity 100.0% at 210-370 nm, 9.5 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₂₄H₃₁N₃O₅S+H⁺, 474.20572; found (ESI, [M+H]⁺), 474.206.

### Example 77

### 3-[2-(1,4-diazepan-1-yl)ethyl]-1-phenyl-1H-4,2,1-benzoxathiazine-2,2-dioxide Dihydrochloride

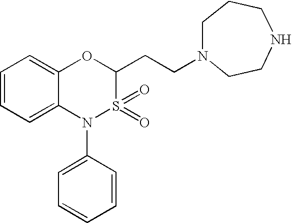

Step 1: A solution of 3-(2-bromoethyl)-1-phenyl-3,4-dihydro-1H-2,1-benzothiazine 2,2-dioxide (example 23, step 2) (100 mg) in dimethylformamide (1 mL) was treated with N-Boc-homopiperazine (0.106 mL) and i-Pr₂NEt (0.90 mL) and heated in a capped vial at 80° C. for 16 hours. The reaction mixture was cooled to room temperature, diluted with dichloromethane, washed with H₂O, dried (Na₂SO₄), and evaporated. Flash chromatography (SiO₂, 0-100% ethyl acetate/hexanes) provided tert-butyl 4-[2-(2,2-dioxido-1-phenyl-1H-4,2,1-benzoxathiazin-3-yl)ethyl]-1,4-diazepane-1-carboxylate (175 mg) as a yellow oil:

HPLC purity 100.0% at 210-370 nm, 8.7 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₂₅H₃₃N₃O₅S+H⁺, 488.22137; found (ESI, [M+H]⁺), 488.2211

Step 2: A solution of tert-butyl 4-[2-(2,2-dioxido-1-phenyl-1H-4,2,1-benzoxathiazin-3-yl)ethyl]-1,4-diazepane-1-carboxylate (118 mg) in dichloromethane (2 mL) was treated with 4 M HCl-dioxane (4 mL) and stirred at 22° C. for 16 hours. The reaction mixture was evaporated to provide 3-[2-(1,4-diazepan-1-yl)ethyl]-1-phenyl-1H-4,2,1-benzoxathiazine 2,2-dioxide dihydrochloride (77 mg) as a white powder:

HPLC purity 100.0% at 210-370 nm, 7.0 minutes; Xterra RP18, 3.5 u, 150×4.6 mm column, 1.2 mL/minutes, 85/15-5/95 (ammonium formate buffer Ph=3.5/ACN+MeOH)

for 10 minutes, hold 4 minutes.

HRMS: calculated for C₂₀H₂₅N₃O₃S+H⁺, 388.16894; found (ESI, [M+H]⁺), 388.1687.

### Example 78

### 1-phenyl-3-(2-piperidin-1-ylethyl)-1H-4,2,1-benzoxathiazine 2,2-dioxide hydrochloride

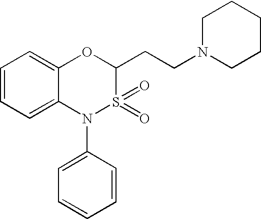

A solution of 3-(2-bromoethyl)-1-phenyl-3,4-dihydro-1H-2,1-benzothiazine 2,2-dioxide (example 23, step 2) (150 mg) in dimethylformamide (1 mL) was treated with piperidine (0.122 mL) and heated in a capped vial at 80° C. for 16 hours. The reaction mixture was cooled to room temperature, diluted with dichloromethane, washed with H₂O, dried (Na₂SO₄), and evaporated. Flash chromatography (SiO₂, 0-10% 7 M NH₃ in methanol/dichloromethane) provided the purified free base that was dissolved in ether and treated with 2 M HCl-ether (0.5 mL) to provide 1-phenyl-3-(2-piperidin-1-ylethyl)-1H-4,2,1-benzoxathiazine 2,2-dioxide hydrochloride (117 mg) as a white powder:

HPLC purity 100.0% at 210-370 nm, 7.3 minutes; Xterra RP18, 3.5 u, 150×4.6 mm column, 1.2 mL/minutes, 85/15-5/95 (ammonium formate buffer pH=3.5/ACN+MeOH)

for 10 minutes, hold 4 minutes.

HRMS: calculated for C₂₀H₂₄N₂O₃S+H⁺, 373.15804; found (ESI, [M+H]⁺), 373.1577.

### Example 79

### 3-[2-(3,5-dimethylpiperazin-1-yl)ethyl]-1-phenyl-1H-4,2,1-benzoxathiazine 2,2-dioxide hydrochloride

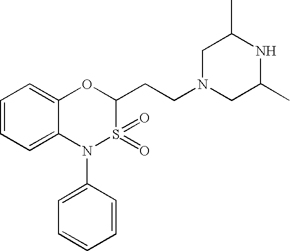

A solution of 3-(2-bromoethyl)-1-phenyl-3,4-dihydro-1H-2,1-benzothiazine 2,2-dioxide (example 23, step 2) (100 mg) in dimethylformamide (1 mL) was treated with 2,6-dimethylpiperidine (predominantly trans, 94 mg) and heated in a capped vial at 80° C. for 16 hours. The reaction mixture was cooled to room temperature, diluted with dichloromethane, washed with H₂O, dried (Na₂SO₄), and evaporated. Flash chromatography (SiO₂, 0-10% 7 M NH₃ in methanol/dichloromethane) provided the purified free base that was dissolved in ether and treated with 2 M HCl-ether (0.5 mL) to provide 1-phenyl-3-(2-piperidin-1-ylethyl)-1H-4,2,1-benzoxathiazine 2,2-dioxide hydrochloride (117 mg) as a white powder:

HPLC purity 99.2% at 210-370 nm, 8.3 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₂₅H₃₁N₃O₅S+H⁺, 486.20572; found (ESI, [M+H]⁺), 486.2072.

### Example 80

### 3-(2,2-dioxido-1-phenyl-1H-4,2,1-benzoxathiazin-3-yl)-N,N-dimethylpropan-1-amine hydrochloride

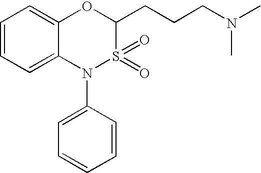

A solution of 3-(3-bromopropyl)-1-phenyl-1H-4,2,1-benzoxathiazine 2,2-dioxide (0.2 g, 0.534 mmol) and dimethylamine (0.110 mL of a 5.6 M ethanol solution, 0.56 mmol) where combined in 1 mL of ethanol and stirred in a capped vial for 16 hours. The content of the vial was absorbed on silica gel and flash chromatography (SiO₂, 0-15% NH₃-methanol/dichloromethane) providing a tan residue. The residue was dissolved in ethyl ether (5 mL) and treated with hydrogen chloride (1.0 mL of a 2 M solution in ethyl ether), resulting in a white precipitate that was isolated by decantation and dried under vacuum to afford 3-(2,2-dioxido-1-phenyl-1H-4,2,1-benzoxathiazin-3-yl)-N,N-dimethylpropan-1-amine hydrochloride (0.0873 g, 47%) as a white solid:

MS (ES) m/z 347.1;

HPLC purity 100.0% at 210-370 nm, 7.0 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₁₈H₂₂N₂O₃S+H⁺, 347.14239; found (ESI, [M+H]⁺), 347.1418;

### Example 81

### 3-(2,2-dioxido-1-phenyl-1H-4,2,1-benzoxathiazin-3-yl)-N-ethyl-N-methylpropan-1-amine hydrochloride

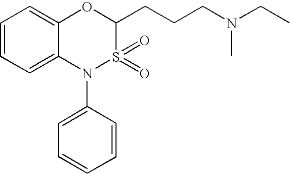

In an analogous manner to Example 80, 3-(3-bromopropyl)-1-phenyl-1H-4,2,1-benzoxathiazine 2,2-dioxide (0.2 g, 0.534 mmol) was treated with N-ethylmethylamine (0.070 mL, 0.815 mmol) to provide 3-(2,2-dioxido-1-phenyl-1H-4,2,1-benzoxathiazin-3-yl)-N-ethyl-N-methylpropan-1-amine hydrochloride (0.1073 g, 55%) as a white solid:

MS (ES) m/z 361.2;

HPLC purity 100.0% at 210-370 nm, 7.2 minutes; Xterra RP18, 3.5 u, 150×4.6 mm column, 1.2 mL/minutes, 85/15-5/95 (Ammonium Formate Buffer pH=3.5/ACN+MeOH)

for 10 minutes, hold 4 minutes.

HRMS: calculated for C₁₉H₂₄N₂O₃S+H⁺, 361.15804; found (ESI, [M+H]⁺), 361.1573;

### Example 82

### 3-(2,2-dioxido-1-phenyl-1H-4,2,1-benzoxathiazin-3-yl)-N,N-diethylpropan-1-amine hydrochloride

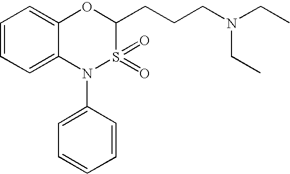

In an analogous manner to Example 80, 3-(3-bromopropyl)-1-phenyl-1H-4,2,1-benzoxathiazine 2,2-dioxide (0.2 g, 0.534 mmol) was treated with diethylamine (0.085 mL, 0.815 mmol) to provide 3-(2,2-dioxido-1-phenyl-1H-4,2,1-benzoxathiazin-3-yl)-N,N-diethylpropan-1-amine hydrochloride (0.0937 g, 47%) as a white solid:

MS (ES) m/z 374.8;

HPLC purity 100.0% at 210-370 nm, 7.4 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₂₀H₂₆N₂O₃S+H⁺, 375.17369; found (ESI, [M+H]⁺), 375.1725;

### Example 83

### 2-{[3-(2,2-dioxido-1-phenyl-1H-4,2,1-benzoxathiazin-3-yl)propyl](ethyl)amino}ethanol hydrochloride

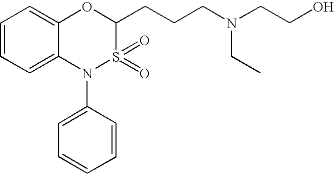

In an analogous manner to Example 80, 3-(3-bromopropyl)-1-phenyl-1H-4,2,1-benzoxathiazine 2,2-dioxide (0.2 g, 0.534 mmol) was treated with N-Ethylethanolamine (0.080 mL, 0.82 mmol) to provide 2-{[3-(2,2-dioxido-1-phenyl-1H-4,2,1-benzoxathiazin-3-yl)propyl](ethyl)amino}ethanol (0.1231 g, 58%) as a white solid:

MS (ES) m/z 391.1;

HPLC purity 100.0% at 210-370 nm, 7.1 minutes; Xterra RP18, 3.5 u, 150×4.6 mm column, 1.2 mL/minutes, 85/15-5/95 (Ammonium Formate Buffer Ph=3.5/ACN+MeOH) for 10 minutes, hold 4 minutes.

HRMS: calculated for C20H26N2O4S+H+, 391.16860; found (ESI, [M+H]+), 391.1671;

### Example 84

### 3-(2,2-dioxido-1-phenyl-1H-4,2,1-benzoxathiazin-3-yl)-N-isopropyl-N-methylpropan-1-amine Hydrochloride

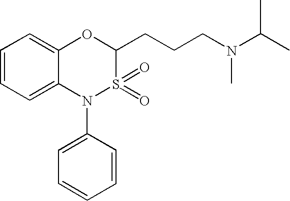

In an analogous manner to Example 80, 3-(3-bromopropyl)-1-phenyl-1H-4,2,1-benzoxathiazine 2,2-dioxide (0.2 g, 0.534 mmol) was treated with N-isopropylmethylamine (0.085 mL, 0.82 mmol) to provide 3-(2,2-dioxido-1-phenyl-1H-4,2,1-benzoxathiazin-3-yl)-N-isopropyl-N-methylpropan-1-amine hydrochloride (0.1142 g, 57%) as a white solid:

MS (ES) m/z 375.3;

HPLC purity 100.0% at 210-370 nm, 7.3 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₂₀H₂₆N₂O₃S+H⁺, 375.17369; found (ESI, [M+H]⁺), 375.1732.

### Example 85

### N-[3-(2,2-dioxido-1-phenyl-1H-4,2,1-benzoxathiazin-3-yl)propyl]-N-methylcyclo hexanamine Hydrochloride

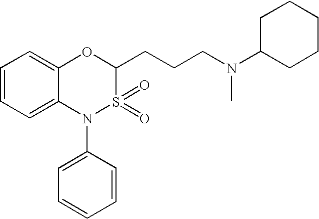

In an analogous manner to Example 80, 3-(3-bromopropyl)-1-phenyl-1H-4,2,1-benzoxathiazine 2,2-dioxide (0.2 g, 0.534 mmol) was treated with N-cyclohexyllmethylamine (0.105 mL, 0.795 mmol) to provide N-[3-(2,2-dioxido-1-phenyl-1H-4,2,1-benzoxathiazin-3-yl)propyl]-N-methylcyclohexanamine hydrochloride (0.0814 g, 37%) as a white solid:

MS (ES) m/z 414.8;

HPLC purity 100.0% at 210-370 nm, 8.3 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

### Example 86

### 1-Phenyl-3-(2-pyrrolidin-1-yl-ethyl)-1H-benzo[1,3,4]oxathiazine 2,2-dioxide Hydrochloride

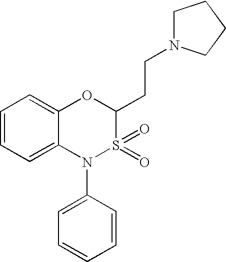

Prepared as the HCl salt according to the procedure for Example 34: 101 mg (94% Yield)

HPLC purity 95.1% at 210-370 nm, 7.1 minutes; Xterra RP18, 3.5 u, 150×4.6 mm column, 1.2 mL/minutes, 85/15-5/95 (ammonium bicarbonate buff. pH=9.5/ACN+MeOH) for 10 minutes, hold 4 minutes.

HRMS: calculated for C₁₉H₂₂N₂O₃S+H⁺, 359.14239; found (ESI, [M+H]⁺), 359.1428.

### Example 87

### 3-[2-(4-Methyl-piperazin-1-yl)-ethyl]-1-phenyl-1H-benzo[1,3,4]oxathiazine 2,2-dioxide hydrochloride

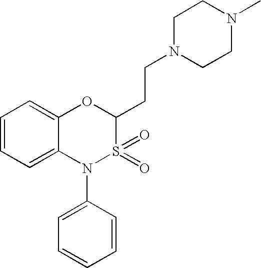

Prepared as the HCl salt according to procedure for Example 34: 100 mg (87% Yield)

HPLC purity 98.6% at 210-370 nm, 7.2 minutes; Xterra RP18, 3.5 u, 150×4.6 mm column, 1.2 mL/minutes, 85/15-5/95 (ammonium bicarbonate buffer pH=9.5/ACN+MeOH) for 10 minutes, hold 4 minutes.

HRMS: calculated for C₂₀H₂₅N₃O₃S+H⁺, 388.16894; found (ESI, [M+H]⁺), 388.1709.

### Example 88

### N-butyl-[2-(2,2-dioxo-1-phenyl-2,3-dihydro-1H-2λ⁶-benzo[1,3,4]oxathiazin-3-yl)-ethyl]-amine Hydrochloride

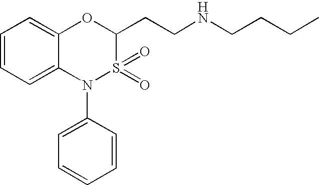

Prepared as the HCl salt according to procedure for Example 34: 41 mg (38% Yield)

HPLC purity 100.0% at 210-370 nm, 7.8 minutes; Xterra RP18, 3.5 u, 150×4.6 mm column, 1.2 mL/minutes, 85/15-5/95 (ammonium formate buffer pH=3.5/ACN+MeOH) for 10 minutes, hold 4 minutes.

HRMS: calculated for C₁₉H₂₄N₂O₃S+H⁺, 361.15804; found (ESI, [M+H]⁺), 361.1588.

### Example 89

### 3-[1-(2-fluorophenyl)-2,2-dioxido-3,4-dihydro-1H-2,1-benzothiazin-3-yl]-N-methylpropan-1-amine

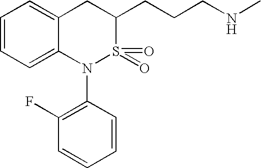

Step 1: A solution of 2-bromophenethylalcohol (10 g, 50 mmol) in thionyl chloride (45 mL) was treated with dimethylformamide (0.1 mL) and heated to reflux for 6 h. The reaction mixture was cooled to 0° C., quenched by the addition of H₂O (100 mL), extracted with ethyl ether (250 mL), dried (Na₂SO₄), and evaporated to provide 2-bromophenethylchloride (9.0 g, 82%) as a yellow oil:

HPLC Retention time 10.4 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₈H₈BrCl, 217.94979; found (EI, M⁺), 217.9503.

Step 2: A suspension of 2-bromophenethylchloride (2.2 g, 10 mmol) in H₂O (12 mL) was treated with sodium sulfite (1.3 g, 10 mmol), and sodium iodide (100 mg) and heated in a sealed glass tube at 130° C. for 16 h. The reaction mixture was cooled to 0° C., forming a white precipitate, which was isolated by filtration, and washed with ice-water and hexanes to provide sodium 2-bromophenethylsulfonate (1.7 g, 60%) as a white solid:

HPLC Retention time 5.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calc'd for C₈H₉BrO₃S−H+, 262.93830; found (ESI, [M−H]⁻), 262.939;

Step 3: A suspension of sodium 2-bromophenethylsulfonate (2.87 g, 10 mmol) in toluene (70 mL) was treated with thionyl chloride (50 mL) and dimethylformamide (0.3 mL) and heated to 80° C. for 16 h. The reaction mixture was cooled to room temperature, and evaporated. The residue was suspended in dichloromethane (100 mL) and filtered to remove inorganic material. Concentration provided 2-bromophenethylsulfonyl chloride (2.5 g, 88%) as a yellow oil:

HPLC Retention time 5.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

Step 4: A solution of 2-bromophenethylsulfonyl chloride (2.6 g, 9.2 mmol) in dichloromethane (25 mL) was added dropwise to a solution of 2-fluoroaniline (1.65 mL, 17.1 mmol) and pyridine (1.2 mL, 14.8 mmol) in dichloromethane (25 mL) over 30 minutes, and stirred at 22° C. for 14 h. The reaction mixture was diluted with 2 N hydrochloric acid (150 mL), extracted with dichloromethane (2×150 mL), dried (Na₂SO₄), and evaporated. Flash chromatography (SiO₂, 10-100% dichloromethane/hexanes) provided 2-(2-bromophenyl)-N-(2-fluorophenyl)ethanesulfonamide (2.1 g, 64%) as a white solid:

HPLC Retention time 9.8 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₄H₁₃BrFNO₂S−H⁺, 355.97616; found (ESI, [M−H]⁻), 355.9760.

Step 5: A solution of 2-(2-bromophenyl)-N-(2-fluorophenyl)ethanesulfonamide (1.79 g, 5 mmol) in dimethylsulfoxide (40 mL) and benzene (4 mL) was treated with cesium acetate (4.8 g, 25 mmol) and copper(I) iodide (1.9 g, 10 mmol) and stirred at 22° C. for 22 h. The reaction mixture was diluted with ethyl ether (200 mL), washed with sat. ammonium hydroxide (200 mL), dried (Na₂SO₄), and evaporated. Flash chromatography (SiO₂, 5-50% ethyl acetate/hexanes) provided 1-(2-fluorophenyl)-3,4-dihydro-1H-benzo[c][1,2]thiazine 2,2-dioxide (1.1 g, 80%) as a white solid:

HPLC Retention time 8.5 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calculated for C₁₄H₁₃BrFNO₂S+H⁺, 277.05728; found (ESI, [M+H—SO₂]⁺), 277.0569.

Step 6: A solution of 1-(2-fluorophenyl)-3,4-dihydro-1H-benzo[c][1,2]thiazine 2,2-dioxide (0.52 g, 1.9 mmol) in tetrahydrofuran (8 mL) was treated with lithium hexamethyldisilazide (2. 2 mL of a 1.0 M solution in tetrahydrofuran, 2.2 mmol) at −78° C. After 3 h, 3-bromo-1-chloropropane (0.4 mL, 4 mmol) was added and the reaction mixture was allowed to warm to room temperature. After 3 h, the reaction mixture was quenched by the addition of hydrogen chloride (2 mL of a 2 M solution in ethyl ether, 4 mmol) at −78° C., and the mixture concentrated. Flash chromatography (SiO₂, 5-30% ethyl acetate/hexanes) provided

3-(3-chloropropyl)-1-(2-fluorophenyl)-3,4-dihydro-1H-benzo[c][1,2]thiazine 2,2-dioxide (0.55 g, 83%) as a yellow oil:

HPLC Retention time 8.5 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calculated for C₁₇H₁₇ClFNNaO₂S+Na⁺, 376.0550; found (ESI, [M+Na]⁺), 376.054.

Step 7: A solution of 3-(3-chloropropyl)-1-(2-fluorophenyl)-3,4-dihydro-1H-benzo[c][1,2]thiazine 2,2-dioxide (1.1 g, 3.1 mmol) in an 8 M methyl amine solution in ethanol (80 mL) was heated to 70° C. in a sealed tube for 24 h. The reaction mixture was cooled to room temperature and evaporated to provide crude 3-[1-(2-fluorophenyl)-2,2-dioxido-3,4-dihydro-1H-2,1-benzothiazin-3-yl]-N-methylpropan-1-amine hydrochloride (WYE-103688) (1.2 g, 100%) as a tan solid:

HPLC Retention time 6.7 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₁FN₂O₂S+H+, 349.13805; found (ESI, [M+H]+), 349.1382.

### Example 90

### 3-[(3S)-1-(2-fluorophenyl)-2,2-dioxido-3,4-dihydro-1H-2,1-benzothiazin-3-yl]-N-methylpropan-1-amine

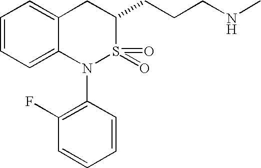

and

### Example 91

### 3-[(3R)-1-(2-fluorophenyl)-2,2-dioxido-3,4-dihydro-1H-2,1-benzothiazin-3-yl]-N-methylpropan-1-amine

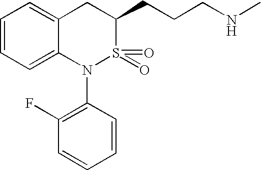

Approximately 1.1 g of racemic mixture of 3-[1-(2-fluorophenyl)-2,2-dioxido-3,4-dihydro-1H-2,1-benzothiazin-3-yl]-N-methylpropan-1-amine hydrochloride was dissolved in 17 mL of methanol. 0.4 mL of the resulting solution was repetitively injected onto the Supercritical Fluid Chromatography instrument, and the baseline resolved enantiomers were collected using a Berger MultiGram Prep SFC (Berger Instruments, Inc. Newark, Del.) under the following conditions: Chiralpak AD-H column ( [symbol] micron, 250 mm L×20 mm ID, Chiral Technologies, Inc, Exton, Pa.), 35° C. column temperature, 25% methanol w/0.2% dimethylethylamine/75% CO₂, 50 mL/min flow rate, 100 bar outlet pressure, 220 nm UV detection. The chiral purity of each enantiomer was determined under the same SFC conditions using a Chiralcel AD-H column (5 micron, 250 mm L×4.6 mm ID) at 2.0 mL/min flow rate on a Berger Analytical SFC instrument. Both compounds were determined to be >99.9% chiraly pure (Rₜ 4.5 and 6.6 min).

Enantiomer 1 (SFC Rt 4.5 minutes), arbitrarily assigned as 3-[(3S)-1-(2-fluorophenyl)-2,2-dioxido-3,4-dihydro-1H-2,1-benzothiazin-3-yl]-N-methylpropan-1-amine: Recovered sample from SFC purification was dissolved in methanol (10 mL) and treated with 2 M HCl-ether (1 mL) and evaporated to a white powder (360 mg):

HPLC Retention time 6.8 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₁FN₂O₂S+H+, 349.13805; found (ESI, [M+H]⁺), 349.1388.

Enantiomer 2: (SFC Rt 6.6.min), arbitrarily assigned as (3R)-3-allyl-1-phenyl-3,4-dihydro-1H-2,1-benzothiazine 2,2-dioxide: Recovered sample from SFC purification was dissolved in methanol (10 mL) and treated with 2 M HCl-ether (1 mL) and evaporated to a white powder: (420 mg):

HPLC Retention time 6.8 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₁FN₂O₂S+H⁺, 349.13805; found (ESI, [M+H]⁺), 349.1383.

### Example 92

### 3-[6-fluoro-1-(4-fluoro-2-methylphenyl)-2,2-dioxido-3,4-dihydro-1H-2,1-benzothiazin-3-yl]-N-methylpropan-1-amine

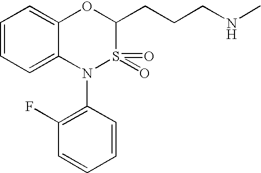

Step 1: A solution of 1H-4,2,1-benzoxathiazine 2,2-dioxide (2.78 g, 15 mmol) in dichloromethane (40 mL) was treated with pyridine (1.8 mL, 23 mmol), 2-fluorophenylboronic acid (2.1 g, 15 mmol) and copper(II) acetate (2.7 g, 15 mmol) and stirred at 22° C. for 48 h. The reaction mixture was washed with 2 M hydrochloric acid (50 mL), dried (Na₂SO₄), and evaporated. Flash chromatography (SiO₂, 10-50% ethyl acetate/hexanes) provided 1-(2-fluorophenyl)-1H-4,2,1-benzoxathiazine 2,2-dioxide (0.24 g, 7%) as a yellow oil:

MS (ES) m/z 216.0;

HPLC Retention time 8.6 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

Step 2: A solution of 1-(2-fluorophenyl)-1H-4,2,1-benzoxathiazine 2,2-dioxide (0.23 g, 0.82 mmol) in tetrahydrofuran (4 mL) was treated with lithium hexamethyldisilazide (1 mL of a 1.0 M solution in tetrahydrofuran, 1 mmol) at −78° C. After 2 h, 3-bromo-1-chloropropane (0.32 mL, 3.3 mmol) was added and the reaction mixture was allowed to warm to room temperature. After 16 h, the reaction mixture was quenched by the addition of H₂O (50 mL), extracted into ethyl acetate (2×50 mL) and evaporated. Flash chromatography (SiO₂, 10-50% ethyl acetate/hexanes) provided

3-(3-chloropropyl)-1-(2-fluorophenyl)-1H-4,2,1-benzoxathiazine 2,2-dioxide (0.12 g) as a yellow oil.

A solution of 3-(3-chloropropyl)-1-(2-fluorophenyl)-1H-4,2,1-benzoxathiazine 2,2-dioxide (70 mg, 0.2 mmol) in an 8 M methyl amine solution in ethanol (30 mL) was heated to 70° C. in a sealed tube for 16 h. The reaction mixture was cooled to room temperature and evaporated to provide crude 3-[1-(2-fluorophenyl)-2,2-dioxido-1H-4,2,1-benzoxathiazin-3-yl]-N-methylpropan-1-amine hydrochloride (60 mg, 78%) as a white solid:

HPLC Retention time 6.7 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C17H19FN2O3S+H+, 351.11732; found (ESI, [M+H]+ Obs'd), 351.1180

### Example 93

### 3-[(3S)-6-fluoro-1-(4-fluoro-2-methylphenyl)-2,2-dioxido-3,4-dihydro-1H-2,1-benzothiazin-3-yl]-N-methylpropan-1-amine

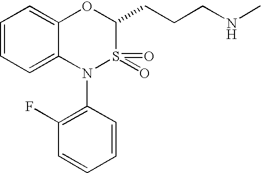

and

### Example 94

### 3-[(3R)-6-fluoro-1-(4-fluoro-2-methylphenyl)-2,2-dioxido-3,4-dihydro-1H-2,1-benzothiazin-3-yl]-N-methylpropan-1-amine

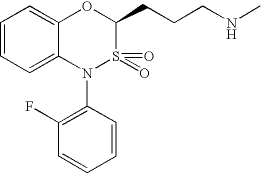

A methanolic solution of approximately 0.06 g of racemic mixture of 3-[1-(2-fluorophenyl)-2,2-dioxido-1H-4,2,1-benzoxathiazin-3-yl]-N-methylpropan-1-amine hydrochloride was injected onto a Supercritical Fluid Chromatography instrument, and the baseline resolved enantiomers were collected using a Berger MultiGram Prep SFC (Berger Instruments, Inc. Newark, Del.) under the following conditions: Chiralcel OJ column ( [symbol] micron, 250 mm L×20 mm ID, Chiral Technologies, Inc, Exton, Pa.), 35° C. column temperature, 25% methanol w/0.2% dimethylethylamine/75% CO₂, 50 mL/min flow rate, 100 bar outlet pressure, 220 nm UV detection. The chiral purity of each enantiomer was determined under the same SFC conditions using a Chiralcel OJ column ( [symbol] micron, 250 mm L×4.6 mm ID) at 2.0 mL/min flow rate on a Berger Analytical SFC instrument. Both compounds were determined to be >99.9% chiraly pure (Rₜ 3.3 and 4.2 min).

Enantiomer 1 (SFC Rt 3.3 min.), arbitrarily assigned as 3-[(3S)-1-(2-fluorophenyl)-2,2-dioxido-1H-4,2,1-benzoxathiazin-3-yl]-N-methylpropan-1-amine: Recovered sample from SFC purification was dissolved in methanol (10 mL) and treated with 2 M HCl-ether (1 mL) and evaporated to a white powder:

HPLC Retention time 6.7 min.; Xterra RP18, 3.5 u, 150×4.6 mm column, 1.2 mL/min, 85/15-5/95 (Ammonium formate buffer, pH 3.5/CH₃CN +MeOH)

for 10 min., hold for 4 min.

HRMS: calcd for C₁₇H₁₉FN₂O₃S+H⁺, 351.11732; found (ESI, [M+H]⁺ Obs'd), 351.1178.

Enantiomer 2 (SFC Rt 4.2.min.) arbitrarily assigned as 3-[(3R)-1-(2-fluorophenyl)-2,2-dioxido-1H-4,2,1-benzoxathiazin-3-yl]-N-methylpropan-1-amine: Recovered sample from SFC purification was dissolved in methanol (10 mL) and treated with 2 M HCl-ether (1 mL) and evaporated to a white powder:

HPLC purity 99.1% at 210-370 nm, 6.8 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₁₉FN₂O₃S+H⁺, 351.11732; found (ESI, [M+H]⁺ Obs'd), 351.1187.

### Example 95

### (2S)-1-[(3R)-2,2-dioxido-1-phenyl-1H-4,2,1-benzoxathiazin-3-yl]-3-(methylamino)propan-2-ol

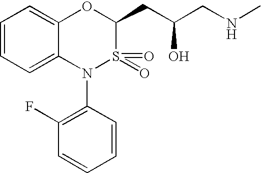

### Example 96

### (2R)-1-[(3R)-2,2-dioxido-1-phenyl-1H-4,2,1-benzoxathiazin-3-yl]-3-(methylamino)propan-2-ol

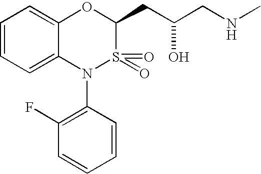

### Example 97

### (2R)-1-[(3S)-2,2-dioxido-1-phenyl-1H-4,2,1-benzoxathiazin-3-yl]-3-(methylamino)propan-2-ol

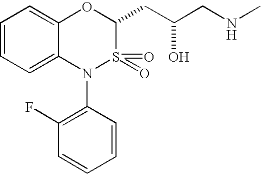

### Example 98

### (2S)-1-[(3S)-2,2-dioxido-1-phenyl-1H-4,2,1-benzoxathiazin-3-yl]-3-(methylamino)propan-2-ol

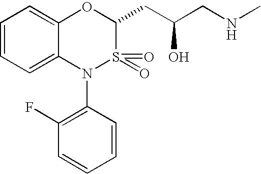

A solution of 1-phenyl-1H-4,2,1-benzoxathiazine 2,2-dioxide (1.96 g, 7.5 mmol) in tetrahydrofuran (30 mL) was treated with lithium hexamethyldisilazide (8.3 mL of a 1.0 M solution in tetrahydrofuran, 8.3 mmol) at −78° C. The reaction mixture was warmed to 0° C. for 15 min, then cooled back to −78° C. After 15 additional min, epichlorohydrin (4.4 mL, 56 mmol was added and the reaction mixture was allowed to warm to room temperature. After 3 h, the reaction mixture was quenched by the addition of 2N hydrochloric acid (100 mL) and extracted into ethyl ether (200 mL) and evaporated. Flash chromatography (SiO₂, 5-40% ethyl acetate/hexanes) provided

[2,2-dioxido-1-phenyl-1H-4,2,1-benzoxathiazin-3-yl]-methyloxirane (1.2 g, as a mixture of diastereomers) as a yellow oil.

A solution of [2,2-dioxido-1-phenyl-1H-4,2,1-benzoxathiazin-3-yl]-methyloxirane (1.2 g, 3.6 mmol) in an 8 M methyl amine solution in ethanol (50 mL) was heated to 70° C. in a sealed tube for 3 h. The reaction mixture was cooled to room temperature and evaporated to provide crude 1-[2,2-dioxido-1-phenyl-1H-4,2,1-benzoxathiazin-3-yl]-3-(methylamino)propan-2-ol (500 mg, tan solid) as a mixture of diastereomers

A methanolic solution of this mixture of diastereomers was separated on a Supercritical Fluid Chromatography instrument, and the baseline resolved isomers were collected using a Berger MultiGram Prep SFC (Berger Instruments, Inc. Newark, Del.) under the following conditions: 1. Chiralcel OJ-H column ( [symbol] μ, 250 mm L×20 mm ID, Chiral Technologies, Inc, Exton, Pa.), 35° C. column temperature, 20% methanol w/0.2% dimethylethylamine/80% CO₂, 50 mL/min flow rate, 100 bar outlet pressure, 220 nm UV detection, resulting in two peaks, each a pair of diastereomers: (Rₜ 2.9 and 3. 7 min). 2. The pairs of diastereomers where further purified by separation on an AD-H column ( [symbol] micron, 250 mm L×20 mm ID, Chiral Technologies, Inc, Exton, Pa.), 35° C. column temperature, 30% methanol w/0.2% dimethylethylamine/70% CO₂, 50 mL/min flow rate, 100 bar outlet pressure, 220 nm UV detection. The first diastereomer pair (Rt 2.9 min.) injected yielded isomers 1 (Rₜ 3.8 min) and 2 (Rₜ 4.6 min). The second diastereomer pair (Rₜ 3.7 min.) yielded isomers 3 (Rₜ 2.7 min) and 4 (Rₜ 4.2 min). Each compound was determined to be >99.9% chiraly pure.

Isomer 1 (Rt 3.8 min), arbitrarily assigned as (2S)-1-[(3R)-2,2-dioxido-1-phenyl-1H-4,2,1-benzoxathiazin-3-yl]-3-(methylamino)propan-2-ol: Recovered sample from SFC purification was dissolved in methanol (10 mL) and treated with 2 M HCl-ether (1 mL) and evaporated to a white powder:

HPLC Retention time 6.6 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₀N₂O₄S+H⁺, 349.12165; found (ESI, [M+H]⁺ Obs'd), 349.1216.

Isomer 2 (Rt 4.6 min) arbitrarily assigned as (2R)-1-[(3R)-2,2-dioxido-1-phenyl-1H-4,2,1-benzoxathiazin-3-yl]-3-(methylamino)propan-2-ol: Recovered sample from SFC purification was dissolved in methanol (10 mL) and treated with 2 M HCl-ether (1 mL) and evaporated to a white powder:

HPLC Retention time 6.5 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₀N₂O₄S+H⁺, 349.12165; found (ESI, [M+H]+Obs'd), 349.1219.

Isomer 3 (Rt 2.7 min) arbitrarily assigned as (2R)-1-[(3S)-2,2-dioxido-1-phenyl-1H-4,2,1-benzoxathiazin-3-yl]-3-(methylamino)propan-2-ol: Recovered sample from SFC purification was dissolved in methanol (10 mL) and treated with 2 M HCl-ether (1 mL) and evaporated to a white solid:

HPLC Retention time 6.6 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₀N₂O₄S+H⁺, 349.12165; found (ESI, [M+H]⁺ Obs'd), 349.1217.

Isomer 4 (Rt 4.2 min) arbitrarily assigned as (2S)-1-[(3S)-2,2-dioxido-1-phenyl-1H-4,2,1-benzoxathiazin-3-yl]-3-(methylamino)propan-2-ol: Recovered sample from SFC purification was dissolved in methanol (10 mL) and treated with 2 M HCl-ether (1 mL) and evaporated to a white powder:

HPLC Retention time 6.6 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₀N₂O₄S+H⁺, 349.12165; found (ESI, [M+H]⁺ Obs'd), 349.1218.

### Example 99

### 3-[1-(2,4-difluorophenyl)-2,2-dioxido-3,4-dihydro-1H-2,1-benzothiazin-3-yl]-N-methylpropan-1-amine

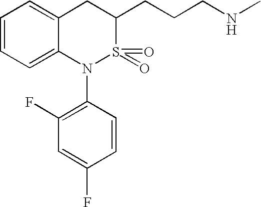

In an analogous manner to the preparation of 3-[1-(2-fluorophenyl)-2,2-dioxido-3,4-dihydro-1H-2,1-benzothiazin-3-yl]-N-methylpropan-1-amine, the title compound was prepared from 2-bromophenethylsulfonyl chloride and 2,4-difluoroaniline; the racemate analogously resolved by chiral SFC. The chiral purity of each enantiomer was determined under the same SFC conditions using a Chiralpak AD-H column ( [symbol] μ, 250 mm L×4.6 mm ID), 35° C. column temperature, 40% methanol with 0.2% dimethylethylamine/60% CO₂, 1 mL/min flow rate, 100 bar outlet pressure, 220 nm UV detection on a Berger Analytical SFC instrument. Both enantiomers were determined to be >99.9% chiraly pure (Rt 2.25 and 3.31 min):

Racemic 3-[1-(2,4-difluorophenyl)-2,2-dioxido-3,4-dihydro-1H-2,1-benzothiazin-3-yl]-N-methylpropan-1-amine:

HPLC retention time 7.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]⁺ Obs'd), 367.1289.

### Example 100

### 3-[(3S)-1-(2,4-difluorophenyl)-2,2-dioxido-3,4-dihydro-1H-2,1-benzothiazin-3-yl]-N-methylpropan-1-amine

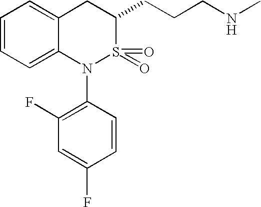

### Example 101

### 3-[(3R)-1-(2,4-difluorophenyl)-2,2-dioxido-3,4-dihydro-1H-2,1-benzothiazin-3-yl]-N-methylpropan-1-amine

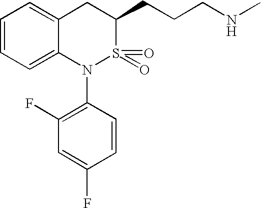

Enantiomer 1, (SFC Rₜ 2.25 min) arbitrarily assigned as 3-[(3S)-1-(2,4-difluorophenyl)-2,2-dioxido-3,4-dihydro-1H-2,1-benzothiazin-3-yl]-N-methylpropan-1-amine:

HPLC retention time, 7.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]⁺ Obs'd), 367.1285.

Enantiomer 2, (SFC Rt 3.31 min) arbitrarily assigned as 3-[(3R)-1-(2,4-difluorophenyl)-2,2-dioxido-3,4-dihydro-1H-2,1-benzothiazin-3-yl]-N-methylpropan-1-amine:

HPLC retention time 7.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]+ Obs'd), 367.1285.

### Example 102

### 2-[(3S)-2,2-dioxido-1-phenyl-1H-4,2,1-benzoxathiazin-3-yl]ethanamine

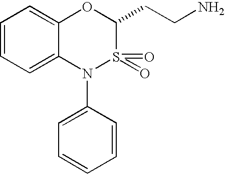

### Example 103

### 2-[(3R)-2,2-dioxido-1-phenyl-1H-4,2,1-benzoxathiazin-3-yl]ethanamine

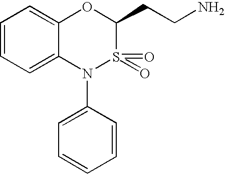

A methanolic solution of approximately 0.03 g of racemic 2-[2,2-dioxido-1-phenyl-1H-4,2,1-benzoxathiazin-3-yl]ethanamine hydrochloride was injected onto a Supercritical Fluid Chromatography instrument, and the baseline resolved enantiomers were collected using a Berger MultiGram Prep SFC (Berger Instruments, Inc. Newark, Del.) under the following conditions: Chiralpak AD-H column ( [symbol] micron, 250 mm L×20 mm ID, Chiral Technologies, Inc, Exton, Pa.), 35° C. column temperature, 30% methanol w/0.2% dimethylethylamine/70% CO₂, 65 mL/min flow rate, 100 bar outlet pressure, 220 nm UV detection. The chiral purity of each enantiomer was determined under the same SFC conditions using a Chiralpak AD-H column ( [symbol] micron, 250 mm L×4.6 mm ID) at 2.0 mL/min flow rate on a Berger Analytical SFC instrument. Both compounds were determined to be >99.9% chiraly pure (Rₜ 2.34 and 3.07 min).

Enantiomer 1, (SFC Rt 2.34 min.) arbitrarily assigned as 2-[(3S)-2,2-dioxido-1-phenyl-1H-4,2,1-benzoxathiazin-3-yl]ethanamine: Recovered sample from SFC purification was dissolved in methanol (10 mL) and treated with 2 M HCl-ether (0.5 mL) and evaporated to a white powder (10 mg):

HPLC retention time 6.5 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₅H₁₆N₂O₃S+H⁺, 305.09544; found (ESI, [M+H]⁺ Obs'd), 305.0956.

Enantiomer 2, (SFC Rt 3.07 min) arbitrarily assigned as 2-[(3R)-2,2-dioxido-1-phenyl-1H-4,2,1-benzoxathiazin-3-yl]ethanamine:

Recovered sample from SFC purification was dissolved in methanol (10 mL) and treated with 2 M HCl-ether (0.5 mL) and evaporated to a white powder (10 mg):

HPLC retention time 6.5 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₅H₁₆N₂O₃S+H⁺, 305.09544; found (ESI, [M+H]⁺ Obs'd), 305.0955.

### Example 104

### 2-[(3S)-2,2-dioxido-1-phenyl-1H-4,2,1-benzoxathiazin-3-yl]-N-methylethanamine

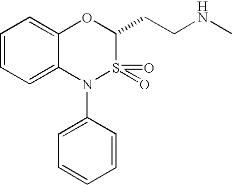

### Example 105

### 2-[(3R)-2,2-dioxido-1-phenyl-1H-4,2,1-benzoxathiazin-3-yl]-N-methylethanamine

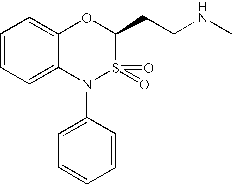

A methanolic solution of approximately 0.03 g of racemic 2-[2,2-dioxido-1-phenyl-1H-4,2,1-benzoxathiazin-3-yl]ethanamine hydrochloride was injected onto a Supercritical Fluid Chromatography instrument, and the baseline resolved enantiomers were collected using a Berger MultiGram Prep SFC (Berger Instruments, Inc. Newark, Del.) under the following conditions: Chiralpak AD-H column ( [symbol] micron, 250 mm L×20 mm ID, Chiral Technologies, Inc, Exton, Pa.), 35° C. column temperature, 30% methanol w/0.2% dimethylethylamine/70% CO₂, 65 mL/min flow rate, 100 bar outlet pressure, 220 nm UV detection. The chiral purity of each enantiomer was determined under the same SFC conditions using a Chiralpak AD-H column (5 micron, 250 mm L×4.6 mm ID) at 2.0 mL/min flow rate on a Berger Analytical SFC instrument. Both compounds were determined to be >99.9% chiraly pure (Rₜ 2.30 and 2.75 min).

Enantiomer 1 (SFC Rt 2.30 min.), arbitrarily assigned as 2-[(3S)-2,2-dioxido-1-phenyl-1H-4,2,1-benzoxathiazin-3-yl]-N-methylethanamine: Recovered sample from SFC purification was dissolved in methanol (10 mL) and treated with 2 M HCl-ether (0.5 mL) and evaporated to a white powder (10 mg):

HPLC retention time 6.6 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₆H₁₈N₂O₃S+H⁺, 319.11109; found (ESI, [M+H]⁺ Obs'd), 319.11

Enantiomer 1 (SFC Rt 2.75 min.) arbitrarily assigned as 2-[(3R)-2,2-dioxido-1-phenyl-1H-4,2,1-benzoxathiazin-3-yl]-N-methylethanamine:

Recovered sample from SFC purification was dissolved in methanol (10 mL) and treated with 2 M HCl-ether (0.5 mL) and evaporated to a white powder (10 mg):

HPLC retention time 6.6 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₆H₁₈N₂O₃S+H⁺, 319.11109; found (ESI, [M+H]⁺ Obs'd), 319.111.

### Example 106

### 3-[(3S)-6-fluoro-2,2-dioxido-1-phenyl-1H-4,2,1-benzoxathiazin-3-yl]-N-methylpropan-1-amine

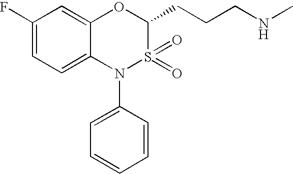

Step 1: Racemic 3-(6-fluoro-2,2-dioxido-1-phenyl-1H-4,2,1-benzoxathiazin-3-yl)-N-methylpropan-1-amine (Example 46) (0.52 g) was resolved by Supercritical Fluid Chromatography (SFC). The baseline resolved enantiomers were collected using a Berger MultiGram Prep SFC (Berger Instruments, Inc. Newark, Del.) under the following conditions: Chiralcel OJ-H (5 micron, 250 mm L×21 mm ID, Chiral Technologies, Inc, West Chester, Pa.), 35° C. column temperature, 20% MeOH/0.2% dimethylethylamine as CO₂ modifier, 60 mL/min flow rate, 100 bar outlet pressure, 235 nm UV detection.

Peak 1 Rt 3.0 min.

Peak 2 Rt 5.1 min.

Step 2: Sample from peak 1 from the previous step was dissolved in dichloromethane (3 mL) and treated with hydrogen chloride (1.0 mL of a 2 M solution in ethyl ether), resulting in a white precipitate that was collected and dried under vacuum to provided 3-[(3S)-6-fluoro-2,2-dioxido-1-phenyl-1H-4,2,1-benzoxathiazin-3-yl]-N-methylpropan-1-amine hydrochloride (0.1899 g) as a tan solid:

HPLC retention time 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₇H₁₉FN₂O₃S+H+, 351.11732; found (ESI, [M+H]+ Obs'd), 351.1176.

### Example 107

### 3-[(3R)-6-fluoro-2,2-dioxido-1-phenyl-1H-4,2,1-benzoxathiazin-3-yl]-N-methylpropan-1-amine

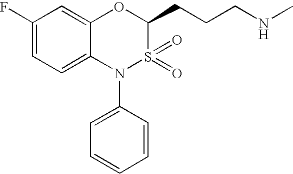

Peak 2 from step 1, Example 105 was dissolved in dichloromethane (3 mL) and treated with hydrogen chloride (1.0 mL of a 2 M solution in ethyl ether), resulting in a white precipitate that was evaporated and dried under vacuum to provided 3-[(3R)-6-fluoro-2,2-dioxido-1-phenyl-1H-4,2,1-benzoxathiazin-3-yl]-N-methylpropan-1-amine hydrochloride (0.1924 g) as a tan solid: HPLC retention time 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₇H₁₉FN₂O₃S+H+, 351.11732; found (ESI, [M+H]+ Obs'd), 351.1176.

### Example 108

### 3-[6-fluoro-1-(2-fluorophenyl)-2,2-dioxido-3,4-dihydro-1H-2,1-benzothiazin-3-yl]-N-methylpropan-1-amine

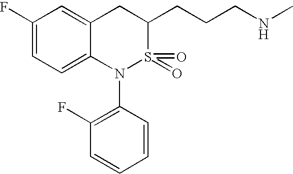

Step 1: A solution of 2-bromo-5-fluorophenethylalcohol (2.2 g, 10 mmol) in thionyl chloride (9 mL) was treated with dimethylformamide (0.2 mL) and heated to reflux for 16 h. The reaction mixture was cooled to 22° C., quenched by the addition of H₂O (100 mL), extracted with ethyl ether (100 mL), dried (Na₂SO₄), and evaporated to provide 1-bromo-2-(2-chloroethyl)-4-fluorobenzene (2.0 g, 84%) as a yellow oil:

HPLC retention time 10.5 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

Step 2: A suspension of 1-bromo-2-(2-chloroethyl)-4-fluorobenzene (1.8 g, 7.6 mmol) in H₂O (9 mL) was treated with sodium sulfite (1.2 g, 9.1 mmol), and sodium iodide (114 mg) and heated in a sealed glass tube at 140° C. for 24 h. The reaction mixture was cooled to 0° C., forming a white precipitate, which was isolated by filtration, and washed with ice-water and ethyl acetate to provide sodium 2-(2-bromo-5-fluorophenyl)ethanesulfonic acid (1.85 g, 80%) as a white solid:

HPLC retention time 5.5 min.; Xterra RP18, 3.5 u, 150×4.6 mm column, 1.2 mL/min, 85/15-5/95 (Ammonium formate buffer, pH 3.5/CH₃CN +MeOH) for 10 min., hold for 4 min.

MS (ES) m/z 280.6.

Step 3: A suspension of sodium 2-(2-bromo-5-fluorophenyl)ethanesulfonic acid (4.5 g, 15 mmol) in toluene (121 mL) was treated with thionyl chloride (86 mL) and dimethylformamide (0.6 mL) and heated to 80° C. for 16 h. The reaction mixture was cooled to room temperature, and filtered to remove inorganic material. Concentration provided 2-(2-bromo-5-fluorophenyl)ethanesulfonyl chloride (4.4 g, 98%) as a yellow solid:

HPLC retention time 10.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₈H₇BrClFO₂S, 299.90227; found (EI, M⁺.), 299.9021.

Step 4: A solution of 2-(2-bromo-5-fluorophenyl)ethanesulfonyl chloride (6.7 g, 22 mmol) in dichloromethane (45 mL) was added dropwise to a solution of 2-fluoroaniline (8.5 mL, 88 mmol) and pyridine (1.8 mL, 22 mmol) in dichloromethane (40 mL) over 30 minutes, and stirred at 22° C. for 16 h. The reaction mixture was diluted with 2 N hydrochloric acid (200 mL), extracted with dichloromethane (200 mL), and evaporated. Flash chromatography (SiO₂, 50% ethyl acetate/hexanes) provided 2-(2-bromo-5-fluorophenyl)-N-(2-fluorophenyl)ethanesulfonamide (2.1 g, 64%) as a yellow solid:

HPLC retention time 9.9 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₄H₁₂BrF₂NO₂S−H⁺, 373.96674; found (ESI, [M−H]−Obs'd), 373.9663.

Step 5: A solution of 2-(2-bromo-5-fluorophenyl)-N-(2-fluorophenyl)ethanesulfonamide (7 g, 18.7 mmol) in dimethylsulfoxide (170 mL) was treated with cesium acetate (18 g, 94 mmol) and copper(I) iodide (7.1 g, 37 mmol) and stirred at 22° C. for 16 h. The reaction mixture was diluted with ethyl ether (500 mL), washed with sat. ammonium hydroxide (100 mL), H₂O (500 mL) and evaporated. Flash chromatography (SiO₂, 10-50% ethyl acetate/hexanes) provided 6-fluoro-1-(2-fluorophenyl)-3,4-dihydro-1H-2,1-benzothiazine 2,2-dioxide (5.4 g, 97%) as a yellow solid:

HPLC retention time 8.7 min.; Xterra RP18, 3.5 u, 150×4.6 mm column, 1.2 mL/min, 85/15-5/95 (Ammonium formate buffer, pH 3.5/CH₃CN +MeOH)

for 10 min., hold for 4 min.

HRMS: calcd for C₁₄H₁₁F₂NO₂S+H⁺, 296.05513; found (ESI, [M+H−SO₂]+), 232.0927.

Step 6: A solution of 6-fluoro-1-(2-fluorophenyl)-3,4-dihydro-1H-2,1-benzothiazine 2,2-dioxide (0.6 g, 2 mmol) in tetrahydrofuran (5 mL) was treated with lithium hexamethyldisilazide (2. 4 mL of a 1.0 M solution in tetrahydrofuran, 2.4 mmol) at −78° C. After 1 h, 1,3-dibromopropane (2.4 mL, 2.4 mmol) was added and the reaction mixture was allowed to warm to room temperature. After 3 h, the reaction mixture was quenched by the addition of hydrobromic acid (50 mL of a 1 M aqueous solution). The reaction mixture was extracted into ethyl ether (200 mL) and evaporates. Flash chromatography (SiO₂, 10-30% ethyl acetate/hexanes) provided

3-(3-bromopropyl)-6-fluoro-1-(2-fluorophenyl)-3,4-dihydro-1H-2,1-benzothiazine 2,2-dioxide (0.36 g, 43%) as a colorless oil:

HPLC retention time 10.2 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₁₆BrF₂NO₂S+H⁺, 416.01259; found (ESI, [M+H—SO₂]+), 352.0515.

Step 7: A solution of 3-(3-bromopropyl)-6-fluoro-1-(2-fluorophenyl)-3,4-dihydro-1H-2,1-benzothiazine 2,2-dioxide (0.36 g, 0.87 mmol) in an 8 M methyl amine solution in ethanol (50 mL) was stirred at 22° C. in a sealed tube for 3 h. The reaction mixture was evaporated to provide crude 3-[6-fluoro-1-(2-fluorophenyl)-2,2-dioxido-3,4-dihydro-1H-2,1-benzothiazin-3-yl]-N-methylpropan-1-amine hydrobromide (0.4 g, 100%):

HPLC retention time 6.9 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]⁺ Obs'd), 367.1288.

### Example 109

### 3-[(3S)-6-fluoro-1-(2-fluorophenyl)-2,2-dioxido-3,4-dihydro-1H-2,1-benzothiazin-3-yl]-N-methylpropan-1-amine

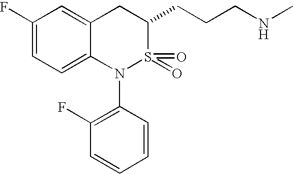

### Example 110

### 3-[(3R)-6-fluoro-1-(2-fluorophenyl)-2,2-dioxido-3,4-dihydro-1H-2,1-benzothiazin-3-yl]-N-methylpropan-1-amine

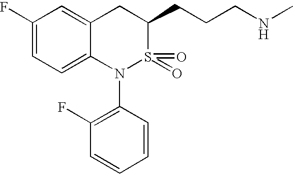

Approximately 0.4 g of racemic mixture of 3-[1-(2-fluorophenyl)-2,2-dioxido-3,4-dihydro-1H-2,1-benzothiazin-3-yl]-N-methylpropan-1-amine hydrobromide was dissolved in 17 mL of methanol. 0.75 mL of the resulting solution was repetitively injected onto the Supercritical Fluid Chromatography instrument, and the baseline resolved enantiomers were collected using a Berger MultiGram Prep SFC (Berger Instruments, Inc. Newark, Del.) under the following conditions: Chiralpak AD-H column ( [symbol] micron, 250 mm L×20 mm ID, Chiral Technologies, Inc, Exton, Pa.), 35° C. column temperature, 20% methanol w/0.2% dimethylethylamine/80% CO₂, 50 mL/min flow rate, 100 bar outlet pressure, 220 nm UV detection. The chiral purity of each enantiomer was determined under the same SFC conditions using a Chiralcel AD-H column ( [symbol] micron, 250 mm L×4.6 mm ID) at 2.0 mL/min flow rate on a Berger Analytical SFC instrument. Both compounds were determined to be >99.9% chiraly pure (Rₜ 6.8 and 9.0 min).

Enantiomer 1: (SFC Rt 6.8 minutes) (114 mg) arbitrarily assigned as 3-[(3S)-6-fluoro-1-(2-fluorophenyl)-2,2-dioxido-3,4-dihydro-1H-2,1-benzothiazin-3-yl]-N-methylpropan-1-amine: Recovered sample from SFC purification was dissolved in methanol (10 mL) and treated with 2 M HCl-ether (1 mL) and evaporated to a white powder:

HPLC retention time 6.9 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]⁺ Obs'd), 367.1289.

Enantiomer 2: (SFC Rt 9.0.minutes), (180 mg) arbitrarily assigned as 3-[(3R)-6-fluoro-1-(2-fluorophenyl)-2,2-dioxido-3,4-dihydro-1H-2,1-benzothiazin-3-yl]-N-methylpropan-1-amine: Recovered sample from SFC purification was dissolved in methanol (10 mL) and treated with 2 M HCl-ether (1 mL) and evaporated to a white powder:

HPLC retention time 6.9 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]+ Obs'd), 367.1289.

### Example 111

### 3-[1-(2,4-difluorophenyl)-6-fluoro-2,2-dioxido-3,4-dihydro-1H-2,1-benzothiazin-3-yl]-N-methylpropan-1-amine

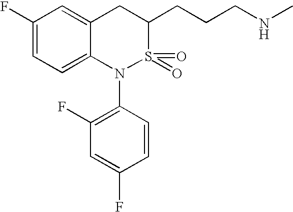

In an analogous manner to the preparation of 3-[6-fluoro-1-(2-fluorophenyl)-2,2-dioxido-3,4-dihydro-1H-2,1-benzothiazin-3-yl]-N-methylpropan-1-amine, the title compound was prepared from 2-(2-bromo-5-fluorophenyl)ethanesulfonyl chloride and 2,4-difluoroaniline; the racemate analogously resolved by chiral SFC: The chiral purity of each enantiomer was determined under the same SFC conditions using a Chiralpak AD-H column (5μ, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 5.2 and 8.6 min):

Racemate, 3-[1-(2,4-difluorophenyl)-6-fluoro-2,2-dioxido-3,4-dihydro-1H-2,1-benzothiazin-3-yl]-N-methylpropan-1-amine:

HPLC retention time 7.2 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H⁺, 385.11921; found (ESI, [M+H]⁺ Obs'd), 385.1193.

### Example 112

### 3-[(3S)-1-(2,4-difluorophenyl)-6-fluoro-2,2-dioxido-3,4-dihydro-1H-2,1-benzothiazin-3-yl]-N-methylpropan-1-amine

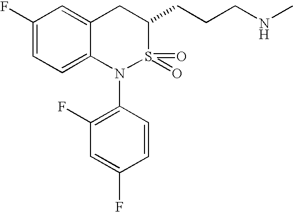

### Example 113

### 3-[(3R)-1-(2,4-difluorophenyl)-6-fluoro-2,2-dioxido-3,4-dihydro-1H-2,1-benzothiazin-3-yl]-N-methylpropan-1-amine

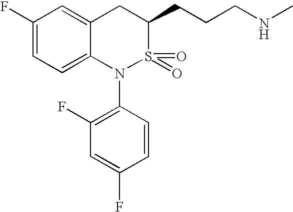

Enantiomer 1, (SFC Rt 5.2 min) arbitrarily assigned as 3-[(3S)-1-(2,4-difluorophenyl)-6-fluoro-2,2-dioxido-3,4-dihydro-1H-2,1-benzothiazin-3-yl]-N-methylpropan-1-amine:

HPLC purity 100.0% at 210-370 nm, 7.1 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₁₉F₃N₂O₂S+H⁺, 385.11921; found (ESI, [M+H]⁺ Obs'd), 385.1195.

Enantiomer 2 (SFC Rt 8.6 min) arbitrarily assigned as 3-[(3R)-1-(2,4-difluorophenyl)-6-fluoro-2,2-dioxido-3,4-dihydro-1H-2,1-benzothiazin-3-yl]-N-methylpropan-1-amine:

HPLC purity 100.0% at 210-370 nm, 7.1 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₁₉F₃N₂O₂S+H⁺, 385.11921; found (ESI, [M+H]⁺ Obs'd), 385.1194.

### Example 114

### (1S)-1-[(3S)-2,2-dioxido-1-phenyl-3,4-dihydro-1H-2,1-benzothiazin-3-yl]-3-(methylamino)propan-1-ol

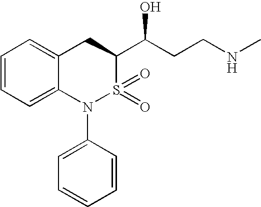

### Step 1: A solution of 1-phenyl-1H-4,2,1-benzoxathiazine 2,2-dioxide (0.26 g, 1

mmol) in tetrahydrofuran (10 mL) was cooled to −78° C., treated with lithium bis(trimethylsilyl)-amide (1.2 mL of a 1.0 M tetrahydrofuran solution, 1.2 mmol), stirred at −78° C. for 30 min and then was allowed to warm to 0° C. for 30 min. The reaction mixture was cooled to −78° C. and then tert-butyl methyl(3-oxopropyl)carbamate (0.25 g, 1.3 mmol) in tetrahydrofuran (2 mL) was slowly added and the reaction was warmed to room temperature. The reaction mixture was evaporated and the crude reaction product, was obtained after flash chromatography (SiO₂, 3-50% ethyl acetate/heptane).

This racemic mixture of Boc-protected diastereomers (0.754 g, 1.7 mmol) was taken up in formic acid (10 mL) and stirred overnight at room temperature. The reaction mixture was evaporated with high heat and chased with toluene (2×20 mL) to provide a mixture of diastereomers of deprotected amine.

This racemic mixture of diastereomers (0.2 g, 0.58 mmol) was separated and resolved by Supercritical Fluid Chromatography. The baseline resolved diastereomers were collected using a Berger MultiGram Prep SFC (Berger Instruments, Inc. Newark, Del.) under the following conditions: Chiralcel AD-H (5 micron, 250 mm L×21 mm ID, Chiral Technologies, Inc, West Chester, Pa.), 35° C. column temperature, 17% MeOH/0.2% dimethylethylamine as CO₂ modifier, 60 mL/min flow rate, 100 bar outlet pressure, 220 nm UV detection to provide the four desired diastereomers.

Peak 1 Rt 9.76 min

Peak 2 Rt 10.98 min

Peak 3 Rt 12.29 min

Peak 4 Rt 12.45 min.

Step 2: Sample from peak 1 from the previous step was dissolved in dichloromethane (3 mL) and treated with hydrogen chloride (1.0 mL of a 2 M solution in ethyl ether), resulting in a white precipitate that was evaporated and dried under vacuum to provided (1S)-1-[(3S)-2,2-dioxido-1-phenyl-3,4-dihydro-1H-2,1-benzothiazin-3-yl]-3-(methylamino)propan-1-ol hydrochloride (0.0265 g) as a white solid:

HPLC retention time 6.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₈H₂₂N₂O₃S+H⁺, 347.14239; found (ESI, [M+H]+ Obs'd), 347.1425.

### Example 115

(1R)-1-[(3S)-2,2-dioxido-1-phenyl-3,4-dihydro-1H-2,1-benzothiazin-3-yl]-3-(methylamino)propan-1-ol

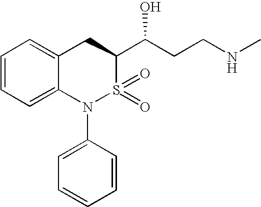

Peak 2 from Example 114, step 1, was dissolved in dichloromethane (3 mL) and treated with hydrogen chloride (1.0 mL of a 2 M solution in ethyl ether), resulting in a white precipitate that was evaporated and dried under vacuum to provided (1R)-1-[(3S)-2,2-dioxido-1-phenyl-3,4-dihydro-1H-2,1-benzothiazin-3-yl]-3-(methylamino)propan-1-ol hydrochloride (0.0195 g) as a white solid:

HPLC retention time 6.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₂N₂O₃S+H+, 347.14239; found (ESI, [M+H]+ Obs'd), 347.1425.

### Example 116

(1R)-1-[(3R)-2,2-dioxido-1-phenyl-3,4-dihydro-1H-2,1-benzothiazin-3-yl]-3-(methylamino)propan-1-ol

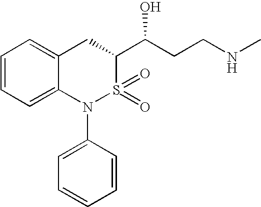

Peak 3 from Example 114, step 1, was dissolved in dichloromethane (3 mL) and treated with hydrogen chloride (1.0 mL of a 2 M solution in ethyl ether), resulting in a white precipitate that was evaporated and dried under vacuum to provided (1R)-1-[(3R)-2,2-dioxido-1-phenyl-3,4-dihydro-1H-2,1-benzothiazin-3-yl]-3-(methylamino)propan-1-ol hydrochloride (0.031 g) as a white solid:

HPLC retention time 6.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₂N₂O₃S+H⁺, 347.14239; found (ESI, [M+H]⁺ Obs'd), 347.1425.

### Example 117

### (1S)-1-[(3R)-2,2-dioxido-1-phenyl-3,4-dihydro-1H-2,1-benzothiazin-3-yl]-3-(methylamino)propan-1-ol

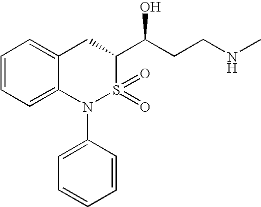

Peak 4 from Example 114, step 1, was dissolved in dichloromethane (3 mL) and treated with hydrogen chloride (1.0 mL of a 2 M solution in ethyl ether), resulting in a white precipitate that was evaporated and dried under vacuum to provided (1S)-1-[(3R)-2,2-dioxido-1-phenyl-3,4-dihydro-1H-2,1-benzothiazin-3-yl]-3-(methylamino)propan-1-ol hydrochloride (0.0211 g) as a white solid:

HPLC retention time 6.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₂N₂O₃S+H⁺, 347.14239; found (ESI, [M+H]⁺ Obs'd), 347.1425.

### Example 118

### 2-[(3S)-6-fluoro-2,2-dioxido-1-phenyl-1H-4,2,1-benzoxathiazin-3-yl]-N-methylethanamine

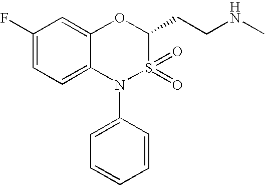

Step 1: A solution of 6-fluoro-1-phenyl-1H-4,2,1-benzoxathiazine 2,2-dioxide (0.8218 g, 3 mmol) in tetrahydrofuran (20 mL) was cooled to −78° C., treated with lithium bis(trimethylsilyl)-amide (3.5 mL of a 1.0 M tetrahydrofuran solution, 3.5 mmol) and stirred at −78° C. for 1 h. Allyl bromide (0.33 mL, 3.8 mmol) was added, and the reaction was allowed to warm to 23° C. and stir overnight. The reaction mixture was evaporated, diluted with dichloromethane (20 mL) and extracted with 2N HCl (3×20 mL). The organic layer was isolated, dried with MgSO₄ and evaporated. The crude reaction product was purified by flash chromatography (SiO₂, 3-50% ethyl acetate/hexane) provided 3-allyl-6-fluoro-1-phenyl-1H-4,2,1-benzoxathiazine 2,2-dioxide (0.73 g, 77%) as a yellow oil:

HPLC retention time 10.2 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

HRMS: calcd for C₁₆H₁₄FNO₃S, 319.06784; found (EI, M+.), 319.0681.

Step 2: A solution of 3-allyl-6-fluoro-1-phenyl-1H-4,2,1-benzoxathiazine 2,2-dioxide (0.46 g, 1.4 mmol) in MeOH (20 mL) was cooled to −78° C. and ozone was bubbled into the flask for 30 min, at which time the solution became blue. Sodium borohydride (0.07 g, 2 mmol) was added and the reaction mixture was allowed to warm to 23° C. and stir overnight. The reaction mixture was evaporated and the crude reaction product was purified by flash chromatography (SiO₂, 3-50% ethyl acetate/hexane) to afford the intermediate alcohol (0.33 g, 72%) as a yellow oil.

A solution of this alcohol (0.27 g, 0.8 mmol) in tetrahydrofuran (10 mL) was cooled to 0° C., treated with triphenylphosphine (0.33 g, 1.3 mmol) and N-bromosuccinimide (0.23 g, 1.3 mmol). The reaction was allowed to warm to 23° C. and stir overnight. The reaction mixture was evaporated and the crude reaction product was purified by flash chromatography (SiO₂, 3-50% ethyl acetate/hexane) to afford the intermediate bromide (0.26 g, 80%) as a yellow solid.

This intermediate bromide (0.25 g, 0.8 mmol) was treated with solution of methylamine (8 M in THF, 10 mL, 80 mmol) and stirred overnight. The reaction mixture was evaporated and the crude reaction product was purified by flash chromatography (SiO₂, 0-5% 7 M NH₃-methanol/dichloromethane) to provide the racemic 2-[(3)-6-fluoro-2,2-dioxido-1-phenyl-1H-4,2,1-benzoxathiazin-3-yl]-N-methylethanamine (0.24, 95%) as a brown amorphous solid.

Racemic 2-[(3)-6-fluoro-2,2-dioxido-1-phenyl-1H-4,2,1-benzoxathiazin-3-yl]-N-methylethanamine (0.22 g) was purified by super critical Fluid Chromatography (SFC). Baseline resolved enantiomers were collected using a Berger MultiGram Prep SFC (Berger Instruments, Inc. Newark, Del.) under the following conditions: Chiralcel AD-H (5 micron, 250 mm L×21 mm ID, Chiral Technologies, Inc, West Chester, Pa.), 35° C. column temperature, 20% MeOH/0.2% dimethylethylamine as CO₂ modifier, 50 mL/min flow rate, 100 bar outlet pressure, 220 nm UV detection.

Peak 1 Rt 4.7 min.

Peak 2 Rt 7.0 min.

Step 3 Peak 1 dissolved in dichloromethane (3 mL) and treated with hydrogen chloride (1.0 mL of a 2 M solution in ethyl ether), resulting in a white precipitate that was evaporated and dried under vacuum to provided 2-[(3S)-6-fluoro-2,2-dioxido-1-phenyl-1H-4,2,1-benzoxathiazin-3-yl]-N-methylethanamine hydrochloride (0.035 g) as a white solid: HPLC retention time 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₆H₁₇FN₂O₃S+H+, 337.10167; found (ESI, [M+H]+ Obs'd), 337.1021.

### Example 119

### 2-[(3R)-6-fluoro-2,2-dioxido-1-phenyl-1H-4,2,1-benzoxathiazin-3-yl]-N-methylethanamine

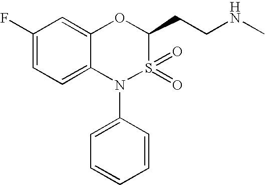

Peak 2, from Example 118, step 3, was dissolved in dichloromethane (3 mL) and treated with hydrogen chloride (1.0 mL of a 2 M solution in ethyl ether), resulting in a white precipitate that was evaporated and dried under vacuum to provided 2-[(3R)-6-fluoro-2,2-dioxido-1-phenyl-1H-4,2,1-benzoxathiazin-3-yl]-N-methylethanamine hydrochloride (0.0363 g) as a white solid: HPLC retention time 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₆H₁₇FN₂O₃S+H+, 337.10167; found (ESI, [M+H]+ Obs'd), 337.1019.

### Example 120

### 3-[1-(2,6-difluorophenyl)-2,2-dioxido-3,4-dihydro-1H-2,1-benzothiazin-3-yl]-N-methylpropan-1-amine

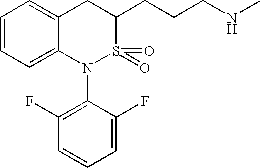

In an analogous manner to the preparation of 3-[1-(2-fluorophenyl)-2,2-dioxido-3,4-dihydro-1H-2,1-benzothiazin-3-yl]-N-methylpropan-1-amine, the title compound was prepared from 2-bromophenethylsulfonyl chloride and 2,6-difluoroaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 5.4 and 7.3 min):

Racemic product, 3-[1-(2,6-difluorophenyl)-2,2-dioxido-3,4-dihydro-1H-2,1-benzothiazin-3-yl]-N-methylpropan-1-amine

HPLC retention time 6.7 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₀F₂N₂O₂S+H⁺, 367.12863; found (ESI, [M+H]⁺ Obs'd), 367.1289.

### Example 121

### 3-[(3S)-1-(2,6-difluorophenyl)-2,2-dioxido-3,4-dihydro-1H-2,1-benzothiazin-3-yl]-N-methylpropan-1-amine

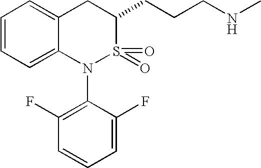

### Example 122

### 3-[(3R)-1-(2,6-difluorophenyl)-2,2-dioxido-3,4-dihydro-1H-2,1-benzothiazin-3-yl]-N-methylpropan-1-amine

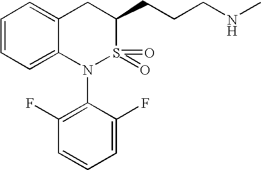

Enantiomer 1, (SFC Rₜ 5.4 min) arbitrarily assigned as 3-[(3S)-1-(2,6-difluorophenyl)-2,2-dioxido-3,4-dihydro-1H-2,1-benzothiazin-3-yl]-N-methylpropan-1-amine

HPLC purity 96.8% at 210-370 nm, 6.7 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₀F₂N₂O₂S+H⁺, 367.12863; found (ESI, [M+H]⁺), 367.1285.

Enantiomer 2, (SFC R Rₜ 7.3 min) arbitrarily assigned as 3-[(3R)-1-(2,6-difluorophenyl)-2,2-dioxido-3,4-dihydro-1H-2,1-benzothiazin-3-yl]-N-methylpropan-1-amine

HPLC retention time 6.7 min.; Xterra RP18, 3.5 u, 150×4.6 mm column, 1.2 mL/min, 85/15-5/95 (Ammonium formate buffer, pH 3.5/CH₃CN +MeOH)

for 10 min., hold for 4 min.

HRMS: calcd for C₁₈H₂₀F₂N₂O₂S+H⁺, 367.12863; found (ESI, [M+H]+), 367.1290.

### Example 123

### 3-(2,2-dioxido-1-pyridin-3-yl-3,4-dihydro-1H-2,1-benzothiazin-3-yl)-N-methylpropan-1-amine

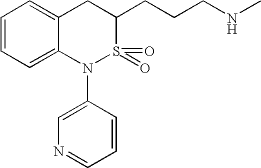

In an analogous manner to the preparation of 3-[1-(2-fluorophenyl)-2,2-dioxido-3,4-dihydro-1H-2,1-benzothiazin-3-yl]-N-methylpropan-1-amine, the title compound was prepared from 2-bromophenethylsulfonyl chloride and 3-aminopyridine; the racemate analogously resolved by chiral SFC: The chiral purity of each enantiomer was determined under the same SFC conditions using a Chiralpak OD-H column (5 micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 6.94 and 8.40 min):

Racemate, 3-(2,2-dioxido-1-pyridin-3-yl-3,4-dihydro-1H-2,1-benzothiazin-3-yl)-N-methylpropan-1-amine:

HPLC retention time 5.1 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₁N₃O₂S+H⁺, 332.14272; found (ESI, [M+H]⁺ Obs'd), 332.1433.

### Example 124

### 3-[(3S)-2,2-dioxido-1-pyridin-3-yl-3,4-dihydro-1H-2,1-benzothiazin-3-yl]-N-methylpropan-1-amine

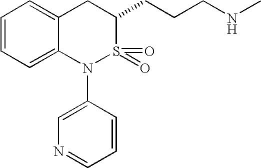

### Example 125

### 3-[(3R)-2,2-dioxido-1-pyridin-3-yl-3,4-dihydro-1H-2,1-benzothiazin-3-yl]-N-methylpropan-1-amine

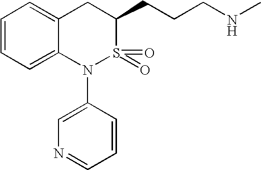

Enantiomer 1, (SFC Rₜ 6.94 min): arbitrarily assigned as 3-[(3S)-2,2-dioxido-1-pyridin-3-yl-3,4-dihydro-1H-2,1-benzothiazin-3-yl]-N-methylpropan-1-amine:

HPLC purity 96.1% at 210-370 nm, 5.1 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₁N₃O₂S+H⁺, 332.14272; found (ESI, [M+H]⁺ Obs'd), 332.1431.

Enantiomer 2, (SFC Rₜ 8.40 min): arbitrarily assigned as 3-[(3R)-2,2-dioxido-1-pyridin-3-yl-3,4-dihydro-1H-2,1-benzothiazin-3-yl]-N-methylpropan-1-amine

HPLC purity 100.0% at 210-370 nm, 5.1 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₁N₃O₂S+H⁺, 332.14272; found (ESI, [M+H]⁺ Obs'd), 332.1430.

### Example 126

### 3-(2,2-dioxido-1-pyridin-2-yl-3,4-dihydro-1H-2,1-benzothiazin-3-yl)-N-methylpropan-1-amine

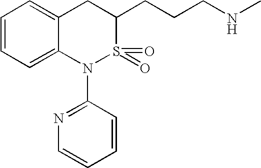

In an analogous manner to the preparation of 3-[1-(2-fluorophenyl)-2,2-dioxido-3,4-dihydro-1H-2,1-benzothiazin-3-yl]-N-methylpropan-1-amine, the title compound was prepared from 2-bromophenethylsulfonyl chloride and 2-aminopyridine; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 6.83 and 11.97 min):

Racemate, 3-(2,2-dioxido-1-pyridin-2-yl-3,4-dihydro-1H-2,1-benzothiazin-3-yl)-N-methylpropan-1-amine:

HPLC retention time 5.2 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₁N₃O₂S+H⁺, 332.14272; found (ESI, [M+H]+ Obs'd), 332.1432.

### Example 127

### 3-[(3S)-2,2-dioxido-1-pyridin-2-yl-3,4-dihydro-1H-2,1-benzothiazin-3-yl]-N-methylpropan-1-amine

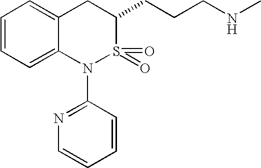

### Example 128

### 3-[(3R)-2,2-dioxido-1-pyridin-2-yl-3,4-dihydro-1H-2,1-benzothiazin-3-yl]-N-methylpropan-1-amine

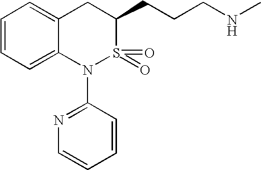

Enantiomer 1, (SFC Rₜ 6.83 min) arbitrarily assigned as 3-[(3S)-2,2-dioxido-1-pyridin-2-yl-3,4-dihydro-1H-2,1-benzothiazin-3-yl]-N-methylpropan-1-amine:

HPLC retention time 5.3 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₁N₃O₂S+H⁺, 332.14272; found (ESI, [M+H]⁺ Obs'd), 332.1430.

Enantiomer 2, (SFC Rₜ 11.97 min) arbitrarily assigned as 3-[(3R)-2,2-dioxido-1-pyridin-2-yl-3,4-dihydro-1H-2,1-benzothiazin-3-yl]-N-methylpropan-1-amine

HPLC retention time 5.3 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₇H₂₁N₃O₂S+H⁺, 332.14272; found (ESI, [M+H]⁺ Obs'd), 332.1431.

### Example 129

### 3-[6-fluoro-1-(2-fluorophenyl)-2,2-dioxido-1H-4,2,1-benzoxathiazin-3-yl]-N-methylpropan-1-amine hydrochloride

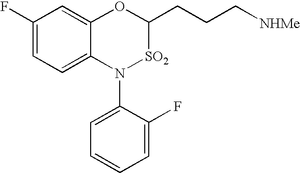

In an analogous manner to Example 92, step 3, 1.2 g of 3-(3-chloropropyl)-6-fluoro-1-(2-fluorophenyl)-1H-4,2,1-benzoxathiazine 2,2-dioxide was prepared from 4.5 g of 3-(3-chloropropyl)-6-fluoro-1H-4,2,1-benzoxathiazine 2,2-dioxide and 2-fluorophenylboronic acid. MS (ESI) m/z 310; HRMS: calcd for C16H14ClF2NO3S+Na+, 396.02432; found (ESI, [M+Na]+ Calc'd), 396.0243

In an analogous manner to Example 92, step 4, 0.25 g of 3-(6-fluoro-2,2-dioxido-1-(2-fluorophenyl)-2,2-dioxido-1H-4,2,1-benzoxathiazin-3-yl]-N-methylpropan-1 amine hydrochloride was prepared from 0.5 g of 3-(3-chloropropyl)-6-fluoro-1-(2-fluorophenyl)-1H-4,2,1-benzoxathiazine 2,2-dioxide and methyl amine. HRMS: calcd for C17H18F2N2O3S+H+, 369.10789; found (ESI, [M+H]+ Obs'd), 369.1085. HPLC purity 92.9% at 210-370 nm, 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### Example 130

### 3-[(3R)-6-fluoro-1-(2-fluorophenyl)-2,2-dioxido-1H-4,2,1-benzoxathiazin-3-yl]-N-methylpropan-1-amine Hydrochloride

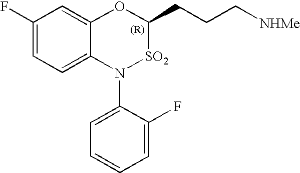

and

### Example 131

### 3-[(3S)-6-fluoro-1-(2-fluorophenyl)-2,2-dioxido-1H-4,2,1-benzoxathiazin-3-yl]-N-methylpropan-1-amine Hydrochloride

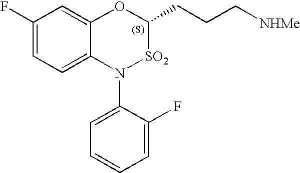

The racemic mixture from Example 129 was dissolved in methanol and enantiomers separated by injection into the Supercritical Fluid Chromatography (SFC) instrument using Chiralpak AD-H SFC column at 35° C. eluting with 25% MeOH with 0.2% DMEA as CO₂ modifier, 50 mL/min flow rate and 220 nM detection.

Peak 1, Example 130 arbitrarily assigned as 3-[(3R)-6-fluoro-1-(2-fluorophenyl)-2,2-dioxido-1H-4,2,1-benzoxathiazin-3-yl]-N-methylpropan-1-amine hydrochloride: t_{R}=4.2 min; MS (ES) m/z 369; HPLC purity 100.0% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min; HRMS: calcd for C17H18F2N2O3S+H+, 369.10789; found (ESI, [M+H]+ Obs'd), 369.1084

Peak 2: Example 131 arbitrarily assigned as 3-[(3S)-6-fluoro-1-(2-fluorophenyl)-2,2-dioxido-1H-4,2,1-benzoxathiazin-3-yl]-N-methylpropan-1-amine hydrochloride t_{R}=5.8 min; MS (ES) m/z 368.8; HPLC purity 100.0% at 210-370 nm, 7.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min; HRMS: calcd for C17H18F2N2O3S+H+, 369.10789; found (ESI, [M+H]+ Obs'd), 369.1084;

### Example 132

### 2-[(2,2-dioxido-1-phenyl-3,4-dihydro-1H-2,1-benzothiazin-3-yl)methyl]-N-methylprop-2-en-1-amine

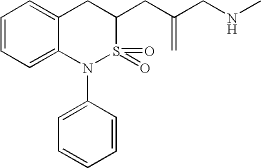

Step 1: A solution of 1-phenyl-3,4-dihydro-1H-2,1-benzothiazine 2,2-dioxide (0.79 g, 3 mmol) in tetrahydrofuran (6 mL) was cooled to −78° C. and treated with n-butyllithium (1.4 mL of a 2.5 M solution in hexanes, 3.6 mL), stirred for 1 h, then treated with 3-chloro1-bromopropane (3.1 mL, 30 mmol) and warmed to room temperature for 6 h. The reaction mixture was diluted with dichloromethane (50 mL), and washed with 2 M hydrochloric acid (50 mL), dried (Na₂SO₄) and evaporated. Flash chromatography (SiO₂, 10-50% ethyl acetate/hexanes) provided 3-[2-(chloromethyl)prop-2-en-1-yl]-1-phenyl-3,4-dihydro-1H-2,1-benzothiazine 2,2-dioxide (0.35 g, 30%) as a yellow oil:

HPLC retention time 10.2 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₁₈ClNO₂S+H⁺, 348.08195; found (ESI, [M+H−SO₂]⁺), 284.1207.

Step 2: A solution of 3-[2-(chloromethyl)prop-2-en-1-yl]-1-phenyl-3,4-dihydro-1H-2,1-benzothiazine 2,2-dioxide (0.14 g, 0.4 mmol) in an 8 M methyl amine solution in ethanol (10 mL) was stirred at 22° C. in a sealed tube for 8 h. The reaction mixture was evaporated to provide crude 2-[(2,2-dioxido-1-phenyl-3,4-dihydro-1H-2,1-benzothiazin-3-yl)methyl]-N-methylprop-2-en-1-amine hydrochloride as a white solid (150 mg, 93%):

HPLC retention time 7.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₉H₂₂N₂O₂S+H⁺, 343.14747; found (ESI, [M+H]⁺), 343.1485.

### Example 133

### 3-(2,2-dioxido-1-pyridin-4-yl-3,4-dihydro-1H-2,1-benzothiazin-3-yl)-N-methylpropan-1-amine

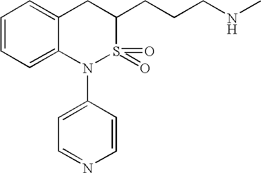

In an analogous manner to the preparation of 3-[1-(2-fluorophenyl)-2,2-dioxido-3,4-dihydro-1H-2,1-benzothiazin-3-yl]-N-methylpropan-1-amine, the title compound was prepared from 2-bromophenethylsulfonyl chloride and 2-aminopyridine;

Racemate: 3-(2,2-dioxido-1-pyridin-4-yl-3,4-dihydro-1H-2,1-benzothiazin-3-yl)-N-methylpropan-1-amine:

HPLC purity area %=100 by COL1MPH3 method; Xterra RP C18 4.6×150 column, 1.2 mL/min, A=water w/10 mm ammonium formate pH=3.5 B=acetonitrile:methanol (50:50).

HPLC retention time 4.8 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

### Example 134

### 1-(2,2-dioxido-1-phenyl-1H-4,2,1-benzoxathiazin-3-yl)-N,N-dimethylmethanamine

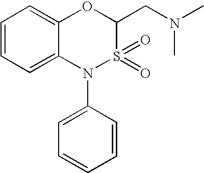

A solution of 1-phenyl-1H-4,2,1-benzoxathiazine 2,2-dioxide (0.52 g, 2 mmol) in tetrahydrofuran (20 mL) was cooled to −78° C., treated with lithium bis(trimethylsilyl)-amide (2.4 mL of a 1.0 M tetrahydrofuran solution, 2.4 mmol) and stirred at −78° C. for 1 h. Dimethylmethylideneammonium iodide (1.35 g, 7.3 mmol) was added and the reaction mixture was allowed to warm to 23° C. and stirred overnight. The reaction mixture was evaporated, diluted with ethyl acetate (20 mL) and extracted with NaHCO₃ (3×10 mL). The organic layer was isolated, dried with MgSO₄ and evaporated. The crude reaction product was purified by flash chromatography (SiO₂, 3-50% ethyl acetate/heptane) to provide 1-(2,2-dioxido-1-phenyl-1H-4,2,1-benzoxathiazin-3-yl)-N,N-dimethylmethanamine (0.45 g, 69%) as a white solid:

HPLC retention time, 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₆H₁₈N₂O₃S+H⁺, 319.11109; found (ESI, [M+H]+ Obs'd), 319.1122;

### Example 135

### 1-phenyl-3-(piperazin-1-ylmethyl)-1H-4,2,1-benzoxathiazine 2,2-dioxide

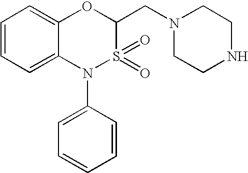

Step 1: A solution of 1-(2,2-dioxido-1-phenyl-1H-4,2,1-benzoxathiazin-3-yl)-N,N-dimethylmethanamine (1.77 g, 5.6 mmol) in ethyl ether (100 mL) was cooled to 0° C. and treated with iodomethane (1.75 mL, 28 mmol) and then warmed to room temperature and stirred for an additional 12 h. The reaction mixture was evaporated to provide 2,2-dioxido-1-phenyl-1H-4,2,1-benzoxathiazin-3-yl)-N,N,N-trimethylmethanaminium iodide (2.52 g) as white solid:

HPLC retention time 6.4 min.; Xterra RP18, 3.5 u, 150×4.6 mm column, 1.2 mL/min, 85/15-5/95 (Ammon. Form. Buff. Ph=3.5/ACN+MeOH) for 10 min,

hold 4 min.

HRMS: calcd for C₁₇H₂₁N₂O₃S, 333.12729; found (ESI, M+), 333.1277;

Step 2: A solution of 2,2-dioxido-1-phenyl-1H-4,2,1-benzoxathiazin-3-yl)-N,N,N-trimethylmethanaminium iodide (0.2 g, 0.43 mmol) in DMF (3 mL) was treated with piperazine (0.22 mL, 2.6 mmol) and stirred in a capped vial for 16 h. The reaction mixture was diluted with H₂O (5 mL) and extracted with ethyl ether (3×10 mL), dried with (MgSO₄), and purified by flash chromatography (SiO₂, 0-5% 7 M NH₃-methanol/dichloromethane). The free-base was dissolved in dichloromethane (3 mL) and treated with hydrogen chloride (1.0 mL of a 2 M solution in ethyl ether), resulting in a white precipitate that was evaporated and dried under vacuum to provided 1-phenyl-3-(piperazin-1-ylmethyl)-1H-4,2,1-benzoxathiazine 2,2-dioxide hydrochloride (0.116 g, 58%) as a white solid:

HPLC retention time 6.8 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₁N₃O₃S+H⁺, 360.13764; found (ESI, [M+H]⁺), 360.1383;

### Example 136

### N-[(2,2-dioxido-1-phenyl-1H-4,2,1-benzoxathiazin-3-yl)methyl]ethanamine

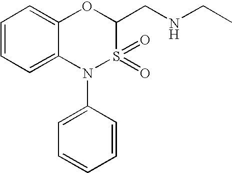

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with 2 M solution of ethylamine, (6 mL, 12 mmol) and then treated with HCl to provide N-[(2,2-dioxido-1-phenyl-1H-4,2,1-benzoxathiazin-3-yl)methyl]ethanamine hydrochloride (0.123 g, 79%) as a white solid: HPLC retention time 6.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₆H₁₈N₂O₃S+H+, 319.11109; found (ESI, [M+H]+ Obs'd), 319.1118;

### Example 137

### N-[(2,2-dioxido-1-phenyl-1H-4,2,1-benzoxathiazin-3-yl)methyl]ethane-1,2-diamine

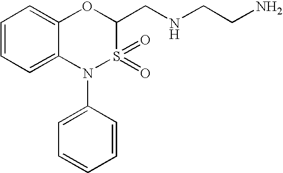

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with ethane-1,2-diamine (neat), (0.6 mL, 9 mmol) and then treated with HCl to provide N-[(2,2-dioxido-1-phenyl-1H-4,2,1-benzoxathiazin-3-yl)methyl]ethane-1,2-diamine hydrochloride (0.103 g, 64%) as a white solid: HPLC retention time 6.5 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₆H₁₉N₃O₃S+H+, 334.12199; found (ESI, [M+H]+ Obs'd), 334.1225;

### Example 138

### N′-[(2,2-dioxido-1-phenyl-1H-4,2,1-benzoxathiazin-3-yl)methyl]-N,N-dimethylethane-1,2-diamine

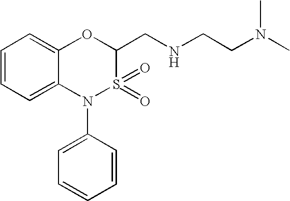

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with N,N-dimethylethylenediamine (neat) (3 mL, 27 mmol) and then treated with HCl to provide N′-[(2,2-dioxido-1-phenyl-1H-4,2,1-benzoxathiazin-3-yl)methyl]-N,N-dimethylethane-1,2-diamine hydrochloride (0.103 g, 60%) as a white solid:

HPLC retention time 6.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₈H₂₃N₃O₃S+H+, 362.15329; found (ESI, [M+H]+ Obs'd), 362.1539;

### Example 139

### N-[(2,2-dioxido-1-phenyl-1H-4,2,1-benzoxathiazin-3-yl)methyl]-N,N′-dimethylethane-1,2-diamine

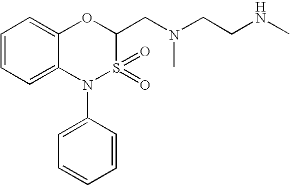

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with N,N′-dimethylethylenediamine (neat) (3 mL, 27 mmol) and then treated with HCl to provide N-[(2,2-dioxido-1-phenyl-1H-4,2,1-benzoxathiazin-3-yl)methyl]-N,N′-dimethylethane-1,2-diamine hydrochloride (0.099 g, 57%) as a white solid:

HPLC retention time 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₈H₂₃N₃O₃S+H+, 362.15329; found (ESI, [M+H]+ Obs'd), 362.1538;

### Example 140

### N-[(2,2-dioxido-1-phenyl-1H-4,2,1-benzoxathiazin-3-yl)methyl]-N,N′,N′-trimethylethane-1,2-diamine

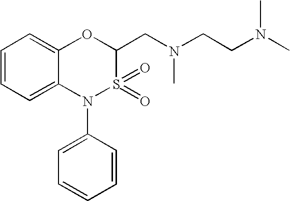

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with N,N,N′-trimethylethylenediamine (neat) (5 mL, 38 mmol) and then treated with HCl to provide N-[(2,2-dioxido-1-phenyl-1H-4,2,1-benzoxathiazin-3-yl)methyl]-N,N′,N′-trimethylethane-1,2-diamine hydrochloride (0.132 g, 74%) as a white solid:

HPLC retention time 7.1 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₉H₂₅N₃O₃S+H+, 376.16894; found (ESI, [M+H]+ Obs'd), 376.1697;

### Example 141

### 3-[(4-methylpiperazin-1-yl)methyl]-1-phenyl-1H-4,2,1-benzoxathiazine 2,2-dioxide

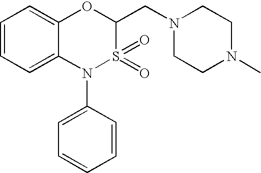

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with 1-methylpiperazine (neat) (3 mL, 27 mmol) and then treated with HCl to provide 3-[(4-methylpiperazin-1-yl)methyl]-1-phenyl-1H-4,2,1-benzoxathiazine 2,2-dioxide hydrochloride (0.096 g, 54%) as a white solid: HPLC retention time 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₉H₂₃N₃O₃S+H+, 374.15329; found (ESI, [M+H]+ Obs'd), 374.1541;

### Example 142

### 3-[(3,5-dimethylpiperazin-1-yl)methyl]-1-phenyl-1H-4,2,1-benzoxathiazine 2,2-dioxide

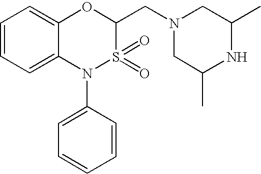

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) in DMF (3 mL) was reacted with 2,6-dimethylpiperazine (0.25 g, 2.2 mmol) and then treated with HCl to provide 3-[(3,5-dimethylpiperazin-1-yl)methyl]-1-phenyl-1H-4,2,1-benzoxathiazine 2,2-dioxide hydrochloride (0.142 g, 77%) as a white solid: HPLC retention time 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₂₀H₂₅N₃O₃S+H+, 388.16894; found (ESI, [M+H]+ Obs'd), 388.1697;

### Example 143

### 3-(2,5-diazabicyclo[2.2.1]hept-2-ylmethyl)-1-phenyl-1H-4,2,1-benzoxathiazine 2,2-dioxide

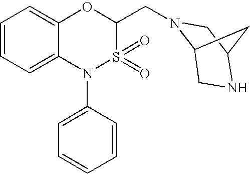

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) in DMF (3 mL) was reacted to (1S,4S)-(−)-2-boc-2,5-diazabicyclo[2.2.1]heptane (0.35 g, 1.8 mmol) and then treated with HCl to provide 3-(2,5-diazabicyclo[2.2.1]hept-2-ylmethyl)-1-phenyl-1H-4,2,1-benzoxathiazine 2,2-dioxide hydrochloride (0.027 g, 15%) as a white solid. HPLC retention time 6.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₉H₂₁N₃O₃S+H+, 372.13764; found (ESI, [M+H]+ Obs'd), 372.1383;

### Example 144

### 1-(2,2-dioxido-1-phenyl-1H-4,2,1-benzoxathiazin-3-yl)-N-methylmethanamine

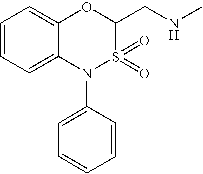

In an analogous manner as Example 135 step 2, 2,2-dioxido-1-phenyl-1H-4,2,1-benzoxathiazin-3-yl)-N,N,N-trimethylmethanaminium iodide (0.2 g, 0.43 mmol) was reacted with an 8 M ethanolic solution of methylamine (3 mL, 24 mmol) and then treated with HCl to provide 1-(2,2-dioxido-1-phenyl-1H-4,2,1-benzoxathiazin-3-yl)-N-methylmethanamine hydrochloride (0.104 g, 70%) as a white solid: HPLC retention time 6.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₅H₁₆N₂O₃S+H⁺, 305.09544; found (ESI, [M+H]⁺), 305.0968.

### Example 145

### 3-[1-(2,3-difluorophenyl)-6-fluoro-2,2-dioxido-3,4-dihydro-1H-2,1-benzothiazin-3-yl]-N-methylpropan-1-amine

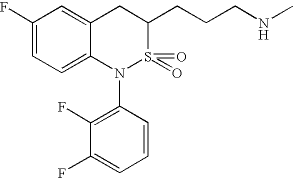

In an analogous manner to the preparation of 3-[6-fluoro-1-(2-fluorophenyl)-2,2-dioxido-3,4-dihydro-1H-2,1-benzothiazin-3-yl]-N-methylpropan-1-amine, the title compound was prepared from 2-(2-bromo-5-fluorophenyl)ethanesulfonyl chloride and 2,3-difluoroaniline; the racemate analogously resolved by chiral SFC: The chiral purity of each enantiomer was determined under the same SFC conditions using a Chiralpak AD-H column (5 micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 5.5 and 7.4 min):

Racemate, 1-(2,3-difluorophenyl)-6-fluoro-2,2-dioxido-3,4-dihydro-1H-2,1-benzothiazin-3-yl]-N-methylpropan-1-amine:

HPLC retention time 7.3 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H⁺, 385.11921; found (ESI, [M+H]+ Obs'd), 385.1196.

### Example 146

### 3-[(3S)-1-(2,3-difluorophenyl)-6-fluoro-2,2-dioxido-3,4-dihydro-1H-2,1-benzothiazin-3-yl]-N-methylpropan-1-amine

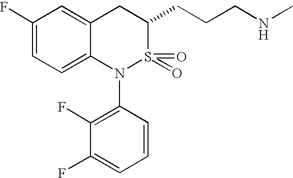

### Example 147

### 3-[(3R)-1-(2,3-difluorophenyl)-6-fluoro-2,2-dioxido-3,4-dihydro-1H-2,1-benzothiazin-3-yl]-N-methylpropan-1-amine

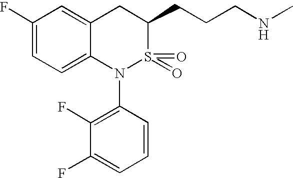

Enantiomer 1 (SFC Rt=5.5 min) arbitrarily assigned as (3S)-1-(2,3-difluorophenyl)-6-fluoro-2,2-dioxido-3,4-dihydro-1H-2,1-benzothiazin-3-yl]-N-methylpropan-1-amine

HPLC retention time 7.2 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H⁺, 385.11921; found (ESI, [M+H]⁺ Obs'd), 385.1198;

Enantiomer 2 (SFC Rt=7.4 min), arbitrarily assigned as 3-[(3R)-1-(2,3-difluorophenyl)-6-fluoro-2,2-dioxido-3,4-dihydro-1H-2,1-benzothiazin-3-yl]-N-methylpropan-1-amine:

HPLC retention time 7.2 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H⁺, 385.11921; found (ESI, [M+H]⁺ Obs'd), 385.1198;

### Example 148

### 3-[6-fluoro-1-(2-fluoro-4-methylphenyl)-2,2-dioxido-3,4-dihydro-1H-2,1-benzothiazin-3-yl]-N-methylpropan-1-amine

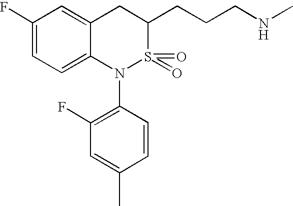

In an analogous manner to the preparation of 3-[6-fluoro-1-(2-fluorophenyl)-2,2-dioxido-3,4-dihydro-1H-2,1-benzothiazin-3-yl]-N-methylpropan-1-amine, the title compound was prepared from 2-(2-bromo-5-fluorophenyl)ethanesulfonyl chloride and 2-fluoro-4-methylaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 25% methanol with 0.2% dimethylethylamine/75% CO₂, 2 mL/min flow rate, 100 bar outlet pressure, 220 nm UV detection on a Berger Analytical SFC instrument. Both enantiomers were determined to be >99.9% chiraly pure (Rₜ 6.04 and 10.91 min):

Racemate 3-[6-fluoro-1-(2-fluoro-4-methylphenyl)-2,2-dioxido-3,4-dihydro-1H-2,1-benzothiazin-3-yl]-N-methylpropan-1-amine:

HPLC retention time 7.6 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₂F₂N₂O₂S+H⁺, 381.14428; found (ESI, [M+H]+ Obs'd), 381.1446.

### Example 149

### 3-[(3S)-6-fluoro-1-(2-fluoro-4-methylphenyl)-2,2-dioxido-3,4-dihydro-1H-2,1-benzothiazin-3-yl]-N-methylpropan-1-amine

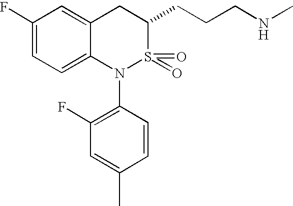

### Example 150

### 3-[(3R)-6-fluoro-1-(2-fluoro-4-methyl phenyl)-2,2-dioxido-3,4-dihydro-1H-2,1-benzothiazin-3-yl]-N-methylpropan-1-amine

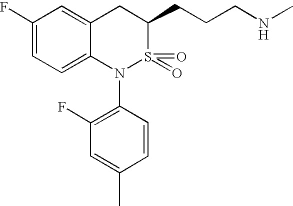

Enantiomer 1 (SFC Rt 6.04 min) arbitrarily assigned as 3-[(3S)-6-fluoro-1-(2-fluoro-4-methylphenyl)-2,2-dioxido-3,4-dihydro-1H-2,1-benzothiazin-3-yl]-N-methylpropan-1-amine

HPLC retention time 7.5 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₂F₂N₂O₂S+H+, 381.14428; found (ESI, [M+H]⁺ Obs'd), 381.1448.

Enantiomer 2 (SFC Rₜ 10.91 min) arbitrarily assigned as 3-[(3R)-6-fluoro-1-(2-fluoro-4-methylphenyl)-2,2-dioxido-3,4-dihydro-1H-2,1-benzothiazin-3-yl]-N-methylpropan-1-amine:

HPLC retention time 7.5 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₂F₂N₂O₂S+H⁺, 381.14428; found (ESI, [M+H]+ Obs'd), 381.1447.

### Example 151

### 3-[1-(2,5-difluorophenyl)-6-fluoro-2,2-dioxido-3,4-dihydro-1H-2,1-benzothiazin-3-yl]-N-methylpropan-1-amine

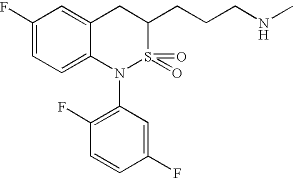

In an analogous manner to the preparation of 3-[6-fluoro-1-(2-fluorophenyl)-2,2-dioxido-3,4-dihydro-1H-2,1-benzothiazin-3-yl]-N-methylpropan-1-amine, the title compound was prepared from 2-(2-bromo-5-fluorophenyl)ethanesulfonyl chloride and 2,5-difluoroaniline; the racemate analogously resolved by chiral SFC: The chiral purity of each enantiomer was determined under the same SFC conditions using a Chiralpak AS-H column (5 micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 6.7 and 9.0 min):

Racemate 3-[1-(2,5-difluorophenyl)-6-fluoro-2,2-dioxido-3,4-dihydro-1H-2,1-benzothiazin-3-yl]-N-methylpropan-1-amine:

HPLC retention time 7.2 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H⁺, 385.11921; found (ESI, [M+H]+ Obs'd), 385.1197.

### Example 152

### 3-[(3S)-1-(2,5-difluorophenyl)-6-fluoro-2,2-dioxido-3,4-dihydro-1H-2,1-benzothiazin-3-yl]-N-methylpropan-1-amine

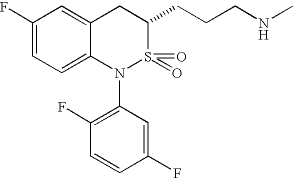

### Example 153

### 3-[(3R)-1-(2,5-difluorophenyl)-6-fluoro-2,2-dioxido-3,4-dihydro-1H-2,1-benzothiazin-3-yl]-N-methylpropan-1-amine

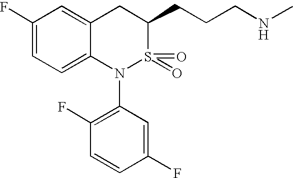

Enantiomer 1 (SFC Rt 6.7 min) arbitrarily assigned as 3-[(3S)-1-(2,5-difluorophenyl)-6-fluoro-2,2-dioxido-3,4-dihydro-1H-2,1-benzothiazin-3-yl]-N-methylpropan-1-amine

MS (ES) m/z 384.9;

HPLC retention time 7.1 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H⁺, 385.11921; found (ESI, [M+H]+ Obs'd), 385.1198;

Enantiomer 2 (SFC Rt 9.0 min) arbitrarily assigned as 3-[(3R)-1-(2,5-difluorophenyl)-6-fluoro-2,2-dioxido-3,4-dihydro-1H-2,1-benzothiazin-3-yl]-N-methylpropan-1-amine:

HPLC retention time 7.1 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₁₉F₃N₂O₂S+H⁺, 385.11921; found (ESI, [M+H]+ Obs'd), 385.1197;

### Example 154

### 3-[1-(2,6-difluorophenyl)-6-fluoro-2,2-dioxido-3,4-dihydro-1H-2,1-benzothiazin-3-yl]-N-methylpropan-1-amine

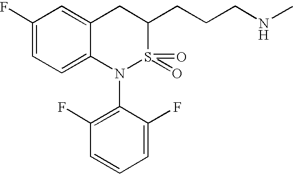

In an analogous manner to the preparation of 3-[6-fluoro-1-(2-fluorophenyl)-2,2-dioxido-3,4-dihydro-1H-2,1-benzothiazin-3-yl]-N-methylpropan-1-amine, the title compound was prepared from 2-(2-bromo-5-fluorophenyl)ethanesulfonyl chloride and 2,6-dufluoroaniline; the racemate analogously resolved by chiral SFC: The chiral purity of each enantiomer was determined under the same SFC conditions using a Chiralpak AD-H column (5 micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 5.6 and 6.5 min):

Racemate 3-[1-(2,6-difluorophenyl)-6-fluoro-2,2-dioxido-3,4-dihydro-1H-2,1-benzothiazin-3-yl]-N-methylpropan-1-amine:

HRMS: calcd for C₁₈H₁₉F₃N₂O₂S+H⁺, 385.11921; found (ESI, [M+H]+ Obs'd), 385.1196.

### Example 155

### 3-[(3S)-1-(2,6-difluorophenyl)-6-fluoro-2,2-dioxido-3,4-dihydro-1H-2,1-benzothiazin-3-yl]-N-methylpropan-1-amine

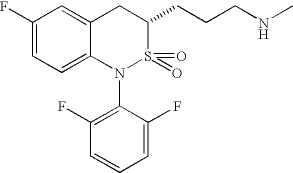

### Example 156

### 3-[(3R)-1-(2,6-difluorophenyl)-6-fluoro-2,2-dioxido-3,4-dihydro-1H-2,1-benzothiazin-3-yl]-N-methylpropan-1-amine

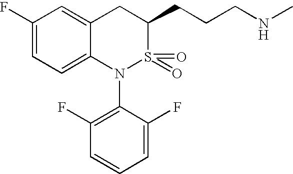

Enantiomer 1 (SFC Rt 5.6 min) arbitrarily assigned as 3-[(3S)-1-(2,6-difluorophenyl)-6-fluoro-2,2-dioxido-3,4-dihydro-1H-2,1-benzothiazin-3-yl]-N-methylpropan-1-amine:

HPLC retention time 6.9 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H+, 385.11921; found (ESI, [M+H]+ Obs'd), 385.1199;

Enantiomer 2 (SFC Rt 6.5 min) arbitrarily assigned as 3-[(3R)-1-(2,6-difluorophenyl)-6-fluoro-2,2-dioxido-3,4-dihydro-1H-2,1-benzothiazin-3-yl]-N-methylpropan-1-amine:

HPLC retention time 6.9 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₁₉F₃N₂O₂S+H+, 385.11921; found (ESI, [M+H]+ Obs'd), 385.1198;

### Example 157

### 3-[1-(2-chlorophenyl)-6-fluoro-2,2-dioxido-3,4-dihydro-1H-2,1-benzothiazin-3-yl]-N-methylpropan-1-amine

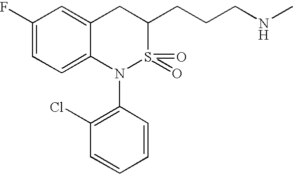

In an analogous manner to the preparation of 3-[6-fluoro-1-(2-fluorophenyl)-2,2-dioxido-3,4-dihydro-1H-2,1-benzothiazin-3-yl]-N-methylpropan-1-amine, the title compound was prepared from 2-(2-bromo-5-fluorophenyl)ethanesulfonyl chloride and 2-chloroaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 25% methanol with 0.2% dimethylethylamine/75% CO₂, 2 mL/min flow rate, 100 bar outlet pressure, 220 nm UV detection on a Berger Analytical SFC instrument. Both enantiomers were determined to be >99.9% chiraly pure (Rₜ 5.69 and 8.10 min):

Racemate 3-[1-(2-chlorophenyl)-6-fluoro-2,2-dioxido-3,4-dihydro-1H-2,1-benzothiazin-3-yl]-N-methylpropan-1-amine:

HPLC retention time 7.3 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₀ClFN₂O₂S+H⁺, 383.09908; found (ESI, [M+H]+ Obs'd), 383.0993;

### Example 158

### 3-[(3S)-1-(2-chlorophenyl)-6-fluoro-2,2-dioxido-3,4-dihydro-1H-2,1-benzothiazin-3-yl]-N-methylpropan-1-amine

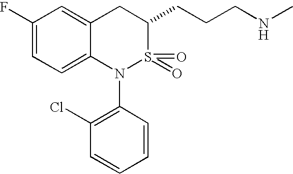

### Example 159

### 3-[(3R)-1-(2-chlorophenyl)-6-fluoro-2,2-dioxido-3,4-dihydro-1H-2,1-benzothiazin-3-yl]-N-methylpropan-1-amine

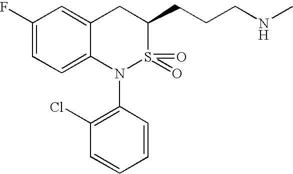

Enantiomer 1 (SFC Rt 5.69 min) arbitrarily assigned as 3-[(3S)-1-(2-chlorophenyl)-6-fluoro-2,2-dioxido-3,4-dihydro-1H-2,1-benzothiazin-3-yl]-N-methylpropan-1-amine:

HPLC retention time 7.3 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₀ClFN₂O₂S+H⁺, 383.09908; found (ESI, [M+H]+ Obs'd), 383.0996;

Enantiomer 2 (SFC Rt 8.1 min) arbitrarily assigned as 3-[(3R)-1-(2-chlorophenyl)-6-fluoro-2,2-dioxido-3,4-dihydro-1H-2,1-benzothiazin-3-yl]-N-methylpropan-1-amine:

HPLC retention time 7.4 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₀ClFN₂O₂S+H⁺, 383.09908; found (ESI, [M+H]+ Obs'd), 383.0996;

### Example 160

### 3-[6-fluoro-1-(2-methylphenyl)-2,2-dioxido-3,4-dihydro-1H-2,1-benzothiazin-3-yl]-N-methylpropan-1-amine

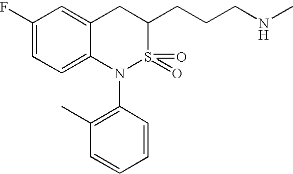

In an analogous manner to Example 108, the title compound of the present example was prepared from 2-(2-bromo-5-fluorophenyl)ethanesulfonyl chloride and 2-methylaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 6.8 and 12.2 min):

Racemate 3-[6-fluoro-1-(2-methylphenyl)-2,2-dioxido-3,4-dihydro-1H-2,1-benzothiazin-3-yl]-N-methylpropan-1-amine:

HPLC retention time 7.5 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₃FN₂O₂S+H⁺, 363.15370; found (ESI, [M+H]+ Obs'd), 363.1540;

### Example 161

### 3-[(3S)-6-fluoro-1-(2-methylphenyl)-2,2-dioxido-3,4-dihydro-1H-2,1-benzothiazin-3-yl]-N-methylpropan-1-amine

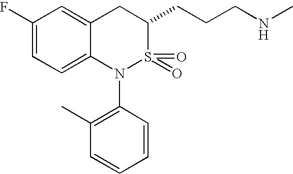

### Example 162

### 3-[(3R)-6-fluoro-1-(2-methylphenyl)-2,2-dioxido-3,4-dihydro-1H-2,1-benzothiazin-3-yl]-N-methylpropan-1-amine

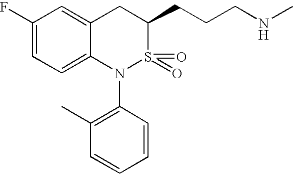

Enantiomer 1 (SFC Rt 6.8 min) (Example 161)), arbitrarily assigned as 3-[(3S)-6-fluoro-1-(2-methylphenyl)-2,2-dioxido-3,4-dihydro-1H-2,1-benzothiazin-3-yl]-N-methylpropan-1-amine:

HPLC retention time 7.4 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₃FN₂O₂S+H⁺, 363.15370; found (ESI, [M+H]+ Obs'd), 363.1540;

Enantiomer 2 (SFC Rt=12.2 min) (Example 162), arbitrarily assigned as 3-[(3R)-6-fluoro-1-(2-methylphenyl)-2,2-dioxido-3,4-dihydro-1H-2,1-benzothiazin-3-yl]-N-methylpropan-1-amine:

HPLC Retention time 7.5 min; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₃FN₂O₂S+H⁺, 363.15370; found (ESI, [M+H]+ Obs'd), 363.1541;

### Example 163

### 2-{6-fluoro-3-[3-(methylamino)propyl]-2,2-dioxido-3,4-dihydro-1H-2,1-benzothiazin-1-yl}benzonitrile

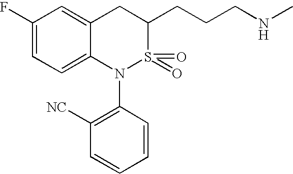

In an analogous manner to Example 108, the title compound of the present example was prepared from 2-(2-bromo-5-fluorophenyl)ethanesulfonyl chloride and 2-cyanoaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 20% methanol with 0.2% dimethylethylamine/80% CO₂, 2 mL/min flow rate, 100 bar outlet pressure, 220 nm UV detection on a Berger Analytical SFC instrument. Both enantiomers were determined to be >99.9% chiraly pure (Rₜ 5.91 and 6.91 min):

Racemate, 2-{6-fluoro-3-[3-(methylamino)propyl]-2,2-dioxido-3,4-dihydro-1H-2,1-benzothiazin-1-yl}benzonitrile:

HPLC Retention time 6.4 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₀FN₃O₂S+H⁺, 374.13330; found (ESI, [M+H]+ Obs'd), 374.1337

### Example 164

### 2-{(3S)-6-fluoro-3-[3-(methylamino)propyl]-2,2-dioxido-3,4-dihydro-1H-2,1-benzothiazin-1-yl}benzonitrile

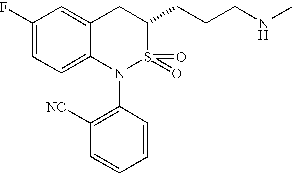

### Example 165

### 2-{(3R)-6-fluoro-3-[3-(methylamino)propyl]-2,2-dioxido-3,4-dihydro-1H-2,1-benzothiazin-1-yl}benzonitrile

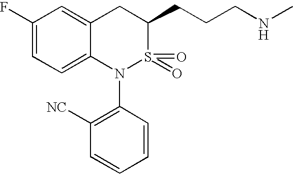

Enantiomer 1 (SFC Rt 5.91 min) (Example 164) arbitrarily assigned as 2-{(3S)-6-fluoro-3-[3-(methylamino)propyl]-2,2-dioxido-3,4-dihydro-1H-2,1-benzothiazin-1-yl}benzonitrile:

HPLC Retention time 6.4 min; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₀FN₃O₂S+H⁺, 374.13330; found (ESI, [M+H]+ Obs'd), 374.1336;

Enantiomer 2 (SFC Rt 6.91 min) (Example 165) arbitrarily assigned as 2-{(3R)-6-fluoro-3-[3-(methylamino)propyl]-2,2-dioxido-3,4-dihydro-1H-2,1-benzothiazin-1-yl}benzonitrile:

HPLC Retention time 6.4 min; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C19H20FN3O2S+H+, 374.13330; found (ESI, [M+H]+ Obs'd), 374.1340;

### Example 166

### 3-[6-fluoro-1-(2-methoxyphenyl)-2,2-dioxido-3,4-dihydro-1H-2,1-benzothiazin-3-yl]-N-methylpropan-1-amine

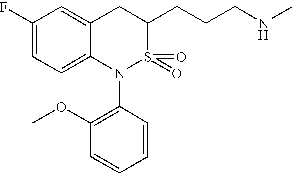

In an analogous manner to Example 108, the title compound was prepared from 2-(2-bromo-5-fluorophenyl)ethanesulfonyl chloride and 2-methoxyaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 30% methanol with 0.2% dimethylethylamine/70% CO₂, 2 mL/min flow rate, 100 bar outlet pressure, 220 nm UV detection on a Berger Analytical SFC instrument. Both enantiomers were determined to be >99.9% chiraly pure (Rₜ 3.43 and 7.26 min):

Racemate, 3-[6-fluoro-1-(2-methoxyphenyl)-2,2-dioxido-3,4-dihydro-1H-2,1-benzothiazin-3-yl]-N-methylpropan-1-amine:

HPLC Retention time 7.0 min.; Xterra RP18, 3.5 u, 150×4.6 mm column, 1.2 mL/min, 85/15-5/95 (Ammonium formate buffer, pH 3.5/CH3CN+MeOH)

for 10 min., hold for 4 min.

HRMS: calcd for C₁₉H₂₃FN₂O₃S+H⁺, 379.14862; found (ESI, [M+H]+ Obs'd), 379.1489;

### Example 167

### 3-[(3S)-6-fluoro-1-(2-methoxyphenyl)-2,2-dioxido-3,4-dihydro-1H-2,1-benzothiazin-3-yl]-N-methylpropan-1-amine

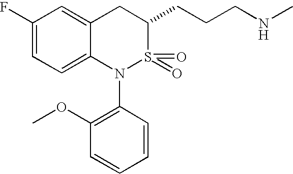

### Example 168

### 3-[(3R)-6-fluoro-1-(2-methoxyphenyl)-2,2-dioxido-3,4-dihydro-1H-2,1-benzothiazin-3-yl]-N-methylpropan-1-amine

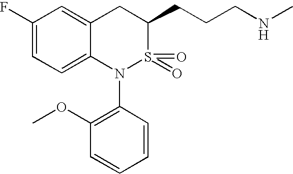

Enantiomer 1 (SFC Rt 3.43 min) (Example 167), arbitrarily assigned as 3-[(3S)-6-fluoro-1-(2-methoxyphenyl)-2,2-dioxido-3,4-dihydro-1H-2,1-benzothiazin-3-yl]-N-methylpropan-1-amine:

HPLC Retention time 7.0 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₃FN₂O₃S+H⁺, 379.14862; found (ESI, [M+H]+ Obs'd), 379.1491;

Enantiomer 2 (SFC Rₜ 7.26 min) (Example 168), arbitrarily assigned as 3-[(3R)-6-fluoro-1-(2-methoxyphenyl)-2,2-dioxido-3,4-dihydro-1H-2,1-benzothiazin-3-yl]-N-methylpropan-1-amine:

HPLC Retention time 7.0 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C19H23FN2O3S+H+, 379.14862; found (ESI, [M+H]+ Obs'd), 379.1490;

### Example 169

### 3-[6-fluoro-1-(4-fluoro-2-methylphenyl)-2,2-dioxido-3,4-dihydro-1H-2,1-benzothiazin-3-yl]-N-methylpropan-1-amine

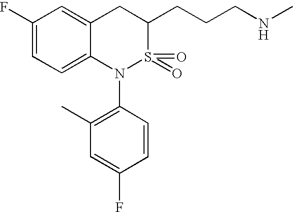

In an analogous manner to Example 108, the title compound of the present example was prepared from 2-(2-bromo-5-fluorophenyl)ethanesulfonyl chloride and 4-fluoro-2-methylaniline; the racemate analogously resolved by chiral SFC: The chiral purity of each enantiomer was determined under the same SFC conditions using a Chiralpak AD-H column ( [symbol] micron, 250 mm L×4.6 mm ID), 35° C. column temperature, 30% methanol with 0.2% dimethylethylamine/70% CO₂, 2 mL/min flow rate, 100 bar outlet pressure, 220 nm UV detection on a Berger Analytical SFC instrument. Both enantiomers were determined to be >99.9% chiraly pure (Rₜ 3.36 and 9.19 min):

Racemate, 3-[6-fluoro-1-(4-fluoro-2-methylphenyl)-2,2-dioxido-3,4-dihydro-1H-2,1-benzothiazin-3-yl]-N-methylpropan-1-amine:

HPLC Retention time 7.7 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₂F₂N₂O₂S+H⁺, 381.14428; found (ESI, [M+H]+ Obs'd), 381.1447;

### Example 170

### 3-[(3S)-6-fluoro-1-(4-fluoro-2-methylphenyl)-2,2-dioxido-3,4-dihydro-1H-2,1-benzothiazin-3-yl]-N-methylpropan-1-amine

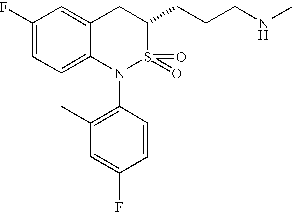

### Example 171

### 3-[(3R)-6-fluoro-1-(4-fluoro-2-methyl phenyl)-2,2-dioxido-3,4-dihydro-1H-2,1-benzothiazin-3-yl]-N-methylpropan-1-amine

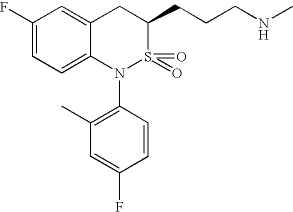

Enantiomer 1 (SFC 3.36 min) (Example 170), arbitrarily assigned as 3-[(3S)-6-fluoro-1-(4-fluoro-2-methylphenyl)-2,2-dioxido-3,4-dihydro-1H-2,1-benzothiazin-3-yl]-N-methylpropan-1-amine:

HPLC Retention time 7.6 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₂F₂N₂O₂S+H⁺, 381.14428; found (ESI, [M+H]+ Obs'd), 381.1448;

Enantiomer 2 (SFC Rt 9.19 min) (Example 171), arbitrarily assigned as 3-[(3R)-6-fluoro-1-(4-fluoro-2-methylphenyl)-2,2-dioxido-3,4-dihydro-1H-2,1-benzothiazin-3-yl]-N-methylpropan-1-amine:

HPLC Retention time 7.7 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₉H₂₂F₂N₂O₂S+H⁺, 381.14428; found (ESI, [M+H]+ Obs'd), 381.1446;

### Example 172

### 3-(6-fluoro-2,2-dioxido-1-phenyl-3,4-dihydro-1H-2,1-benzothiazin-3-yl)-N-methylpropan-1-amine

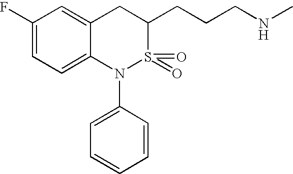

In an analogous manner to Example 108, the title compound was prepared from 2-(2-bromo-5-fluorophenyl)ethanesulfonyl chloride and aniline; the racemate analogously resolved by chiral SFC: The chiral purity of each enantiomer was determined under the same SFC conditions using a Chiralpak AD-H column (5 micron, 250 mm L×4.6 mm ID), 35° C. column temperature, 30% methanol with 0.2% dimethylethylamine/70% CO₂, 2 mL/min flow rate, 100 bar outlet pressure, 220 nm UV detection on a Berger Analytical SFC instrument. Both enantiomers were determined to be >99.9% chiraly pure (Rₜ 3.96 and 6.87 min):

Racemate, 3-(6-fluoro-2,2-dioxido-1-phenyl-3,4-dihydro-1H-2,1-benzothiazin-3-yl)-N-methylpropan-1-amine:

HPLC Retention time 7.0 min.; Xterra RP18, 3.5 u, 150×4.6 mm column, 1.2 mL/min, 85/15-5/95 (Ammonium formate buffer, pH 3.5/CH3CN +MeOH) for 10 min., hold for 4 min.

HRMS: calcd for C₁₈H₂₁FN₂O₂S+H⁺, 349.13805; found (ESI, [M+H]+ Obs'd), 349.1384;

### Example 173

### 3-[(3S)-6-fluoro-2,2-dioxido-1-phenyl-3,4-dihydro-1H-2,1-benzothiazin-3-yl]-N-methylpropan-1-amine

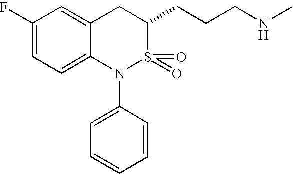

### Example 174

### 3-[(3R)-6-fluoro-2,2-dioxido-1-phenyl-3,4-dihydro-1H-2,1-benzothiazin-3-yl]-N-methylpropan-1-amine

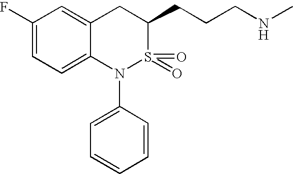

Enantiomer 1 (SFC Rt 3.96 min) (Example 173), arbitrarily assigned as 3-[(3S)-6-fluoro-2,2-dioxido-1-phenyl-3,4-dihydro-1H-2,1-benzothiazin-3-yl]-N-methylpropan-1-amine:

HPLC Retention time 7.0 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₁FN₂O₂S+H⁺, 349.13805; found (ESI, [M+H]+ Obs'd), 349.1388;

Enantiomer 2 (SFC Rt 6.87 min) (Example 174), arbitrarily assigned as 3-[(3R)-6-fluoro-2,2-dioxido-1-phenyl-3,4-dihydro-1H-2,1-benzothiazin-3-yl]-N-methylpropan-1-amine:

HPLC Retention time 7.0 min.; Xterra RP18, 3.5 u, 150×4.6 mm column, 1.2 mL/min, 85/15-5/95 (Ammonium formate buffer, pH 3.5/CH3CN +MeOH)

for 10 min., hold for 4 min.

HRMS: calcd for C₁₈H₂₁FN₂O₂S+H⁺, 349.13805; found (ESI, [M+H]+ Obs'd), 349.1384;

### Example 175

### 3-[6-chloro-1-(2-fluorophenyl)-2,2-dioxido-3,4-dihydro-1H-2,1-benzothiazin-3-yl]-N-methylpropan-1-amine

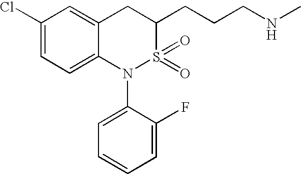

Step 1: A solution of 2-bromo-5-chlorophenylacetic acid (1.0 g, 4.0 mmol) in tetrahydrofuran (16 mL) was cooled to 0° C. and was treated with 1.0 M borane tetrahydrofuran complex solution (6 mL, 6.0 mmol) and then warmed to room temperature for 2 h. The reaction mixture was slowly quenched with H₂O (10 mL) and then diluted with ethyl acetate (30 mL) and washed with 2N HCl (3×15 mL). The organic layer was isolated, dried with MgSO₄ and evaporated to provide 2-(2-bromo-5-chlorophenyl)ethanol (0.91 g, 96%) as a yellow solid.

HPLC Retention time 8.7 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

HRMS: calcd for C₈H₈BrClO, 233.94470; found (EI, M+*), 233.9448;

Step 2: A solution of 2-(2-bromo-5-chlorophenyl)ethanol (11.5 g, 49 mmol) in thionyl chloride (90 mL, 1.24 mol) was refluxed for 16 h. The reaction mixture was slowly quenched with H₂O (10 mL) and the reaction mixture was extracted with ethyl ether (3×30 mL). The organic layer was isolated, dried with MgSO₄ and evaporated to provide 1-bromo-4-chloro-2-(2-chloroethyl)benzene (8.84 g, 71%) as a tan amorphous solid:

HPLC Retention time 11.0 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

HRMS: calcd for C₈H₇BrCl₂, 251.91082; found (EI, M⁺.), 251.9112;

Step 3: A solution of 1-bromo-4-chloro-2-(2-chloroethyl)benzene (3.02 g, 12 mmol) in H₂O (15 mL) was treated with sodium iodide (0.18 g, 1.9 mmol) and sodium sulfite (1.79, 14.2 mmol) in a 50 mL pressure vessel. The vessel was heated to 140° C. for 16 h, and cooled to room temperature resulting in a white precipitate. The solid was collected by filtration and then vacuum dried overnight to provide 2-(2-bromo-5-chlorophenyl)ethanesulfonic acid (2.91 g, 76%) as a white solid.

HPLC Retention time 6.2 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

Step 4: A suspension of 2-(2-bromo-5-chlorophenyl)ethanesulfonic acid (2.55 g, 7.93 mmol) in toluene (75 mL) was treated with thionyl chloride (50 mL, 687 mmol) and dimethylformamide (0.5 mL, 6.4 mmol) and refluxed for 14 h. The reaction mixture was filtered and rinsed with toluene. The filtrate was evaporated to provide 2-(2-bromo-5-chlorophenyl)ethanesulfonyl chloride (2.45 g, 97%) as a yellow oil, which was directly taken on to the next step.

Step 5: A solution of 2-(2-bromo-5-chlorophenyl)ethanesulfonyl chloride (1.15 g, 3.6 mmol) in dichloromethane (10 mL) and was added to a solution of 2-fluoroaniline (1.4 mL. 14.5 mmol) and pyridine (0.7 mL, 8.6 mmol) in dichloromethane (25 mL). The reaction mixture stirred at room temperature for 48 h and then at 35° C. for 3 h. The reaction mixture was washed with 2 N HCl (2×15 mL), dried with MgSO₄ and evaporated. The crude reaction product was purified by flash chromatography (SiO₂, 3-50% ethyl acetate/heptane) to provide 2-(2-bromo-5-chlorophenyl)-N-(2-fluorophenyl)ethanesulfonamide (1.12 g, 79%) as a white solid:

HPLC Retention time 10.4 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

HRMS: calcd for C₁₄H₁₂BrClFNO2S+H⁺, 391.95174; found (ESI, [M+H]+ Obs'd), 391.9523;

Step 6: A solution of 2-(2-bromo-5-chlorophenyl)-N-(2-fluorophenyl)ethanesulfonamide (1.106 g, 2.82 mmol) in dimethyl sulfoxide (30 mL) and benzene (3 mL) was treated with cesium acetate (1.1 g, 5.72 mmol) and copper(I) iodide (2.7 g, 14.1 mmol). The reaction mixture stirred at room temperature for 5 h. and then at 40° C. for 14 h. The reaction mixture was diluted with ethyl ether (50 mL) and washed with 2 N ammonium hydroxide (2×20 mL), the organic layer was isolated, dried with MgSO₄ and evaporated. The crude reaction product was purified by flash chromatography (SiO₂, 3-50% ethyl acetate/heptane) to provided 6-chloro-1-(2-fluorophenyl)-3,4-dihydro-1H-2,1-benzothiazine 2,2-dioxide (0.72 g, 82%) as a yellow oil:

HPLC Retention time 9.4 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

HRMS: calcd for C₁₄H₁₁ClFNO₂S, 311.01830; found (EI, M+.), 311.0180;

Step 7: A solution of 6-chloro-1-(2-fluorophenyl)-3,4-dihydro-1H-2,1-benzothiazine 2,2-dioxide (0.32 g, 1.1 mmol) in tetrahydrofuran (10 mL) was cooled to −78° C., treated with lithium bis(trimethylsilyl)-amide (1.15 mL of a 1.0 M tetrahydrofuran solution, 1.15 mmol), stirred at −78° C. for 30 min and then was allowed to warm to 0° C. for thirty min. The reaction was cooled to −78° C. and then treated with 1,3 dibromopropane (1.6 mL, 15.7 mmol) and then allowed to warm to room temperature and stir for 14 h. The reaction mixture was evaporated and the crude reaction product was purified by flash chromatography (SiO₂, 3-50% ethyl acetate/heptane) to provide 3-(3-bromopropyl)-6-chloro-1-(2-fluorophenyl)-3,4-dihydro-1H-2,1-benzothiazine 2,2-dioxide (0.22 g, 50%) as a tan solid:

HPLC Retention time 10.7 min.; Xterra RP18, 3.5 u, 150×4.6 mm column, 1.2 mL/min, 85/15-5/95 (Ammon. Form. Buff. Ph=3.5/ACN+MeOH)

for 10 min, hold 4 min.

HRMS: calcd for C₁₇H₁₆BrClFNO₂S, 430.97576; found (EI, M+.), 430.9769;

Step 8: 3-(3-bromopropyl)-6-chloro-1-(2-fluorophenyl)-3,4-dihydro-1H-2,1-benzothiazine 2,2-dioxide (0.21, 0.45 mmol) was dissolved in an 8 M solution of methylamine in ethanol (8 mL, 64 mmol) and was stirred in a capped vial at room temperature for 2 h. The reaction mixture was evaporated and the residue purified by flash chromatography (SiO₂, 0-5% 7 M NH₃-methanol/dichloromethane). The purified free-base was dissolved in dichloromethane (3 mL) and treated with hydrogen chloride (1.0 mL of a 2 M solution in ethyl ether), resulting in a white precipitate that was evaporated and dried under vacuum to provide 3-[6-chloro-1-(2-fluorophenyl)-2,2-dioxido-3,4-dihydro-1H-2,1-benzothiazin-3-yl]-N-methylpropan-1-amine (0.2209, 96%) as a white solid:

HPLC Retention time, 7.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₀ClFN₂O₂S+H+, 383.09908; found (ESI, [M+H]+ Obs'd), 383.0995;

### Example 176

### 3-[(3S)-6-chloro-1-(2-fluorophenyl)-2,2-dioxido-3,4-dihydro-1H-2,1-benzothiazin-3-yl]-N-methylpropan-1-amine

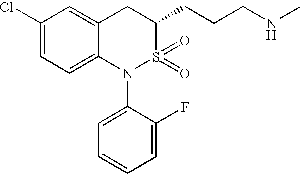

Step 1: Racemic) 3-[6-chloro-1-(2-fluorophenyl)-2,2-dioxido-3,4-dihydro-1H-2,1-benzothiazin-3-yl]-N-methylpropan-1-amine (0.21 g) was resolved by Supercritical Fluid Chromatography (SFC). The baseline resolved enantiomers were collected using a Berger MultiGram Prep SFC (Berger Instruments, Inc. Newark, Del.) under the following conditions: Chiralpak AD-H (5 micron, 250 mm L×21 mm ID, Chiral Technologies, Inc, West Chester, Pa.), 35° C. column temperature, 25% MeOH/0.2% dimethylethylamine as CO₂ modifier, 60 mL/min flow rate, 100 bar outlet pressure, 250 nm UV detection.

Peak 1 Rt 7.3 min.

Peak 2 Rt 10.1 min.

Step 2: Sample from peak 1 was dissolved in dichloromethane (3 mL) and treated with hydrogen chloride (1.0 mL of a 2 M solution in ethyl ether), resulting in a white precipitate that was evaporated and dried under vacuum to provided 3-[(3S)-6-chloro-1-(2-fluorophenyl)-2,2-dioxido-3,4-dihydro-1H-2,1-benzothiazin-3-yl]-N-methylpropan-1-amine (0.0729 g) as a white solid: HPLC purity 97.5% at 210-370 nm, 7.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

HRMS: calcd for C₁₈H₂₀ClFN₂O₂S+H+, 383.09908; found (ESI, [M+H]+ Obs'd), 383.0997;

### Example 177

### 3-[(3R)-6-chloro-1-(2-fluorophenyl)-2,2-dioxido-3,4-dihydro-1H-2,1-benzothiazin-3-yl]-N-methylpropan-1-amine

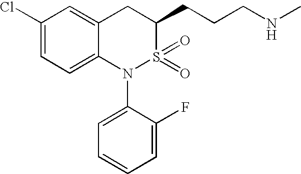

Peak 2 from Example 176, step 2, was dissolved in dichloromethane (3 mL) and treated with hydrogen chloride (1.0 mL of a 2 M solution in ethyl ether), resulting in a white precipitate that was evaporated and dried under vacuum to provided 3-[(3R)-6-chloro-1-(2-fluorophenyl)-2,2-dioxido-3,4-dihydro-1H-2,1-benzothiazin-3-yl]-N-methylpropan-1-amine (0.0672 g) as a white solid:

HPLC purity 100.0% at 210-370 nm, 7.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calcd for C₁₈H₂₀ClFN₂O₂S+H+, 383.09908; found (ESI, [M+H]+ Obs'd), 383.0997.

### Cell Lines, Culture Reagents, and Assays

### Compounds

For screening, 25 μl aliquots of compound solution at a 1 μM or 10 μM final concentration was delivered directly to cells.

For IC₅₀ determinations, stock compounds were prepared at 10 mM from powder. The stock solution was diluted according to compound testing range. Typically, the compound testing range was from 6 nM to 6 μM by half log dilutions. On the day of assay, 25 μl of compound solution at the specified concentrations were added to the plates containing cells. A DMSO stock of desipramine was prepared at 10 mM in DMSO and diluted for a final concentration of 20 μM to determine the non-specific reuptake. The radioligand in this assay was ³H-norepinephrine (NE) (PerkinElmer; NET678; 40-80 Ci/mmol) and was delivered at approximately 16 nM final concentration for both single point testing and compound IC₅₀ determinations.

### Tissue Culture Conditions

MDCK-Net6 cells, stably transfected with human hNET (Pacholczyk, 1991), were maintained in growth media [high glucose DMEM (Gibco Cat. 11995), 10% FBS (dialyzed, heat-inactivated, Sigma, dialysed, heat inactivated, Lot# K0922 or equivalent) 1xPen/Strep, and 500 μg/ml G418 (Gibco Cat. 10131)]. Cells were plated at 300,000/T75 flask and cells split twice weekly.

### Functional Reuptake Assay

Cells were plated at 50,000 cells/well (Procedure A) or 6,000 cells/well (Procedure B) on day 1 in BD Falcon Microtest 96-well sterile cell culture plates, Optilux White/Clear Bottom TC plate (VWR; # 62406-466 or equivalent) in growth media and maintained in a cell incubator (37° C., 5% CO₂). On Day 2, cells were removed from the cell incubator and the growth media is replaced by 200 μl of assay buffer (25 mm HEPES 120 mM NaCL; 5 mM KCl; 2.5 mM CaCl₂; 1.2 mM MgSO₄; 2 mg/ml glucose (pH 7.4, 37° C.)) containing 0.2 mg/ml ascorbic acid and 1 μM parglyine. For screening, 25 μl of compound in 4% DMSO was added directly to each well and the plate was incubated for 5 minutes (37° C.). To initiate the norepinephrine reuptake, 16 nM (final concentration) of ³H norepinephrine (specific activity; 40-80 Ci/mmol) in assay buffer was delivered in 25 μl aliquots to each well, and the plates were incubated for 5 min at 37° C. The reaction is aspirated from the plate and the cells washed with 250 μl of 50 mM Tris Buffer (4° C.). The plates were left to dry for 1 hour. The cells were lysed using 0.25 M NaOH solution then placed on a shake table and vigorously shaken for 10 minutes. After cell lysis, 100 μl of Microscint 20 (PerkinElmer; #87-051101) was added to the plates and the plates were sealed with film tape and replaced on the shake table for a minimum of 10 minutes. The plates were counted in a TopCount counter (PerkinElmer).

### Analysis of Results

For screening single point determinations, each compound plate contained at least 3 control wells (maximum NE reuptake determinant) and 3 non-specific wells determined by adding 20 μM of desipramine (minimum NE reuptake determinant). Determination of active compounds was calculated using a Microsoft Excel spread sheet applying the following formula:

% inhibition=[1−((mean cpm test compound wells−mean cpm non-specific wells)/(mean cpm control wells−mean cpm non-specific wells))]×100

For IC₅₀ determination, raw cpm values were generated in a data file from the TopCount counter. The data was organized using the Microsoft Excel and transferred into PRIZM graphing and statistical program, which calculated the estimated IC₅₀ value. Calculation of IC₅₀ values was made using non-linear regression analysis with a sigmoidal dose response with variable slope. The statistical program used wells containing ³H norepinephrine only as the maximal NE reuptake determinant and wells containing ³H norepinephrine plus 20 μM desipramine (AHR-9543) as the minimal NE reuptake determinant (non-specific determinant). Estimation of the IC₅₀ value was completed on a log scale and the line was fit between the maximal and minimal NE reuptake values. In the event that the highest test concentration does not exceed 50% reuptake inhibition, data will be reported as percent maximal NE reuptake at the highest concentration tested. The results are reported in Table 1.

TABLE 1

| | Procecedure | Procedure A | Procedure A | Procedure B | Procedure B | Procedure B |
|---|---|---|---|---|---|---|
| | A NE Uptake | [3H] NE Uptake′ | [3H] NE Uptake | hNET uptake′ | hNET uptake′ | hNET uptake |
| Example | IC₅₀ (nM) | Conc (nM) | Displacement % | IC₅₀ (nM) | Conc (nM) | (% inhibition) |
| 1 | 33.5 | | | 18.5 | | |
| 2 | 233 | | | | | |
| 3 | 484 | | | | | |
| 4 | 1876 | | | | | |
| 5 | 166 | | | | | |
| 6 | 133 | | | | | |
| 7 | 3011 | | | | | |
| 8 | | 2000 | 17 | | | |
| 9 | 113 | | | | | |
| 10 | | | | 96 | | |
| 11 | | | | 17.9 | | |
| 12 | 33.1 | | | 0.7 | | |
| 13 | | | | 38.7 | | |
| 14 | | | | 23.9 | | |
| 15 | | | | 492 | | |
| 16 | | | | 361 | | |
| 17 | | | | 26.35 | | |
| 18 | | | | 353 | | |
| 19 | | | | 1203.5 | | |
| 20 | | | | 106.2 | | |
| 21 | | | | 4567 | | |
| 22 | | | | 4148 | | |
| 23 | | | | 244 | | |
| 24 | | | | 583 | | |
| 25 | | | | 162.5 | | |
| 26 | | | | 97.1 | | |
| 27 | | | | 4413 | | |
| 28 | | | | 1759 | | |
| 29 | | | | 1637 | | |
| 30 | | | | | 6000 | 35 |
| 31 | | | | 1664 | | |
| 32 | | | | | 6000 | 54 |
| 33 | | | | 597 | | |
| 34 | | | | 2581.5 | | |
| 35 | | | | 5104 | | |
| 36 | | | | | 6000 | 57 |
| 37 | | | | | 6000 | 15 |
| 38 | | | | | 6000 | 63 |
| 39 | | | | | 6000 | 32 |
| 40 | | | | 1075 | | |
| 41 | | | | | 6000 | 50 |
| 42 | | | | | 6000 | 48 |
| 43 | | | | | 6000 | 67 |
| 44 | | | | 875.5 | | |
| 45 | | | | 113.8 | | |
| 46 | | | | 92.7 | | |
| 47 | | | | 430.5 | | |
| 48 | | | | 636 | | |
| 49 | | | | 900 | | |
| 50 | | | | 113.8 | | |
| 51 | | | | 248.5 | | |
| 52 | | | | 607 | | |
| 53 | | | | 187.5 | | |
| 54 | | | | 407 | | |
| 55 | | | | 47.8 | | |
| 56 | | | | 345 | | |
| 57 | | | | | 6000 | 41 |
| 58 | | | | 822 | | |
| 59 | | | | 122.4 | | |
| 60 | | | | 465 | | |
| 61 | | | | 80.7 | | |
| 62 | | | | 337.9 | | |
| 63 | | | | 156.5 | | |
| 64 | | | | 1004.5 | | |
| 65 | | | | | 6000 | 58 |
| 66 | | | | 2020 | | |
| 67 | | | | 1502.5 | | |
| 68 | | | | 471 | | |
| 69 | | | | 733 | | |
| 70 | | | | 119 | | |
| 71 | | | | 69.4 | | |
| 72 | | | | 115 | | |
| 73 | | | | 17.7 | | |
| 74 | | | | 58.5 | | |
| 75 | | | | 3.7 | | |
| 76 | | | | 5045 | | |
| 77 | | | | | 6000 | 33% |
| 78 | | | | | 6000 | 45% |
| 79 | | | | | 6000 | 21% |
| 80 | | | | 4162 | | |
| 81 | | | | | 6000 | 27% |
| 82 | | | | | 6000 | 47% |
| 83 | | | | | 6000 | 41% |
| 84 | | | | | 6000 | 27% |
| 85 | | | | 1925 | 6000 | 62% |
| 86 | | | | 1637 | | |
| 87 | | | | | 6000 | 35% |
| 88 | | | | 1664 | | |
| 89 | | | | 18 | | |
| 90 | | | | 59 | | |
| 91 | | | | 4 | | |
| 92 | | | | 19 | | |
| 93 | | | | 16 | | |
| 94 | | | | 22 | | |
| 95 | | | | 270 | | |
| 96 | | | | 118 | | |
| 97 | | | | 369 | | |
| 98 | | | | 77 | | |
| 99 | | | | 62 | | |
| 100 | | | | 343 | | |
| 101 | | | | 49 | | |
| 102 | | | | 1268 | | |
| 103 | | | | 3323 | | |
| 104 | | | | 302 | | |
| 105 | | | | 1218 | | |
| 106 | | | | 97 | | |
| 107 | | | | 74 | | |
| 108 | | | | 24 | | |
| 109 | | | | 93 | | |
| 110 | | | | 11 | | |
| 111 | | | | 92 | | |
| 112 | | | | 100 | | |
| 113 | | | | 34 | | |
| 114 | | | | 101 | | |
| 115 | | | | 546 | | |
| 116 | | | | 85 | | |
| 117 | | | | 79 | | |
| 118 | | | | 143 | | |
| 119 | | | | 494 | | |
| 120 | | | | 10 | | |
| 121 | | | | 52 | | |
| 122 | | | | 5 | | |
| 123 | | | | 256 | | |
| 124 | | | | 1709 | | |
| 125 | | | | 238 | | |
| 126 | | | | 98 | | |
| 127 | | | | 1542 | | |
| 128 | | | | 89 | | |
| 129 | | | | 95 | | |
| 130 | | | | 45 | | |
| 131 | | | | 31 | | |
| 132 | | | | 16 | | |
| 133 | | | | | | 35% at 6 μM |
| 134 | | | | 788 | | |
| 135 | | | | 165 | | |
| 136 | | | | 1364 | | |
| 137 | | | | 193 | | |
| 138 | | | | 3748 | | |
| 139 | | | | 644 | | |
| 140 | | | | 4325 | | |
| 141 | | | | | | 30% at 6 μM |
| 142 | | | | 1149 | | |
| 143 | | | | 231 | | |
| 144 | | | | | | 53% at 6 μM |
| 145 | | | | 74 | | |
| 146 | | | | 66 | | |
| 147 | | | | 34 | | |
| 148 | | | | 51 | | |
| 149 | | | | 74 | | |
| 150 | | | | 16 | | |
| 151 | | | | 31 | | |
| 152 | | | | 40 | | |
| 153 | | | | 43 | | |
| 154 | | | | 31 | | |
| 155 | | | | 25 | | |
| 156 | | | | 27 | | |
| 157 | | | | 128 | | |
| 158 | | | | 145 | | |
| 159 | | | | 133 | | |
| 160 | | | | 142 | | |
| 161 | | | | 143 | | |
| 162 | | | | 135 | | |
| 163 | | | | 189 | | |
| 164 | | | | 288 | | |
| 165 | | | | 206 | | |
| 166 | | | | 304 | | |
| 167 | | | | 587 | | |
| 168 | | | | 341 | | |
| 169 | | | | 96 | | |
| 170 | | | | 343 | | |
| 171 | | | | 322 | | |
| 172 | | | | 16 | | |
| 173 | | | | 171 | | |
| 174 | | | | 26 | | |
| 175 | | | | 42 | | |
| 176 | | | | 20 | | |
| 177 | | | | 43 | | |

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A compound of formula I: 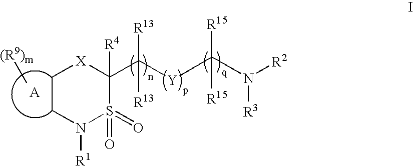
or a pharmaceutically acceptable salt thereof;
wherein:
m is an integer from 0 to 4;
n is an integer from 0 to 2;
p is an integer from 0 to 1;
q is an integer from 1 to 2;
v is an integer from 0 to 2;
X is C(R¹¹)₂, N(R¹²), O, or S(O)ᵥ; 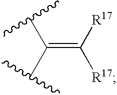
Y is C(R¹¹)₂, N(R¹²), O, or
R¹ is aryl substituted with 0-3 R⁵ or heteroaryl substituted with 0-3 R⁵;
R² is H, straight or branched C₁-C₆ alkyl, C₃-C₆ cycloalkyl, or aryl-C₁-C₆ alkyl where said aryl portion is substituted with 0-3 R⁶;
R³ is H, straight or branched C₁-C₆ alkyl, C₁-C₆ alkanol, C₃-C₆ cycloalkyl, or aryl-C₁-C₆ alkyl where said aryl portion is substituted with 0-3 R⁷; or
R² and R³, together with the nitrogen through which they are attached, form a mono- or bicyclic heterocyclic ring of 3 to 12 ring atoms, where one carbon may be optionally replaced with N, O, S, or SO₂, and where any carbon ring atom may be optionally substituted with C₁-C₄ alkyl, F, or CF₃ or where any additional N atom may be optionally substituted with C₁-C₄ alkyl with the proviso that if Y is O or N(R¹²), q is not 2;
R⁴ is H, straight or branched C₁-C₆ alkyl, aryl substituted with 0-3 R⁸, aryl-C₁-C₆ alkyl where said aryl portion is substituted with 0-3 R⁸, heteroaryl substituted with 0-3 R⁸, or heteroaryl-C₁-C₆ alkyl where said heteroaryl portion is substituted with 0-3 R⁸;
R⁵ is, independently at each occurrence, C₁-C₆ alkyl, alkoxy, halo, CF₃, OCF₃, hydroxy, alkanoyloxy, nitro, nitrile, alkenyl, alkynyl, aryl substituted with 0-3 R⁵ or heteroaryl substituted with 0-3 R⁵;
R⁶ is, independently at each occurrence, C₁-C₄ alkyl, halo or H;
R⁷ is, independently at each occurrence, C₁-C₄ alkyl, halo or H;
R⁸ is, independently at each occurrence, C₁-C₄ alky, halo or H;
R⁹ is, independently at each occurrence, C₁-C₆ alkyl, alkoxy, halo, H, CF₃, OCF₃, hydroxy, alkanoyloxy, nitro, nitrile, alkenyl, alkynyl, aryl substituted with 0-3 R¹⁰, heteroaryl substituted with 0-3 R¹⁰, alkylsulfoxide, alkylsulfone, alkylsulfonamide, phenylsulfonamide substituted with 0-3 R¹⁰, alkylamido, or arylamido substituted with 0-3 R¹⁰; or
two adjacent R⁹, together with the ring atoms to which they are attached, form a fused ring of 5 or 6 ring atoms;
R¹⁰ is, independently at each occurrence, H, C₁-C₆ alkyl, or halo;
R¹¹ is, independently at each occurrence, H, C₁-C₆ alkyl, halo, hydroxy, O—(C₁-C₆ alkyl), or aryl substituted with 0-3 R⁸;
R¹² is, independently at each occurrence, H, C₁-C₆ alkyl, or aryl substituted with 0-3 R⁸;
R¹³ is, independently at each occurrence, H, C₁-C₆ alkyl, halide, hydroxy, or aryl substituted with 0-3 R¹⁶;
R¹⁵ is, independently at each occurrence, H, C₁-C₄ alkyl or halide;
R¹⁶ is, independently at each occurrence, H, C₁-C₄ alkyl, or halo;
R¹⁷ is, independently at each occurrence, H or C₁-C₄ alkyl;
wherein ring A is phenyl, naphthyl, pyridyl, pyrimidinyl, thienyl, thiazolyl, or pyrrolyl.

2. A compound according to claim 1,
wherein:
A is phenyl.

3. A compound according to claim 1,
wherein:
n is 1.

4. A compound according to claim 1,
wherein:
p is 1.

5. A compound according to claim 1,
wherein:
q is 1.

6. A compound according to claim 1,
wherein:
m is an integer from 0 to 2.

7. A compound according to claim 1,
wherein:
X is C(R¹¹)₂, O, or S.

8. A compound according to claim 1,
wherein:
Y is C(R¹¹)₂ or O.

9. A compound according to claim 1,
wherein:
R¹ is aryl substituted with 0-3 R⁵.

10. A compound according to claim 1,
wherein:
R¹ is phenyl, methyl-phenyl, dimethyl-phenyl, methoxy-phenyl, fluoro-phenyl, chloro-phenyl, fluoro-chloro-phenyl, trifluoromethyl-phenyl, naphthyl, or fluoro-fluoro-phenyl.

11. A compound according to claim 1,
wherein:
R¹ is heteroaryl substituted with 0-3 R⁵.

12. A compound according to claim 1,
wherein:
R¹ is pyridinyl or quinolinyl.

13. A compound according to claim 1,
wherein:
R² is H or straight or branched C₁-C₆ alkyl.

14. A compound according to claim 1,
wherein:
R² is H, methyl, ethyl, n-propyl, isopropyl, cyclopropyl, n-butyl, isobutyl, cyclobutyl, cyclopentyl, cyclohexyl, or benzyl.

15. A compound according to claim 1,
wherein:
R² is methyl.

16. A compound according to claim 1,
wherein:
R³ is H, methyl, ethyl, n-propyl, isopropyl, cyclopropyl, n-butyl, isobutyl, cyclobutyl, cyclopentyl, cyclohexyl, benzyl, or propanol.

17. A compound according to claim 1,
wherein:
R³ is H.

18. A compound according to claim 1,
wherein:
R² and R³, together with the nitrogen through which they are attached, form pyrrolidinyl, piperidinyl, or piperazinyl.

19. A compound according to claim 1,
wherein:
R⁴ is H or straight or branched C₁-C₆ alkyl.

20. A compound according to claim 1,
wherein:
R⁴ is H or methyl.

21. A compound according to claim 1,
wherein:
R⁵ is, independently at each occurrence, C₁-C₆ alkyl or halo.

22. A compound according to claim 1,
wherein:
R⁵ is methyl, methoxy, fluoro, chloro, or trifluoromethyl.

23. A compound according to claim 1,
wherein:
R⁶ is methyl, fluoro, or chloro.

24. A compound according to claim 1,
wherein:
R⁷ is methyl, fluoro, chloro or hydrogen.

25. A compound according to claim 1,
wherein:
R⁹ is C₁-C₆ alkyl, halo or hydrogen.

26. A compound according to claim 1,
wherein:
R⁹ is methyl, methoxy, fluoro, chloro, trifluoromethyl or hydrogen.

27. A compound according to claim 1,
wherein:
R¹¹ is, independently at each occurrence, H, C₁-C₆ alkyl, or halo.

28. A compound according to claim 1,
wherein:
R¹² is, independently at each occurrence, H or C₁-C₆ alkyl.

29. A compound according to claim 1,
wherein:
R¹³ is, independently at each occurrence, H, C₁-C₆ alkyl, or aryl.

30. A compound according to claim 1,
wherein:
n is 1;
p is 1;
q is 1;
X is C(R¹¹)₂ or O;
Y is C(R¹¹)₂; and
R³ is H.

31. A compound according to claim 1,
wherein:
n is 1;
p is 1;
q is 1;
X is C(R¹¹)₂ or O;
Y is C(R¹¹)₂;
R¹ is phenyl; and
R³ is H.

32. A compound according to claim 1,
wherein:
n is 1;
p is 1;
q is 1;
X is C(R¹¹)₂ or O;
Y is C(R¹¹)₂;
R¹ is phenyl;
R² is methyl;
R³ is H; and
R⁵ is H or F.

33. A compound according to claim 1,
wherein:
n is 1;
p is 1;
q is 1;
X is C(R¹¹)₂ or O;
Y is C(R¹¹)₂;
R¹ is phenyl;
R² is methyl;
R³ is H;
R⁴ is H;
R⁵ is H or F; and
R⁹ is H or F.

34. A compound according to claim 1, selected from the group consisting of:
3-(2,2-dioxido-1-phenyl-3,4-dihydro-1H-2,1-benzothiazin-3-yl)-N-methylpropan-1-amine;
3-(2,2-dioxido-1-phenyl-3,4-dihydro-1H-2,1-benzothiazin-3-yl)propan-1-amine;
3-(2,2-dioxido-1-phenyl-3,4-dihydro-1H-2,1-benzothiazin-3-yl)-N,N-dimethylpropan-1-amine;
3-(2,2-dioxido-1-phenyl-3,4-dihydro-1H-2,1-benzothiazin-3-yl)-N-ethylpropan-1-amine;
3-(2,2-dioxido-1-phenyl-3,4-dihydro-1H-2,1-benzothiazin-3-yl)-N-isopropyl propan-1-amine;
1-phenyl-3-(3-pyrrolidin-1-ylpropyl)-3,4-dihydro-1H-2,1-benzothiazine 2,2-dioxide;
N-benzyl-3-(2,2-dioxido-1-phenyl-3,4-dihydro-1H-2,1-benzothiazin-3-yl)propan-1-amine;
N-[3-(2,2-dioxido-1-phenyl-3,4-dihydro-1H-2,1-benzothiazin-3-yl)propyl]cyclohexanamine;
N-methyl-3-[3-methyl-2,2-dioxido-1-phenyl-3,4-dihydro-1H-2,1-benzothiazin-3-yl]propan-1-amine;
3-[2,2-dioxido-1-phenyl-3,4-dihydro-1H-2,1-benzothiazin-3-yl]-N-methylpropan-1-amine;
3-(2,2-dioxido-1-phenyl-1H-4,2,1-benzoxathiazin-3-yl)-N-methylpropan-1-amine;
3-[2,2-dioxido-1-phenyl-1H-4,2,1-benzoxathiazin-3-yl]-N-methylpropan-1-amine;
3-[2,2-dioxido-1-phenyl-1H-4,2,1-benzoxathiazin-3-yl]-N-methylpropan-1-amine;
3-[1-(4-fluorophenyl)-2,2-dioxido-1H-4,2,1-benzoxathiazin-3-yl]-N-methylpropan-1-amine;
N-methyl-3-[1-(3-methylphenyl)-2,2-dioxido-1H-4,2,1-benzoxathiazin-3-yl]propan-1-amine;
N-methyl-3-[1-(4-methylphenyl)-2,2-dioxido-1H-4,2,1-benzoxathiazin-3-yl]propan-1-amine;
3-[1-(3-methoxyphenyl)-2,2-dioxido-1H-4,2,1-benzoxathiazin-3-yl]-N-methylpropan-1-amine;
3-[1-(4-methoxyphenyl)-2,2-dioxido-1H-4,2,1-benzoxathiazin-3-yl]-N-methylpropan-1-amine;
3-[1-(3-fluorophenyl)-2,2-dioxido-1H-4,2,1-benzoxathiazin-3-yl]-N-methylpropan-1-amine;
N-methyl-3-[1-(2-naphthyl)-2,2-dioxido-1H-4,2,1-benzoxathiazin-3-yl]propan-1-amine;
3-[1-(3,5-dimethylphenyl)-2,2-dioxido-1H-4,2,1-benzoxathiazin-3-yl]-N-methylpropan-1-amine;
2-(2,2-dioxido-1-phenyl-1H-4,2,1-benzoxathiazin-3-yl)-N-ethylethanamine;
2-(2,2-dioxido-1-phenyl-1H-4,2,1-benzoxathiazin-3-yl)ethanamine;
2-(2,2-dioxido-1-phenyl-1H-4,2,1-benzoxathiazin-3-yl)-N-methylethanamine;
2-(2,2-dioxido-1-phenyl-1H-4,2,1-benzoxathiazin-3-yl)-N,N-dimethylethanamine;
N-[2-(2,2-dioxido-1-phenyl-1H-4,2,1-benzoxathiazin-3-yl)ethyl]propan-1-amine;
N-[2-(2,2-dioxido-1-phenyl-1H-4,2,1-benzoxathiazin-3-yl)ethyl]-2-methylpropan-1-amine;
1-phenyl-3-(2-pyrrolidin-1-ylethyl)-1H-4,2,1-benzoxathiazine 2,2-dioxide;
3-[2-(4-methylpiperazin-1-yl)ethyl]-1-phenyl-1H-4,2,1-benzoxathiazine 2,2-dioxide;
N-[2-(2,2-dioxido-1-phenyl-1H-4,2,1-benzoxathiazin-3-yl)ethyl]butan-1-amine;
N-[2-(2,2-dioxido-1-phenyl-1H-4,2,1-benzoxathiazin-3-yl)ethyl]cyclobutanamine;
3-(2,2-dioxido-1-phenyl-1H-4,2,1-benzoxathiazin-3-yl)propan-1-amine;
3-(2,2-dioxido-1-phenyl-1H-4,2,1-benzoxathiazin-3-yl)-N-ethylpropan-1-amine;
3-(2,2-dioxido-1-phenyl-1H-4,2,1-benzoxathiazin-3-yl)-N-propylpropan-1-amine;
N-[3-(2,2-dioxido-1-phenyl-1H-4,2,1-benzoxathiazin-3-yl)propyl]butan-1-amine;
3-(2,2-dioxido-1-phenyl-1H-4,2,1-benzoxathiazin-3-yl)-N-isopropylpropan-1-amine;
N-[3-(2,2-dioxido-1-phenyl-1H-4,2,1-benzoxathiazin-3-yl)propyl]-2-methylpropan-1-amine;
3-{[3-(2,2-dioxido-1-phenyl-1H-4,2,1-benzoxathiazin-3-yl)propyl]amino}propan-1-ol;
N-[3-(2,2-dioxido-1-phenyl-1H-4,2,1-benzoxathiazin-3-yl)propyl]cyclo propanamine;
N-[3-(2,2-dioxido-1-phenyl-1H-4,2,1-benzoxathiazin-3-yl)propyl]cyclobutanamine;
N-[3-(2,2-dioxido-1-phenyl-1H-4,2,1-benzoxathiazin-3-yl)propyl]cyclopentanamine;
N-[3-(2,2-dioxido-1-phenyl-1H-4,2,1-benzoxathiazin-3-yl)propyl]cyclohexanamine;
3-(7-fluoro-2,2-dioxido-1-phenyl-1H-4,2,1-benzoxathiazin-3-yl)-N-methylpropan-1-amine;
3-(6-chloro-2,2-dioxido-1-phenyl-1H-4,2,1-benzoxathiazin-3-yl)-N-methylpropan-1-amine;
3-(6-fluoro-2,2-dioxido-1-phenyl-1H-4,2,1-benzoxathiazin-3-yl)-N-methylpropan-1-amine;
3-(5-fluoro-2,2-dioxido-1-phenyl-1H-4,2,1-benzoxathiazin-3-yl)-N-methylpropan-1-amine;
3-(8-fluoro-2,2-dioxido-1-phenyl-1H-4,2,1-benzoxathiazin-3-yl)-N-methylpropan-1-amine;
N-methyl-3-(8-methyl-2,2-dioxido-1-phenyl-1H-4,2,1-benzoxathiazin-3-yl)propan-1-amine;
N-methyl-3-(7-methyl-2,2-dioxido-1-phenyl-1H-4,2,1-benzoxathiazin-3-yl)propan-1-amine;
N-methyl-3-(6-methyl-2,2-dioxido-1-phenyl-1H-4,2,1-benzoxathiazin-3-yl)propan-1-amine;
3-(6-methoxy-2,2-dioxido-1-phenyl-1H-4,2,1-benzoxathiazin-3-yl)-N-methylpropan-1-amine;
3-(7-chloro-2,2-dioxido-1-phenyl-1H-4,2,1-benzoxathiazin-3-yl)-N-methylpropan-1-amine;
3-(2,2-dioxido-1,7-diphenyl-1H-4,2,1-benzoxathiazin-3-yl)-N-methylpropan-1-amine;
3-(2,2-dioxido-1-phenyl-1H-naphtho[2,3-e][1,3,4]oxathiazin-3-yl)-N-methylpropan-1-amine;
3-(2,2-dioxido-1-pyridin-3-yl-1H-4,2,1-benzoxathiazin-3-yl)-N-methylpropan-1-amine;
3-(2,2-dioxido-1-quinolin-3-yl-1H-4,2,1-benzoxathiazin-3-yl)-N-methylpropan-1-amine;
N-benzyl-3-(2,2-dioxido-1-phenyl-1H-4,2,1-benzoxathiazin-3-yl)-N-methylpropan-1-amine;
3-(2,2-dioxido-1-phenyl-1H-2,4,1-benzodithiazin-3-yl)-N-methylpropan-1-amine;
N-methyl-3-[1-(3-methylphenyl)-2,2-dioxido-1H-2,4,1-benzodithiazin-3-yl]propan-1-amine;
3-[1-(3-fluorophenyl)-2,2-dioxido-1H-2,4,1-benzodithiazin-3-yl]-N-methylpropan-1-amine;
3-[1-(3-methoxyphenyl)-2,2-dioxido-1H-2,4,1-benzodithiazin-3-yl]-N-methylpropan-1-amine;
N-methyl-3-[1-(4-methylphenyl)-2,2-dioxido-1H-2,4,1-benzodithiazin-3-yl]propan-1-amine;
3-[1-(4-fluorophenyl)-2,2-dioxido-1H-2,4,1-benzodithiazin-3-yl]-N-methylpropan-1-amine;
3-[1-(4-methoxyphenyl)-2,2-dioxido-1H-2,4,1-benzodithiazin-3-yl]-N-methylpropan-1-amine;
3-[1-(3-chlorophenyl)-2,2-dioxido-1H-2,4,1-benzodithiazin-3-yl]-N-methylpropan-1-amine;
3-[1-(4-chlorophenyl)-2,2-dioxido-1H-2,4,1-benzodithiazin-3-yl]-N-methylpropan-1-amine;
3-[1-(3-chloro-4-fluorophenyl)-2,2-dioxido-1H-2,4,1-benzodithiazin-3-yl]-N-methylpropan-1-amine;
N-methyl-3-[1-(4-methylphenyl)-2,2-dioxido-1H-naphtho[2,3-e][1,3,4]oxathiazin-3-yl]propan-1-amine;
3-[1-(3-fluorophenyl)-2,2-dioxido-1H-naphtho[2,3-e][1,3,4]oxathiazin-3-yl]-N-methylpropan-1-amine;
3-[2,2-dioxido-1-phenyl-1H-naphtho[2,3-e][1,3,4]oxathiazin-3-yl]-N-methylpropan-1-amine;
3-[2,2-dioxido-1-phenyl-1H-naphtho[2,3-e][1,3,4]oxathiazin-3-yl]-N-methylpropan-1-amine;
3-[1-(2-fluorophenyl)-2,2-dioxido-3,4-dihydro-1H-2,1-benzothiazin-3-yl]-N-methylpropan-1-amine;
3-[1-(2-fluorophenyl)-2,2-dioxido-3,4-dihydro-1H-2,1-benzothiazin-3-yl]-N-methylpropan-1-amine;
3-[1-(2-fluorophenyl)-2,2-dioxido-3,4-dihydro-1H-2,1-benzothiazin-3-yl]-N-methylpropan-1-amine;
1-phenyl-3-(3-piperazin-1-ylpropyl)-1H-4,2,1-benzoxathiazine-2,2-dioxide;
3-[2-(1,4-diazepan-1-yl)ethyl]-1-phenyl-1H-4,2,1-benzoxathiazine-2,2-dioxide;
1-phenyl-3-(2-piperidin-1-ylethyl)-1H-4,2,1-benzoxathiazine 2,2-dioxide;
3-[2-(3,5-dimethylpiperazin-1-yl)ethyl]-1-phenyl-1H-4,2,1-benzoxathiazine 2,2-dioxide;
3-(2,2-dioxido-1-phenyl-1H-4,2,1-benzoxathiazin-3-yl)-N,N-dimethylpropan-1-amine;
3-(2,2-dioxido-1-phenyl-1H-4,2,1-benzoxathiazin-3-yl)-N-ethyl-N-methylpropan-1-amine;
3-(2,2-dioxido-1-phenyl-1H-4,2,1-benzoxathiazin-3-yl)-N,N-diethylpropan-1-amine;
2-{[3-(2,2-dioxido-1-phenyl-1H-4,2,1-benzoxathiazin-3-yl)propyl](ethyl)amino}ethanol;
3-(2,2-dioxido-1-phenyl-1H-4,2,1-benzoxathiazin-3-yl)-N-isopropyl-N-methylpropan-1-amine;
N-[3-(2,2-dioxido-1-phenyl-1H-4,2,1-benzoxathiazin-3-yl)propyl]-N-methylcyclohexanamine;
1-phenyl-3-(2-pyrrolidin-1-yl-ethyl)-1H-benzo[1,3,4]oxathiazine 2,2-dioxide;
3-[2-(4-methyl-piperazin-1-yl)-ethyl]-1-phenyl-1H-benzo[1,3,4]oxathiazine 2,2-dioxide;
N-butyl-[2-(2,2-dioxo-1-phenyl-2,3-dihydro-1H-2λ⁶-benzo[1,3,4]oxathiazin-3-yl)-ethyl]-amine;
3-[1-(2-fluorophenyl)-2,2-dioxido-3,4-dihydro-1H-2,1-benzothiazin-3-yl]-N-methylpropan-1-amine;
3-[6-fluoro-1-(4-fluoro-2-methylphenyl)-2,2-dioxido-3,4-dihydro-1H-2,1-benzothiazin-3-yl]-N-methylpropan-1-amine;
1-(2,2-dioxido-1-phenyl-1H-4,2,1-benzoxathiazin-3-yl)-3-(methylamino)propan-2-ol;
3-[1-(2,4-difluorophenyl)-2,2-dioxido-3,4-dihydro-1H-2,1-benzothiazin-3-yl]-N-methylpropan-1-amine;
2-(2,2-dioxido-1-phenyl-1H-4,2,1-benzoxathiazin-3-yl)ethanamine;
3-(6-fluoro-2,2-dioxido-1-phenyl-1H-4,2,1-benzoxathiazin-3-yl)-N-methylpropan-1-amine;
3-[6-fluoro-1-(2-fluorophenyl)-2,2-dioxido-3,4-dihydro-1H-2,1-benzothiazin-3-yl]-N-methylpropan-1-amine;
3-[1-(2,4-difluorophenyl)-6-fluoro-2,2-dioxido-3,4-dihydro-1H-2,1-benzothiazin-3-yl]-N-methylpropan-1-amine;
1-(2,2-dioxido-1-phenyl-3,4-dihydro-1H-2,1-benzothiazin-3-yl)-3-(methylamino)propan-1-ol;
2-(6-fluoro-2,2-dioxido-1-phenyl-1H-4,2,1-benzoxathiazin-3-yl)-N-methylethanamine;
3-[1-(2,6-difluorophenyl)-2,2-dioxido-3,4-dihydro-1H-2,1-benzothiazin-3-yl]-N-methylpropan-1-amine;
3-(2,2-dioxido-1-pyridin-3-yl-3,4-dihydro-1H-2,1-benzothiazin-3-yl)-N-methylpropan-1-amine;
3-(2,2-dioxido-1-pyridin-2-yl-3,4-dihydro-1H-2,1-benzothiazin-3-yl)-N-methylpropan-1-amine;
3-[6-fluoro-1-(2-fluorophenyl)-2,2-dioxido-1H-4,2,1-benzoxathiazin-3-yl]-N-methylpropan-1-amine hydrochloride;
2-[(2,2-dioxido-1-phenyl-3,4-dihydro-1H-2,1-benzothiazin-3-yl)methyl]-N-methylprop-2-en-1-amine;
3-(2,2-dioxido-1-pyridin-4-yl-3,4-dihydro-1H-2,1-benzothiazin-3-yl)-N-methylpropan-1-amine;
1-(2,2-dioxido-1-phenyl-1H-4,2,1-benzoxathiazin-3-yl)-N,N-dimethylmethanamine;
1-phenyl-3-(piperazin-1-ylmethyl)-1H-4,2,1-benzoxathiazine 2,2-dioxide;
N-[(2,2-dioxido-1-phenyl-1H-4,2,1-benzoxathiazin-3-yl)methyl]ethanamine;
N-[(2,2-dioxido-1-phenyl-1H-4,2,1-benzoxathiazin-3-yl)methyl]ethane-1,2-diamine;
N′-[(2,2-dioxido-1-phenyl-1H-4,2,1-benzoxathiazin-3-yl)methyl]-N,N-dimethylethane-1,2-diamine;
N-[(2,2-dioxido-1-phenyl-1H-4,2,1-benzoxathiazin-3-yl)methyl]-N,N′-dimethylethane-1,2-diamine;
N-[(2,2-dioxido-1-phenyl-1H-4,2,1-benzoxathiazin-3-yl)methyl]-N,N′,N′-trimethylethane-1,2-diamine;
3-[(4-methylpiperazin-1-yl)methyl]-1-phenyl-1H-4,2,1-benzoxathiazine 2,2-dioxide;
3-[(3,5-dimethylpiperazin-1-yl)methyl]-1-phenyl-1H-4,2,1-benzoxathiazine 2,2-dioxide;
3-(2,5-diazabicyclo[2.2.1]hept-2-ylmethyl)-1-phenyl-1H-4,2,1-benzoxathiazine 2,2-dioxide;
1-(2,2-dioxido-1-phenyl-1H-4,2,1-benzoxathiazin-3-yl)-N-methylmethanamine;
3-[1-(2,3-difluorophenyl)-6-fluoro-2,2-dioxido-3,4-dihydro-1H-2,1-benzothiazin-3-yl]-N-methylpropan-1-amine;
3-[6-fluoro-1-(2-fluoro-4-methylphenyl)-2,2-dioxido-3,4-dihydro-1H-2,1-benzothiazin-3-yl]-N-methylpropan-1-amine;
3-[1-(2,5-difluorophenyl)-6-fluoro-2,2-dioxido-3,4-dihydro-1H-2,1-benzothiazin-3-yl]-N-methylpropan-1-amine;
3-[1-(2,6-difluorophenyl)-6-fluoro-2,2-dioxido-3,4-dihydro-1H-2,1-benzothiazin-3-yl]-N-methylpropan-1-amine;
3-[1-(2-chlorophenyl)-6-fluoro-2,2-dioxido-3,4-dihydro-1H-2,1-benzothiazin-3-yl]-N-methylpropan-1-amine;
3-[6-fluoro-1-(2-methylphenyl)-2,2-dioxido-3,4-dihydro-1H-2,1-benzothiazin-3-yl]-N-methylpropan-1-amine;
2-{6-fluoro-3-[3-(methylamino)propyl]-2,2-dioxido-3,4-dihydro-1H-2,1-benzothiazin-1-yl}benzonitrile;
3-[6-fluoro-1-(2-methoxyphenyl)-2,2-dioxido-3,4-dihydro-1H-2,1-benzothiazin-3-yl]-N-methylpropan-1-amine;
3-[6-fluoro-1-(4-fluoro-2-methylphenyl)-2,2-dioxido-3,4-dihydro-1H-2,1-benzothiazin-3-yl]-N-methylpropan-1-amine;
3-(6-fluoro-2,2-dioxido-1-phenyl-3,4-dihydro-1H-2,1-benzothiazin-3-yl)-N-methylpropan-1-amine; and
3-[6-chloro-1-(2-fluorophenyl)-2,2-dioxido-3,4-dihydro-1H-2,1-benzothiazin-3-yl]-N-methylpropan-1-amine;
and
pharmaceutically acceptable salts thereof.

35. A compound according to claim 1, selected from the group consisting of:
N-methyl-3-[(3R)-3-methyl-2,2-dioxido-1-phenyl-3,4-dihydro-1H-2,1-benzothiazin-3-yl]propan-1-amine;
3-[(3S)-2,2-dioxido-1-phenyl-3,4-dihydro-1H-2,1-benzothiazin-3-yl]-N-methylpropan-1-amine;
3-[(3R)-2,2-dioxido-1-phenyl-3,4-dihydro-1H-2,1-benzothiazin-3-yl]-N-methylpropan-1-amine;
3-[(3S)-2,2-dioxido-1-phenyl-1H-4,2,1-benzoxathiazin-3-yl]-N-methylpropan-1-amine;
3-[(3R)-2,2-dioxido-1-phenyl-1H-4,2,1-benzoxathiazin-3-yl]-N-methylpropan-1-amine;
3-[(3S)-2,2-dioxido-1-phenyl-1H-naphtho[2,3-e][1,3,4]oxathiazin-3-yl]-N-methylpropan-1-amine;
3-[(3R)-2,2-dioxido-1-phenyl-1H-naphtho[2,3-e][1,3,4]oxathiazin-3-yl]-N-methylpropan-1-amine;
3-[(3S)-1-(2-fluorophenyl)-2,2-dioxido-3,4-dihydro-1H-2,1-benzothiazin-3-yl]-N-methylpropan-1-amine;
3-[(3R)-1-(2-fluorophenyl)-2,2-dioxido-3,4-dihydro-1H-2,1-benzothiazin-3-yl]-N-methylpropan-1-amine;
3-[(3S)-1-(2-fluorophenyl)-2,2-dioxido-3,4-dihydro-1H-2,1-benzothiazin-3-yl]-N-methylpropan-1-amine;
3-[(3R)-1-(2-fluorophenyl)-2,2-dioxido-3,4-dihydro-1H-2,1-benzothiazin-3-yl]-N-methylpropan-1-amine;
3-[(3S)-6-fluoro-1-(4-fluoro-2-methylphenyl)-2,2-dioxido-3,4-dihydro-1H-2,1-benzothiazin-3-yl]-N-methylpropan-1-amine;
3-[(3R)-6-fluoro-1-(4-fluoro-2-methyl phenyl)-2,2-dioxido-3,4-dihydro-1H-2,1-benzothiazin-3-yl]-N-methylpropan-1-amine;
(2S)-1-[(3R)-2,2-dioxido-1-phenyl-1H-4,2,1-benzoxathiazin-3-yl]-3-(methylamino)propan-2-ol;
(2R)-1-[(3R)-2,2-dioxido-1-phenyl-1H-4,2,1-benzoxathiazin-3-yl]-3-(methylamino)propan-2-ol;
(2R)-1-[(3S)-2,2-dioxido-1-phenyl-1H-4,2,1-benzoxathiazin-3-yl]-3-(methylamino)propan-2-ol;
(2S)-1-[(3S)-2,2-dioxido-1-phenyl-1H-4,2,1-benzoxathiazin-3-yl]-3-(methylamino)propan-2-ol;
3-[(3S)-1-(2,4-difluorophenyl)-2,2-dioxido-3,4-dihydro-1H-2,1-benzothiazin-3-yl]-N-methylpropan-1-amine;
3-[(3R)-1-(2,4-difluorophenyl)-2,2-dioxido-3,4-dihydro-1H-2,1-benzothiazin-3-yl]-N-methylpropan-1-amine;
2-[(3S)-2,2-dioxido-1-phenyl-1H-4,2,1-benzoxathiazin-3-yl]ethanamine;
2-[(3R)-2,2-dioxido-1-phenyl-1H-4,2,1-benzoxathiazin-3-yl]ethanamine;
2-[(3S)-2,2-dioxido-1-phenyl-1H-4,2,1-benzoxathiazin-3-yl]-N-methylethanamine;
2-[(3R)-2,2-dioxido-1-phenyl-1H-4,2,1-benzoxathiazin-3-yl]-N-methylethanamine;
3-[(3S)-6-fluoro-2,2-dioxido-1-phenyl-1H-4,2,1-benzoxathiazin-3-yl]-N-methylpropan-1-amine;
3-[(3R)-6-fluoro-2,2-dioxido-1-phenyl-1H-4,2,1-benzoxathiazin-3-yl]-N-methylpropan-1-amine;
3-[(3S)-6-fluoro-1-(2-fluorophenyl)-2,2-dioxido-3,4-dihydro-1H-2,1-benzothiazin-3-yl]-N-methylpropan-1-amine;
3-[(3R)-6-fluoro-1-(2-fluorophenyl)-2,2-dioxido-3,4-dihydro-1H-2,1-benzothiazin-3-yl]-N-methylpropan-1-amine;
3-[(3S)-1-(2,4-difluorophenyl)-6-fluoro-2,2-dioxido-3,4-dihydro-1H-2,1-benzothiazin-3-yl]-N-methylpropan-1-amine
3-[(3R)-1-(2,4-difluorophenyl)-6-fluoro-2,2-dioxido-3,4-dihydro-1H-2,1-benzothiazin-3-yl]-N-methylpropan-1-amine;
(1S)-1-[(3S)-2,2-dioxido-1-phenyl-3,4-dihydro-1H-2,1-benzothiazin-3-yl]-3-(methylamino)propan-1-ol;
(1R)-1-[(3S)-2,2-dioxido-1-phenyl-3,4-dihydro-1H-2,1-benzothiazin-3-yl]-3-(methylamino)propan-1-ol;
(1R)-1-[(3R)-2,2-dioxido-1-phenyl-3,4-dihydro-1H-2,1-benzothiazin-3-yl]-3-(methylamino)propan-1-ol
(1S)-1-[(3R)-2,2-dioxido-1-phenyl-3,4-dihydro-1H-2,1-benzothiazin-3-yl]-3-(methylamino)propan-1-ol;
2-[(3S)-6-fluoro-2,2-dioxido-1-phenyl-1H-4,2,1-benzoxathiazin-3-yl]-N-methylethanamine;
2-[(3R)-6-fluoro-2,2-dioxido-1-phenyl-1H-4,2,1-benzoxathiazin-3-yl]-N-methylethanamine;
3-[(3S)-1-(2,6-difluorophenyl)-2,2-dioxido-3,4-dihydro-1H-2,1-benzothiazin-3-yl]-N-methylpropan-1-amine;
3-[(3R)-1-(2,6-difluorophenyl)-2,2-dioxido-3,4-dihydro-1H-2,1-benzothiazin-3-yl]-N-methylpropan-1-amine;
3-[(3S)-2,2-dioxido-1-pyridin-3-yl-3,4-dihydro-1H-2,1-benzothiazin-3-yl]-N-methylpropan-1-amine;
3-[(3R)-2,2-dioxido-1-pyridin-3-yl-3,4-dihydro-1H-2,1-benzothiazin-3-yl]-N-methylpropan-1-amine;
3-[(3S)-2,2-dioxido-1-pyridin-2-yl-3,4-dihydro-1H-2,1-benzothiazin-3-yl]-N-methylpropan-1-amine;
3-[(3R)-2,2-dioxido-1-pyridin-2-yl-3,4-dihydro-1H-2,1-benzothiazin-3-yl]-N-methylpropan-1-amine;
3-[(3S)-1-(2,3-difluorophenyl)-6-fluoro-2,2-dioxido-3,4-dihydro-1H-2,1-benzothiazin-3-yl]-N-methylpropan-1-amine;
3-[(3R)-1-(2,3-difluorophenyl)-6-fluoro-2,2-dioxido-3,4-dihydro-1H-2,1-benzothiazin-3-yl]-N-methylpropan-1-amine;
3-[(3S)-6-fluoro-1-(2-fluoro-4-methylphenyl)-2,2-dioxido-3,4-dihydro-1H-2,1-benzothiazin-3-yl]-N-methylpropan-1-amine;
3-[(3R)-6-fluoro-1-(2-fluoro-4-methyl phenyl)-2,2-dioxido-3,4-dihydro-1H-2,1-benzothiazin-3-yl]-N-methylpropan-1-amine;
3-[(3S)-1-(2,5-difluorophenyl)-6-fluoro-2,2-dioxido-3,4-dihydro-1H-2,1-benzothiazin-3-yl]-N-methylpropan-1-amine;
3-[(3R)-1-(2,5-difluorophenyl)-6-fluoro-2,2-dioxido-3,4-dihydro-1H-2,1-benzothiazin-3-yl]-N-methylpropan-1-amine;
3-[(3S)-1-(2,6-difluorophenyl)-6-fluoro-2,2-dioxido-3,4-dihydro-1H-2,1-benzothiazin-3-yl]-N-methylpropan-1-amine;
3-[(3R)-1-(2,6-difluorophenyl)-6-fluoro-2,2-dioxido-3,4-dihydro-1H-2,1-benzothiazin-3-yl]-N-methylpropan-1-amine;
3-[(3S)-1-(2-chlorophenyl)-6-fluoro-2,2-dioxido-3,4-dihydro-1H-2,1-benzothiazin-3-yl]-N-methylpropan-1-amine;
3-[(3R)-1-(2-chlorophenyl)-6-fluoro-2,2-dioxido-3,4-dihydro-1H-2,1-benzothiazin-3-yl]-N-methylpropan-1-amine;
3-[(3S)-6-fluoro-1-(2-methylphenyl)-2,2-dioxido-3,4-dihydro-1H-2,1-benzothiazin-3-yl]-N-methylpropan-1-amine;
3-[(3R)-6-fluoro-1-(2-methylphenyl)-2,2-dioxido-3,4-dihydro-1H-2,1-benzothiazin-3-yl]-N-methylpropan-1-amine;
2-{(3S)-6-fluoro-3-[3-(methylamino)propyl]-2,2-dioxido-3,4-dihydro-1H-2,1-benzothiazin-1-yl}benzonitrile;
2-{(3R)-6-fluoro-3-[3-(methylamino)propyl]-2,2-dioxido-3,4-dihydro-1H-2,1-benzothiazin-1-yl}benzonitrile;
3-[(3S)-6-fluoro-1-(2-methoxyphenyl)-2,2-dioxido-3,4-dihydro-1H-2,1-benzothiazin-3-yl]-N-methylpropan-1-amine;
3-[(3R)-6-fluoro-1-(2-methoxyphenyl)-2,2-dioxido-3,4-dihydro-1H-2,1-benzothiazin-3-yl]-N-methylpropan-1-amine;
3-[(3S)-6-fluoro-1-(4-fluoro-2-methylphenyl)-2,2-dioxido-3,4-dihydro-1H-2,1-benzothiazin-3-yl]-N-methylpropan-1-amine;
3-[(3R)-6-fluoro-1-(4-fluoro-2-methyl phenyl)-2,2-dioxido-3,4-dihydro-1H-2,1-benzothiazin-3-yl]-N-methylpropan-1-amine;
3-[(3S)-6-fluoro-2,2-dioxido-1-phenyl-3,4-dihydro-1H-2,1-benzothiazin-3-yl]-N-methylpropan-1-amine;
3-[(3R)-6-fluoro-2,2-dioxido-1-phenyl-3,4-dihydro-1H-2,1-benzothiazin-3-yl]-N-methylpropan-1-amine;
3-[(3S)-6-chloro-1-(2-fluorophenyl)-2,2-dioxido-3,4-dihydro-1H-2,1-benzothiazin-3-yl]-N-methylpropan-1-amine; and
3-[(3R)-6-chloro-1-(2-fluorophenyl)-2,2-dioxido-3,4-dihydro-1H-2,1-benzothiazin-3-yl]-N-methylpropan-1-amine;
and
pharmaceutically acceptable salts thereof.

36. A compound according to claim 1,
wherein said pharmaceutically acceptable salt is a hydrochloride or a dihydrochloride.

37. A composition, comprising:
a. at least one compound according to claim 1; and
b. at least one pharmaceutically acceptable carrier.

38. A method for treating or preventing a condition selected from the group consisting of a vasomotor symptom, sexual dysfunction, gastrointestinal disorder, genitourinary disorder, chronic fatigue syndrome, fibromyalgia syndrome, depression disorder, endogenous behavioral disorder, cognitive disorder, diabetic neuropathy, pain, and combinations thereof in a subject in need thereof, comprising the step of:
administering to said subject an effective amount of a compound according to claim 1 or pharmaceutically acceptable salt thereof.

39. A method according to claim 38,

40. A method according to claim 38,
wherein said sexual dysfunction is desire-related or arousal-related.

41. A method according to claim 38,
wherein said gastrointestinal disorder or said genitourinary disorder is stress

42. A method according to claim 38,
wherein said condition is chronic fatigue syndrome.

43. A method according to claim 38,
wherein said condition is fibromyalgia syndrome.

44. A method according to claim 38,
wherein said condition is a depression disorder selected from the group consisting of major depressive disorder, generalized anxiety disorder, panic disorder, attention deficit disorder with or without hyperactivity, sleep disturbance, social phobia, and combinations thereof.

45. A method according to claim 38,
wherein said condition is diabetic neuropathy.

46. A method according to claim 38,
wherein said condition is pain.

47. A method according to claim 46,
wherein said pain is acute centralized pain, acute peripheral pain, or a combination thereof.

48. A method according to claim 46,
wherein said pain is chronic centralized pain, chronic peripheral pain, or a combination thereof.

49. A method according to claim 46,
wherein said pain is neuropathic pain, visceral pain, musculoskeletal pain, bony pain, cancer pain, inflammatory pain, or a combination thereof.

50. A method according to claim 49,
wherein said neuropathic pain is associated with diabetes, post traumatic pain of amputation, lower back pain, cancer, chemical injury, toxins, major surgery, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions, or a combination thereof.

51. A method according to claim 50,
wherein said neuropathic pain is post-herpetic neuralgia.

52. A method according to claim 49,
wherein said visceral pain is associated with ulcerative colitis, irritable bowel syndrome, irritable bladder, Crohn's disease, rheumatologic (arthralgias), tumors, gastritis, pancreatitis, infections of the organs, biliary tract disorders, or a combination thereof.

53. A method according to claim 46,
wherein said pain is female-specific pain.

54. A process for the preparation of a compound according to formula Ia 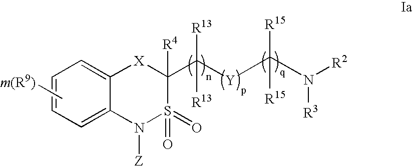
or a pharmaceutically acceptable salt thereof;
wherein:
m is an integer from 0 to 4;
n is an integer from 0 to 2;
p is an integer from 0 to 1;
q is an integer from 1 to 2;
v is an integer from 0 to 2;
X is C(R¹¹)₂, N(R¹²), O, or S(O)ᵥ;
Y is C(R¹¹)₂, N(R¹²), O, or 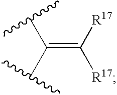
Z is H or an aryl substituted with 0-3 R⁵ or heteroaryl substituted with 0-3 R⁵;
R² is H, straight or branched C₁-C₆ alkyl, C₃-C₆ cycloalkyl, or aryl-C₁-C₆ alkyl where said aryl portion is substituted with 0-3 R⁶;
R³ is H, straight or branched C₁-C₆ alkyl, C₁-C₆ alkanol, C₃-C₆ cycloalkyl, or aryl-C₁-C₆ alkyl where said aryl portion is substituted with 0-3 R⁷; or
R² and R³, together with the nitrogen through which they are attached, form a mono- or bicyclic heterocyclic ring of 3 to 12 ring atoms, where one carbon may be optionally replaced with N, O, S, or SO₂, and where any carbon ring atom may be optionally substituted with C₁-C₄ alkyl, F, or CF₃ or where any additional N atom may be optionally substituted with C₁-C₄ alkyl, with the proviso that if Y is O or N(R¹²), q is not 2;
R⁴ is H, straight or branched C₁-C₆ alkyl, aryl substituted with 0-3 R⁸, aryl-C₁-C₆ alkyl where said aryl portion is substituted with 0-3 R⁸, heteroaryl substituted with 0-3 R⁸, or heteroaryl-C₁-C₆ alkyl where said heteroaryl portion is substituted with 0-3 R⁸;
R⁵ is, independently at each occurrence, C₁-C₆ alkyl, alkoxy, halo, CF₃, OCF₃, hydroxy, alkanoyloxy, nitro, nitrile, alkenyl, alkynyl, aryl substituted with 0-3 R⁵ or heteroaryl substituted with 0-3 R⁵;
R⁶ is, independently at each occurrence, C₁-C₄ alkyl, halo or H;
R⁷ is, independently at each occurrence, C₁-C₄ alkyl, halo or H;
R⁸ is, independently at each occurrence, C₁-C₄ alkyl, halo or H;
R⁹ is, independently at each occurrence, C₁-C₆ alkyl, alkoxy, halo, H, CF₃, OCF₃, hydroxy, alkanoyloxy, nitro, nitrile, alkenyl, alkynyl, aryl substituted with 0-3 R¹⁰, heteroaryl substituted with 0-3 R¹⁰, alkylsulfoxide, alkylsulfone, alkylsulfonamide, phenylsulfonamide substituted with 0-3 R¹⁰, alkylamido, or arylamido substituted with 0-3 R¹⁰; or
two adjacent R⁹, together with the ring atoms to which they are attached, form a fused ring of 5 or 6 ring atoms;
R¹⁰ is, independently at each occurrence, H, C₁-C₆ alkyl, or halo;
R¹¹ is, independently at each occurrence, H, C₁-C₆ alkyl, halo, hydroxy, O—(C₁-C₆ alkyl), or aryl substituted with 0-3 R⁸;
R¹² is, independently at each occurrence, H, C₁-C₆ alkyl, or aryl substituted with 0-3 R⁸;
R¹³ is, independently at each occurrence, H, C₁-C₆ alkyl, halide, hydroxy, or aryl substituted with 0-3 R¹⁶;
R¹⁵ is, independently at each occurrence, H, C₁-C₄ alkyl or halide;
R¹⁶ is, independently at each occurrence, H, C₁-C₄ alkyl, or halo; and
R¹⁷ is, independently at each occurrence, H or C₁-C₄ alkyl.
which process comprises:
(a) performing either of i) or ii) below:
i) coupling a compound according to formula II below with an electrophile according to formula a below: 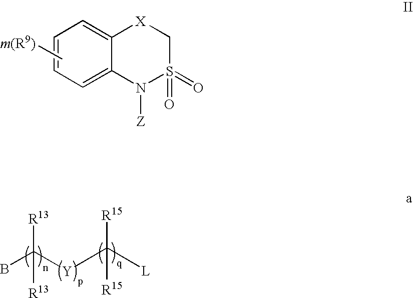
thereby forming a compound according to formula III below: 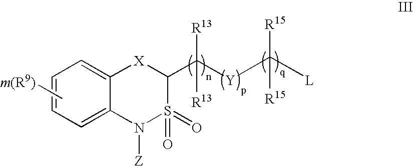
ii) coupling a compound according to formula II hereinabove with an allyl halide according to formula b below: 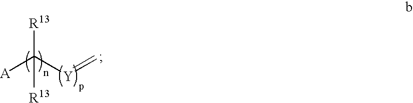
subjecting the compound formed by the coupling with the allyl halide to hydroboration-oxidation to form an alcohol; and
activating the alcohol with an activating agent, thereby forming a compound according to formula III hereinabove;
wherein R⁹, R¹³, R¹⁵, m, n, p, q, Z, X and Y are as defined hereinabove for formula Ia, B is a functional group, L is a leaving group and A is a halogen; and
b) reacting the compound of formula III formed in a) with an amine: 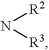
thereby forming a compound according to formula Ia hereinabove, wherein R² and R³ of the amine are as defined hereinabove for formula Ia.

55. The process of claim 54, wherein Z of the compound of formula II is H, and the process further comprises subjecting said compound to an N-arylation reaction prior to or subsequent to said coupling i) or said coupling ii).

56. The process of claim 54, wherein L of the compound of formula II is a chloride, a bromide, an iodide, a mesylate or a tosylate derived from the activating agent
